(19) United States
(12) Patent Application Publication
(10) Pub. No.: US 2026/0234234 A1
(43) Pub. Date: Aug. 13, 2026

(54) ANTI-IL33 AND ANTI-TSLP ANTIBODIES, AND MIXTURES

(71) Applicant: QILU PUGET SOUND BIOTHERAPEUTICS CORPORATION, Bothell, WA (US)

(72) Inventors: Zhi Liu, Shoreline, WA (US); Christine Vissinga, Seattle, WA (US); Jamil Haque, Seattle, WA (US); Wei Yan, Sammamish, WA (US)

(21) Appl. No.: 18/959,550

(22) Filed: Nov. 25, 2024

Related U.S. Application Data
(60) Provisional application No. 63/604,036, filed on Nov. 29, 2023.

(51) Int. Cl. C07K 16/24; A61K 39/00; A61P 37/08

(52) U.S. Cl. CPC C07K 16/244; A61P 37/08; A61K 2039/505; C07K 2317/24; C07K 2317/565; C07K 2317/73; C07K 2317/76; C07K 2317/92

(57) ABSTRACT

Described herein are anti-IL33 and/or anti-TSLP antibodies, polynucleotides encoding anti-IL33 and/or anti-TSLP antibodies, host cells containing such polynucleotides, methods of making and using such anti-IL33 and/or anti-TSLP antibodies or polynucleotides, pharmaceutical compositions containing such anti-IL33 and/or anti-TSLP antibodies or polynucleotides, as well as mixtures or bispecific antibodies comprising such anti-IL33 and/or anti-TSLP antibodies or polynucleotides encoding such mixtures or bispecific antibodies.

### FIELD

This invention is in the field of antibodies and their use to treat various human diseases.

### BACKGROUND

Asthma is a chronic inflammatory disease of the respiratory tract, usually characterized by variable airflow limitation and affecting more than 300 million people worldwide. This disease is the result of complex interactions between the environment, lung epithelium and the ensuing immune response leading to (allergic) airway inflammation. Within this pathobiological context, the bronchial epithelium exerts pivotal functions by producing innate cytokines named alarmins, such as thymic stromal lymphopoietin (TSLP), interleukin-33 (IL-33). The secretion of these cytokines occurs when airway epithelial cells undergo injuries caused by several environmental triggers such as allergens, respiratory viruses, bacteria, cigarette smoking, and airborne pollutants.

Upon release of alarmins, a broad array of innate and adaptive immune cells—including dendritic cells, naïve and differentiated T cells, ILC2 cells, Eosinophils, Basophils and mast cells—respond to TSLP and IL33 via their respective receptors. TSLP specifically acts through TSLPR (aka CRLF2) and signals through a heterodimeric receptor complex consisting of TSLPR and interleukin-7 receptor a chain. TSLP-mediated signaling results in activation of Janus kinases (JAKs) and signal transducers and activators of transcription (STATs) leading to transcription of target genes and subsequent tightly coordinated immune responses.

IL33 specifically acts through the ST2 receptor and signals through a receptor complex containing ST2 and the co-receptor IL-1R accessory protein (ILIR-AcP). IL-33 binding results in activation of NF-κB signaling pathway via the classical MyD88/IRAK/TRAF6 module leading to phosphorylation and activation of ERK1/2, JNK, p38 and PI3K/AKT signaling modules, resulting in the production and release of pro-inflammatory cytokines.

Genome-wide association studies have shown strong associations of asthma and genetic polymorphisms in TSLP, IL33, and ST2. In addition, mouse studies also support targeting TSLP and IL33 in asthma. Mice deficient in TSLPR or IL33 pathway had significantly decreased airway inflammation and airway hyperresponsiveness (AHR) in chronic models of adaptive immunity-mediated allergic airway inflammation. Furthermore, intranasal administration of IL33 or TSLP induced allergic airway inflammation in mice. Thus, both TSLP and IL33 can activate diverse downstream inflammatory cascades, explaining why they are considered major disease drivers of asthma.

There is an unmet need for therapeutics that can treat or ameliorate inflammatory diseases, such as chronic airway inflammatory diseases. The present invention addresses this need.

### SUMMARY

Described herein are anti-Interleukin 33 (IL33) antibodies, anti-Thymic stromal lymphopoietin (TSLP) antibodies, mixtures containing anti-IL33 and anti-TSLP antibodies, bispecific antibodies containing anti-IL33 and/or anti-TSLP antibodies. nucleic acids encoding these antibodies and mixtures, host cells containing these nucleic acids, pharmaceutical compositions comprising these antibodies, mixtures, and nucleic acids, and methods of treatment comprising administering these antibodies, mixtures, nucleic acids, or pharmaceutical compositions to patients.

For example, asthma and COPD (Chronic Obstructive Pulmonary Disease) are chronic inflammatory airway diseases characterized by obstructive airflow limitation. Both diseases are significant burdens on patients and healthcare systems. While asthma affects 262 million people with 461,000 deaths globally [see Lancet 2020; 396:1204-1222], COPD carries an even greater burden of disease and is the third leading cause of death worldwide, responsible for ~3.2 million deaths in 2019. Both diseases are heterogeneous in terms of their clinical presentation and underlying inflammatory mechanisms, therefore, therapy by single drug with single MOA (mechanism of action) may not be effective enough to treat all patients.

For both diseases, common triggers for acute exacerbations of airflow obstruction include viral or bacterial infections, cigarette smoke, allergens, and environmental factors such as air pollution. These triggers induce the epithelial cells in lung to secrete IL-33 (interleukin-33), TSLP (thymic stromal lymphopoietin), and IL-25 (interleukin-25, or IL-17E), three alarmins which drive Type-2 inflammation. For asthma in particular, genome-wide association studies have shown strong associations of the disease and genetic polymorphisms in TSLP, IL33, and ST2. Thus, TSLP and IL-33 both have emerged as particularly attractive targets due to their strong genetic links to asthma and its broad effects on airway inflammation.

ILC2s are a relatively newly identified immune cell. They are tissue-resident cells that are predominantly distributed in mucosal tissues such as lung, small intestine, skin, and adipose tissue. Due to their locations, they are considered to play a pivotal role in allergic diseases and in the development of Type-2 inflammation. After engagement by alarmins, ILC2s rapidly release cytokines such as IL-4, IL-5, and IL-13 to mediate responses by eosinophils, mast cells, basophils, DCs, B cells, and Th2 cells.

Currently available standard-of-care treatment with combined inhaled β-adrenergic agonists and inhaled corticosteroids (ICS) mainly provide symptomatic relief with control of disease and can often improve lung function and reduce exacerbation rates. However, the response is generally better in asthma compared to COPD and those with eosinophilic inflammation respond best to ICS. A subset of patients with asthma and COPD are insensitive to ICS. Side effects such as the increased frequency with increased dozing, muscle cramps, muscle twisting are common to patients with a long-term use of steroids and β-adrenergic agonists.

Patients with non-Type-2 asthma, as assessed by a low blood eosinophil count and low exhaled nitric oxide fraction, are not suitable candidates for current biologic therapies. In addition, while azithromycin and the phosphodiesterase-4 inhibitor, roflimulast, are available for reducing COPD exacerbations, these therapies have limited adoption due to side-effects. For azithromycin, side-effects are primarily gastrointestinal with potential for arrhythmias and development of antibiotic resistance; while with roflimulast, gastrointestinal side-effects with nausea predominate.

Chronic obstructive pulmonary disease (COPD) is currently defined as chronic disease state showing irreversible obstruction of airways resulting from the progression of two major underlying diseases including chronic bronchitis and emphysema. Chronic bronchitis is defined clinically as the persistence of cough, sputum, and difficult breathing, and emphysema is defined histopathologically as an irreversible change of airway walls distal to the terminal bronchiole and clinically shows slowly progressive respiratory difficulties. COPD is currently the fourth leading cause of death in the United States and Europe, and causes of death in patients with COPD are complications of the disease such as respiratory failure or infection (GOLD workshop summary, Am J Respir Crit Care Med 2001; 163:1256-1276).

In some portion of patients with long-standing asthma, irreversible obstruction of airway which is difficult to distinguish with COPD can be developed, and thus bronchial asthma can be progressed to COPD (Celli B R, et al., Eur Respir J 2004; 23:932-46). Significant reversible improvement of airway obstruction after short-term or long-term pharmacological treatments with inhalations of bronchodilators and steroids has been documented in a significant number of patients meeting current diagnostic criteria for COPD (GOLD workshop summary, Am J Respir Crit Care Med 2001; 163:1256-1276). Therefore, there is a significant number of patients satisfying definition criteria for both bronchial asthma and COPD (Guerra S, Curr Opin Pulm Med 2005; 11:17-13).

In humans, both TSLP and IL33 have been associated with severity of disease and have been shown to upregulate each other's receptor expression levels (Pebbles et al, Allergy, 2020), implying a regulatory feedback loop. Furthermore, TSLP and IL33 activate and recruit a variety of immune cells with many of them expressing both TSLP and IL33 receptors and as such these alarmins have the potential to amplify downstream (adaptive) immune responses and airway inflammation.

Thus, a large unmet need remains for treating and/or preventing chronic inflammatory diseases, including COPD and asthma. It is contemplated herein that blocking both IL-33 and TSLP alarmins could improve the efficacy for treating chronic inflammatory disease, such as asthma, COPD, and the like, since blocking only one alarmin is not sufficient. Dual blockade of IL-33 and TSLP is contemplated herein to provide advantages over targeting a single pathway, for example, anti-IL-5 therapy only ameliorates Type-2 inflammation. Dual blockade of IL-33 and TSLP would decrease the secretion of several downstream cytokines, i.e. IL-4, IL-5, IL-13, which mediate inflammation responses from eosinophils, B cells, mast cells etc.

It is contemplated herein that the invention anti-hIL33 and anti-hTSLP MabPair antibodies provide a safety advantage over small molecules. The invention MabPair compositions provided herein are contemplated to avoid the side effects of long-term use of inhaled β-adrenergic agonists and inhaled corticosteroids. The invention MabPair compositions provided herein are contemplated to address the unmet need for treating asthma, COPD, and the like.

Accordingly, also provided herein are methods of preventing or treating inflammation and/or inflammatory diseases, including asthma, COPD, and the like. Simultaneous or concurrent inhibition of TSLP and IL-33 has the potential to achieve superior efficacy compared to single agent in moderate to severe inflammation and/or inflammatory diseases, such as asthma, COPD, and the like.

The numbered items below describe these compositions and methods.
1. An anti-human IL33 (anti-hIL33) antibody comprising a heavy chain variable domain (VH) and a light chain variable region (VL), each comprising a complementarity determining region 1 (CDR1), a CDR2, and a CDR3,
   wherein the anti-hIL33 antibody comprises a VH CDR1, VH CDR2, VH CDR3, VL CDR1, VL CDR 2, and VL CDR3 comprising, respectively, the following amino acid sequences: SEQ ID NOs: 80, 81, 82, 77, 78, and 79, and
   wherein the anti-hIL33 antibody inhibits the interaction of human IL-33 and ST2/IL1AcP complex.
2. The anti-hIL33 antibody of item 1, wherein the VH comprises an amino acid sequence which comprises no more than four alterations relative to an amino acid sequence selected from the group consisting of SEQ ID NOs: 22, and 29, and/or the VL comprises an amino acid sequence which comprises no more than four amino acid alterations relative to an amino acid sequence selected from the group consisting of SEQ ID Nos: 20, and 25.
3. The anti-hIL33 antibody of item 2,
   wherein the VH and the VL of the anti-hIL33 antibody each comprise an amino acid sequence, which, together, comprise two sequences,
   wherein one of the two sequences comprises not more than four amino acid alterations relative to one sequence in a VH/VL pair of sequences, and the other of the two sequences comprises not more than four amino acid alterations relative to the other sequence in the VH/VL pair of sequences, and
   wherein the VH/VL pair of sequences is selected from the group consisting of: SEQ ID NOs: 29 (VH) and 25 (VL); and SEQ ID NOs: 22 (VH) and 20 (VL).
4. The anti-hIL33 antibody of item 1, wherein
   the VH CDR1, VH CDR2, VH CDR3, VL CDR1, VL CDR 2, and VL CDR3 comprise, respectively, the amino acid sequences of SEQ ID NOs: 80, 81, 82, 77, 78, and 79,
   the VH comprises an amino acid sequence which comprises no more than four alterations relative to the amino acid sequence of SEQ ID NO: 29, and
   the VL comprises an amino acid sequence which comprises no more than four alterations relative to the amino acid sequence of SEQ ID NO: 25.
5. One or more polynucleotide(s) encoding an anti-hIL33 antibody comprising a VH and a VL, each comprising a CDR1, a CDR2, and a CDR3,
   wherein the anti-hIL33 antibody comprises a VH CDR1, VH CDR2, VH CDR3, VL CDR1, VL CDR 2, and VL CDR3 comprising, respectively, the following amino acid sequences: SEQ ID NOs: 80, 81, 82, 77, 78, and 79; and
   wherein the anti-hIL33 antibody inhibits the interaction of human IL-33 and ST2/IL1AcP complex.
6. A host cell comprising one or more polynucleotide(s) encoding an anti-hIL33 antibody comprising a VH and a VL, each comprising a CDR1, a CDR2, and a CDR3,
   wherein the anti-hIL33 antibody comprises a VH CDR1, VH CDR2, VH CDR3, VL CDR1, VL CDR 2, and VL CDR3 comprising, respectively, the following sequences: SEQ ID NOs: 80, 81, 82, 77, 78, and 79; and
   wherein the anti-hIL33 antibody inhibits the interaction of human IL-33 and ST2/IL1AcP complex.
7. A method of treating a patient, in need thereof, having inflammation, an inflammatory disease, a chronic inflammatory airway disease, asthma, COPD, and/or Type 2 inflammation, said method comprising:
   (a) administering to the patient an anti-hIL33 antibody comprising a VH and a VL, each comprising a CDR1, a CDR2, and a CDR3, wherein: (1) the anti-hIL33 antibody comprises a VH CDR1, VH CDR2, VH CDR3, VL CDR1, VL CDR 2, and VL CDR3 comprising, respectively, the following sequences: SEQ ID NOs: 80, 81, 82, 77, 78, and 79; and (2) the anti-hIL33 antibody inhibits the interaction of human IL-33 and ST2/IL1AcP complex; or
   (b) administering to the patient one or more polynucleotide(s) encoding the anti-hIL33 antibody of (a).
8. An anti-human TSLP (anti-hTSLP) antibody comprising a VH and a VL, each comprising a CDR1, a CDR2, and a CDR3,
   wherein the anti-hTSLP antibody comprises a VH CDR1, VH CDR2, VH CDR3, VL CDR1, VL CDR 2, and VL CDR3 comprising, respectively, the following sequences: SEQ ID NOs: 86, 87, 88, 83, 84, and 85; SEQ ID NOs: 86, 87, 88, 83, 89, and 90; SEQ ID NOs: 94, 95, 96, 91, 92, and 93, and
   wherein the anti-hTSLP antibody inhibits hTSLP binding to hTSLPR or hTSLP interacting with an hTSLPR/hIL7Rζ complex.
9. The anti-hTSLP antibody of item 8,
   wherein the anti-hTSLP VH comprises an amino acid sequence which comprises no more than four alterations relative to an amino acid sequence selected from the group consisting of SEQ ID NOs: 43, 50, 65, and 70, and/or the anti-hTSLP VL comprises an amino acid sequence which comprises no more than four alterations relative to an amino acid sequence selected from the group consisting of SEQ ID NOs: 45, 47, 54, 67, and 74.
10. The anti-hTSLP antibody of item 9,
   wherein the VH and the VL of the anti-hTSLP antibody each comprise an amino acid sequence, which, together, comprise two sequences,
   wherein one of the two sequences comprises not more than four amino acid alterations relative to one sequence in a VH/VL pair of sequences, and the other of the two sequences comprises not more than four amino acid alterations relative to the other sequence in the VH/VL pair of sequences, and
   wherein the VH/VL pair of sequences is selected from the group consisting of: SEQ ID NOs: 43 (VH) and 45 (VL); SEQ ID NOs: 43 (VH) and 47 (VL); SEQ ID NOs: 50 (VH) and 54 (VL); SEQ ID NOs: 65 (VH) and 67 (VL); and SEQ ID NOs: 70 (VH) and 74 (VL).
11. The anti-hTSLP antibody of item 8, wherein
   the VH CDR1, VH CDR2, VH CDR3, VL CDR1, VL CDR 2, and VL CDR3 comprise, respectively, the amino acid sequences of SEQ ID NOs: 94, 95, 96, 91, 92, and 93,
   the VH comprises an amino acid sequence which comprises no more than four alterations relative to the amino acid sequence of SEQ ID NO: 70, and
   the VL comprises an amino acid sequence which comprises no more than four alterations relative to the amino acid sequence of SEQ ID NO: 74.
12. One or more polynucleotide(s) encoding anti-hTSLP antibody comprising a VH and a VL, each comprising a CDR1, a CDR2, and a CDR3,
   wherein the anti-hTSLP antibody comprises a VH CDR1, VH CDR2, VH CDR3, VL CDR1, VL CDR 2, and VL CDR3 comprising, respectively, the following sequences: SEQ ID NOs: 86, 87, 88, 83, 84, and 85; SEQ ID NOs: 86, 87, 88, 83, 89, and 90; SEQ ID NOs: 94, 95, 96, 91, 92, and 93, and
   wherein the anti-hTSLP antibody inhibits hTSLP binding to hTSLPR or hTSLP interacting with an hTSLPR/hIL7Rζ complex.
13. A host cell comprising one or more polynucleotide(s) encoding an anti-hTSLP antibody comprising a VH and a VL, each comprising a CDR1, a CDR2, and a CDR3,
   wherein the anti-hTSLP antibody comprises a VH CDR1, VH CDR2, VH CDR3, VL CDR1, VL CDR 2, and VL CDR3 comprising, respectively, the following sequences: SEQ ID NOs: 86, 87, 88, 83, 84, and 85; SEQ ID NOs: 86, 87, 88, 83, 89, and 90; SEQ ID NOs: 94, 95, 96, 91, 92, and 93, and
   wherein the anti-hTSLP antibody inhibits hTSLP binding to hTSLPR or hTSLP interacting with an hTSLPR/hIL7Rζ complex.
14. A method of treating a patient, in need thereof, having inflammation, an inflammatory disease, a chronic inflammatory airway disease, asthma, COPD, and/or Type 2 inflammation, said method comprising:
   (a) administering to the patient an anti-hTSLP antibody comprising a VH and a VL, each comprising a CDR1, a CDR2, and a CDR3, wherein: (1) the anti-hTSLP antibody comprises a VH CDR1, VH CDR2, VH CDR3, VL CDR1, VL CDR 2, and VL CDR3 comprising, respectively, the following sequences: SEQ ID NOs: 86, 87, 88, 83, 84, and 85; SEQ ID NOs: 86, 87, 88, 83, 89, and 90; SEQ ID NOs: 94, 95, 96, 91, 92, and 93; and (2) the anti-hTSLP antibody inhibits hTSLP binding to hTSLPR or hTSLP interacting with an hTSLPR/hIL7Rζ complex; or
   (b) administering to the patient one or more polynucleotide(s) encoding the anti-hTSLP antibody of (a).
15. A mixture comprising an anti-hIL33 antibody and an anti-hTSLP antibody, wherein:
   (a) (1) the anti-hIL33 antibody comprises a VH and a VL, each comprising a CDR1, a CDR2, and a CDR3, (2) the anti-hIL33 antibody comprises a VH CDR1, VH CDR2, VH CDR3, VL CDR1, VL CDR 2, and VL CDR3 comprising, respectively, the following sequences: SEQ ID NOs: 80, 81, 82, 77, 78, and 79, and (3) the anti-hIL33 antibody inhibits the interaction of human IL-33 and ST2/IL1AcP complex; and
   (b) (1) the anti-hTSLP antibody comprises a VH and a VL, each comprising a CDR1, a CDR2, and a CDR3, (2) anti-hTSLP antibody comprises a VH CDR1, VH CDR2, VH CDR3, VL CDR1, VL CDR 2, and VL CDR3 comprising, respectively, the following sequences: SEQ ID NOs: 86, 87, 88, 83, 84, and 85; SEQ ID NOs: 86, 87, 88, 83, 89, and 90; SEQ ID NOs: 94, 95, 96, 91, 92, and 93; and (3) the anti-hTSLP antibody inhibits hTSLP binding to hTSLPR or hTSLP interacting with an hTSLPR/hIL7Rζ complex.
16. The mixture of item 15, wherein
   (a) (1) the VH CDR1, VH CDR2, VH CDR3, VL CDR1, VL CDR 2, and VL CDR3 of the anti-hIL33 antibody comprise, respectively, the amino acid sequences of SEQ ID NOs: 80, 81, 82, 77, 78, and 79, (2) the VH of the anti-hIL33 antibody comprises an amino acid sequence which comprises no more than four alterations relative to the amino acid sequence of SEQ ID NO: 29, and (3) the VL of the anti-hIL33 antibody comprises an amino acid sequence which comprises no more than four alterations relative to the amino acid sequence of SEQ ID NO: 25, and
   (b) (1) the VH CDR1, VH CDR2, VH CDR3, VL CDR1, VL CDR 2, and VL CDR3 of the anti-hTSLP antibody comprise, respectively, the amino acid sequences of SEQ ID NOs: 94, 95, 96, 91, 92, and 93, (2) the VH comprises an amino acid sequence which comprises no more than four alterations relative to the amino acid sequence of SEQ ID NO: 70, and (3) the VL comprises an amino acid sequence which comprises no more than four alterations relative to the amino acid sequence of SEQ ID NO: 74.
17. One or more polynucleotide(s) encoding a mixture comprising an anti-hIL33 antibody and an anti-hTSLP antibody, wherein
   (a) (1) the anti-hIL33 antibody comprises a VH and a VL, each comprising a CDR1, a CDR2, and a CDR3, (2) the anti-hIL33 antibody comprises a VH CDR1, VH CDR2, VH CDR3, VL CDR1, VL CDR 2, and VL CDR3 comprising, respectively, the following sequences: SEQ ID NOs: 80, 81, 82, 77, 78, and 79, and (3) the anti-hIL33 antibody inhibits the interaction of human IL-33 and ST2/IL1AcP complex; and
   (b) (1) the anti-hTSLP antibody comprises a VH and a VL, each comprising a CDR1, a CDR2, and a CDR3, (2) anti-hTSLP antibody comprises a VH CDR1, VH CDR2, VH CDR3, VL CDR1, VL CDR 2, and VL CDR3 comprising, respectively, the following sequences: SEQ ID NOS: 86, 87, 88, 83, 84, and 85; SEQ ID NOs: 86, 87, 88, 83, 89, and 90; SEQ ID NOs: 94, 95, 96, 91, 92, and 93; and (3) the anti-hTSLP antibody inhibits hTSLP binding to hTSLPR or hTSLP interacting with an hTSLPR/hIL7Rζ complex.
18. A host cell comprising one or more polynucleotides encoding a mixture comprising an anti-hIL33 antibody and an anti-hTSLP antibody, wherein
   (a) (1) the anti-hIL33 antibody comprises a VH and a VL, each comprising a CDR1, a CDR2, and a CDR3, (2) the anti-hIL33 antibody comprises a VH CDR1, VH CDR2, VH CDR3, VL CDR1, VL CDR 2, and VL CDR3 comprising, respectively, the following sequences: SEQ ID NOs: 80, 81, 82, 77, 78, and 79, and (3) the anti-hIL33 antibody inhibits the interaction of human IL-33 and ST2/IL1AcP complex; and
   (b) (1) the anti-hTSLP antibody comprises a VH and a VL, each comprising a CDR1, a CDR2, and a CDR3, (2) anti-hTSLP antibody comprises a VH CDR1, VH CDR2, VH CDR3, VL CDR1, VL CDR 2, and VL CDR3 comprising, respectively, the following sequences: SEQ ID NOs: 86, 87, 88, 83, 84, and 85; SEQ ID NOs: 86, 87, 88, 83, 89, and 90; SEQ ID NOs: 94, 95, 96, 91, 92, and 93; and (3) the anti-hTSLP antibody inhibits hTSLP binding to hTSLPR or hTSLP interacting with an hTSLPR/hIL7Rζ complex.
19. A method of treating a patient, in need thereof, having inflammation, an inflammatory disease, a chronic inflammatory airway disease, asthma, COPD, and/or Type 2 inflammation, said method comprising:
   (a) administering to the patient a mixture comprising an anti-hIL33 antibody and an anti-hTSLP antibody, wherein:
   (1) (A) the anti-hIL33 antibody comprises a VH and a VL, each comprising a CDR1, a CDR2, and a CDR3, (B) the anti-hIL33 antibody comprises a VH CDR1, VH CDR2, VH CDR3, VL CDR1, VL CDR 2, and VL CDR3 comprising, respectively, the following sequences: SEQ ID NOs: 80, 81, 82, 77, 78, and 79, and (C) the anti-hIL33 antibody inhibits the interaction of human IL-33 and ST2/IL1AcP complex; and
   (1) (B) the anti-hTSLP antibody comprises a VH and a VL, each comprising a CDR1, a CDR2, and a CDR3, (B) anti-hTSLP antibody comprises a VH CDR1, VH CDR2, VH CDR3, VL CDR1, VL CDR 2, and VL CDR3 comprising, respectively, the following sequences: SEQ ID NOs: 86, 87, 88, 83, 84, and 85; SEQ ID NOs: 86, 87, 88, 83, 89, and 90; SEQ ID NOs: 94, 95, 96, 91, 92, and 93; and (C) the anti-hTSLP antibody inhibits hTSLP binding to hTSLPR or hTSLP interacting with an hTSLPR/hIL7Rζ complex; or
   (b) administering to the patient one or more polynucleotide(s) encoding the mixture of (a).
20. A mixture of antibodies comprising:
   (a) an anti-hIL33 antibody comprising a heavy chain (HC) and a light chain (LC), wherein (1) the HC of the anti-hIL33 antibody is encoded by a nucleic acid sequence which encodes the amino acid sequence of SEQ ID NO: 31, and (2) the LC of the anti-hIL33 antibody is encoded by a nucleic acid sequence which encodes the amino acid sequence of SEQ ID NO: 27; and
   (b) an anti-hTSLP antibody comprising an HC and an LC, wherein (1) the HC of the anti-hTSLP antibody is encoded by a nucleic acid sequence which encodes the amino acid sequence of SEQ ID NO: 72, and (2) the LC of the anti-hTSLP antibody is encoded by a nucleic acid sequence which encodes the amino acid sequence of SEQ ID NO: 76.
21 The mixture of item 20, wherein:
   the amino acid sequences of the HC and LC of the anti-hIL33 antibody are encoded by the nucleic acid sequences of SEQ ID NOs: 30 and 26, respectively; and
   the amino acid sequences of the HC and LC of the anti-hTSLP antibody are encoded by the nucleic acid sequences of SEQ ID NOs: 71 and 75, respectively.
22. A pharmaceutical composition comprising the mixture of any one of items 20 to 21.
23. One or more polynucleotide(s) encoding the mixture of any one of items 20 to 21.
24 The polynucleotide(s) of item 23, wherein the polynucleotide(s) comprise the nucleic acid sequences of SEQ ID NOs: 30, 26, 71, and 75.
25 One or more vector(s) comprising the polynucleotide(s) of item 23 or 24.
26. The vector(s) of item 25, which is (are) (a) viral vector(s).
27. The vector(s) of item 26, which is (are) (an) oncolytic viral vector(s).
28 The vector(s) of item 26 or 27, which is (are) (a) retroviral, adenoviral, adeno-associated viral (AAV), vaccinia viral, modified vaccina viral Ankara (MVA), herpes viral, lentiviral, measles viral, coxsackie viral, Newcastle Disease viral, reoviral, or poxviral vector(s).
29. A host cell comprising the polynucleotide(s) of item 23 or 24 and/or the vector(s) of item 5, wherein the host cell can produce the mixture of any one of items 20 to 21.
30. The host cell of item 29, which is a CHO cell or a mouse myeloma cell.
31. A method for making a mixture of antibodies comprising the following steps:
   culturing the host cell of any one of items 29 to 30; and
   recovering the mixture of antibodies from the culture supernatant or the host cell mass.
32. A method for treating a patient, in need thereof, having inflammation, an inflammatory disease, a chronic inflammatory airway disease, asthma, COPD, and/or Type 2 inflammation, said method comprising:
   (a) administering to the patient a dose of the mixture of any one of items 20 to 21 or the pharmaceutical composition of item 22 to the patient, or
   (b) administering to the patient a dose of the polynucleotide(s) of item 23 or 24 or the vector(s) of any one of items 26 to 28.
33. An anti-hIL33 antibody, wherein
   the HC comprises an amino acid sequence which comprises no more than four, three, two or one alteration(s) relative to the amino acid sequence of SEQ ID NOs: 7, 11, 15, 19, 23, or 31, and
   the LC comprises an amino acid sequence which comprises no more than four, three, two or one alteration(s) relative to the amino acid sequence of SEQ ID NOs: 5, 9, 13, 17, 21, or 27.
34 The anti-hIL33 antibody of item 33, wherein the HC comprises the amino acid sequence of SEQ ID NO: 31, and the LC comprises the amino acid sequence of SEQ ID NO: 27.
35 An anti-hTSLP antibody, wherein
   the HC comprises an amino acid sequence which comprises no more than four, three, two or one alteration(s) relative to the amino acid sequence of SEQ ID NOs: 44, 52, 62, 66, or 72, and
   the LC comprises an amino acid sequence which comprises no more than four, three, two or one alteration(s) relative to the amino acid sequence of SEQ ID NOs: 46, 48, 56, 64, 68, or 76.
36. The anti-hTSLP antibody of item 35, wherein the HC comprises the amino acid sequence of SEQ ID NO: 72, and the LC comprises the amino acid sequence of SEQ ID NO: 76.
37. A mixture of anti-hIL33 and anti-hTSLP antibodies, wherein
   the anti-hIL33 HC comprises an amino acid sequence which comprises no more than four, three, two or one alteration(s) relative to the amino acid sequence of SEQ ID NOs: 7, 11, 15, 19, 23, or 31, and
   the anti-hIL33 LC comprises an amino acid sequence which comprises no more than four, three, two or one alteration(s) relative to the amino acid sequence of SEQ ID NOs: 5, 9, 13, 17, 21, or 27; and
   the anti-hTSLP HC comprises an amino acid sequence which comprises no more than four, three, two or one alteration(s) relative to the amino acid sequence of SEQ ID NOs: 44, 52, 62, 66, or 72, and
   the anti-hTSLP LC comprises an amino acid sequence which comprises no more than four, three, two or one alteration(s) relative to the amino acid sequence of SEQ ID NOs: 46, 48, 56, 64, 68, or 76.

### BRIEF DESCRIPTION OF THE FIGURES

FIG. 1: Binding of hIL-33 to ST2-Fc and blocking of IL-33/ST2 interaction by anti-hIL-33 antibodies. (A) Binding assessment of active hIL-33 at different concentrations to the captured ST2-Fc on CM5 chip, the association time was set at either 1 min or 2 min while the dissociation time was kept at 5 min. (B) Blocking analysis of different anti-hIL-33 antibodies. The active hIL-33 at 10 nM was premixed with different concentrations of each antibody, then the mixture was flowed over the CM5 chip surface on which ST2-Fc protein was captured. (C) Blocking activity analysis of chimeric anti-hIL-33 antibody 10998, 5 variants of humanized anti-hIL-33 antibody, and an isotype control antibody 10825. Assessment was performed by SPR (Surface Plasmon Resonance) using Biacore 2000. The x-axis represents antibody concentration (nM), the y-axis represents the Response Units (RU) after correction by blank buffer.

FIG. 2: Mass spectrometry analysis to assess the deamidation susceptibility of humanized anti-hIL-33 antibodies QB11061 and QB11119. The anti-hIL-33 antibodies QB11061 (A and B) and QB11119 (C, D, and E) were denatured, reduced, alkylated, and digested with typsin for the accelerated deamidation. Pre-treated antibody samples (A and C) and treated antibody samples (B and D) were digested with trypsin, the eluted peptides were detected under UV 214 nm, each peptide was analyzed by mass spectrometry to assess potential deamidation. A peak labeled with an arrow and L3 (N→D) indicates the deamidated peptide at ²⁸NG²⁹ motif of VL-CDR2. FIG. 1E is the zoom-in of FIG. 1D. The x-axis represents elution time, the y-axis represents Response Units.

FIG. 3: Schematic description of the construction of yeast display library for affinity maturation. (A) Four DNA fragments including PCR-amplified DNA fragments encoding (1) yeast signal peptide (SP1) and VL sequence; (2) middle fragment containing human c-kappa (Ck), c-MYC tag, furin-pep2A cleavage site (F2A), yeast signal peptide 2 (SP2); (3) SP2, VH sequence (VH), human CHI sequence (CH1); (4) Bgl II and Nhe I double digested pFab1.6 vector containing SP1, hemagglutinin tag (HA), and yeast Aga2 protein (Aga2) were mixed for the electroporation of competent yeast cells. Homologous recombination in yeast cells enables the DNA fragments to assemble into a full-length construct which is transcriptional under the Galactose inducible promoter. (B) After translation of polypeptides, the LC (VL+Ck) is cleaved by furin (after c-MYC tag), the LC is then secreted and assembled with Fd (VH+CH1) to form Fab fragment. The Aga2 protein from the introduced construct forms two disulfide bonds with the native Aga1 protein expressed on yeast, therefore, the Fab fragments are anchored (or displayed) on yeast surface.

FIG. 4: Binding of 94 individual yeast colonies after the final round of FACS sorting with 2 nM biotinylated hIL-33 antigen. Wells A1 and H12 are the parental QB11119 yeast clone to serve as an internal control. 15 clones with strong antigen binding (boxed) were picked for PCR amplification and DNA sequencing. The x-axis indicates binding strength of FITC-conjugated anti-HA antibody to show the display level of Fab fragments on the yeast surface. The y-axis measures the antigen binding capacity of displayed Fab fragments by APC-conjugated streptavidin bound with the biotinylated hIL-33.

FIG. 5A: Surface plasmon resonance sensorgrams of the captured anti-hIL-33 antibodies interacting with hIL-33 using 1:1 kinetic model fit overlays on a Biacore T200. 5B: Surface plasmon resonance sensorgrams of the captured anti-hIL-33 antibodies interacting with cyIL-33 using 1:1 kinetic model fit overlays on a Biacore T200. The polyclonal goat-anti-human IgG (Fc specific) antibodies were immobilized on a CM4 chip surface using a standard amine coupling method. Anti-hIL-33 antibodies QB11004, QB11061, QB11119, QB11421, QB11465, and the comparator-3 antibody QB11094 were captured, and active hIL-33 (50 nM, 16.67 nM, 5.56 nM, 1.85 nM, 0.62 nM) or cyIL-33 (100 nM, 33.33 nM, 11.11 nM, 3.7 nM, 1.23 nM, 0.41 nM) at 1:3 series dilution were injected at 50 uL/min, association time was 3 min, disassociation time was monitored for up to 1 hr but only the data from the first 5 min were evaluated. For evaluation of the hIL-33 antigen, the cycles were run at 37° C., for cyIL-33 antigen the cycles were run at 25° C. After each cycle, the chip was regenerated with 10 mM glycine-HCl, pH 1.5. The results were analyzed using Biacore T200 evaluation software V3.2. Results were double referenced and fit to a 1:1 interaction model. The values of the calculated parameters for the kinetic analysis are listed in Table 9 and Table 10.

FIG. 6: Screening antagonistic anti-hTSLP polyclonal rabbit antibodies by Biacore T200. (A) Comparisons of the purified rabbit-anti-human TSLP polyclonal antibodies from rabbit 7182, 7183, 7184, 7186 along with comparator antibody 10985 and the isotype control antibody 10987. Anti-His6 antibody was immobilized on CM5 chip surface, the premixed His6-hTSLP antigen at various concentrations and 2 μg/ml purified rabbit antibody were injected, finally the hTSLPR-Fc at 100 nM was flowed through the surface of CM5 chip. (B) Binding sensorgrams of the captured polyclonal antibodies from rabbit 7186 interacting with hTSLP. (C) Binding sensorgrams of the captured polyclonal antibodies from rabbit 7186 interacting with cyTSLP. (D) Demonstration of direct interaction between hTSLP and hTSLPR-Fc. Anti-His6 antibody was immobilized on CM5 chip surface, the His6-hTSLP antigen at 5 μg/ml was captured, blank buffer was injected for 1 min followed by a second injection of either blank buffer, hTSLPR-Fc at 100 nM, anti-hTSLP comparator blocking antibody 10985, or an anti-hTSLP non-blocking antibody 10987 over the surface of CM5 chip for 3 min. (E) Polyclonal antibodies from rabbit 7186 can block the direct interaction between hTSLP and hTSLPR-Fc. Anti-His6 antibody was immobilized on CM5 chip surface, the His6-hTSLP antigen at 5 μg/ml was captured, polyclonal antibodies from rabbit 7186 at 30 g/ml was injected for 1 min followed by a second injection of either blank buffer, hTSLPR-Fc at 100 nM, anti-hTSLP comparator blocking antibody 10985, or an anti-hTSLP non-blocking antibody 10987 over the surface of CM5 chip for 3 min. The Response Difference between the testing protein and blank buffer was recorded as y-axis, time in seconds was recorded in x-axis.

FIG. 7: Confirmation of antigen binding of individual rabbit anti-hTSLP antibodies by plate-based ELISA analysis. The hTSLP, cyTSLP, or irrelevant antigen each at 2 μg/ml was separately immobilized in each well of 96-w Maxisorp plates in PBS overnight, the plates were washed for 3 times and blocked with 2% BSA in 1×PBST. The supernatant from 177 rabbit hybridomas was separately added and incubated for 1 hr with shaking at RT. The HRP-conjugated Goat anti-Rabbit IgG (H+L) secondary antibody was added, the plates were shaken for 1 hr and washed before developed with substrate 3,3′,5,5′-tetramethylbenzidine (TMB). The plates were read at 450 nm in Perkin-Elmer plate reader. The x-axis represents clone name, the y-axis represents the value of developed color at 450 nm.

FIG. 8: Competition ELISA assay to confirm the blocking activity of the top 10 rabbit anti-hTSLP mAbs. The His6-hTSLP antigen at 2 μg/ml was coated in the 96-w Maxisorp plates, The individual anti-hTSLP rabbit antibodies or a rabbit isotype control antibody as duplicates were added at 5 μg/ml followed by the addition of the biotinylated hTSLPR at 2 μg/ml, streptavidin-conjugated HRP then substrate TMB were added for color development. The plates were shaken for 15 min before the H₂SO₄ solution was added to stop the reaction. The plates were read in Perkin-Elmer plate reader. The percentage of inhibition was plotted against blank 1×PBST buffer in which no antibody was included.

FIG. 9: Screen by Biacore T200 (A) and CCL17 release assay (B) to compare the blocking activity of individual rabbit anti-hTSLP mAbs. (A) The hTSLPR-Fc protein was immobilized on CM5 chip surface, the hTSLP at 20 nM and various concentrations of anti-TSLP rabbit antibody were premixed then flowed over the CM5 surface. The association was run for 3 min, the binding level of hTSLP (as Response Units) to the immobilized hTSLPR was recorded as y-axis, the antibody concentration (nM) at log₁₀ scale was plotted in x-axis. (B) Primary human monocytes purified from PBMCs were seeded in 96-w plates and treated for 24 hrs with a constant amount of hTSLP (at concentrations close to EC₅₀ values) plus a titration of top anti-hTSLP rabbit antibody 60E10, 135F1, 139F11, isotype controls 11176, or 10861. The CCL-17 chemokine levels (pg/ml) in supernatants were determined using a human CCL-17 ELISA kit from R&D Systems (cat. no. DY364). The results were plotted using GraphPad Prism software. IC₅₀ values of top 3 clones #66 (clone 60E10), #70 (clone 135F1), #72 (clone 139F11) are listed in the small box-table below the figure.

FIG. 10: Mechanism of action of anti-hTSLP antibodies QB11341 and QB11237 by Biacore assays. (A) Direct ligand-receptor interaction between hTSLP and hTSLPR. The hTSLPR-Fc protein was immobilized on CM5 chip surface using a standard amine coupling method, monomeric hTSLP antigen at 0, 0.62, 1.85, 5.56, 16.7, 50 nM was flowed over the CM5 surface, the association was run for 3 min and dissociation time was run for 5 min. The kinetic sensorgram was analyzed with BIAevaluation 4.1.1 to measure the binding affinity between hTSLP and hTSLPR. (B) Blocking of hTSLP-hTSLPR axis by antibody QB11341 and the blocking of hTSLP-hIL7Rζ axis by antibody QB11237. The hTSLPR-Fc protein was immobilized on CM5 chip surface, monomeric antigen hTSLP alone, or a premixed hTSLP-antibody complex with either antibody QB11341 or QB11237 was flowed over CM5 surface for 3 min, finally 50 nM of hIL7Rζ or blank buffer was injected with an association time for 2 min and a dissociation time for 5 min. (C) Estimation of the inhibition level of hTSLP-hIL7Rζ interaction by antibody QB11237. The hTSLPR-Fc was immobilized on CM5 chip, 20 nM of monomeric hTSLP was injected with an association for 2 min, and a dissociation for 100 sec, anti-hTSLP antibody QB11341 or QB11237 each at 20 nM was subsequently flowed over the CM5 surface with an association time of 3 min and a quick dissociation time of 1 min, finally 50 nM of hIL7Rζ or the blank running buffer was injected with an association time of 2 min and a dissociation time of 2 min. The level of inhibiting the hTSLP-hIL7Rζ interaction by antibody QB11237 was estimated as “100% minus the percentage of the peak height difference (in the presence and absence of the antibody QB11237) divided by the peak height in the absence of antibody QB11237”.

FIG. 11: Chain drop-out experiments to assess the LC-HC pairing of anti-hIL-33 and anti-hTSLP antibodies before and after engineering. ExpiCHO cells were co-transfected with plasmid DNAs encoding the LC1 and/or HC1 of anti-hTSLP QB11548 in combination with the LC2 and/or HC2 of anti-hIL33 QB11465 (A and C), or with plasmid DNAs encoding the LC1 and/or HC1 of anti-hTSLP QB11237 in combination with the LC2 and/or HC2 of anti-hIL33 QB11465 (B and D). The ExpiCHO cells were shaken for 12 days in tissue culture incubator, 10 μl/lane of cleared supernatant was loaded in 4-15% Criterion™ TGX Stain-Free™ Precast SDS-PAGE gel, and antibodies were visualized with a CHEMIDOC™ XRS+ imager. The lanes loaded with protein molecular weight standards (marked with M) were run in the leftmost of each gel, sizes in kilodaltons (kDa) are indicated. (A). Lane 1 contains plasmid DNAs encoding the LC1 and HC1 of anti-hTSLP antibody QB11548; lane 2 contains plasmid DNAs encoding the HC1 of anti-hTSLP antibody QB11548 and LC2 of anti-hIL-33 antibody QB11465; lane 3 contains plasmid DNAs encoding the LC2 and HC2 of anti-hIL-33 antibody QB11465; lane 4 contains plasmid DNAs encoding the LC1 of anti-hTSLP antibody QB11548 and HC2 of anti-hIL-33 antibody QB11465. (B). Lane 1 contains plasmid DNAs encoding the LC1 and HC1 of anti-hTSLP antibody QB11237; lane 2 contains plasmid DNAs encoding the HC1 of anti-hTSLP antibody QB11237 and LC2 of anti-hIL-33 antibody QB11465; lane 3 contains plasmid DNAs encoding the LC2 and HC2 of anti-hIL-33 antibody QB11465; lane 4 contains plasmid DNAs encoding the LC1 of anti-hTSLP antibody QB11237 and HC2 of anti-hIL-33 antibody QB11465. (C). Lane 1 contains plasmid DNAs encoding the LC1 and HC1 of anti-hTSLP antibody QB11764; lane 2 contains plasmid DNAs encoding the HC1 of anti-hTSLP antibody QB11764 and LC2 of anti-hIL-33 antibody QB11465; lane 3 contains plasmid DNAs encoding the LC2 and HC2 of anti-hIL-33 antibody QB11465; lane 4 contains plasmid DNAs encoding the LC1 of anti-hTSLP antibody QB11764 and HC2 of anti-hIL-33 antibody QB11465; lane 5 contains four plasmid DNAs encoding for LC1, HC1, LC2, HC2 of both anti-hTSLP antibody QB11764 and anti-hIL-33 antibody QB11465; lane 6 contains the empty pSB01 plasmid DNA to serve as a mock transfection. (D). Lane 1 contains plasmid DNAs encoding the LC1 and HC1 of anti-hTSLP antibody QB11718; lane 2 contains plasmid DNAs encoding the HC1 of anti-hTSLP antibody QB11718 and LC2 of anti-hIL-33 antibody QB11465; lane 3 contains plasmid DNAs encoding the LC2 and HC2 of anti-hIL-33 antibody QB11465; lane 4 contains plasmid DNAs encoding the LC1 of anti-hTSLP antibody QB11718 and HC2 of anti-hIL-33 antibody QB11465; lane 5 contains four plasmid DNAs encoding for LC1, HC1, LC2, HC2 of both anti-hTSLP antibody QB11718 and anti-hIL-33 antibody QB11465; lane 7 contains the plasmid DNAs encoding the LC and HC of anti-HER2 trastuzumab to monitor the transfection efficiency.

FIG. 12: Mass spectrometry analysis of MabPair antibody mixture QB11750 consisting of anti-hIL-33 IgG4 antibody QB11465 and anti-hTSLP IgG1-D265A antibody QB11718. Procedures are described in Example 10. The x axes show the deconvoluted mass, and the y axes show counts, which are reflective of the abundance of protein of a given mass. (A). After deglycosylation by PNGase F treatment, the MabPair mixture QB11750 was subject to UV detection. (B). The intact (non-reduced) masses of anti-hTSLP IgG1-D265A antibody QB11718 (top) and anti-hIL-33 IgG4 antibody QB11465 (bottom). (C). After deglycosylation by PNGase F treatment, the MabPair mixture QB11750 was reduced by incubation at 55° C. in a buffer containing 4 M Guanidine Hydrochloride, 50 mM Tris pH8.0, 50 mM DTT for 30 min. The reduced LCs and HCs were subject to TIC (Total Ion Chromatogram) scan, which is a chromatogram created by summing up intensities of all mass spectral peaks belonging to the same scan. (D). The mass of deconvoluted peaks matched to the LC of anti-hIL-33 antibody QB11465 (theoretical mass 24026.53 Da), the LC of anti-hTSLP antibody QB11718 (theoretical mass 23664.31 Da), the HC of anti-hTSLP antibody QB11718 (theoretical mass 49187.03 Da), and the HC of anti-hIL-33 antibody QB11465 (theoretical mass 48987.43 Da) sequentially from top to bottom.

FIG. 13: Fab fragments of the MabPair antibody mixture QB11750 consisting of anti-hIL-33 IgG4 antibody QB11465 and anti-hTSLP IgG1-D265A antibody QB11718 by mass spectrometry analysis. (A). 400 μg of MabPair antibody mixture QB11750 were digested with 100 Units of IdeS at 37° C. overnight, diluted at 1:2 in the 2-MEA containing Reduction Buffer and heated up at 37° C. for 1 hr before the reaction was quenched with 10% formic acid. The treated sample was injected into Agilent 6224 accurate-mass TOF mass spectrometer for analysis under UV detection. (B). The mass of all individual deconvoluted peaks as shown in (A). The main peaks 3, 6, 10, 11, 12, 13, and shoulder peak 14 are annotated inside the respective small figures. The x axes show the deconvoluted mass, and the y axes show counts, which are reflective of the abundance of protein of a given mass.

FIG. 14: Mass spectrometry analysis of MabPair antibody mixture QB11823 consisting of anti-hIL-33 IgG4 antibody QB11465 and anti-hTSLP IgG1-D265A antibody QB11764. Experimental procedures are described in Example 11. (A). After deglycosylation by PNGase F treatment, the non-reduced MabPair mixture QB11823 was subjected to UV detection. (B). After deglycosylation by PNGase F treatment, the MabPair mixture QB11823 was digested with 100 Units of IdeS at 37° C. overnight to generate F(ab′) 2 and Fc/2 fragments, then subjected to UV detection. (C). The mass of the 4 deconvoluted peaks. The peak 1 matches to the half Fc (Fc/2) of anti-hTSLP antibody QB11764, peak 2 matches to the half Fc (Fc/2) of anti-hIL-33 antibody QB11465, peak 3 matches to the F(ab′) 2 fragment of anti-hIL-33 antibody QB11465, peak 4 matches to the F(ab′) 2 fragment of anti-hTSLP antibody QB11764. The x axes show the deconvoluted mass, and the y axes show counts, which are reflective of the abundance of protein of a given mass.

FIG. 15: Fab fragments of the MabPair antibody mixture QB11823 consisting of anti-hIL-33 IgG4 antibody QB11465 and anti-hTSLP IgG1-D265A antibody QB11764 by mass spectrometry analysis. (A). 400 μg of MabPair antibody mixture QB11823 were digested with 100 Units of IdeS at 37° C. overnight, diluted at 1:2 in the 2-MEA containing Reduction Buffer and heated up at 37° C. for 1 hr before the reaction was quenched with 10% formic acid. The treated sample was injected into Agilent 6224 accurate-mass TOF mass spectrometer for analysis under UV detection. (B). The mass of the deconvoluted peaks B and D as shown in (A). Peaks 1 and 2 are the nonreduced F(ab′) 2 of anti-hIL-33 antibody QB11465 and anti-hTSLP antibody QB11764 respectively. The minor peaks A and C are the 2-MEA adducted species of Fab fragment from anti-hIL-33 antibody QB11465 and anti-hTSLP antibody QB11764 respectively. The x axes show the deconvoluted mass, and the y axes show counts, which are reflective of the abundance of protein of a given mass.

FIG. 16: Surface plasmon resonance sensorgrams of the captured anti-hTSLP antibodies interacting with hTSLP (A) and cyTSLP (B) and 1:1 kinetic model fit overlays in Biacore T200. (A). The polyclonal goat-anti-human IgG (Fc specific) antibodies were immobilized on CM4 chip surface using a standard amine coupling method. Anti-hTSLP antibodies QB10985 (comparator antibody), QB11781, QB11764 were captured, E. coli derived monomeric hTSLP antigen from R&D Systems (cat no. 1398-TS-010) at 1.0; 0.5; 0.25; 0.13; 0.063; 0.031 nM, or in-house monomeric hTSLP antigen QB11630 (SEQ ID NO: 32) at 10; 4; 1.6; 0.64; 0.256; 0.1024 nM, or in-house monomeric hTSLP antigen QB11631 (SEQ ID NO: 34) at 8; 4; 2; 1; 0.5; 0.25; 0.13 nM were injected at 50 uL/min, association time was 3-10 min, disassociation time was monitored for up to 12 min, the cycles were run at 25° C. After each cycle, the chip was regenerated with 10 mM glycine-HC1, pH 1.5. (B). The experiments were done in the same way as (A) with exception of in house monomeric cyTSLP antigen QB11632 (SEQ ID NO: 35) at 200; 66.7; 22.2; 7.4; 2.47; 0.823 nM or QB11633 (SEQ ID NO: 37) at 50; 16.7; 5.56; 1.85; 0.617; 0.206 nM. The results were analyzed in the Biacore T200 evaluation software V3.2. Results were double referenced and fit to a 1:1 Langmuir interaction model. The values of the calculated parameters for the kinetic analysis are listed in Table 30 and Table 31.

FIG. 17: Assessment of blocking activities of the anti-hIL-33 IgG4 antibody QB11465 by cell-based assays. (A). Inhibition of IFNγ cytokine secretion from primary human NK cells by anti-hIL-33 antibodies. The purified human NK cells were seeded at 30,000 cells/well in complete RPMI1640 medium, treated for 24 hr with a constant amount of hIL-33 at concentrations close to EC₅₀ values and IL-12 at 1 ng/ml in combination with a 1:3 serially titrated anti-hIL-33 antibody. IFNγ cytokine levels at pg/ml in the supernatants were determined using human IFNγ ELISA kit from R&D Systems as per the manufacturer's instructions. (B). Inhibition of IL-5 secretion from primary human ILC2 cells by anti-hIL-33 antibodies. The purified ILC2s were cultured in the RPMI1640 complete medium in the presence of human IL-2, IL-25, TSLP and IL-33 cytokines each at 10 ng/ml for expansion. ILC2 cells were washed then seeded at 30,000 cells/well and treated for 48 hr with an amount of hIL-33 at concentrations close to EC₉₀ values in combination with a 1:3 serially titrated anti-hIL-33 antibody or isotype control IgG4 antibody. The levels of hIL-5 at pg/ml in supernatants were determined using human IL-5 ELISA kit as per the manufacturer's instructions. (C). Inhibition of p38 MAPK phosphorylation of primary human ILC2 cells by anti-hIL-33 antibodies. ILC2s were seeded at 100,000 cells/well and treated for 15 min with an amount of hIL-33 at concentrations close to EC₉₀ values plus a 1:3 serially titrated anti-hIL-33 antibodies. The p38 MAPK phosphorylation levels as MFI (Mean Fluorescence Intensity) were determined using an AlphaLISA SureFire Ultra p-38 MAPK HV Assay Kit as per the manufacturer's instructions. (D). Inhibition of p38 MAPK phosphorylation of primary human ILC2 cells induced by hIL-33 or cyIL-33. The experiments were done in the same way as in (C) with the exception that hIL-33 and cyIL-33 were separately used to determine the cross-species blocking activity of antibody QB11465. The IC₅₀ values were calculated using GraphPad Prism software.

FIG. 18: Inhibition of hTSLP-induced proliferation of BaF3 cells stably expressing hTSLPR/hIL7Rζ by anti-hTSLP antibodies. BaF3 cells stably expressing hTSLPR/hIL-7Rα were seeded at 10,000 cells/well and treated for 72 hr with an amount of hTSLP at concentrations close to EC₅₀ values plus titrated anti-hTSLP antibodies or isotype control antibody. Proliferation as RLU was determined using a CellTiter-Glo Luminescent Cell Viability Assay kit from Promega according to the manufacturer's instructions. (A). In-house hTSLP antigen QB11630 (SEQ ID NO: 32); (B). In-house hTSLP antigen QB11631 (SEQ ID NO: 34); (C). In-house cyTSLP antigen QB11632 (SEQ ID NO: 35); (D). In-house cyTSLP QB11633 (SEQ ID NO: 37); (E). Native hTSLP derived from healthy donor 1; (F). Native hTSLP derived from healthy donor 2. Native hTSLP was derived from human Small Airway Epithelial Cells (SAECs) purchased from Lonza. The x-axes represent the antibody concentration (nM) employed in the assay, the y-axes represent the luminescence output as RLU (relative light units). The IC₅₀ values were calculated using GraphPad Prism software.

FIG. 19: Inhibition of hTSLP-induced pSTAT5 of BaF3 cells stably expressing hTSLPR/hIL7Rα by anti-hTSLP antibodies. BaF3 cells stably expressing hTSLPR/hIL-7Rα were seeded at 10,000 cells/well and treated with different forms of TSLP in combination with the titrated anti-hTSLP antibodies for 15 min. (A). In-house hTSLP QB11630 (SEQ ID NO: 32); (B). In-house hTSLP QB11631 (SEQ ID NO: 34); (C). In-house cyTSLP QB11632 (SEQ ID NO: 35). The pSTAT5 phosphorylation level was determined using an AlphaLISA SureFire Ultra p-STAT5 HV Assay Kit as per the manufacturer's instructions. The plates were read in EnVision Multilabel Plate Reader from Perkin Elmer, the x-axes represent the antibody concentration (nM) employed in the assay, the y-axes represent the phosphorylated pSTAT5 output as MFI (Mean Fluorescence Intensity). The IC₅₀ values were calculated using GraphPad Prism software.

FIG. 20: Inhibition of hTSLP-induced CCL17 release from human monocytes by anti-hTSLP antibodies. The purified human monocytes were seeded at 150,000 cells/well and treated for 24 hr with a constant amount of TSLP at concentrations close to EC₅₀ values plus the titrated anti-TSLP antibodies or isotype control antibody QB11571. Then the CCL17 chemokine levels in supernatants were determined using a Human CCL17/TARC DuoSet ELISA kit as per the manufacturer's instructions. (A). in-house recombinant hTSLP QB11630, (B). in-house hTSLP QB11631, (C). in-house cyTSLP QB11632, were used in this assay. The x-axes represent the antibody concentration (nM) added in the assay, the y-axes represent the determined CCL17 at pg/ml in supernatants. The IC₅₀ values were calculated using GraphPad Prism software.

FIG. 21: Inhibition of hTSLP-induced IL-5 secretion from human ILC2 cells by anti-hTSLP antibodies. The purified human ILC2s were cultured in complete RPMI 1640 medium containing human IL-2, IL-25, TSLP, and IL-33 each at 10 ng/ml for expansion. ILC2 cells were washed then seeded at 30,000 cells/well and treated for 48 hr with a fixed amount of in-house recombinant hTSLP QB11630 (A) or hTSLP QB11631 (B) at concentrations close to EC₉₀ values in combination with a 1:4 serially titrated anti-hTSLP antibody or isotype control antibody. The levels of hIL-5 in supernatants were determined using a Human IL-5 DuoSet ELISA kit from R&D Systems (cat. no. DY205) as per the manufacturer's instructions. The x-axes represent the antibody concentration (nM) added in the assay, the y-axes represent the determined IL-5 at pg/ml in supernatants. The IC₅₀ values were calculated using GraphPad Prism software.

FIG. 22: Inhibition of hTSLP-induced pSTAT5 from human ILC2 cells by anti-hTSLP antibodies. Purified human ILC2s were cultured in complete RPMI 1640 medium containing human IL-2, IL-25, TSLP, and IL-33 each at 10 ng/ml for expansion. ILC2 cells were washed then seeded at 30,000 cells/well and treated for 48 hr with a fixed amount of in-house recombinant hTSLP QB11630 (A) or hTSLP QB11631 (B) or cyTSLP QB11632 (C) at concentrations close to EC₉₀ values in combination with a 1:4 serially titrated anti-hTSLP antibody or isotype control antibody. The pSTAT5 phosphorylation level was determined using an AlphaLISA SureFire Ultra p-STAT5 HV Assay Kit as per the manufacturer's instructions. The plates were read in EnVision Multilabel Plate Reader from PerkinElmer, the x-axes represent the antibody concentration (nM) employed in the assay, the y-axes represent the phosphorylated pSTAT5 output as MFI (Mean Fluorescence Intensity). The IC₅₀ values were calculated using GraphPad Prism software.

FIG. 23: Synergistic inhibition of IL-5 secretion by the combination of anti-hIL-33 IgG4 antibody QB11465 and anti-TSLP IgG1-D265A antibody QB11718. The purified human ILC2s were cultured in complete RPMI 1640 medium containing human IL-2, IL-25, TSLP, and IL-33 each at 10 ng/ml for expansion. Expanded ILC2 cells were washed then seeded at 30,000 cells/well and treated for 48 hr with recombinant hTSLP and hIL-33 each at 5 ng/ml in combination with 4×, 2×, 1×, 0.5× or 0.25×of IC₅₀ concentrations of anti-hTSLP antibody alone, anti-hIL-33 antibody alone, or the combination of anti-hTSLP antibody and anti-hIL-33 antibody. The levels of hIL-5 in supernatants were determined using a Human IL-5 DuoSet ELISA kit from R&D Systems (cat. no. DY205) as per the manufacturer's instructions. (A). Synergistic effect of anti-hTSLP antibody QB11718 and anti-hIL-33 antibody QB11465. (B). Synergistic effect of anti-hTSLP comparator antibody QB10985 and anti-hIL-33 antibody QB11465. (C). The calculated CI (Combination Index) from (A) and (B).

FIG. 24: The lead anti-hIL-33 IgG4 antibody QB11465 significantly inhibited allergen-induced lung eosinophilic inflammation and IL-4 production in hIL-33 knock-in mice. To induce airway inflammation, purified House Dust Mite (HDM) at 25 ug/15 ul was administered intranasally to female C57BL/6 hIL-33 knock-in mice under isoflurane anesthesia, once a day, for 5 consecutive days for a total period of 6 weeks Starting at Day 22 into the study, mice in group 3 and group 4 were dosed intraperitoneally twice a week, for 3 consecutive weeks with lead anti-hIL-33 IgG4 antibody QB11465 at 5 mg/kg (group 3), or anti-hIL-33 comparator-3 antibody QB11094 at 5 mg/kg (group 4). Group 1 did not receive HDM, Group 2 received HDM but without anti-IL-33 antibody treatment. (A). Percentage of BALF eosinophils in group 1-4. (B). IL-4 levels (pg/ml) in BALF from mice group 1-4.

### REFERENCE TO SEQUENCE LISTING

This application includes a sequence listing appended hereto. An electronic version of the Sequence Listing is filed herewith, the contents of which are incorporated by reference in their entirety. The electronic file was created on Jan. 23, 2025 and is 142,392 bytes in size and is titled 126861-0009UT01.xml.

BRIEF DESCRIPTION OF THE SEQUENCE LISTING

| SEQ ID NO | DESCRIPTION OF THE SEQUENCE |
|---|---|
| SEQ ID NO: 1 | Amino acid sequence of His6-tagged hIL-33 (Ser112 - Thr270), QB10975 |
| SEQ ID NO: 2 | Amino acid sequence of His6-tagged hIL-33 (Ser112 - Thr270) with C208S + |
| | C232S, QB11921 |
| SEQ ID NO: 3 | Amino acid sequence of His6-tagged cyIL-33 (Ser112 - Ile270), QB10976 |
| SEQ ID NO: 4 | Amino acid sequence of murine anti-hIL-33 QB10998 VL |
| SEQ ID NO: 5 | Amino acid sequence of chimeric anti-hIL-33 QB10998 LC |
| SEQ ID NO: 6 | Amino acid sequence of murine anti-hIL-33 QB10998 VH |
| SEQ ID NO: 7 | Amino acid sequence of chimeric anti-hIL-33 QB10998 HC |
| SEQ ID NO: 8 | Amino acid sequence of anti-hIL-33 QB11004 VL |
| SEQ ID NO: 9 | Amino acid sequence of anti-hIL-33 QB11004 LC |
| SEQ ID | Amino acid sequence of anti-hIL-33 QB11004 VH |
| NO: 10 | |
| SEQ ID | Amino acid sequence of anti-hIL-33 QB11004 HC |
| NO: 11 | |
| SEQ ID | Amino acid sequence of anti-hIL-33 QB11061 VL |
| NO: 12 | |
| SEQ ID | Amino acid sequence of anti-hIL-33 QB11061 LC |
| NO: 13 | |
| SEQ ID | Amino acid sequence of anti-hIL-33 QB11061 VH |
| NO: 14 | |
| SEQ ID | Amino acid sequence of anti-hIL-33 QB11061 HC |
| NO: 15 | |
| SEQ ID | Amino acid sequence of anti-hIL-33 QB11119 VL |
| NO: 16 | |
| SEQ ID | Amino acid sequence of anti-hIL-33 QB11119 LC |
| NO: 17 | |
| SEQ ID | Amino acid sequence of anti-hIL-33 QB11119 VH |
| NO: 18 | |
| SEQ ID | Amino acid sequence of anti-hIL-33 QB11119 HC |
| NO: 19 | |
| SEQ ID | Amino acid sequence of anti-hIL-33 QB11421 VL |
| NO: 20 | |
| SEQ ID | Amino acid sequence of anti-hIL-33 QB11421 LC |
| NO: 21 | |
| SEQ ID | Amino acid sequence of anti-hIL-33 QB11421 VH |
| NO: 22 | |
| SEQ ID | Amino acid sequence of anti-hIL-33 QB11421 HC |
| NO: 23 | |
| SEQ ID | Nucleotide sequence encoding anti-hIL-33 QB11465 VL |
| NO: 24 | |
| SEQ ID | Amino acid sequence of anti-hIL-33 QB11465 VL |
| NO: 25 | |
| SEQ ID | Nucleotide sequence encoding anti-hIL-33 QB11465 LC |
| NO: 26 | |
| SEQ ID | Amino acid sequence of anti-hIL-33 QB11465 LC |
| NO: 27 | |
| SEQ ID | Nucleotide sequence encoding anti-hIL-33 QB11465 VH |
| NO: 28 | |
| SEQ ID | Amino acid sequence of anti-hIL-33 QB11465 VH |
| NO: 29 | |
| SEQ ID | Nucleotide sequence encoding anti-hIL-33 QB11465 IgG4 HC |
| NO: 30 | |
| SEQ ID | Amino acid sequence of anti-hIL-33 QB11465 IgG4 HC |
| NO: 31 | |
| SEQ ID | Amino acid sequence of Avitag_c-MYC_His6_hTSLP (wild type) QB11630 |
| NO: 32 | |
| SEQ ID | Amino acid sequence of Avitag_c-MYC_His6_hTSLP QB11033 with deletion of |
| NO: 33 | ¹²⁶KRRKR¹³⁰ |
| SEQ ID | Amino acid sequence of Avitag_c-MYC_His6_hTSLP QB11631 with mutations of |
| NO: 34 | R127A + R130S |
| SEQ ID | Amino acid sequence of Avitag_FLAG_His6_cyTSLP QB11632 (wild type) |
| NO: 35 | |
| SEQ ID | Amino acid sequence of Avitag_FLAG_His6_cyTSLP QB10974 with deletion of |
| NO: 36 | ¹²⁶KRRKR¹³⁰ |
| SEQ ID | Amino acid sequence of Avitag_FLAG_His6_cyTSLP QB11633 with mutations of |
| NO: 37 | R127A + R130S |
| SEQ ID | Amino acid sequence of hTSLPR-Fc fusion protein QB11034 |
| NO: 38 | |
| SEQ ID | Amino acid sequence of anti-hTSLP VH of clone 135F1 from rabbit hybridoma |
| NO: 39 | |
| SEQ ID | Amino acid sequence of anti-hTSLP HC of clone 135F1 from rabbit hybridoma |
| NO: 40 | |
| SEQ ID | Amino acid sequence of anti-hTSLP VL of clone 135F1 from rabbit hybridoma |
| NO: 41 | |
| SEQ ID | Amino acid sequence of anti-hTSLP LC of clone 135F1 from rabbit hybridoma |
| NO: 42 | |
| SEQ ID | Amino acid sequence of anti-hTSLP QB11341 and QB11548 VH of humanized |
| NO: 43 | clone 135F1 |
| SEQ ID | Amino acid sequence of anti-hTSLP QB11341 and QB11548 HC of humanized |
| NO: 44 | clone 135F1 |
| SEQ ID | Amino acid sequence of anti-hTSLP QB11341 VL of humanized clone 135F1 |
| NO: 45 | |
| SEQ ID | Amino acid sequence of anti-hTSLP QB11341 LC of humanized clone 135F1 |
| NO: 46 | |
| SEQ ID | Amino acid sequence of anti-hTSLP QB11548 VL of humanized clone 135F1 after |
| NO: 47 | yeast display |
| SEQ ID | Amino acid sequence of anti-hTSLP QB11548 LC of humanized clone 135F1 after |
| NO: 48 | yeast display |
| SEQ ID | Nucleotide sequence encoding anti-hTSLP QB11764 VH of the final clone |
| NO: 49 | |
| SEQ ID | Amino acid sequence of anti-hTSLP QB11764 VH of the final clone |
| NO: 50 | |
| SEQ ID | Nucleotide sequence encoding anti-hTSLP QB11764 IgG1-D265A of the final clone |
| NO: 51 | for MabPair production |
| SEQ ID | Amino acid sequence of anti-hTSLP QB11764 IgG1-D265A of the final clone for |
| NO: 52 | MabPair production |
| SEQ ID | Nucleotide sequence encoding anti-hTSLP QB11764 VL of the final clone |
| NO: 53 | |
| SEQ ID | Amino acid sequence of anti-hTSLP QB11764 VL of the final clone |
| NO: 54 | |
| SEQ ID | Nucleotide sequence encoding anti-hTSLP QB11764 LC of the final clone for |
| NO: 55 | MabPair production |
| SEQ ID | Amino acid sequence of anti-hTSLP QB11764 LC of the final clone for MabPair |
| NO: 56 | production |
| SEQ ID | Amino acid sequence of anti-hTSLP QB10987 VH of chimeric antibody clone |
| NO: 57 | 23B12 |
| SEQ ID | Amino acid sequence of anti-hTSLP QB10987 HC of chimeric antibody clone |
| NO: 58 | 23B12 |
| SEQ ID | Amino acid sequence of anti-hTSLP QB10987 VL of chimeric antibody clone |
| NO: 59 | 23B12 |
| SEQ ID | Amino acid sequence of anti-hTSLP QB10987 LC of chimeric antibody clone 23B12 |
| NO: 60 | |
| SEQ ID | Amino acid sequence of anti-hTSLP QB10990 VH of humanized antibody clone hz- |
| NO: 61 | 3C |
| SEQ ID | Amino acid sequence of anti-hTSLP QB10990 HC of humanized antibody clone hz- |
| NO: 62 | 3C |
| SEQ ID | Amino acid sequence of anti-hTSLP QB10990 VL of humanized antibody clone hz- |
| NO: 63 | 3C, identical to QB11060 VL |
| SEQ ID | Amino acid sequence of anti-hTSLP QB10990 LC of humanized antibody clone hz- |
| NO: 64 | 3C, identical to QB11060 LC |
| SEQ ID | Amino acid sequence of anti-hTSLP QB11060 VH of humanized antibody clone hz- |
| NO: 65 | 3C, with D9P + S108L substitutions, identical to QB11237 VH |
| SEQ ID | Amino acid sequence of anti-hTSLP QB11060 HC of humanized antibody clone hz- |
| NO: 66 | 3C, with D9P + S108L substitutions, identical to QB11237 HC |
| SEQ ID | Amino acid sequence of anti-hTSLP QB11237 VL of humanized antibody clone hz- |
| NO: 67 | 3C after yeast display |
| SEQ ID | Amino acid sequence of anti-hTSLP QB11237 LC of humanized antibody clone hz- |
| NO: 68 | 3C after yeast display |
| SEQ ID | Nucleotide sequence encoding anti-hTSLP QB11718 VH of the final clone |
| NO: 69 | |
| SEQ ID | Amino acid sequence of anti-hTSLP QB11718 VH of the final clone |
| NO: 70 | |
| SEQ ID | Nucleotide sequence encoding anti-hTSLP QB11718 IgG1-D265A HC of the final |
| NO: 71 | clone for MabPair production |
| SEQ ID | Amino acid sequence of anti-hTSLP QB11718 IgG1-D265A HC of the final clone |
| NO: 72 | for MabPair production |
| SEQ ID | Nucleotide sequence encoding anti-hTSLP QB11718 VL of the final clone |
| NO: 73 | |
| SEQ ID | Amino acid sequence of anti-hTSLP QB11718 VL of the final clone |
| NO: 74 | |
| SEQ ID | Nucleotide sequence encoding anti-hTSLP QB11718 LC of the final clone for |
| NO: 75 | MabPair production |
| SEQ ID | Amino acid sequence of anti-hTSLP QB11718 LC of the final clone for MabPair |
| NO: 76 | production |
| SEQ ID | Amino acid sequence of CDR1 region of VL of anti-hIL-33 antibody QB11421 and |
| NO: 77 | QB11465 |
| SEQ ID | Amino acid sequence of CDR2 region of VL of anti-hIL-33 antibody QB11421 and |
| NO: 78 | QB11465 |
| SEQ ID | Amino acid sequence of CDR3 region of VL of anti-hIL-33 antibody QB11421 and |
| NO: 79 | QB11465 |
| SEQ ID | Amino acid sequence of CDR1 region of VH of anti-hIL-33 antibody QB11421 and |
| NO: 80 | QB11465 |
| SEQ ID | Amino acid sequence of CDR2 region of VH of anti-hIL-33 antibody QB11421 and |
| NO: 81 | QB11465 |
| SEQ ID | Amino acid sequence of CDR3 region of VH of anti-hIL-33 antibody QB11421 and |
| NO: 82 | QB11465 |
| SEQ ID | Amino acid sequence of CDR1 region of VL of anti-hTSLP antibody QB11341, |
| NO: 83 | QB11548, QB11764 |
| SEQ ID | Amino acid sequence of CDR2 region of VL of anti-hTSLP antibody QB11341 |
| NO: 84 | |
| SEQ ID | Amino acid sequence of CDR3 region of VL of anti-hTSLP antibody QB11341 |
| NO: 85 | |
| SEQ ID | Amino acid sequence of CDR1 region of VH of anti-hTSLP antibody QB11341, |
| NO: 86 | QB11548, QB11764 |
| SEQ ID | Amino acid sequence of CDR2 region of VH of anti-hTSLP antibody QB11341, |
| NO: 87 | QB11548, QB11764 |
| SEQ ID | Amino acid sequence of CDR3 region of VH of anti-hTSLP antibody QB11341, |
| NO: 88 | QB11548, QB11764 |
| SEQ ID | Amino acid sequence of CDR2 region of VL of anti-hTSLP antibody QB11548, |
| NO: 89 | QB11764 |
| SEQ ID | Amino acid sequence of CDR3 region of VL of anti-hTSLP antibody QB11548, |
| NO: 90 | QB11764 |
| SEQ ID | Amino acid sequence of CDR1 region of VL of anti-hTSLP antibody QB11237, |
| NO: 91 | QB11718 |
| SEQ ID | Amino acid sequence of CDR2 region of VL of anti-hTSLP antibody QB11237, |
| NO: 92 | QB11718 |
| SEQ ID | Amino acid sequence of CDR3 region of VL of anti-hTSLP antibody QB11237, |
| NO: 93 | QB11718 |
| SEQ ID | Amino acid sequence of CDR1 region of VH of anti-hTSLP antibody QB11237, |
| NO: 94 | QB11718 |
| SEQ ID | Amino acid sequence of CDR2 region of VH of anti-hTSLP antibody QB11237, |
| NO: 95 | QB11718 |
| SEQ ID | Amino acid sequence of CDR3 region of VH of anti-hTSLP antibody QB11237, |
| NO: 96 | QB11718 |

### DETAILED DESCRIPTION

Provided herein are antibodies that bind to human IL33 (hIL33), antibodies that bind to human TSLP (hTSLP), mixtures of these antibodies, polynucleotides encoding these antibodies and mixtures, host cells containing such polynucleotides, and methods of treatment with these antibodies, mixtures, and polynucleotides. Further described herein are methods of making mixtures of these anti-hIL33 antibodies and anti-TSLP antibodies utilizing a single host cell line for producing the mixture. The anti-hIL33 antibodies described herein can bind to both human and cynomolgus monkey IL33, can inhibit the interaction of hIL33 with hST2 and/or with ST2/IL1AcP complex. The anti-hTSLP antibodies described herein can inhibit binding of hTSLP to hTSLPR (human Thymic stromal lymphopoietin-receptor) and/or can block the interaction of hTSLP with hIL7Rα and can bind to both human and cynomolgus monkey TSLP. Accordingly, invention of anti-hTSLP antibodies provided herein can inhibit the formation of hTSLP-hTSLPR complex or formation of hTSLP-hIL7Rα complex. A mixture of anti-hIL33 and anti-hTLSP antibodies, compared to either antibody alone, can exhibit greater effects on the decrease of secretion of several downstream cytokines, i.e. IL-4, IL-5, IL-13, which mediate inflammatory responses from eosinophils, B cells, mast cells, and the like. Accordingly, an invention mixture of anti-hIL33 and anti-hTLSP antibodies can treat or prevent inflammatory disease, such as asthma, COPD, and the like, while avoiding the side effects of long-term use of inhaled β-adrenergic agonists and inhaled corticosteroids. The invention mixture of anti-hIL33 and anti-hTLSP antibodies provided herein is also contemplated for use in treating virus infection and cancers.

Also provided herein are methods of making an antibody or a mixture of antibodies described herein utilizing a single host cell line. Further described herein are polynucleotides encoding these antibodies and mixtures, host cells containing such polynucleotides, and methods of treatment utilizing these antibodies, mixtures (including mixtures of antibodies), and polynucleotides.

### Definitions

An “agonist,” as meant herein, is a molecule that mimics or enhances the activity of a particular biologically active molecule or pathway. For example, a protein expressed on a cell surface might mediate downstream effects of a molecule or pathway when a cytokine binds to the protein. An agonist of the protein could elicit similar, or greater or lesser, effects (as compared to those elicited by the cytokine) when it interacts with the protein, although the agonist may or may not compete with the cytokine for binding to the protein.

An “alteration,” as meant herein, is a change in an amino acid sequence or in a nucleotide sequence. Alterations can be insertions, deletions, or substitutions. An “alteration” is the insertion, deletion, or substitution of a single amino acid or nucleotide. If, for example, a deletion removes three amino acids or three nucleotides from an amino acid or nucleotide sequence, then three alterations (in this case, deletions) have occurred. Alterations that are amino acid substitutions can be referred to by stating the amino acid present in the original sequence followed by the position of the amino acid in the original sequence followed by the amino acid replacing the original amino acid. For example, G133M means that the glycine originally present at position 133 in the original sequence is replaced by a methionine. Further, 133M means that the amino acid at position 133 is methionine, but does not specify the identity of the original amino acid, which could be any amino acid including methionine. Finally, G133 means that glycine is the amino acid at position 133 in the original sequence. In addition, G133M/A means that the glycine originally present at position 133 in the original sequence is replaced by either a methionine or an alanine.

An “alteration that disfavors heterodimers,” as meant herein, is a substitution, insertion, or deletion of a single amino acid within a third heavy chain constant domain (C_{H}3) amino acid sequence, optionally a human or primate C_{H}3 amino acid sequence, where the substitution, insertion, or deletion disfavors the formation of heterodimeric HC/HC pairs in the context of a mixture of antibodies. An antibody can comprise more than one alteration that disfavors heterodimers, and multiple alterations that disfavor heterodimers can occur at multiple sites in one or more antibodies in a mixture of antibodies. In some cases, an alteration that disfavors heterodimers may have little or no effect alone but can inhibit heterodimer formation when one or more other alteration that disfavors heterodimer formation is present in the same antibody or in a different antibody in a mixture of antibodies. Included among the alterations can be the substitution of a charged residue for the residue present in the wild type sequence, which may or may not be charged. Alternatively, a substitution can create a steric clash in heterodimeric HC/HC pairs that interferes with proper heavy chain/heavy chain (HC/HC) pairing such as a “protuberance” abutting against another “protuberance” or a “hole” abutting against another “hole.” Protuberances (or knobs) and holes are described in U.S. Pat. No. 8,679,785, col. 12, line 12 to col. 13, line 2, which is incorporated herein by reference. An example of a pair alterations in an IgG heavy chain that can, together, disfavor heterodimer formation is D399K/R plus K409D/E.

As used herein, the phrase “inhibits the interaction”, in the context of invention antibodies, refers to an agent or “antagonist” that blocks or inhibits the activity or binding of a particular biologically active molecule. For example, a particular protein may activate a biological pathway with known downstream effects when it interacts with its binding partner. An invention antibody acts as an antagonist or inhibitor of that protein and/or its binding partner to lessen or eliminate those downstream effects, optionally by blocking or inhibiting interaction of the protein and its binding partner. Exemplary antagonists are the invention anti-IL33 and anti-TSLP antibodies set forth herein.

An “antibody,” as meant herein, is a protein that contains at least one V_{H} or V_{L}. An antibody often contains both a V_{H} and a V_{L}. V_{H}s and V_{L}s are described in full detail in, e.g., Kabat et al., Sequences of Proteins of Immunological Interest, Fifth Edition, U.S. Department of Health and Human Services, Public Health Service, National Institutes of Health, NIH Publication No. 91-3242, 1991, pp. xvi-xix and pp. 103-533, which are incorporated by reference herein. “Antibody” includes molecules having different formats such as single chain Fv (scFv) antibodies (which contain a V_{H} and a V_{L} joined by a linker), Fab, F (ab′) 2, Fab′, and scFv: Fc antibodies (as described in Carayannopoulos and Capra, Ch. 9 in FUNDAMENTAL IMMUNOLOGY, 3.sup.rd ed., Paul, ed., Raven Press, New York, 1993, pp. 284-286, which is incorporated herein by reference), BiTE® antibodies, single domain antibodies, bispecific antibodies, Fab-scFv, DVD-IgG, IgG (H)-scFv, nanobody, nanobody-HSA, diabody, DART, TandAb, scDiabody, miniantibody, minibody, etc. (see, e.g., Spiess et al. (2015), Alternative molecular formats and therapeutic applications for bispecific antibodies, Molecular Immunology 67:95-106), and IgG antibodies as defined below, among many other possible formats.

A “bispecific antibody,” as meant herein, is an antibody that comprises at least one variable domain from a first antibody that binds to a first epitope or antigen and at least one variable domain from a second antibody that binds to a second epitope or antigen. In some cases, the first and second epitopes will reside on different molecules, optionally on different proteins. Thus, in some cases the first and second antibodies will bind to different antigens. A bispecific antibody can have a variety of formats. For example, a bispecific antibody can be a Bispecific T cell engager (BiTE), a Dual-Affinity Retargeting Protein (DART), a diabody, a Tandem Diabody (TandAb), or an IgG antibody, among many possible formats. See, e.g., Wang et al. (2019), Design and Production of Bispecific Antibodies, Antibodies 8, 43 (30 pages), which is incorporated herein by reference in its entirety and Spiess et al. (2015), Alternative molecular formats and therapeutic applications for bispecific antibodies, Mol. Immunol. 67:95-106, which is incorporated herein by reference in its entirety. Each of these exemplary formats comprises both a V_{H} and a V_{L} from two different antibodies that bind to different epitopes. A bispecific antibody can comprise alterations that encourage cognate pairing of V_{H}s and V_{L}s, which are called herein partner-directing alterations and discussed above and below. If the bispecific antibody is an IgG antibody consisting of two heavy chains, each from two different antibodies, and two light chains, each from one of the two different antibodies, then it can also comprise alterations that can encourage formation of heterodimeric HC/HC pairing. Many such alterations are known in the art.

A “cancer antigen,” as meant herein, is a molecule, optionally a protein, that is abundantly expressed on the surface of a cancer cell. The expression of a cancer antigen is sufficiently high that it can be detected by typical immunohistochemistry (IHC). See, e.g., Parra et al. (2018), Appl. Immunohistochem. Mol. Morphol. 26 (2): 83-93. Cancer antigens can be expressed at variable levels in different cancer cells and may also be expressed on normal cells, at least to some extent. In some cases, a cancer antigen is expressed only on cancer cells. For example, a rearranged form of Epidermal Growth Factor Receptor (EGFR) called EGFRvIII is expressed on glioblastoma cells, but not on normal cells. In another example, carcinoembryonic antigen (CEA) is expressed in normal tissue during fetal development, but not after birth. CEA is expressed in some cancer cells. Thus, both EGFRVIII and CEA are cancer antigens as meant herein. Other examples of cancer antigens include proteins encoded by genes including EGFR, V-ERB-B2 Avian Erythroblastic Leukemia Viral Oncogene Homolog 2 (HER2), Epithelial Cellular Adhesion Molecule (EpCAM), Glypican 3 (GPC3), Tumor Necrosis Factor Receptor Superfamily, Member 17 (TMFRSF17, called BCMA herein), Claudin-18.2, CD20, and Prostate-Specific Antigen (PSA), among many others.

A “charged” amino acid, as meant herein, is an acidic or basic amino acid that can have a charge at near-physiologic pH. These include the acidic amino acids glutamic acid (E) and aspartic acid (D), which are negatively charged at physiologic pH, and the basic amino acids arginine (R) and lysine (K), which are positively charged at physiologic pH. The weakly basic amino acid histidine, which can be partially charged at near-physiologic pH, is not within the definition of “charged” amino acid herein. To avoid confusion, a positive charge is considered to be “opposite” to a negative charge, as meant herein. Thus, for example, the amino acids glutamate (E) and arginine (R) are opposite in charge.

“Clearance” of an antibody in vivo refers to elimination of the antibody, which can be detected as elimination or a lessening in amount of the antibody in the bloodstream or in other tissues of a mammal. Generally, to determine a rate of clearance, the antibody will be administered to the mammal, and subsequently blood or tissue of the mammal will be periodically sampled and quantitatively tested for the presence of the antibody. From such tests, an in vivo half-life (T_{1/2}) and/or an Area Under the Curve (AUC) value can be derived. A decrease in T_{1/2} or AUC indicates an increase in clearance, as meant herein. An exemplary method for determining whether clearance of an altered human IgG antibody in a mouse has increased or decreased relative to the unaltered antibody includes the following steps. The unaltered and altered antibodies can each be injected subcutaneously, e.g., under the skin over the shoulders, into separate mice. Whole blood samples of about 0.1 mL can be collected at each time point by retro-orbital sinus puncture. The blood can be clotted and processed to obtain serum. Serum samples can be assayed for the presence of human antibody using an antibody specific for a human Fc, for example a commercially sold immunoassay system such as one of those available from Gyros U.S., Inc., Warren, NJ, USA. Blood samples can be collected, for example, at 0, 0.5, 2, 8, 24, 72, 120, 168, 240, 312, 384, and 480 hours after injection. Pharmacokinetic parameters can be estimated from serum concentrations using, for example, Phoenix® 6.3 software (Pharsight, Sunnyvale, CA, USA).

A “chemotherapeutic agent” targets dividing cells and interferes with processes that are tied to cell division, for example, DNA replication, RNA synthesis, protein synthesis, the assembly, disassembly, or function of the mitotic spindle, and/or the synthesis or stability of molecules that play a role in these processes, such as nucleotides or amino acids. Thus, a chemotherapeutic agent can kill both cancer cells and other dividing cells. Chemotherapeutic agents are well-known in the art. They include, for example, the following agents: alkylating agents (e.g., busulfan, temozolomide, cyclophosphamide, lomustine (CCNU), streptozotocin, methyllomustine, cis-diamminedi-chloroplatinum, thiotepa, and aziridinylbenzo-quinone); inorganic ions (e.g., cisplatin and carboplatin); nitrogen mustards (e.g., melphalan hydrochloride, chlorambucil, ifosfamide, and mechlorethamine HC1); nitrosoureas (e.g., carmustine (BCNU)); anti-neoplastic antibiotics (e.g., adriamycin (doxorubicin), daunomycin, mithramycin, daunorubicin, idarubicin, mitomycin C, and bleomycin); plant derivatives (e.g., vincristine, vindesine, vinblastine, vinorelbine, paclitaxel, docetaxel, VP-16, and VM-26); antimetabolites (e.g., methotrexate with or without leucovorin, 5-fluorouracil with or without leucovorin, 5-fluorodeoxyuridine, 6-mercaptopurine, 6-thioguanine, gemcitabine, cytarabine, 5-azacytidine, hydroxyurea, deoxycoformycin, and fludarabine); podophyllotoxins (e.g., etoposide, irinotecan, and topotecan); as well as actinomycin D, dacarbazine (DTIC), mAMSA, procarbazine, hexamethylmelamine, pentamethylmelamine, L-asparaginase, and mitoxantrone. See, e.g., Cancer: Principles and Practice of Oncology, 4.sup.th Edition, DeVita et al., eds., J.B. Lippincott Co., Philadelphia, Pa. (1993), the relevant portions of which are incorporated herein by reference.

Other chemotherapeutic agents include those that act by the same general mechanism as those listed above. For example, agents that act by alkylating DNA, as do, for example, alkylating agents and nitrogen mustards, are considered chemotherapeutic agents. Agents that interfere with nucleotide synthesis, like, for example, methotrexate, cytarabine, 6-mercaptopurine, 5-fluorouracil, and gemcitabine, are considered to be chemotherapeutic agents. Mitotic spindle poisons are considered chemotherapeutic agents, as are, for, example, paclitaxel and vinblastine. Topoisomerase inhibitors (e.g., podophyllotoxins), which interfere with DNA replication, are considered to be chemotherapeutic agents. Antibiotics that interfere with DNA synthesis by various mechanisms, examples of which are doxorubicin, bleomycin, and mitomycin, are considered to be chemotherapeutic agents. Agents that carbamoylate amino acids (e.g., lomustine, carmustine) or deplete asparagine pools (e.g., asparaginase) are also considered chemotherapeutic agents. Merck Manual of Diagnosis and Therapy, 17.sup.th Edition, Section 11, Hematology and Oncology, 144. Principles of Cancer Therapy, Table 144-2 (1999). Specifically included among chemotherapeutic agents are those that directly affect the same cellular processes that are affected by the chemotherapeutic agents listed above.

A “cognate” HC in the context of a mixture of antibodies, as meant herein, is the HC that a particular LC is known to pair with to form a binding site for a particular antigen. For example, if a known full-length IgG Antibody X binds to Antigen X, the Antibody X HC is the cognate HC of the Antibody X LC, and vice versa. Further, if the mixture also comprises an Antibody Y that binds to Antigen Y, the antibody Y HC is “non-cognate” with respect to the Antibody X LC and vice versa, and the Antibody Y LC is “non-cognate” with respect to the Antibody X HC and vice versa.

A “complementarity determining region” (CDR) is a hypervariable region within a V_{H} or V_{L}. Each V_{H} and V_{L} contains three CDRs called CDR1, CDR2, and CDR3. The CDRs form loops on the surface of the antibody and are primarily responsible for determining the binding specificity of an antibody. The CDRs are interspersed between four more conserved framework regions (called FR1, FR2, FR3, and FR4) as follows: FR1-CDR1-FR2-CDR2-FR3-CDR3-FR4.

Positions of CDRs are indicated in Examples 2, 5, 6, and 7, VH CDRs are defined as follows: CDR1 is at positions 31-35 (with possible insertions numbered 35a and 35b); CDR2 is at positions 50-65 (with possible insertions numbered 52a-52c); and CDR3 is at positions 95-102 (with possible insertions numbered 100A-100K). Kabat et al., supra, at xvii, which is incorporated herein by reference. VL CDRs are defined as follows: CDR1 is at positions 24-34 (with possible insertions numbered 27A-27F); CDR2 is at positions 50-56; and CDR3 is at positions 89-97 (with possible insertions numbered 95A-95F). These definitions of the VH and VL CDRs are used herein.

A treatment or drug is considered to be administered “concurrently” with another treatment or drug if the two treatments/drugs are administered within the same, small time frame, for example on the same day, or within the same more extended time frame. Such a more extended time frame can include a situation where, for example, one treatment/drug is administered once per week and the other is administered every 4 days. Although the two treatments/drugs may never or rarely be administered on the same day, the two treatments/drugs are administered on an ongoing basis during a common period of weeks, months, or a longer time period. Similarly, if one drug is administered once per year and the other is administered weekly, they are considered to be administered “concurrently” if the drug administered weekly is administered during the year before and/or after the administration of the drug that is administered once per year. Hence, as meant herein, “concurrent” administration of the two treatments/drugs includes ongoing treatment with two different treatments/drugs that goes on in a common time period.

A “conservative” amino acid substitution, as meant herein, is the substitution of an amino acid with a different amino acid having similar properties, such as similar polarity, hydrophobicity, or volume. Conservative substitutions include replacement of an amino acid with another amino acid within the same group, wherein the groups of amino acids include the following: (1) hydrophobic amino acids, which include alanine, leucine, isoleucine, valine, proline, phenylalanine, tryptophan, and methionine; (2) uncharged polar amino acids, which include glycine, serine, threonine, cysteine, tyrosine, asparagine, and glutamine; (3) basic amino acids, which include arginine, lysine, and histidine; and (4) acidic amino acids, which include aspartic acid and glutamic acid. Conservative substitutions also include the substitution of (1) A with V, L, or I, (2) R with K, Q, or N, (3) N with Q, H, K, R, (4) D with E, (5) C with S or A, (6) Q with N, (7) E with D, (8), G with P or A, (9) H with N, Q, K, or R, (10) I with L, V, M, A, or F, (11) L with I, V, M, A, or F, (12) K with R, Q, or N, (13) M with L, F, or I, (14) F with L, V, I, A, or Y, (15) P with A, (16) S with T, A, or G, (17) T with S, (18) W with Y or F, (19) Y with W, F, T, or S, and (20) V with I, M, L, F, or A.

A “cysteine substitution,” as meant herein, is an amino acid substitution where a cysteine replaces another amino acid.

Two or more antibodies are “different,” as meant herein, if the amino acid sequences of all the polypeptide chains included in the antibody are not “the same,” as meant herein.

Two amino acid sequences are “the same,” as meant herein, if the two sequences could be encoded by the same DNA sequence. That is, amino acid sequences that differ only as a result of post-translational modifications, e.g., elimination of a carboxyl-terminal lysine or cyclization of N-terminal glutamate or glutamine residues, are “the same” as meant herein.

Amino acid sequences are “different,” as meant herein, if they have one or more amino acid substitution, deletion, or insertion relative to each other, with the caveat that such “different” amino acid sequences are not considered different if the differences are due solely to post-translational modifications, that is, if the amino sequences could be encoded by the same DNA sequence.

An amino acid sequence is “encoded by a nucleotide sequence,” as meant herein, when the amino acid sequence could, theoretically, be encoded by the nucleotide sequence, given the known genetic code. Such a polypeptide chain need not be actually made from such a nucleic acid to be “encoded” by the nucleotide sequence, as meant herein, and the nucleotide sequence need not comprise all accessory sequences necessary for transcriptional and/or translational stopping and starting to “encode” an amino acid sequence. As in known in the art, a given amino acid sequence is “encoded” by a defined collection of nucleic acid sequences due to the degeneracy of the genetic code. Further, an amino acid sequence that is “encoded” by a nucleotide sequence, as described above, is still considered herein to be “encoded” by the nucleic acid sequence (as meant herein) if it is altered due to post-translational modification so as to change its amino acid sequence. Thus, for example, if an amino acid sequence would be “encoded” by a nucleotide sequence except that an amino acid in the sequence is altered or deleted, it is considered herein to be “encoded” by the nucleotide sequence if the alteration or deletion can be shown to be due to post-translational modification. For example, a recombinant humanized IgG antibody produced in Chinese hamster ovary (CHO) cells will commonly lack the carboxy-terminal (C-terminal) lysine of the heavy chain, even though the nucleotide sequence encoding such an antibody may encode the C-terminal lysine. This lysine is usually removed post-translationally. Such an antibody is considered herein to be “encoded” by a nucleotide sequence that includes the C-terminal lysine.

An “Fc fragment,” “Fc region,” or “Fc portion,” as meant herein, consists essentially of a hinge domain (hinge), a second heavy chain constant domain (C_{H}2), and a C_{H}3 from an HC, although it may further comprise regions downstream from the C_{H}3 in some isotypes such as IgA or IgM.

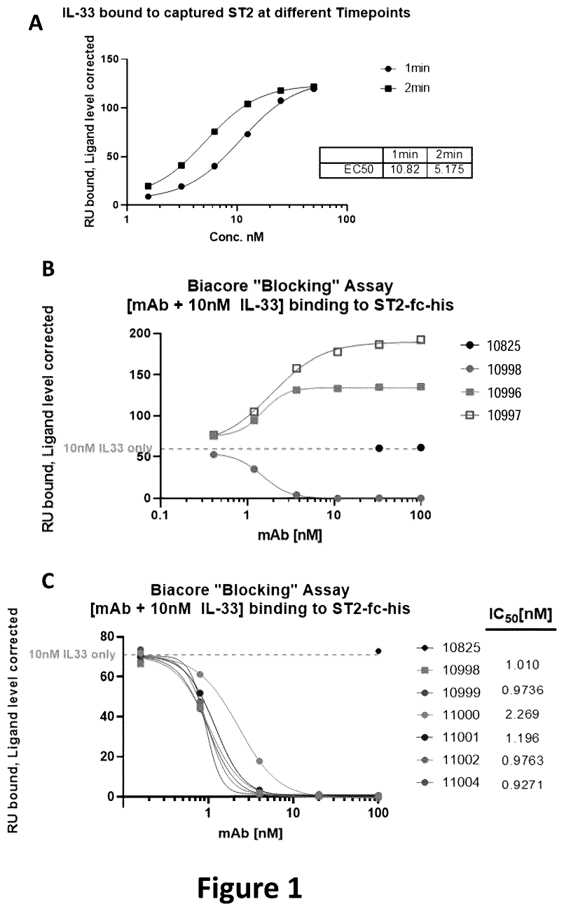

A “heavy chain (HC),” as meant herein, comprises at least a V_{H}, C_{H}1, hinge, C_{H}2, and C_{H}3. An HC including all of these domains could also be referred to as a “full-length HC” or, in some embodiments, an “IgG HC.” Some isotypes such as IgA or IgM can contain additional sequences, such as, for example, the IgM C_{H}4 domain. The numbering system of Kabat et al., supra, is used for the V_{H} (see FIG. 1), and the EU system (Edelman et al. (1969), Proc. Natl. Acad. Sci. USA 63:78-85, which is incorporated herein by reference in its entirety) is used for the C_{H}1, hinge, C_{H}2, and C_{H}3. The use of these well-known numbering systems can lead to a difference between an actual amino acid position in a sequence disclosed herein and a number assigned to that position using the Kabat or Edelman numbering system. However, one of skill in the art can assign a Kabat or Edelman number to any particular position in a disclosed antibody sequence with reference to knowledge in the art and to tables disclosed herein below showing how Kabat or Edelman numbers can be assigned with reference to the conserved features of antibody sequences, which can be located in disclosed sequences. Tables 1 and 2 below illustrate this numbering on generalized HC sequences.

TABLE 1
Consensus sequence of human V_{H}s

| | | | | | | | | | | | | | | |
|---|---|---|---|---|---|---|---|---|---|---|---|---|---|---|
| 1 | 2 | 3 | 4 | 5 | 6 | 7 | 8 | 9 | 10 | 11 | 12 | 13 | 14 | 15 |
| | | | L | | | | G | | | | | | P | |
| 16 | 17 | 18 | 19 | 20 | 21 | 22 | 23 | 24 | 25 | 26 | 27 | 28 | 29 | 30 |
| | S | V | | L | S | C | | | | G | | | | |
| | T | L | | V | T | | | | | | | | | |
| 31 | 32 | 33 | 34 | 35 | 35A | 35B | 36 | 37 | 38 | 39 | 40 | 41 | 42 | 43 |
| | | | | | | | W | | R | Q | | | G | K |
| | | | | | | | | | | | | | | Q |
| 44 | 45 | 46 | 47 | 48 | 49 | 50 | 51 | 52 | 52A | 52B | 52C | 53 | 54 | 55 |
| G | L | | W | | | | | | | | | | | |
| 56 | 57 | 58 | 59 | 60 | 61 | 62 | 63 | 64 | 65 | 66 | 67 | 68 | 69 | 70 |
| | | | | | | | | | | R | | | | |
| 71 | 72 | 73 | 74 | 75 | 76 | 77 | 78 | 79 | 80 | 81 | 82 | 82A | 82B | 82C |
| | | | S | | | | | | L | | | | | |
| 83 | 84 | 85 | 86 | 87 | 88 | 89 | 90 | 91 | 92 | 93 | 94 | 95 | 96 | 97 |
| | | | D | | | | Y | | C | | | | | |
| 98 | 99 | 100 | 100A | 100B | 100C | 100D | 100E | 100F | 100G | 100H | 100I | 100J | | |
| 100K | 101 | 102 | 103 | 104 | 105 | 106 | 107 | 108 | 109 | 110 | 111 | 112 | 113 | |
| | | | W | | Q | G | | | V | | V | S | (SEQ ID NO: 45) | |

This table shows “invariant” (according to Kabat et al., supra) amino acids based on the human V_{H} amino acid sequences (subgroups I-III) in Kabat et al. (supra). Numbering is according to Kabat et al., supra. Site numbers within the CDRs are written in bold italics. Position numbers with letters after them, e.g., 100A, with the exception of 82A-82C, may or may not be filled by an amino acid due to the varying lengths of CDRs. Positions 82A-82C, which are in a framework region, are almost always filled by an amino acid in a human V_{H} of subgroups I-III. A single boldface amino acid at a particular position indicates an “invariant” amino acid in all three of subgroups I-III of human V_{H}s as described by Kabat et al. (supra). Positions where no amino acid is designated did not meet this criterion.

Table 1 shows that there are numerous conserved amino acids having conserved spacing that would allow alignment of any V_{H} sequence with the conserved amino acids spaced as shown above by eye. Alternatively, a novel sequence could be aligned with a known V_{H} sequence using alignment software, for example, alignment software available on the International ImMunoGeneTics (IMGT) Information System® (for example, IMGT/DomainGapAlign, which is available at http://www.imgt.org or CLUSTAL Omega (Sievers et al., (2011), Fast, scalable generation of high-quality protein multiple sequence alignments using Clustal Omega, Molecular Systems Biology 7 (1): 539).

Table 2 below shows an alignment of human IgG Fc regions of the four human IgG subclasses, IgG1, IgG2, IgG3, and IgG4. This alignment shows the differences between these subclasses, as well as the high sequence conservation.

```
TABLE 2
IgG1 -----------------------------------------------
IgG2 -----------------------------------------------
IgG3 ELKTPLGDTTHTCPRCPEPKSCDTPPPCPRCPEPKSCDTPPPCERCP
IgG4 -----------------------------------------------
          216 226 236 246 256 266
          * * * * * *
IgG1 EPKSCDKTATCPPCPAPELLGGPSVFLFPPKPKDTLMISRTPEVTCVVVDVSHEDPEVKF
IgG2 ERKCCVE---CPPCPAPPVA-GPSVFLFPPKPKDTEMISRTPEVTCVVVDVSHEDPEVQF
IgG3 EPKSCDTPPPCPRCPAPELLGGPSVFLEPPKFKDTIMISRTPEVTCVVVDVSHEDPEVQE
IgG4 ESKYG---PPCPSCPAPEELGGPSVFLFPPKPKDTLMISETPEVTCVVVDVSQEDPEVQF
          276 286 296 306 316 326
          * * * * * *
IgG1 NWYVDGVEVHNAKTKPREEQYNSTYRVVSVLTVLHQDWINGKEYRCKVSNKALPAPIEKT
IgG2 NWYVDGMEVHNAKIKPREEQFNSTERVVSVLTVVRQDWLNGKEYKCKVSNKGLPAPIEKT
IqG3 KWYVDGVEVENAKIKPREEQYNSTFRVVSVLTVLHQDWINGKEYECKVSNKALPAPIEKT
IgG4 NWYVDGVEVHNAKTKPREEQFNSTYRVVSVLTVLHQDWLNGKEYKCKVSNKGLPSSIEKT
          356 366 376 386 336 346
          * * * * * *
IgG1 ISKAKGQPREPQVYTLPPSREEMTKNOVSLTCLVKGFYPSDIAVEWESNGQPENNYHTTP
IgG2 ISKTKGQPREPQVYTLPPSREEMTKNQVSLTCLVKGFYPSDIAVEWESNGQPENNYKTTP
IgG3 ISKTKGQPREPQVYTLPESREEMTKNQVSLTCLVKGFYPSDIAVENESSGQPENNYNTTP
IgG4 ISKAKGQPREPQVYTLPPSQEEMTKNQVSLTCLVKGFYPSDIAVEWESNGQPENNYKTTP
          396 406 416 426 436 446
          * * * * * *
IgG1 PVLDSDGSFFLYSKLTVDKSRWQQGNVFSCSVMHEALHNEYTQKSLSLSPGK (SEQ ID NO: 46)
IgG2 PMLDSDGSFFLYSKLTVDKSRWQQGNVESCSVMHEALANHYTOKSLSLSPEK (SEQ ID NO: 47)
IgG3 PMLDSDGSFFLYSKLTVDKSRNQQGNIFSCSVMHEALHNRFTQKSLSLSPGK (SEQ ID NO: 48)
IgG4 PVLDSDGSFFLYSRLTVDKSRWQEGNVFSCSVMHEALHNEYTQKSLSLSLGK (SEQ ID NO: 49)
```

A “human,” nucleotide or amino acid sequence, protein, or antibody is one that occurs naturally in a human or one that is identical to such a sequence or protein except for a small number of mutations or alterations as explained below. Many human nucleotide and amino acid sequences are reported in, e.g., Kabat et al., supra, which illustrates the use of the word “human” in the art. A “human” amino acid sequence or antibody, as meant herein, can contain one or more insertions, deletions, or substitutions relative to a naturally occurring sequence, with the proviso that a “human” amino acid sequence does not contain more than 10 insertions, deletions, and/or substitutions of a single amino acid per every 100 amino acids. Similarly, a human nucleotide sequence does not contain more than 30 insertions, deletions, and/or substitutions of a single nucleotide per every 300 nucleotides. In the particular case of a V_{H} or V_{L} amino acid sequence (or a nucleotide sequence encoding such an amino acid sequence), the CDRs are expected to be extremely variable, and, for the purpose of determining whether a particular V_{H} or V_{L} amino acid sequence (or the nucleotide sequence encoding it) is a “human” sequence, the CDRs (or the nucleotides encoding them) are not considered part of the sequence.

A “heterodimer,” as meant herein, is a protein dimer where the two proteins in the dimer have different amino acid sequences. In the particular case of an IgG antibody having a heterodimeric HC/HC pair, the two different HCs in the heterodimeric pair have V_{H} domains having different amino acid sequences.

A “humanized” antibody, as meant herein, is an antibody where the antibody is of non-human origin but has been engineered to be human as much as possible, thereby hopefully reducing immunogenicity in humans while retaining antibody stability and functional properties such as binding. Generally, this means that most or all of the constant domains and the framework regions of the variable domains are human or nearly human sequences, while the CDRs originate from a different organism. However, merely grafting CDRs from, e.g., a mouse antibody, into a human framework may not produce an antibody with the desired properties, and further modification may be required. In recent years, a variety of approaches to streamline and improve the results of humanization have been developed. See, e.g., Choi et al. (2015), mAbs 7 (6): 1045-1057 and references cited therein. However, results of changes made in an effort to improve one or more properties of an antibody are not fully predictable, mainly due to the high flexibility of CDR3 loops. See, e.g., dos Santos et al. (2018), Advances and challenges in therapeutic monoclonal antibodies drug development. Braz. J. Pharm. Sci. 54 (Special): e01007.

An “IgG antibody,” as meant herein, comprises (1) two HCs, each comprising a V_{H}, a C_{H}1, a hinge domain, a C_{H}2, and a C_{H}3 and (2) two light chains (LCs), each comprising a V_{L} and a LC constant domain (C_{L}). The heavy chain constant domains of an IgG antibody are of an IgG isotype, for example, IgG1, IgG2, IgG3, or lgG4 subclass of IgG. These domains are described in, e.g., Kabat et al., supra, pp. xv-xix and 647-699, which pages are incorporated herein by reference. The numbering system of Kabat et al., supra, is used for V_{H}s and V_{L}s (se Tables 1 and 2 herein). The EU system (Edelman et al. (1969), Proc. Natl. Acad. Sci. USA 63:78-85, which is incorporated herein in its entirety) is used for C_{L}s, C_{H}1s, hinges, C_{H}2s, and C_{H}3s. In some embodiments, some portions of the constant domains an IgG antibody may be of one subclass, for example IgG4, and another portion of the same antibody may be of another subclass, for example IgG1. Such antibodies are still IgG antibodies as meant herein. Further, the amino acid sequences of constant domains of an IgG antibody, as meant herein, can diverge from naturally occurring sequences to a limited extent without changing the antibody into something other than an IgG antibody, as meant herein. For example, an IgG antibody can comprise a C_{H}1 to C_{H}3 fragment that comprises no more than 24, 20, 16, 14, 12, ten, nine, eight, seven, six, five, four, three, two, or one amino acid substitution(s), deletion(s), or insertion(s) relative to a naturally occurring IgG amino acid sequence. However, the IgG subclass of an IgG antibody may or may not vary over the length of the C_{H}1 to C_{H}3 fragment such that, for example, a portion of the amino acid sequence of this fragment can be compared to, e.g., an IgG1 antibody sequence and another portion of the amino acid sequence can be compared to, e.g., an IgG4 antibody sequence for the purpose of determining whether an antibody is an IgG antibody as meant herein.

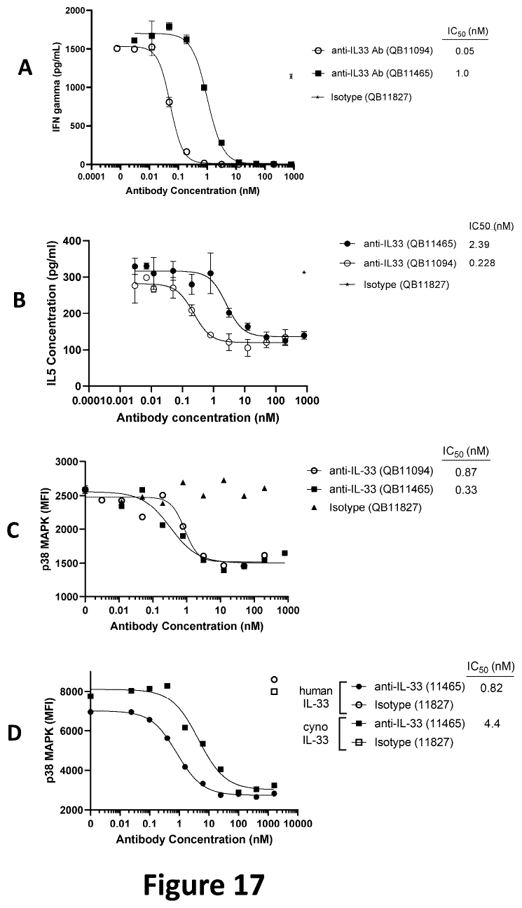

“Inhibition” of the interaction or binding of hIL33 with hST2 and/or with ST2/IL1AcP complex by an antibody, as meant herein, can be determined as set forth in Example 13 using the measurement of IFNγ cytokine secretion from primary human NK cells (FIG. 17A), IL-5 secretion from primary human ILC2 cells (FIG. 17B), and the level of p38 MAPK phosphorylation of primary human ILC2 cells (FIGS. 17C, 17D).

Likewise, “inhibition” of the interaction or binding of hTSLP to hTSLPR (human Thymic stromal lymphopoietin-receptor); the interaction of hTSLP with hIL7Rζ; and/or the interaction of hTSLP with an hTSLPR/hIL7Rα complex can be determined by the Biacore assays set forth in Example 8; and the cell-based assays set forth herein in Examples 14-18.

A “light chain (LC),” as meant herein, comprises a V_{L} and a C_{L}, which can be a kappa (C_{L}κ) or lambda (C_{L}λ) domain. These domains, including exemplary amino acid sequences thereof, are described in, e.g., Kabat et al., supra, pages xiii-lix, 103-309, and 647-660, which are incorporated herein by reference. The numbering system used herein for the V_{L} is that described in Kabat et al., supra, and the EU numbering system used for the C_{L} is that described in Edelman et al., supra. Tables 3 and 4 below illustrate the application of these systems to a variety of light chain sequences. One of skill in the art can use such information to assign Kabat or Edelman numbers to particular positions in the sequences disclosed herein.

TABLE 3
Consensus sequence of human V_{L}s

| | | | | | | | | | | | | | | | | | |
|---|---|---|---|---|---|---|---|---|---|---|---|---|---|---|---|---|---|
| 1 | 2 | 3 | 4 | 5 | 6 | 7 | 8 | 9 | 10 | 11 | 12 | 13 | 14 | 15 | | | |
| 16 | 17 | 18 | 19 | 20 | 21 | 22 | 23 | 24 | 25 | 26 | 27 | 27A | 27B | 27C | 27D | 27E | 27F |
| G | | | | | | | C | | | | | | | | | | |
| 28 | 29 | 30 | 31 | 32 | 33 | 34 | 35 | 36 | 37 | 38 | 39 | 40 | 41 | 42 | 43 | 44 | |
| | | | | | | | W | | | | | | | | A | P | |
| | | | | | | | | | | | | | | | S | | |
| | | | | | | | | | | | | | | | P | | |
| 45 | 46 | 47 | 48 | 49 | 50 | 51 | 52 | 53 | 54 | 55 | 56 | 57 | 58 | 59 | 60 | | |
| | | | | | | | | | | | | | I/V | P | | | |
| 61 | 62 | 63 | 64 | 65 | | 66 | 67 | 68 | 69 | 70 | 71 | 72 | 73 | 74 | 75 | | |
| R | F | S | G | S | | | | | | | | | L | | | | |
| 76 | 77 | 78 | 79 | 80 | 81 | 82 | 83 | 84 | 85 | 86 | 87 | 88 | 89 | 90 | | | |
| | | | | | | | | A/G | | Y | Y/F | | | | | | |
| 91 | 92 | 93 | 94 | 95 | 95A | 96 | 97 | 98 | 99 | 100 | 101 | 102 | 103 | 104 | | | |
| | | | | | | | | F | G | Q/G | G | T | | | | | |
| 105 | 106 | 106A | 107 | 108 | 109 | | | | | | | | | | | | |
| | | | | | (SEQ ID NO: 50) | | | | | | | | | | | | |

The numbering is according to Kabat et al. (supra). Numbers in bold italics indicate the positions of the CDRs. Position numbers with letters after them, e.g., 27A, may or may not be filled by an amino acid, due to the varying lengths of CDRs. Invariant residues for all human light chains in Kabat et al. (supra) are shown as bold letters indicating the amino acid found at that position. At selected sites, one or two amino acids commonly found at that site are indicated in plain text. In addition, many other amino acids are invariant or highly conserved within some subgroups of kappa or lambda V_{L}s, which can aid in categorizing a particular amino acid sequence as a V_{L}. Sites selected for alteration in PCT/US2018/089293 or in PCT/US2017/030676, are indicated by boldface underlined type.

TABLE 4
Consensus sequence and numbering for C_{L}s

| | | | | | | | | | | | | | | | | |
|---|---|---|---|---|---|---|---|---|---|---|---|---|---|---|---|---|
| | 108 | 109 | 110 | 111 | 112 | 113 | 114 | 115 | 116 | 117 | 118 | 119 | 120 | 121 | 122 | 123 |
| κ | | | | | | P | | | | I | | P | P | | | |
| λ | | | | | | P | | | | L | | P | P | | | |
| | 124 | 125 | 126 | 127 | 128 | 129 | 130 | 131 | 132 | 133 | 134 | 135 | 136 | 137 | 138 | 139 |
| κ | | | | | | | | S | | V | C | | | | | |
| λ | | | | | | | | A | | V | C | | | | | |
| | 140 | 141 | 142 | 143 | 144 | 145 | 146 | 147 | 148 | 149 | 150 | 151 | 152 | 153 | 154 | 155 |
| κ | | P | | | | | V | | W | | | | | | | |
| λ | | | | | | | V | | W | | | | | | | |
| | 156 | 157 | 158 | 159 | 160 | 161 | 162 | 163 | 164 | 165 | 166 | 167 | 168 | 169 | 170 | 171 |
| κ | | | | | Q | | S | | T | | | | | | | |
| λ | | | | | E | | T | | P | | | | | | | |
| | 172 | 173 | 174 | 175 | 176 | 177 | 178 | 179 | 180 | 181 | 182 | 183 | 184 | 185 | 186 | 187 |
| κ | | | S | | S | S | T | L | T | L | | | | | | |
| λ | | | A/M | | S | S | Y | L | S | L | | | | | | |
| | 188 | 189 | 190 | 191 | 192 | 193 | 194 | 195 | 196 | 197 | 198 | 199 | 200 | 201 | 202 | 203 |
| κ | | | | | | | C | | | | H | | | | | |
| λ | | | | | | | \| C | | | | H | | | | | |
| | 204 | 205 | 206 | 207 | 208 | 209 | 210 | 211 | 212 | 213 | 214 | | | | | |
| κ | | | | | | F | | | | | C (SEQ ID NO: 51) | | | | | |
| λ | | | | | | V | | | | | C (SEQ ID NO: 52) | | | | | |

The numbering is according to Edelman et al. (supra), which is the same as the numbering of Kabat et al. (supra) for C_{L}s. The amino acids shown in bold below the numbers are “invariant” residues according to Kabat et al. (supra) from alignments of both kappa and lambda C_{L}s from a variety of species. As indicated at selected sites (131, 160, 162, 174, 176, and 178), amino acids conserved in the ten human kappa chains (top) and 28 human lambda chains (below) reported in Kabat et al. (supra) are shown in plain text. In cases where either of two different amino acids are found at one of these sites, the more common amino acid is shown prior to the less common, e.g., A/M. Bold underlined numbers indicate sites that were altered as reported in PCT/US2018/089293 or in PCT/US2017/030676. In addition, many other amino acids are invariant or highly conserved within some subgroups of C_{L}κ or C_{L}λ domains, which can aid in categorizing a particular amino acid sequence as a C_{L}. Positions where no amino acid is designated are not invariant.

A “MabPair” or a “MabPair mixture,” as used herein, refers to a pair of, i.e., two, antibodies that are produced in a culture of a single host cell line into which DNA encoding the antibodies has been introduced. The host cells produce only two major species of antibodies. For further description of how a MabPair is produced, refer to the description in U.S. Pat. No. 11,130,808, Examples 1, 2, 3, 4, 5, 6, and 7, and Figures described therein, all of which are incorporated herein by reference in their entirety for all purposes.

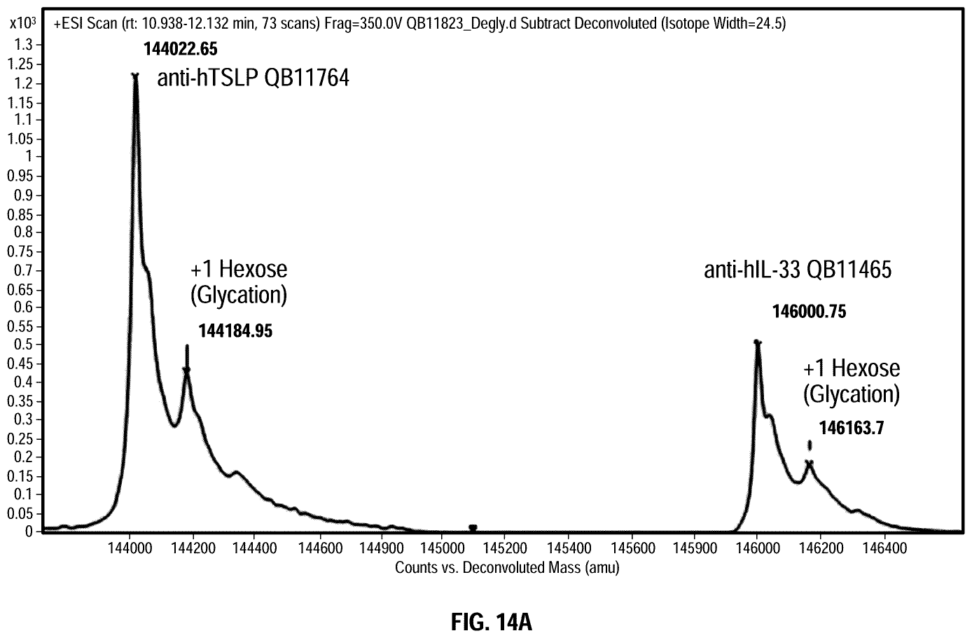

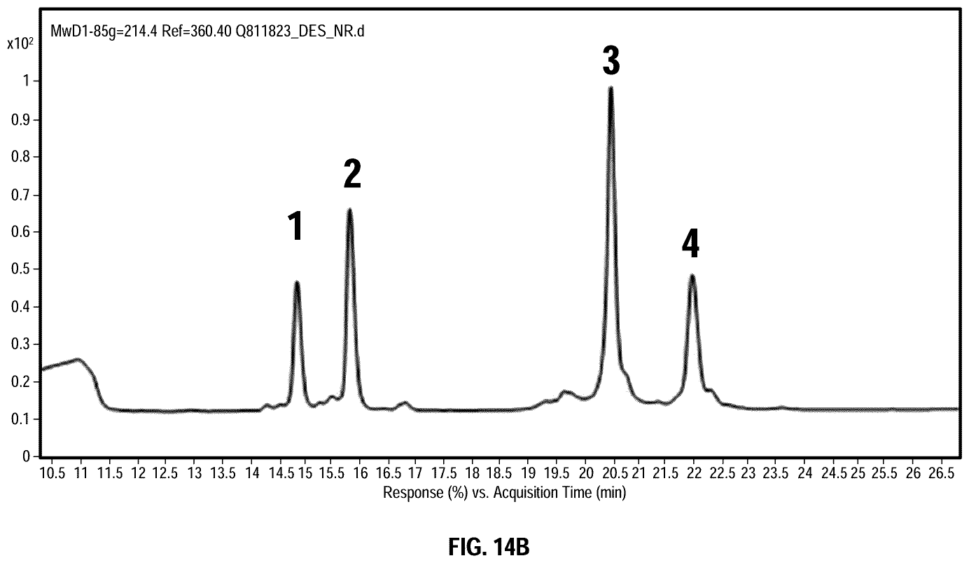

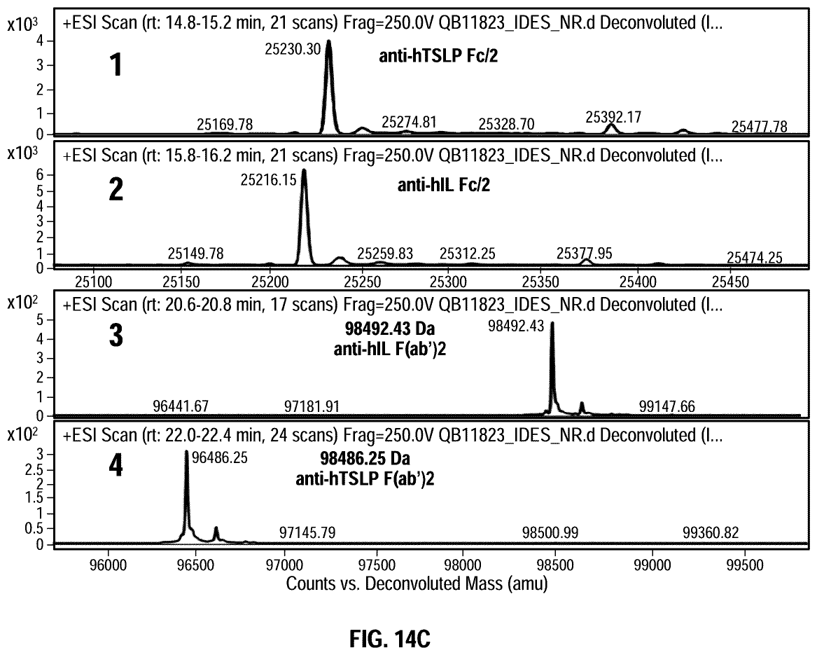

A “major species” of antibody in the context of a mixture of antibodies, as meant herein, is a particular antibody that makes up at least 10% of the total amount of antibodies within the mixture. To determine how many major species are in a mixture of antibodies, low pH cation exchange (CEX) chromatography as described in Example 5 and shown in FIG. 14 of U.S. Pat. No. 11,130,808 (which portions of U.S. Pat. No. 11,130,808 are incorporated herein by reference) can be performed. This method is described by Chen et al. (2010), Protein Science, 19:1191-1204, which is incorporated herein in its entirety. Briefly, it employs a Thermo ProPac™ WCX-10 weak CEX column, 4×250 mm, preceded by a 50 mm guard column (ProPac™ WCX-10G) using a Waters Alliance 2695 high performance liquid chromatography (HPLC) system. Chromatography can be run with a linear gradient from 100% Buffer A (20 mM sodium acetate pH 5.2) to 100% Buffer B (20 mM sodium acetate with 250 mM sodium chloride pH 5.2) over 30 minutes. The column can be washed with high salt (1M sodium chloride) and re-equilibrated to starting condition of Buffer A. Antibodies can be detected in the column outflow by absorbance at 214 nm. Relative amounts of the detected peaks can be determined using EMPOWER™ software (Waters Corp., Milford, MA, USA). Low pH CEX can distinguish between different full-length antibody species and can be used to quantitate relative amounts of specific antibody species in a mixture.

A “minor species” of antibody within a mixture of antibodies, as meant herein, comprises less than 10% of the total amount of antibodies in the mixture. This can be determined by low pH CEX chromatography as described in the definition of “major species.”

The terms “nucleic acid” and “polynucleotide” are used interchangeably herein, as are “nucleic acid sequence,” “nucleotide sequence,” or “polynucleotide sequence.”

A “partner directing alteration,” as meant herein, is is a substitution, insertion, or deletion of a single amino acid at the HC/LC interface within a V_{H}, C_{H}1, V_{L}, or C_{L} amino acid sequence, optionally a substitution of a charged amino acid or a cysteine for the naturally occurring amino acid, which causes an HC and LC, optionally a human and/or primate HC and LC, to associate more strongly. More specifically, an “HC partner-directing alteration” is an alteration in a V_{L} or C_{L} that can, sometimes only in the presence of an “LC partner-directing alteration” at a “contacting” residue in a V_{H} or C_{H}1, cause an HC and LC to associate more strongly. Similarly, an “LC partner-directing alteration” is an alteration in a V_{H} or C_{H}1 that can, sometimes only in the presence of an “HC partner-directing alteration” at a “contacting” residue in a V_{L} or C_{L}, cause an HC and LC to associate more strongly. In some embodiments, a contacting pair of HC and LC partner-directing alterations can be substitutions of charged amino acids having opposite charges. In other embodiments, a charged amino acid already exists at one of the contacting sites of the HC or LC so that alteration of only one chain is required to create a pair of oppositely charged residues at contacting sites in a cognate HC/LC pair, i.e., a charge pair. In other embodiments, cysteine residues can be introduced at contacting sites so that disulfide bridges in a cognate HC/LC pair can form. In further embodiments, HC- and LC-partner-directing alterations can be substitutions or pre-existing amino acids that create a knob and a hole (or a protuberance and a cavity) at contacting residues as described in U.S. Pat. No. 8,679,785, the relevant portions of which are incorporated herein by reference. The HC can be of the IgG, IgA, IgD, IgM, or IgE isotype, optionally IgG1, IgG2, IgG3, or IgG4. HC- and LC-partner-directing alterations occur at contacting amino acid positions that form part of the HC/LC interface. Interface residues in the C_{L}s and C_{H}1s include those within 4.5 Å, as explained in U.S. Pat. No. 8,592,562, Tables 4 and 5 and accompanying text in columns 10 and 11, all of which is incorporated herein by reference. These positions in human C_{H}1s and C_{L}s are catalogued in Table 5 below.

TABLE 5
Contacting residues between C_{H}1 and C_{L}

| C_{H}1 residue | C_{L}κ residue | C_{L}λ residue |
|---|---|---|
| 125 | 123 | 119 |
| 126 | 121, 123, 124 | 117, 119, 120 |
| 127 | 121 | 117, 119 |
| 128 | 118, 133 | 114, 129 |
| 129 | 118 | 114 |
| 130 | 118 | |
| 139 | 116 | |
| 140 | 116 | |
| 141 | 116, 118, 135 | 112, 114 |
| 142 | 118 | 114 |
| 143 | | 114 |
| 145 | 124, 131 | 127, 129, 173 |
| 147 | 124, 131 | 125, 127 |
| 148 | | 125 |
| 168 | 137, 138, 174 | 133, 163, 169 |
| 169 | 164 | |
| 170 | 135, 162, 164, 174, 176 | 131, 133, 169, 171 |
| 171 | 162, 164 | 158, 161, 171 |
| 172 | | 158 |
| 173 | 160, 162 | 156, 158, 173 |
| 174 | 160 | 156 |
| 175 | 160 | 156 |
| 176 | | 156 |
| 181 | | 173 |
| 182 | | 173 |
| 183 | 176 | 129, 131, 173 |
| 185 | 135 | 114, 131 |
| 187 | 137 | |
| 213 | 123 | 119 |
| 218 | 122 | |

In the particular case of contacting residues on the interface between a V_{H} and a V_{L}, pairs of residues, one in the V_{H} and one in the V_{L}, suitable for alteration can be selected using the following criteria: (1) the residues are buried or partially buried, i.e., inaccessible in the tertiary structure of a full-length antibody, (2) the residues are spatially close, that is, where the Cα (Cα is the central carbon of an amino acid, to which the amino group, the carboxyl group, and the side chain are attached) of the two amino acids are within about 12 Å, or where there is at most 5.5 Å between a side chain heavy atom (any atom other than hydrogen) of one amino acid and any heavy atom of the other amino acid according to known structure models, (3) the residues are highly conserved, although they need not be totally invariant, and (4) the residues are not within or interacting with the CDRs. Examples of such contacting residues include, without limitation, the following: position 44 (V_{H}) and position 100 (V_{L}); position 39 (V_{H}) and position 38 (V_{L}); and position 105 (V_{H}) and position 43 (V_{L}).

To a first approximation, a change in the strength of HC/LC association due to HC- and/or LC-partner-directing alterations can be determined by “chain drop out” experiments as described in Example 11 of U.S. Pat. No. 11,124,470 and Figures referred to therein and in Example 3 of U.S. Pat. No. 11,130,808 and Figures referred to therein, all of which is incorporated herein by reference for all uses.

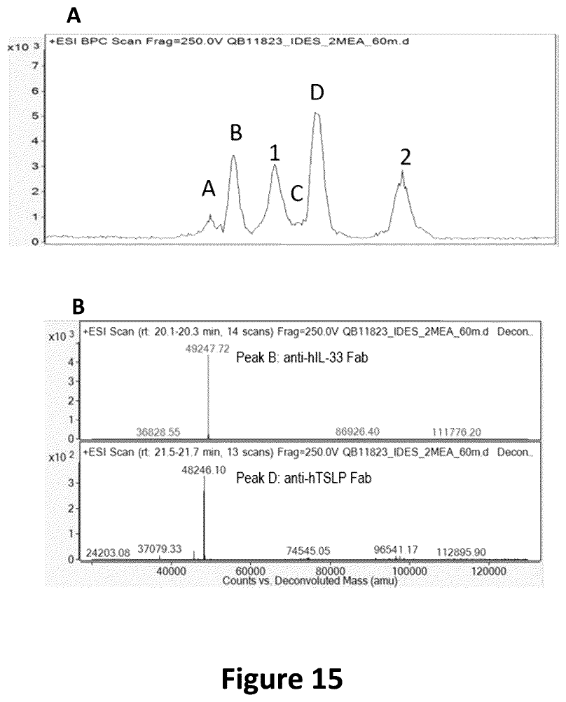

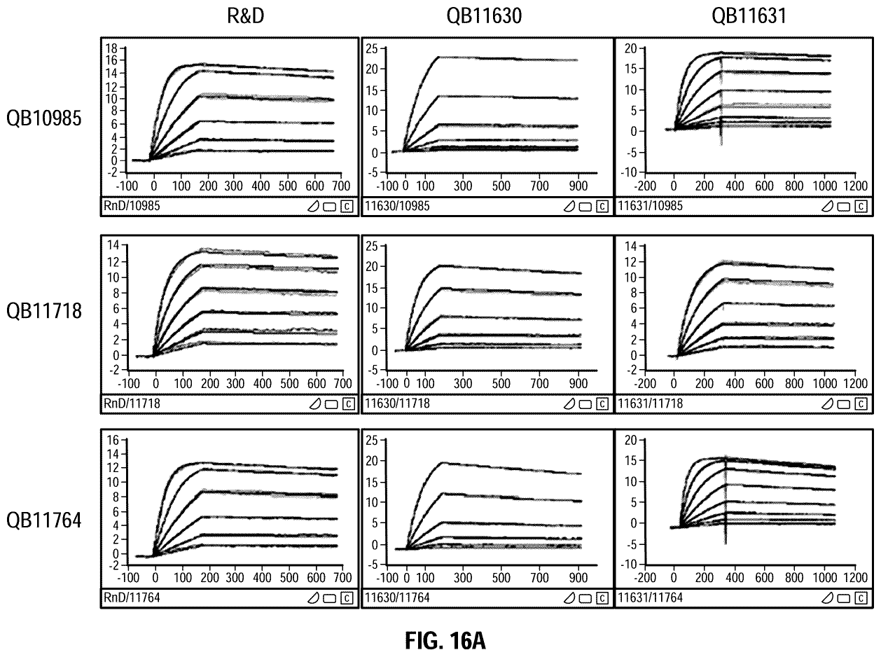

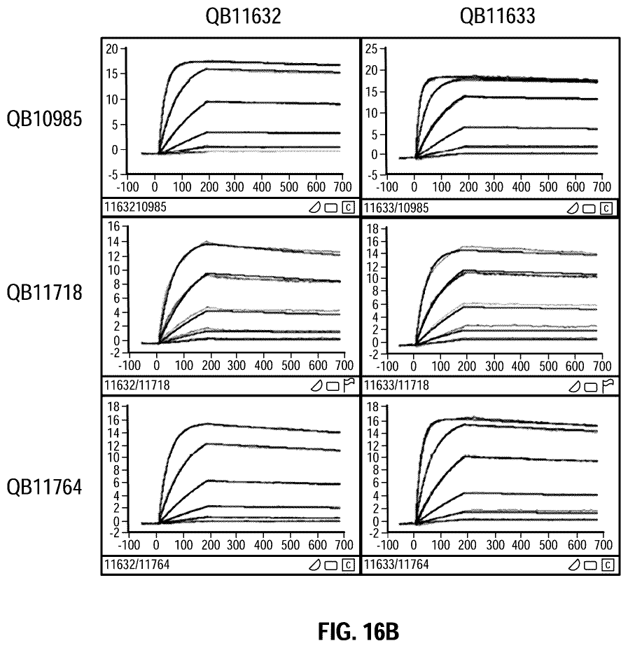

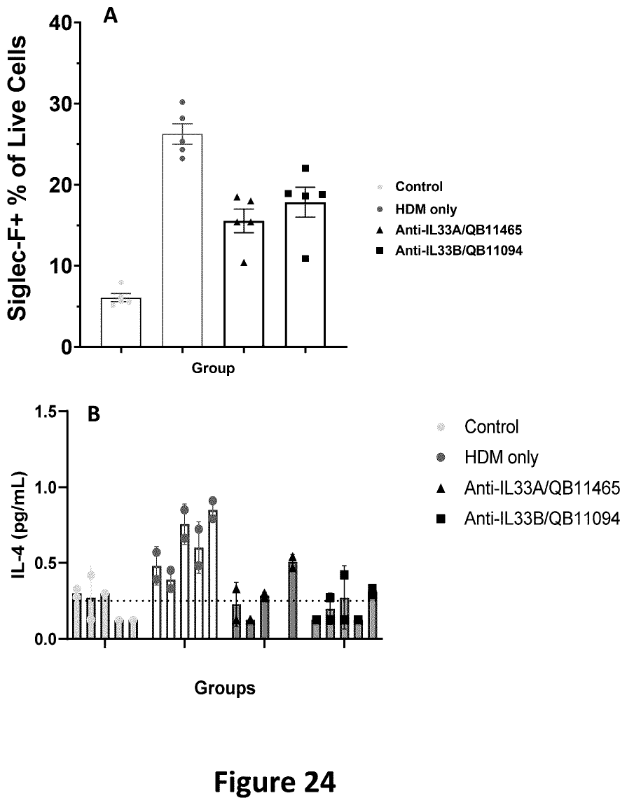

To confirm or, in some cases, clarify results from chain drop out experiments, the sizes Fab fragments arising in transfectants containing DNAs encoding the HC and LC of a first antibody (Mab1) and the HC and LC of a second antibiody (Mab2) can be determined by mass spectrometry as described in Example 12 and FIG. 24 herein, in Thompson et al. (2014), mAbs 6:1, 197-203 (which is incorporated herein in its entirety), and in FIG. 15 and in Example 5 of U.S. Pat. No. 11,130,808 (which are incorporated herein by reference). In most cases, cognate and non-cognate pairs can be distinguished by mass using such techniques. If non-cognate pairs are major species in cells transfected with DNAs encoding an unaltered Mabl HC and LC and an unaltered Mab2 HC and LC and are not major species in cells transfected with DNAs encoding Mab1 HC and LC and Mab2 HC and LC, wherein at least one of these antibodies comprises a partner-directing alteration, then it is considered herein that at least one of the alterations is a favorable partner-directing alteration.

Examples of partner-directing alterations include alterations that create, partially or wholly, any of the following charge pairs: 44D/E (V_{H}) and 100R/K (V_{L}); 44R/K (V_{H}) and 100D/E (V_{L}); 105R/K (V_{H}) and 43D/E (V_{L}); 105D/E (V_{H}) and 43R/K (V_{L}); 147D/E (C_{H}1) and 131R/K (C_{L}); 147R/K (C_{H}1) and 131D/E (C_{L}); 168D/E (C_{H}1) and 174R/K (C_{L}); 168R/K (C_{H}1) and 174D/E (C_{L}); 181R/K (C_{H}1) and 178E/D (C_{L}); and 181E/D (C_{H}1) and 178R/K (C_{L}). In addition, partner-directing alterations include substitutions where cysteine is substituted for another amino acid such that contacting pairs of cysteines exist in the HC and LC of the antibody, for example any of the following pairs: 126C (C_{H}1) and 121C (C_{L}); 126C (C_{H}1) and 124C (C_{L}); 127C (C_{H}1) and 121C (C_{L}); 128C (C_{H}1) and 118C (C_{L}); 133C (C_{H}1) and 117C (C_{L}); 133C (C_{H}1) and 209C (C_{L}); 123C (C_{H}1) and 116C (C_{L}); 141C (C_{H}1) and 116C (C_{L}); 168C (C_{H}1) and 174C (C_{L}); 170C (C_{H}1) and 162C (C_{L}); 183C (C_{H}1) and 176C (C_{L}); 173C (C_{H}1) and 160C (C_{L}); 170C (C_{H}1) and 176C (C_{L}); and 173C (C_{H}1) and 162C (C_{L}).

A “primate,” nucleotide or amino acid sequence or a protein is one which occurs naturally in nucleic acids or proteins found in a primate or one that is identical to such a sequence or protein except for a small number of alterations as explained below. Primates include animals from a number of families including, without limitation, prosimians (including lemurs), new world monkeys, chimpanzees, humans, gorillas, orangutans, gibbons, and old world monkeys. Specific primate species include, without limitation, Homo sapiens, Macaca mulata (rhesus macaque), Macaca fascicularis (cynomolgus monkey), and Pan troglodytes (chimpanzee), among many others. Many primate nucleotide and amino acid sequences are known in the art, e.g., those reported in Kabat et al., supra. Generally, a “primate” amino acid sequence, as meant herein, can contain one or more insertions, deletions, or substitutions relative to a naturally occurring primate sequence, with the proviso that a “primate” amino acid sequence does not contain more than 10 insertions, deletions, and/or substitutions of a single amino acid per every 100 amino acids. Similarly, a primate nucleotide sequence does not contain more than 30 insertions, deletions, and/or substitutions of a single nucleotide relative to a naturally occurring primate sequence per every 300 nucleotides. In the particular case of a V_{H} or V_{L} sequence, the CDRs are expected to be extremely variable, and, for the purpose of determining whether a particular V_{H} or V_{L} amino acid sequence (or the nucleotide sequence encoding it) is a “primate” sequence, the CDRs (or the nucleotides encoding them) are not considered part of the sequence.

A “signal peptide,” as meant herein is amino acid sequence, in many cases an amino-terminal sequence, on a protein which, in conjunction with a signal recognition particle, targets the protein to the endoplasmic reticulum in eukaryotes (and possibly on to the cell surface) or the plasma membrane in prokaryotes. See, e.g., Hegde and Bernstein (2006), Trends in Biochemical Sciences 31 (6): 563-571. Although primary sequences of signal peptides are somewhat variable, many are known in the art. N-terminal signal peptides are often cleaved from the protein in its mature form.

A “targeted biologic,” as meant herein, is a protein that can influence an aspect of a cell's biological status via its interaction with another specific molecule (which can be a protein). For example, a “targeted biologic” may influence a cell's ability to live, to proliferate, to produce specific cytokines or proteins, etc. As an example, the anti-hIL33 and anti-TSLP antibodies described herein are “targeted biologics” since they interact with hIL33 and hTSLP, which causes a number of biological effects as described in the Examples herein.

Similarly, a “targeted inhibitor,” as meant herein, is small molecule that can influence an aspect of a cell's biological status via its interaction with a specific cellular molecule (which can be a protein). For example, a “tyrosine kinase inhibitor” is a small molecule that affects the activity of a tyrosine kinase (which can affect a variety of cell functions) via its interaction with the tyrosine kinase.

As meant herein, a “treatment” for a particular disease or condition refers to a course of action, which can comprise administration of one or more antibodies, polynucleotides encoding one or more antibodies, and/or one or more other molecules, that results in a lessening of one or more symptoms or a decrease or interruption in an expected progression of the disease or condition in a human patient, an animal model system considered to be reflective of the disease or condition, or an in vitro cell-based assay considered to be reflective of the disease or condition. This can be ascertained by an objective measurement of symptoms in humans or animals or by measurement of various parameters in cell-based assays, for example, production of one or more cytokines, e.g., IFNγ, cell proliferation, cell death, etc. For example, for a cancer “treatment,” the treatment can result in a decrease in tumor volume, an absence of expected tumor metastasis in a human or in an animal model system, an increase in survival time, or an increase in progression-free or disease-free survival time in a human or animal suffering from cancer. A cancer treatment may also result in an increase in indices indicating activation of some aspect of the immune system in a cell-based assay, for example, phagocytosis of cancer cells by macrophages, proliferation of T cells, and/or increased production of cytokines, e.g., type I IFN, IFNγ, and/or IL-2, by one or more cells types that play a role in the immune system.

### Anti-Il33 Antibodies

In one aspect, variable domains of anti-IL33 Monoclonal antibodies (Mabs) are provided herein that have unique amino acid sequences; including those sequences set forth in the Sequence Listing. As shown in the Examples below, these Monoclonal antibodies (Mabs) can bind to antigens encoded by human and cynomolgus monkey alleles of IL33, that is, the IL33 protein, and can inhibit the interaction of human IL-33 and ST2/IL1AcP complex. Interleukin 33 (IL-33) is a member of the IL-1 family of cytokines. IL-33 binding the IL-33 receptor (consisting of ST2 and IL-IRAcP) can promote a pro-inflammatory response. IL-33 is secreted as a full length 270 amino pro-form that can be cleaved by proteases secreted by mast cells. In particular embodiments, the anti-IL33 antibodies provided herein inhibit the interaction of IL-33 with ST2 and/or block the binding of IL-33 with ST2.

In one aspect these antibodies can be, for example, human, humanized, or primate IgG antibodies, which can be IgG1, IgG2, IgG3, or IgG4 antibodies. In one aspect, the antibodies are human or humanized IgG1 antibodies.

In one aspect, a VH of an anti-hIL33 antibody contains a VH CDR1, a VH CDR2, and a VH CDR3 which comprise the amino acid sequences SEQ ID NO:80 (CDR1), SEQ ID NO: 81 (CDR2), and SEQ ID NO: 82 (CDR3). Antibodies comprising a VH which comprises any one of these sets of CDR sequences can inhibit the interaction of human IL-33 and ST2/IL1AcP complex.

Further, a VH of an anti-hIL33 antibody can comprise the amino acid sequence of any one of SEQ ID NOs: 29, 22, 6, 10, 14, or 18; or can comprise slightly altered versions of these sequences. For example, a VH can comprise one or more partner-directing alteration(s), which can be (an) amino acid substitution(s) relative to any one of SEQ ID NOs: 29, 22, 6, 10, 14, and/or 18. In some embodiments, a VH can comprise no more than 6, 5, 4, 3, 2, or 1 amino acid alterations relative to any one of any one of SEQ ID NOs: 29, 22, 6, 10, 14, or 18. These amino acid alterations can be substitutions and/or can be partner-directing alterations. In further embodiments, such alterations occur only in framework regions and do not occur in CDRs. VHs comprising such altered sequences can be part of antibodies that can inhibit the interaction of human IL-33 and ST2/IL1AcP complex.

Similarly, a VL of an anti-hIL33 antibody can comprise a VL CDR1, VL CDR2 and VL CDR3, which comprise the amino acid sequences of SEQ ID NO: 77 (CDR1), SEQ ID NO:78 (CDR2), and SEQ ID NO: 79 (CDR3). Antibodies comprising a VH which comprises any one of these sets of CDR sequences can inhibit the interaction of human IL-33 and ST2/IL1AcP complex.

Further, a VL of an anti-hIL33 antibody can comprise the amino acid sequence of any one of SEQ ID NOs: 25, 20, 4, 8, 12, and 16 or can comprise slightly altered versions of these sequences. For example, a VL can comprise one or more partner-directing alteration(s), which can be (an) amino acid substitution(s) relative to any one of SEQ ID NOs: 25, 20, 4, 8, 12, and/or 16. In some embodiments, a VL can comprise no more than 6, 5, 4, 3, 2, or 1 amino acid alterations relative to any one of any one of SEQ ID NOs: 25, 20, 4, 8, 12, and 16. These amino acid alterations can be substitutions and/or can be partner-directing alterations as set forth in U.S. Pat. No. 11,124,570, and the like. VLs comprising such altered sequences can be part of antibodies that can inhibit the interaction of human IL-33 and ST2/IL1AcP complex.

In another aspect, an anti-hIL33 antibody can comprise a VH CDR1, VH CDR2, VH CDR3, VL CDR1, VL CDR2, and VL CDR3, which have the amino acid sequences of SEQ ID NOs: 80, 81, 82, 77, 78, and 79. Antibodies comprising such sets of CDR sequences can inhibit the interaction of human IL-33 and ST2/IL1AcP complex.

In a further aspect, the anti-hIL33 antibody VH and VL can comprise, respectively, the amino acid sequences of any one of the following groups of two amino acid sequences: SEQ ID NOs: 29 (VH) and 25 (VL); SEQ ID NOs: 22 (VH) and 20 (VL); SEQ ID NOs: 6 (VH) and 4 (VL); SEQ ID NOs: 10 (VH) and 8 (VL); SEQ ID NOs: 14 (VH) and 12 (VL); and SEQ ID NOs: 18 (VH) and 16 (VL). In some embodiments, the VH and VL can comprise slightly altered versions of one of these groups of two sequences. For example, a VH and can comprise one or more partner-directing alteration(s), which can be (an) amino acid substitution(s), relative to one sequence in one of the groups of two sequences, and the VL can comprise one or more partner-directing alteration(s), which can be (an) amino acid substitution(s), relative to the other sequence in the same group of two sequences. A VH and VL that form an antibody or a portion thereof can each comprise an amino acid sequence which comprises no more than 6, 5, 4, 3, 2, or 1 amino acid alterations, optionally substitutions, relative to the first (VH) and the second (VL) amino acid sequence in a group of two sequences. These alterations can be partner-directing alterations. VHs and VLs comprising such altered sequences can be part of antibodies that can inhibit the interaction of human IL-33 and ST2/IL1AcP complex.

In a particular embodiment, the VH CDR1, VH CDR2, VH CDR3, VL CDR1, VL CDR 2, and VL CDR3 of the anti-hIL33 antibody comprise, respectively, the amino acid sequences of SEQ ID NOs: 80, 81, 82, 77, 78, and 79; the VH comprises an amino acid sequence which comprises no more than four alterations relative to the amino acid sequence of SEQ ID NO: 29, and the VL comprises an amino acid sequence which comprises no more than four alterations relative to the amino acid sequence of SEQ ID NO: 25. In a more particular embodiment of an invention anti-hIL33 antibody, the HC comprises an amino acid sequence which comprises no more than four, three, two or one alteration(s) relative to the amino acid sequence of SEQ ID NOs: 7, 11, 15, 19, 23, or 31, and the LC comprises an amino acid sequence which comprises no more than four, three, two or one alteration(s) relative to the amino acid sequence of SEQ ID NOs: 5, 9, 13, 17, 21, or 27. In yet a more particular embodiment of an invention anti-hIL33 antibody, the HC comprises the amino acid sequence of SEQ ID NO: 31, and the LC comprises the amino acid sequence of SEQ ID NO: 27.

In other aspects, an anti-hIL33 antibody can be part of a bispecific antibody that binds to hIL33 and another antigen. It is also contemplated herein that an invention anti-hIL33 antibody can have a different format, such as, for example, scFv, scFv-Fc, BiTE®, single domain antibodies, bispecific antibodies, Fab-scFv, DVD-IgG, IgG (H)-scFv, nanobody, nanobody-HAS, diabody, DART, TandAb, scDiabody, miniantibody, minibody, etc. See, e.g., Spiess et al. (2015), Molecular Immunology 67:95-106.

### Anti-Tslp Antibodies

In one aspect, variable domains of anti-TSLP Monoclonal antibodies (Mabs) are provided herein that have unique amino acid sequences; including those sequences set forth in the Sequence Listing. As shown in the Examples below, these Monoclonal antibodies (Mabs) can bind to antigens encoded by human and cynomolgus monkey alleles of TSLP, that is, the TSLP protein, and can inhibit the interaction (e.g., the binding) of hTSLP with hTSLPR or hTSLP with hIL7Rζ; and/or the interaction of hTSLP with an hTSLPR/hIL7Rα complex. Matured human TSLP (NCBI accession number: NP_149024.1) is a polypeptide consisting of 131 amino acids. It was well-known that 36NNT38 and 91NAT93 are N-glycosylated when the antigen is produced from mammalian cells.

In one aspect these antibodies can be, for example, human, humanized, or primate IgG antibodies, which can be IgG1, IgG2, IgG3, or IgG4 antibodies. In one aspect, the antibodies are human or humanized IgGI antibodies.

In one aspect, a VH of an anti-TSLP antibody contains a VH CDR1, a VH CDR2, and a VH CDR3 which comprise the amino acid sequences SEQ ID NO:86 or 94 (CDR1), SEQ ID NO: 87 or 95 (CDR2), and SEQ ID NO: 88 or 96 (CDR3). Accordingly, an anti-hTSLP antibody VH CDR1, VL CDR2, and VL CDR3 can comprise, respectively, the amino acid sequences of SEQ ID NOs: 86, 87 and 88; or SEQ ID NOs: 94, 95 and 96. Antibodies comprising a VH which comprises any one of these sets of CDR sequences can inhibit the interaction of hTSLP with hTSLPR or hTSLP with hIL7Rζ; and/or the interaction of hTSLP with an hTSLPR/hIL7Rα complex.

Further, a VH of an anti-hTSLP antibody can comprise the amino acid sequence of any one of SEQ ID NOs: 70, 65, 50, 43, or 39; or can comprise slightly altered versions of these sequences. For example, a VH can comprise one or more partner-directing alteration(s), which can be (an) amino acid substitution(s) relative to any one of SEQ ID NOs: 70, 65, 50, 43, and/or 39. In some embodiments, a VH can comprise no more than 6, 5, 4, 3, 2, or 1 amino acid alterations relative to any one of any one of SEQ ID NOs: 70, 65, 50, 43, or 39. These amino acid alterations can be substitutions and/or can be partner-directing alterations. In further embodiments, such alterations occur only in framework regions and do not occur in CDRs. VHs comprising such altered sequences can be part of antibodies that can inhibit the interaction of hTSLP with hTSLPR or hTSLP with hIL7Rζ; and/or the interaction of hTSLP with an hTSLPR/hIL7Rα complex.

Similarly, a VL of an anti-hTSLP antibody can comprise a VL CDRI, VL CDR2 and VL CDR3, which comprise the amino acid sequences of SEQ ID NO: 83 or 91 (CDR1), SEQ ID NO:84, 89 or 92 (CDR2), and SEQ ID NO: 85, 90 or 93 (CDR3). Accordingly, an anti-hTSLP antibody VL CDR1, VL CDR2, and VL CDR3 can comprise, respectively, the amino acid sequences of SEQ ID NOs: 83, 84, and 85; SEQ ID NOs: 83, 89 and 90; or SEQ ID NOs: 91, 92 and 93. Antibodies comprising a VH which comprises any one of these sets of CDR sequences can inhibit the interaction of hTSLP with hTSLPR or hTSLP with hIL7Rα; and/or the interaction of hTSLP with an hTSLPR/hIL7Rα complex.

Further, a VL of an anti-hTSLP antibody can comprise the amino acid sequence of any one of SEQ ID NOs: 74, 67, 54, 47, 45, and 41 or can comprise slightly altered versions of these sequences. For example, a VL can comprise one or more partner-directing alteration(s), which can be (an) amino acid substitution(s) relative to any one of SEQ ID NOs: 74, 67, 54, 47, 45, and/or 41. In some embodiments, a VL can comprise no more than 6, 5, 4, 3, 2, or 1 amino acid alterations relative to any one of any one of SEQ ID NOs: 74, 67, 54, 47, 45, and 41. These amino acid alterations can be substitutions and/or can be partner-directing alterations as set forth in U.S. Pat. No. 11,124,570, and the like. VLs comprising such altered sequences can be part of antibodies that can inhibit the interaction of human TSLP and TSLPR/IL7Rα complex.

In another aspect, an anti-hTSLP antibody can comprise a VH CDR1, VH CDR2, VH CDR3, VL CDR1, VL CDR2, and VL CDR3, which have the amino acid sequences of SEQ ID NOs: 86, 87, 88, 83, 84, and 85; SEQ ID NOs: 86, 87, 88, 83, 89, and 90; or SEQ ID NOs: 94, 95, 96, 91, 92, and 93. Antibodies comprising such sets of CDR sequences can inhibit the interaction of hTSLP with hTSLPR or hTSLP with hIL7Rα; and/or the interaction of hTSLP with an hTSLPR/hIL7Rα complex.

In a further aspect, the anti-hTSLP antibody VH and VL can comprise, respectively, the amino acid sequences of any one of the following groups of two amino acid sequences: SEQ ID NOs: 70 (VH) and 74 (VL); SEQ ID NOs: 65 (VH) and 67 (VL); SEQ ID NOs: 50 (VH) and 54 (VL); SEQ ID NOs: 43 (VH) and 47 (VL); SEQ ID NOs: 43 (VH) and 45 (VL); and SEQ ID NOs: 39 (VH) and 41 (VL). In some embodiments, the VH and VL can comprise slightly altered versions of one of these groups of two sequences. For example, a VH and can comprise one or more partner-directing alteration(s), which can be (an) amino acid substitution(s), relative to one sequence in one of the groups of two sequences, and the VL can comprise one or more partner-directing alteration(s), which can be (an) amino acid substitution(s), relative to the other sequence in the same group of two sequences. A VH and VL that form an antibody or a portion thereof can each comprise an amino acid sequence which comprises no more than 6, 5, 4, 3, 2, or 1 amino acid alterations, optionally substitutions, relative to the first (VH) and the second (VL) amino acid sequence in a group of two sequences. These alterations can be partner-directing alterations. VHs and VLs comprising such altered sequences can be part of antibodies that can inhibit the interaction of hTSLP with hTSLPR or hTSLP with hIL7Rα; and/or the interaction of hTSLP with an hTSLPR/hIL7Rα complex.

In a particular embotiment, the VH CDR1, VH CDR2, VH CDR3, VL CDR1, VL CDR 2, and VL CDR3 of the anti-hTSLP antibody comprise, respectively, the amino acid sequences of SEQ ID NOs: 94, 95, 96, 91, 92, and 93; the VH comprises an amino acid sequence which comprises no more than four alterations relative to the amino acid sequence of SEQ ID NO: 70, and the VL comprises an amino acid sequence which comprises no more than four alterations relative to the amino acid sequence of SEQ ID NO: 74. In a more particular embodiment of an invention anti-hTSLP antibody, the HC comprises an amino acid sequence which comprises no more than four, three, two or one alteration(s) relative to the amino acid sequence of SEQ ID NOs: 44, 52, 58, 62, 66, or 72, and the LC comprises an amino acid sequence which comprises no more than four, three, two or one alteration(s) relative to the amino acid sequence of SEQ ID NOs: 46, 48, 56, 60, 64, 68, or 76. In yet a more particular embodiment of an invention anti-hTSLP antibody, the HC comprises the amino acid sequence of SEQ ID NO: 72, and the LC comprises the amino acid sequence of SEQ ID NO: 76.

In yet other aspects, an anti-hTSLP antibody can be part of a bispecific antibody that binds to hTSLP and another antigen. It is also contemplated herein that an invention anti-hTSLP antibody can have a different format, such as, for example, scFv, scFv-Fc, BiTER, single domain antibodies, bispecific antibodies, Fab-scFv, DVD-IgG, IgG (H)-scFv, nanobody, nanobody-HAS, diabody, DART, TandAb, scDiabody, miniantibody, minibody, etc. See, e.g., Spiess et al. (2015), Molecular Immunology 67:95-106.

Additional Aspects of Anti-Hil33 and/or Anti-Htslp Antibodies

The anti-hIL33 and anti-hTSLP antibodies described herein can be human, humanized, or primate antibodies and/or IgG antibodies, for example IgG1, IgG2, IgG3, or IgG4 antibodies. Such IgG antibodies can comprise partner-directing alterations, such as HC and/or LC partner-directing alterations. Further, such IgG antibodies can comprise one or more alteration(s) that disfavor(s) heterodimers. Such alterations can improve the chances that a single host cell line into which DNAs encoding at least two different IgG antibodies have been introduced will produce no more than two or three different major species of antibodies.

The partner-directing alteration(s) can form part of charge pairs or pairs of contacting cysteines within an IgG anti-hIL33 and/or anti-hTSLP antibody as described herein, optionally a human or primate IgG antibody. For example, partner-directing alterations include alterations (including amino acid substitutions) that create, partially or wholly, any one of following charge pairs: 44D/E (VH) and 100R/K (VL); 44R/K (VH) and 100D/E (VL); 105R/K (VH) and 43D/E (VL); 105D/E (VH) and 43R/K (VL); 147D/E (CH1) and 131R/K (CL); 147R/K (CH1) and 131D/E (CL); 168D/E (CH1) and 174R/K (CL); 168R/K (CH1) and 174D/E (CL); 181R/K (CH1) and 178E/D (CL); and 181E/D (CH1) and 178R/K (CL). If a charged amino acid already exists at one of these sites, only one partner-directing alteration will be necessary to create the charge pair. In other situations, two partner-directing alterations, one in the HC and one in the LC, will be needed to create the charge pair. In addition, partner-directing alterations include substitutions where cysteine is substituted for another amino acid such that contacting pairs of cysteines are created, which can form disulfide bridges. In a human IgG1 antibody, these can include the following pairs: 126 (CH1) and 121 (CL), 170C (CH1) and 162C (CL), 170 (CH1) and 176 (CL), 173 (CH1) and 160 (CL), and 183 (CH1) and 176 (CL). In a human IgG4 antibody, these can include the following pairs: 170C (CH1) and 162C (CL), 173C (CH1) and 162C (CL), and 183 (CH1) and 176 (CL). The contacting cysteine pair in a cognate CH1/CL pair in one antibody in mixture of antibodies can be located at a different position than that of the other antibody in the mixture, which can increase the selectivity in formation of cognate HC/LC pairs. U.S. Pat. No. 11,130,808, including those portions that describe partner-directing alterations, including Examples 1-3 and 5, and figures referred to therein, are incorporated herein by reference for all purposes.

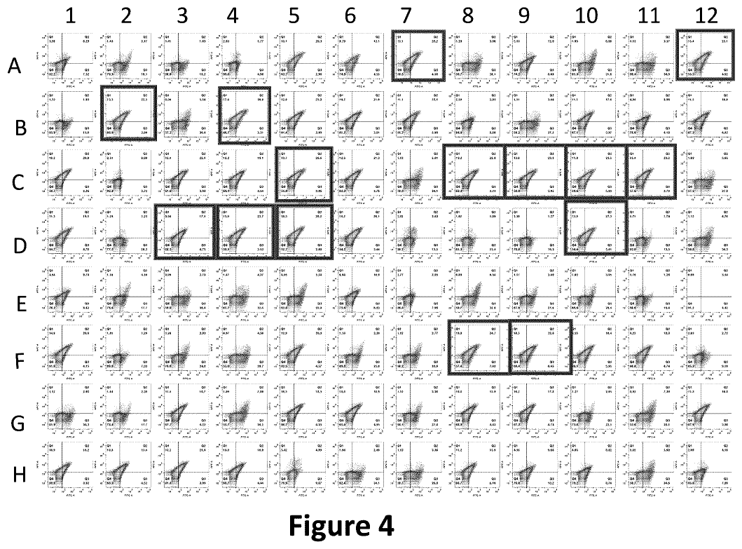

Well known methods can be used to create DNAs encoding HCs and/or LCs containing partner-directing alterations. Such methods are described in U.S. Pat. No. 11,130,808 and include methods such as artificial synthesis of DNA sequences (for example by commercial vendors such as, e.g., Integrated DNA Technologies, Coralville, Iowa, USA or Genewiz, South Plainfield, NJ, USA, among many others) and joining of DNA segments by Gibson reaction (i.e., overlap PCR) as described in, e.g., Gibson Assembly® Master Mix Instruction Manual, New England Biolabs Inc. (NEB), Version 3.3, NEB catalog no. #E2611S/L, NEB Inc., Ipswich, MA, USA. The designing, making, and testing of partner-directing alterations is described in detail in U.S. Pat. No. 11,130,808. Examples 1-3 and 5 and FIGS. 4-7 and 12-15 of U.S. Pat. No. 11,130,808 are incorporated herein by reference. Once DNAs encoding the antibodies are made, the antibodies can be made in transfected host cells as described in, e.g., the Examples herein.

In further embodiments, an anti-hIL33 and/or anti-hTSLP antibody as described herein can comprise one or more alteration(s) that disfavor(s) HC/HC heterodimer formation. In further embodiments, an anti-hIL33 and/or anti-hTSLP antibody as described herein can include one or more alterations that decrease one or more aspects of the effector function of the antibody. Examples of such alterations include the following: (1) D265A or D265X (where X is any amino acid other than D) in the HC of an IgG antibody; (2) E318X, K320X, and/or K322X (where X is any amino acid other than the original amino acid) in an IgG2 antibody; (3) D270X, K322X, P329X, and/or P331X (where X is any amino acid other than the original amino acid) in an IgG1 antibody; (4) P329A; (5) L234A, L235A, and/or P329A in an IgG1 antibody; and/or (6) L234A, L235E, and G237A in an IgG1 constant region.

### Mixtures of Anti-Hil33 and Anti-Htslp Antibodies

Provided herein are mixtures of antibodies comprising the anti-hIL33 and anti-hTSLP antibody antibodies described herein. In some embodiments, such mixtures of antibodies are made in a single host cell line into which one or more DNA(s) encoding the two antibodies has (have) been introduced. This method of making pairs of antibodies is described in detail in WO 2017/205014 (corresponding to U.S. Pat. No. 11,130,808), at e.g, Examples 1-12, pages 68-103, plus the Figures referred to therein, which WO 2017/205014 and U.S. Pat. No. 11,130,808 are incorporated herein by reference in their entirety for all purposes. Mixtures of two antibodies made using these methods are referred to herein as MabPairs.

In more detail, these mixtures can comprise any of the anti-hTSLP antibodies described herein above. In some embodiments, the anti-hTSLP antibody comprises D399R and K409E, among other alterations, in its HC. For example, as described in Example 9, the set of substitutions K147D, V173C, C220G, D265A, D399R, and K409E were introduced in the HC of anti-hTSLP antibody QB11237; whereas substitutions S131K, S162C, C214S were introduced in the LC of anti-hTSLP antibody QB11237, and the new anti-hTSLP antibody with such substitutions was renamed as clone QB11718. SEQ ID NOs: 72 and 71 show the amino acid sequence of anti-hTSLP QB11718 HC and the nucleic acid sequence encoding it, respectively. SEQ ID NOs: 76 and 75 show the amino acid sequence of anti-hTSLP QB11718 LC and the nucleic acid sequence encoding it, respectively. In particular embodiments, the mixture can further comprise any of the anti-hIL33 antibodies described herein, such as, e.g., QB11465 which was converted to an IgG4 antibody (SEQ ID NOs: 25 and 27) from anti-hIL-33 IgG1 antibody QB11421, for co-expression as a MabPair with the lead anti-TSLP IgGI antibody.

Examples of making other MabPair mixtures are described in Example 9 and include an altered version of anti-hTSLP antibody and unaltered anti-hIL-33 antibody that were made as follows. Substitutions K147D, V173C, C220G for strengthening the cognate chain pairing in CH1; substitution D265A for the attenuated effector function (ADCC, ADCP, CDC) in CH2; substitutions D399R and K409E for preventing the formation of heterodimeric HCs in CH3 were introduced into the HC of anti-hTSLP antibody QB11548. Accordingly, substitutions S131K, S162C, C214S were introduced into the LC of anti-hTSLP antibody QB11548, and the altered anti-hTSLP antibody was renamed as clone QB11764. SEQ ID NOs: 52 and 51 show the amino acid sequence of anti-hTSLP QB11764 HC and the nucleic acid sequence encoding it, respectively. SEQ ID NOs: 56 and 55 show the amino acid sequence of anti-hTSLP QB11764 LC and the nucleic acid sequence encoding it, respectively.

Other MabPairs comprising an anti-hIL33 and an anti-hTSLP antibody with other alterations (as described in U.S. Pat. No. 11,130,808) relative to the HC and LC sequences described herein are also included within the mixtures of antibodies provided herein. Further, in some embodiments an anti-hTSLP IgG1 antibody can comprise 147D, 170C, 173C, 220G, and 399R and 409E in its HC and 131K, 160C, 162C, and 214S in its LC, and an anti-hIL33 IgG4 antibody has a natural 409R in its HC.

Exemplary partner-directing alterations, one or more of which can be included in the anti-hIL33 and/or anti-hTSLP antibodies in an antibody mixture, are listed in Table 6 below.

TABLE 6
EXEMPLARY PARTNER-DIRECTING ALTERATIONS

| | Antibody 1* | | | | Antibody 2* | | | |
|---|---|---|---|---|---|---|---|---|
| | HC1 | | LC1 | | HC2 | | LC2 | |
| | V_{H} | C_{H}1^{@} | V_{L} | C_{L} | V_{H} | C_{H}1^{@} | V_{L} | C_{L} |
| 1^{#} | 44E/D | | 100R/K | | 44R/K | | 100E/D | |
| 2 | 105R/K | | 43E/D | | 105E/D | | 43R/K | |
| 3 | | 147R/K | | 131E/D | | 147E/D | | 131R/K |
| 4 | | 168E/D | | 174R/K | | 168R/K | | 174E/D |
| 5 | | 181R/K | | 178E/D | | 181E/D | | 178R/K |
| 6 | | 126C | | 121C | | 133C | | 209C |
| 7 | | 168C | | 174C | | 133C | | 117C |
| 8 | | 170C | | 162C | | 183C | | 176C |
| 9 | | 173C | | 160C | | 170C | | 162C |
| 10 | | 173C | | 160C | | 183C | | 176C |
| 11 | | 170C | | 176C | | 173C | | 160C |
| 12 | | 170C | | 176C | | 183C | | 176C |
| 13 | | 170C | | 162C | | 170C | | 176C |
| 14 | | 173C | | 162C | | 170C | | 176C |
| 15 | | 173C | | 162C | | 173C | | 160C |
| 16 | | 173C | | 162C | | 170C | | 162C |

*Antibodies 1 and 2 are different antibodies. For the purposes of this table, they are interchangeable.
^{#}The alterations listed in a single row for heavy and light chains of a single first antibody (e.g., HC1 and LC1) can occur together as listed. However, the second antibody in the mixture may or may not contain the alterations listed in the same row for Antibody 2. In some embodiments, an antibody can comprise the alterations listed in two or more rows, e.g., 105R/K and 147R/K in a heavy chain and 43E/D and 131E/D in a light chain.
^{@}Not all alterations are suitable for all IgG subtypes.

Alterations that disfavor heterodimers can be included in the anti-hIL33 and/or the anti-hTSLP antibody when they are part of an antibody mixture, assuming that both antibodies are IgG antibodies. In one embodiment, one antibody can be an IgG4 antibody (which has a naturally occurring arginine at position 409) or an IgG1 antibody that has been altered so as to have an arginine at position 409, i.e., has the alteration K409R, and the other antibody has the amino acids 399K/R and 409D/E.

In some embodiments, an anti-hIL33 antibody and an anti-hTSLP antibody, including an anti-hIL33 VH and VL and an anti-hTSLP VH and VL described herein, can be part of a Chimeric Antigen Receptor (CAR), which can also include portions of a T cell receptor and can be used for CAR-T cell therapy. CAR-T cell therapy is explained in, e.g., Yu et al. (2019), Molecular Cancer 18:125 (://doi.org/10.1186/s12943-019-1057-4); and Lemal and Tournilhac (2019), J. ImmunoTher. Cancer 7:202 (https://doi.org/10.1186/s40425-019-0686-x)

### Polynucleotides, Vectors, and Host Cells

Provided are polynucleotides, e.g., DNA or other nucleic acids, encoding the antibodies and mixtures of antibodies described herein. Using the guidance provided herein, one of skill in the art could combine known or novel nucleic acid sequences encoding antibodies and modify them by known methods to create polynucleotides encoding the antibodies and the mixtures of antibodies described herein, which comprise VH and VL amino acid sequences described herein. Such nucleotide sequences encoding VHs, VLs, HCs, or LCs, or portions of such sequences, are disclosed in, e.g., SEQ ID NOs: 24, 26, 28, 30, 49, 51, 53, 55, 69, 71, 73, and 75, as well as throughout this Specification. In some embodiments, (a) polynucleotide(s) can encode an HC and/or LC comprising alterations with respect to the amino acid sequences disclosed herein, such as partner-directing alterations. Such alterations can be amino acid substitutions. In addition, such (a) polynucleotide(s) can encode an HC and/or an LC comprising one or more partner-directing alterations outside of the variable domains and/or one or more alterations that disfavor heterodimers. Numerous nucleic acid sequences encoding human, mammalian, and primate immunoglobulin constant domains, for example the CL, CH1, hinge, CH2, and CH3 are known in the art. See, e.g., Kabat et al., supra. Optionally, polynucleotide sequences encoding variable domains described herein can be combined with polynucleotide sequences encoding such constant domains to create antibodies in any of a variety of formats, e.g., IgG, IgM, IgD, IgE, IgA, bispecific formats, scFv, scFv-Fc, Fabs, BiTE (scFc-linker-scFv), Fab-scFv, IgG-scFv. In some embodiments, polynucleotide sequences can encode HCs in which the hinge, or a portion of the hinge, can be taken from a different isotype or isotype subclass than one or more of the other constant domains, and/or the hinge region can have an altered amino acid sequence relative to a naturally occurring hinge domain. In some embodiments, these antibodies can comprise partner-directing alterations and/or alterations that disfavor heterodimers. In further embodiments, polynucleotide sequences can encode HCs that have been modified so as to increase effector functions such as complement dependent cytotoxicity (CDC) and antibody-dependent cellular cytotoxicity (ADCC). In some embodiments these antibodies can be mammalian antibodies, optionally human, humanized, or primate antibodies.

Methods of modifying polynucleotides are well-known in the art. Perhaps the most straightforward method for creating a modified polynucleotide is to synthesize a polynucleotide having the desired sequence. A number of companies, e.g., DNA 2.0 (Menlo Park, Calif., USA), BlueHeron (Bothell, Washington), Genewiz (South Plainfield, New Jersey), Gen9 (Cambridge, Massachusetts), and Integrated DNA Technologies (Coralville, Iowa), provide this service. Other known methods of introducing mutations, for example site-directed mutagenesis using polymerase chain reaction (PCR), can also be employed. See, e.g., Zoller (1991), Curr. Opin. Biotechnol. 2 (4): 526-531; Reikofski and Tao (1992), Biotechnol. Adv. 10 (4): 535-547.

Vector(s) that contain(s) polynucleotides, optionally DNA, encoding the antibodies and mixtures thereof described herein can be any vector(s) suitable for expression of the antibodies in a chosen host cell. The vector can include a selectable marker for selection of host cells containing the vector and/or for maintenance and/or amplification of the vector in the host cell. Such markers include, for example, (1) genes that confer resistance to antibiotics or other toxins, e.g., ampicillin, tetracycline, or kanamycin for prokaryotic host cells, (2) genes that complement auxotrophic deficiencies of the cell, or (3) genes whose operation supplies critical nutrients not available from complex or defined media. Specific selectable markers can be the kanamycin resistance gene, the ampicillin resistance gene, and the tetracycline resistance gene. A zeocin resistance or neomycin resistance gene may also be used for selection in both prokaryotic and eukaryotic host cells. A dihydrofolate reductase (DHFR) gene and/or a promoterless thymidine kinase gene can be used in mammalian cells, as is known in the art. See, e.g., Kingston et al. 2002, AMPLIFICATION USING CHO CELL EXPRESSION VECTORS, Current Protocols in Molecular Biology, Ch. 16, Unit 16.23, Wiley 2002.

In addition, a vector can contain one or more other sequence elements necessary for the maintenance of the vector and/or the expression of the inserted sequences encoding the antibodies or antibody mixtures described herein. Such elements include, for example, an origin of replication, a promoter, one or more enhancers, a transcriptional terminator, a ribosome binding site, a polyadenylation site, a polylinker insertion site for exogenous sequences (such as the DNA encoding an antibody or mixture of antibodies described herein), and an intervening sequence between two inserted sequences, e.g., DNAs encoding an HC and an LC. These sequence elements can be chosen to function in the desired host cells so as to promote replication and/or amplification of the vector and expression and of the heterologous sequences inserted into the vector. Such sequence elements are well known in the art and available in a large array of commercially available vectors.

In some embodiments, the polynucleotides encoding the antibodies or the mixtures of antibodies can be carried on one or more viral vector(s), optionally oncolytic viral vector(s). Examples of such viral vectors include adenovirus, adeno-associated virus (AAV), retrovirus, vaccinia virus, modified vaccinia virus Ankara (MVA), herpes virus, lentivirus, Newcastle Disease virus, measles virus, coxsackievirus, reovirus, and poxvirus vectors. In such embodiments, these viral vectors containing polynucleotides encoding the antibody or mixture of antibodies described herein can be administered to patients to treat a disease. In a cancer patient, for example, such viral vectors containing polynucleotides encoding an antibody or mixture of antibodies can be administered directly to a tumor or a major site of cancer cells in the patient, for example by injection, inhalation (for a lung cancer), topical administration (for a skin cancer), and/or administration to a mucus membrane (through with the nucleic acids can be absorbed), among many possibilities. Alternatively, such viral vectors can be administered systemically, for example, orally, topically, via a mucus membrane, or by subcutaneous, intravenous, intraarterial, intramuscular, or peritoneal injection as described herein. Similarly, polynucleotides encoding a mixture of antibodies as described herein, which can be encased in liposomes, can be administered to a patient suffering from a disease.

Polynucleotides and/or vectors described herein can be introduced into a host cell, for example for the purpose of producing one or more antibodies. A host cell containing one or more polynucleotide(s) and/or vector(s) encoding one or more antibodies can be any of a variety of cells suitable for the expression of a recombinant protein. These include, for example, gram negative or gram positive prokaryotes, for example, bacteria such as Escherichia coli, Bacillus subtilis, or Salmonella typhimurium. In other embodiments, the host cell can be a eukaryotic cell, including such species as Saccharomyces cerevisiae, Schizosaccharomyces pombe, or eukaryotes of the genus Kluyveromyces, Candida, Spodotera, or any cell capable of expressing heterologous polypeptides. In further embodiments, the host cell can be a mammalian cell. Many mammalian cell lines suitable for expression of heterologous polypeptides are known in the art and can be obtained from a variety of vendors including, e.g., American Type Culture Collection (ATCC). Suitable mammalian host cell lines include, for example, the COS-7 line (ATCC CRL 1651) (Gluzman et al., 1981, Cell 23:175), L cells, C127 cells, 3T3 cells (ATCC CCL 163), Chinese hamster ovary (CHO) cells, or their derivatives such as Veggie CHO and related cell lines, which grow in serum-free media (Rasmussen et al., 1998, Cytotechnology 28:31), CHO-K1 and CHO pro-3 cell lines and their derivatives such as the DUKX-X11 and DG44 cell lines, which are deficient in dihydrofolate reductase (DHFR) activity, HeLa cells, baby hamster kidney (BHK) cells (e.g., ATCC CRL 10), the CVI/EBNA cell line derived from the African green monkey kidney cell line CVI (ATCC CCL 70) as described by McMahan et al., 1991, EMBO J. 10:2821, human embryonic kidney (HEK) cells such as 293, 293 EBNA or MSR 293, human epidermal A431 cells, human Colo205 cells, HL-60 cells, U937 cells, HaK cells, Jurkat cells, HepG2/3B cells, KB cells, NIH 3T3 cells, S49 cells, and mouse myeloma cells, including NSO and Sp2/0 cells. Other prokaryotic, eukaryotic, or mammalian cell types that are capable of expression of a heterologous polypeptide could also be used.

### Methods of Making Antibodies and Mixtures of Antibodies

Generally, individual invention anti-IL33 and/or anti-hTSLP monoclonal antibodies set forth herein, and mixtures thereof, can be produced by introducing DNA encoding the antibody into a host cell, culturing the host cell under conditions suitable for production of the antibody by the cell, and recovering the antibody from the cell mass or the cell supernatant. For example, DNA encoding one or more antibodies can be introduced into a host cell as described above using any appropriate method including, for example, transfection, transduction, lipofection, transformation, bombardment with microprojectiles, microinjection, or electroporation. In some embodiments, DNA encoding two full-length antibodies can be introduced into a host cell. Such methods are known in the art and described in, e.g., Kaestner et al. (2015), Bioorg. Med. Chem. Lett. 25:1171-1176, which is incorporated herein by reference.

The host cell into which the DNA encoding one or more antibodies has been introduced can be cultured, and the antibody or antibodies can be recovered from the cell culture supernatant or the cell mass. The antibody or antibodies can be subjected to further purification steps such as, for example, various kinds of centrifugal sedimentation, precipitation, dialysis, and/or column chromatography, including affinity chromatography, such as Protein A chromatography, anion exchange chromatography, cation exchange chromatography, reverse phase chromatography, hydrophobic interaction chromatography, and size exclusion chromatography, among many possible purification steps.

Antibodies produced individually by the methods described immediately above can be mixed to produce a mixture. Alternatively, mixtures of antibodies can be produced in a similar way except that DNA encoding two different antibodies can be introduced into the host cell, either simultaneously or sequentially. A host cell containing DNAs encoding two different IgG antibodies, i.e., two different heavy and light chains, can potentially produce up to ten different IgG antibody species, due to promiscuous HC/HC and HC/LC pairing. See, e.g., FIG. 4 of U.S. Pat. No. 11,130,808. To limit this number of species, the antibodies can comprise HC and LC partner-directing alterations and/or alterations that disfavor heterodimers. Such alterations can limit the number of major antibody species produced by the host cell. Such mixtures can be purified as described above. Similar issues can arise when producing a bispecific IgG antibody in a single cell line. In this case, partner-directing alterations can be useful to ensure only cognate HC/LC pairing, and alterations favoring heterodimeric HC/HC pairing can also be used. Such alterations are described in, e.g., U.S. Pat. No. 8,592,562. Examples 1 and 2 or U.S. Pat. No. 8,592,562 and the Figures referred therein are incorporated herein by reference.

One of skill in the art will appreciate that producing a mixture of antibodies in a single host cell line, rather than in two host cell lines, represents a significant increase in ease and efficiency of production relative to developing and running two commercial production processes. Development of a commercial production process for any one antibody requires optimization of a myriad of factors including, e.g., the expression system, the host cell line (if a cell line is used for expression), the cell culture process (including physical variables such as using stirred tank vs. perfusion vs. many other culture methods, as well as the medium and feeding strategy used to grow the host cell line), and antibody purification and formulation. Moreover, once a process is developed, it must be characterized and validated and transferred to a manufacturing facility for current good manufacturing practices (cGMP) production. See, e.g., Li et al. (2010), Cell culture processes for monoclonal antibody production, mAbs 2 (5): 466-477. Thus, it is clear that production of an antibody mixture in a single process, versus production in two processes, represents a significant increase in ease and efficiency of production, not to mention a significant decrease in cost.

### Pharmaceutical Compositions and Methods of Administration

The anti-hIL33 and/or anti-hTSLP antibodies, anti-hIL33 and anti-hTSLP antibody mixtures, bispecific antibodies, polynucleotides, and/or vectors described herein can be administered in a pharmaceutically acceptable formulation. With regard to the mixtures of anti-hIL33 and anti-hTSLP antibodies, each antibody can be formulated and administered either separately or together as a MabPair as described here in Examples 9-11. Numerous pharmaceutical formulations are known in the art. Many such formulations are described in Remington: The Science and Practice of Pharmacy, 21^{st} ed., Lippincott Williams & Wilkins, Philadelphia, PA, 2005, the relevant portions of which are incorporated herein by reference.

Polynucleotides and proteins such as antibodies are usually administered parenterally, as opposed to orally. Depending on the formulation, oral administration could subject the protein or polynucleotide to the acidic environment of the stomach, which could inactivate the protein or polynucleotide. In some embodiments, a specific formulation might allow oral administration of a specific protein or polynucleotide where the protein or polynucleotide is either insensitive to stomach acid or is adequately protected from the acidic environment. A formulation could also be administered via a mucus membrane. A formulation could also be administered topically in some embodiments. Commonly, antibodies and polynucleotides are administered by injection of a liquid formulation.

### Methods of Treatment

Provided herein are methods of treating a patient, in need thereof, having inflammation (e.g., Type 2 inflammation, and the like), an inflammatory disease, a chronic inflammatory airway disease, such asthma, COPD, and the like, the method comprising administering to the patient a mixture comprising: (a) an anti-hIL33 antibody and/or an anti-hTSLP antibody; or administering to the patient one or more polynucleotide(s) encoding the mixture of (a). For example, asthma and COPD (Chronic Obstructive Pulmonary Disease) are chronic inflammatory airway diseases characterized by obstructive airflow limitation. Both diseases are significant burdens on patients and healthcare systems. While asthma affects 262 million people with 461,000 deaths globally [see Lancet 2020; 396:1204-1222], COPD carries an even greater burden of disease and is the third leading cause of death worldwide, responsible for ~3.2 million deaths in 2019. Both diseases are heterogeneous in terms of their clinical presentation and underlying inflammatory mechanisms, therefore, therapy by single drug with single MOA (mechanism of action) may not be effective enough to treat all patients.

For both diseases, common triggers for acute exacerbations of airflow obstruction include viral or bacterial infections, cigarette smoke, allergens, and environmental factors such as air pollution. These triggers induce the epithelial cells in lung to secrete IL-33 (interleukin-33), TSLP (thymic stromal lymphopoietin), and IL-25 (interleukin-25, or IL-17E), three alarmins which drive Type-2 inflammation. For asthma in particular, genome-wide association studies have shown strong associations of the disease and genetic polymorphisms in TSLP, IL33, and ST2. Thus, TSLP and IL-33 both have emerged as particularly attractive targets due to their strong genetic links to asthma and its broad effects on airway inflammation.

ILC2s are a relatively newly identified immune cell. They are tissue-resident cells that are predominantly distributed in mucosal tissues such as lung, small intestine, skin, and adipose tissue. Due to their locations, they are considered to play a pivotal role in allergic diseases and in the development of Type-2 inflammation. After engagement by alarmins, ILC2s rapidly release cytokines such as IL-4, IL-5, and IL-13 to mediate responses by eosinophils, mast cells, basophils, DCs, B cells, and Th2 cells.

Currently available standard-of-care treatment with combined inhaled β-adrenergic agonists and inhaled corticosteroids (ICS) mainly provide symptomatic relief with control of disease and can often improve lung function and reduce exacerbation rates. However, the response is generally better in asthma compared to COPD and those with eosinophilic inflammation respond best to ICS. A subset of patients with asthma and COPD are insensitive to ICS. Side effects such as the increased frequency with increased dozing, muscle cramps, muscle twisting are common to patients with a long-term use of steroids and β-adrenergic agonists.

Patients with non-Type-2 asthma, as assessed by a low blood eosinophil count and low exhaled nitric oxide fraction, are not suitable candidates for current biologic therapies. In addition, while azithromycin and the phosphodiesterase-4 inhibitor, roflimulast, are available for reducing COPD exacerbations, these therapies have limited adoption due to side-effects. For azithromycin, side-effects are primarily gastrointestinal with potential for arrhythmias and development of antibiotic resistance; while with roflimulast, gastrointestinal side-effects with nausea predominate.

Chronic obstructive pulmonary disease (COPD) is currently defined as chronic disease state showing irreversible obstruction of airways resulting from the progression of two major underlying diseases including chronic bronchitis and emphysema. Chronic bronchitis is defined clinically as the persistence of cough, sputum, and difficult breathing, and emphysema is defined histopathologicaly as an irreversible change of airway walls distal to the terminal bronchiole and clinically shows slowly progressive respiratory difficulties. COPD is currently the fourth leading cause of death in the United States and Europe, and causes of death in patients with COPD are complications of the disease such as respiratory failure or infection (GOLD workshop summary, Am J Respir Crit Care Med 2001; 163:1256-1276).

In some portion of patients with long-standing asthma, irreversible obstruction of airway which is difficult to distinguish with COPD can be developed, and thus bronchial asthma can be progressed to COPD (Celli B R, et al., Eur Respir J 2004; 23:932-46). Significant reversible improvement of airway obstruction after short-term or long-term pharmacological treatments with inhalations of bronchodilators and steroids has been documented in a significant number of patients meeting current diagnostic criteria for COPD (GOLD workshop summary, Am J Respir Crit Care Med 2001; 163:1256-1276). Therefore, there is a significant number of patients satisfying definition criteria for both bronchial asthma and COPD (Guerra S, Curr Opin Pulm Med 2005; 11:17-13).

Thus, a large unmet need remains in disease maintenance and acute exacerbation reduction for treating and/or preventing chronic inflammatory diseases, including COPD and asthma. It is contemplated herein that blocking both IL-33 and TSLP alarmins could improve the efficacy for treating chronic inflammatory disease, such as asthma, COPD, and the like, since blocking only one alarmin is not sufficient. Dual blockade of IL-33 and TSLP is contemplated herein to provide advantages over targeting a single pathway, for example, anti-IL-5 therapy only ameliorates Type-2 inflammation. Dual blockade of IL-33 and TSLP would decrease the secretion of several downstream cytokines, i.e. IL-4, IL-5, IL-13, which mediate inflammation responses from eosinophils, B cells, mast cells etc.

It is contemplated herein that the invention anti-hIL33 and anti-hTLSP MabPair antibodies provide a safety advantage over small molecules. The invention MabPair compositions provided herein are contemplated to avoid the side effects of long-term use of inhaled β-adrenergic agonists and inhaled corticosteroids. The invention MabPair compositions provided herein are contemplated to address the unmet need for treating COPD since no biologics have yet been approved by FDA for this indication so.

In addition to asthma and COPD, other inflammatory diseases contemplated for treatment herein include, but are not limited to, arthritis, dermatitis, psoriasis, cystic fibrosis, post transplantation late and chronic solid organ rejection, multiple sclerosis, systemic lupus erythematosus, inflammatory bowel diseases, autoimmune diabetes, diabetic retinopathy, diabetic nephropathy, diabetic vasculopathy, ocular inflammation, uveitis, rhinitis, ischemia-reperfusion injury, post-angioplasty restenosis, glomerulonephritis, Graves disease, gastrointestinal allergies, conjunctivitis, atherosclerosis, coronary artery disease, angina, and small artery disease.

Also contemplated herein, the use of the invention anti-hIL33 and anti-hTLSP MabPair antibodies could have potential for treating virus infection and cancers, see Stanbery A.G. et al. J Allergy Clin Immunol. 2022; 150 (6): 1302-1313.

In a particular embodiment of the methods of treatment provided herein, (1) (A) the anti-hIL33 antibody comprises a VH and a VL, each comprising a CDR1, a CDR2, and a CDR3, (B) the anti-hIL33 antibody comprises a VH CDR1, VH CDR2, VH CDR3, VL CDR1, VL CDR 2, and VL CDR3 comprising, respectively, the following sequences: SEQ ID NOs: 80, 81, 82, 77, 78, and 79, and (C) the anti-hIL33 antibody inhibits the interaction of human IL-33 and ST2/IL1AcP complex; and (1) (B) the anti-hTSLP antibody comprises a VH and a VL, each comprising a CDR1, a CDR2, and a CDR3, (B) anti-hTSLP antibody comprises a VH CDR1, VH CDR2, VH CDR3, VL CDR1, VL CDR2, and VL CDR3 comprising, respectively, the following sequences: SEQ ID NOs: 86, 87, 88, 83, 84, and 85; SEQ ID NOs: 86, 87, 88, 83, 89, and 90; SEQ ID NOs: 94, 95, 96, 91, 92, and 93; and (C) the anti-hTSLP antibody inhibits hTSLP binding to hTSLPR/hIL7Rα complex. In another embodiment, the mixture of antibodies comprises: (a) an anti-hIL33 antibody comprising a heavy chain (HC) and a light chain (LC), wherein (1) the HC of the anti-hIL33 antibody is encoded by a nucleic acid sequence which encodes the amino acid sequence of SEQ ID NO: 31, and (2) the LC of the anti-hIL33 antibody is encoded by a nucleic acid sequence which encodes the amino acid sequence of SEQ ID NO: 27; and (b) an anti-hTSLP antibody comprising an HC and an LC, wherein (1) the HC of the anti-hTSLP antibody is encoded by a nucleic acid sequence which encodes the amino acid sequence of SEQ ID NO: 72, and (2) the LC of the anti-hTSLP antibody is encoded by a nucleic acid sequence which encodes the amino acid sequence of SEQ ID NO: 76. In particular embodiments, the anti-hIL33 and anti-hTSLP mixtures of antibodies used in the invention methods correspond to MabPairs QB11750 and QB11823 set forth in Examples 10 and 11. MabPair QB11750 comprises anti-IL-33 IgG4 antibody QB11465 and anti-TSLP IgG1-D265A antibody QB11718; and MabPair QB11823 comprises of anti-IL-33 IgG4 antibody QB11465 and anti-TSLP IgG1-D265A antibody QB11764.

The anti-hIL33 and/or anti-hTSLP mixtures of antibodies comprising an anti-hIL33 and/or anti-hTSLP administered separately or as a mixture (e.g, as MabPairs QB11750 and QB11823, and the like; set forth in Examples 10 and 11), can be administered separately or as a mixture, bispecific antibodies comprising an anti-hIL33 and/or anti-hTSLP and a second antibody, or polynucleotide(s) or vector(s) encoding such antibodies or combinations can be administered with an additional therapy, which is administered before, after, and/or concurrently with the antibody, the combination, or the polynucleotide(s) or vector(s).

With regard to the antibodies or mixtures thereof, they can be administered to a patient in a therapeutically effective dose at appropriate intervals. A therapeutically effective dose can be determined by methods known in the art, including testing in in vitro assays, rodent and/or primate model systems, and/or clinical trials. Doses of antibodies, mixtures of antibodies, or polynucleotides encoding them can be administered once or twice or at time intervals over a period of time. In some cases, dosing can be discontinued and restarted. In some embodiments, a mixture comprising an anti-hIL33 and an anti-hTSLP antibody can be administered so that both antibodies can be administered simultaneously. After one or more doses of the mixture, one of the antibodies alone can be administered. In some embodiments, dosing with this antibody can continue for a period of time. In some embodiments, dosing with the antibody or mixture of antibodies can be discontinued and resumed one or more times.

In the case of one or more polynucleotide(s) or vector(s) encoding the antibody or mixtures of antibodies described herein, doses can, for example, be from about 5×10⁹ copies the of the polynucleotide(s) or vector(s) per kilogram of body weight (copies/kg) to about 10¹⁵ copies/kg, from about 10¹⁰ copies/kg to about 10¹⁴ copies/kg, or from about 5×10¹⁰ copies/kg to about 5×10¹³ copies/kg. Alternatively, doses can be about 10¹⁰, 10¹¹, 10¹², 10¹³, 5×10¹³, 10¹⁴, 2×10¹⁴, 3×10¹⁴, 4×10¹⁴, 5×10¹⁴, 6×10¹⁴, 7×10¹⁴, 8×10¹⁴, 9×10¹⁴, or 10¹⁵ copies of the polynucleotide(s) or vector(s). Frequency of dosing can be adjusted as needed and can be as described above or, for example, every day, every other day, twice a week, once a week, once every ten days, once every two weeks, once every three weeks, once per month, or once every two, three, four, five, six, seven eight, nine, ten, eleven, or twelve months.

Having described the invention in general terms above, the specific Examples below are offered to exemplify the invention, not limit its scope. It is understood that various changes and modifications may be made to the invention that are in keeping with the spirit of the invention described herein and would be apparent to one of skill in the art. Such changes and modifications are within the scope of the invention described herein, including in the appended claims.

### EXAMPLES

### Example 1: Humanization and Engineering of a Mouse-Anti-Human IL-33 Monoclonal Antibody

Interleukin 33 (IL-33) is a member of the IL-1 family of cytokines. Binding of hIL-33 to the hIL-33 receptor consisting of ST2 and IL-1RAcP can promote pro-inflammatory responses. We aimed to develop an anti-hIL-33 antibody that could block the binding of hIL-33 to ST2.

Full length hIL-33 is secreted as a 270 amino acid pro-form that can be cleaved by serine proteases such as tryptase and chymase, which are secreted by mast cells, to generate the more potent cleaved forms of the cytokine, see LeFrancais. E. et al. Proc Natl Acad Sci USA 2014; 111 (43): 15502-15507.

A recombinant hIL-33 antigen as isoform A version of the protein (SEQ ID NO: 1), beginning at Ser112 and ending at amino acid Thr270 along with N-terminal tags including Avitag, c-MYC, and His6 for biotinylation, detection, and purification respectively, was made in-house by transient transfection of Expi293 cells. Briefly, DNA fragments encoding the hIL-33 antigen (SEQ ID NO: 1) were ordered from Integrated DNA Technologies Inc. Coralville, IA, USA. The DNA fragments were assembled into the pSB01 expression vector by Gibson assembly reaction, see Gibson G. Methods Enzymol. 2011; 498:349-361. The insert was confirmed by Sanger sequencing at Genewiz Inc. (219 Terry Ave N, Floor 2, Seattle, WA 98109). The plasmid DNA encoding the recombinant hIL-33 antigen was transiently transfected into Expi293 cells (ThermoFisher Scientific, Waltham, MA) using ExpiFectamine™ 293 Transfection Kit (ThermoFisher Scientific, cat. no. A14525) according to the manufacturer's instructions. After 5 days of shaking in the incubator at 37° C., 120 rpm, 5% CO2, the supernatant was harvested, the recombinant hIL-33 antigen was purified on an AKTA Avant chromatography system (Cytiva Inc., Marlborough, MA) using HisTrap Excel columns (Cytiva Inc., cat. no. 17371205) with standard techniques. Human IL-33 can undergo oxidation resulting in the formation of two intracellular disulfide bonds. This oxidation changes the conformation of hIL-33, leading to the loss of binding to receptor ST2, see Verstraete K et al. Nat. Commun. 2017; 8:14937. To keep the recombinant hIL-33 antigen consistently active in a reduced form, a hIL-33 mutein was designed by introducing two substitutions C208S and C232S QB11935 (SEQ ID NO: 2), see Cohen E. et al. Nat. Comm. 2015; 6:8327. This oxidation resistant hIL-33 was produced with a His6 tag at the N-terminus for purification. Production of this oxidation resistant hIL-33 antigen was carried out by transient transfection of Expi293 cells as described above.

The cynomolgus monkey homolog of IL-33 (cyIL-33) is secreted as a 270 amnio acid full length protein. The engineered anti-hIL-33 antibody was additionally intended to block the binding of cyIL-33/ST2 axis. A recombinant cyIL-33 antigen was produced for assessing cross-species binding of anti-hIL-33 antibodies and for affinity maturation as described in Example 3. This recombinant cyIL-33 antigen was the isoform X1 version of the protein (NCBI access no. XP_005581824), beginning at Ser112 and ending at Ile270. N-terminal tags including Avitag, FLAG, and His6 were added for biotinylation, detection, and purification respectively. The cyIL-33 protein (SEQ ID NO: 3) was produced by transient transfection in Expi293 cells as described above.

A mouse hybridoma campaign was carried out using the reduced active hIL-33 antigen as immunogen followed by boosting with DNA immunization as described by Liu S et al. Hum Gene Ther. 2018; 29 (9): 997-1003. A blocking antibody clone 18G9 was identified to block the interaction of hIL-33 and human ST2 by ELISA. The cDNA sequences encoding the VL and VH were obtained using standard molecular biology techniques (Meyer L et al. PLOS ONE 2019; 14 (6): e0218717). The murine VH region was combined with human IgG4 constant regions and the murine VL region was combined with human Kappa constant region to be expressed as chimeric antibody, which was confirmed to bind hIL-33 and cyIL-33 antigens by Surface Plasmon Resonance (SPR) technology in Biacore T200 from Cytiva as described below.

The receptor hST2-Fc protein from R&D Systems (cat no. 523-ST-100) was directly immobilized on the CM5 surface by amine coupling method in flow cell 2, 3, 4 whereas no hST2-Fc was immobilized in flow cell 1. The running buffer was flowed through flow cell 1, therefore, the background binding from flow cell 1 was a reference which was subtracted from the results of flow cell 2, 3, or 4 to obtain the real binding. The active hIL-33 monomer (after reduction by 2 mM DTT) was injected over the CM5 surface at 0, 0.62, 1.85, 5.56, 16.7, and 50 nM, the association time was set as either 1 min or 2 min, the dissociation time was 5 min. The binding capacity as Response Units (RU) was recorded. As shown in FIG. 1A, the hIL-33 ligand binds to immobilized hST2-Fc in 1:1 mode in a dose dependent manner, the EC₅₀ for 1 min association is 10.82 nM, the EC₅₀ for 2 min association is 5.175 nM.

After the demonstration of direct hIL-33-hST2 interaction on CM5 surface (FIG. 1A), the same hST2-Fc immobilized CM5 chip was injected with a titrated premixed hIL-33-antibody complex: hIL-33 in combination with either isotype control antibody 10825, anti-hIL-33 comparator-1 antibody 10997, anti-hIL-33 comparator-2 antibody 10996, or anti-hIL-33 chimeric antibody 10998 (SEQ ID NOs: 5 and 7). The association time was set for 2 min and the dissociation time for 5 min. As shown in FIG. 1B, when the isotype control antibody 10825 at either 100 nM or 33.3 nM concentration was premixed with 10 nM hIL-33, the binding capacity was identical at ~60 RUs. Both the anti-hIL-33 comparator-1 and comparator-2 antibodies showed additional binding when they were pre-mixed with hIL-33 ligand, indicating that both comparator antibodies do not block the direct interaction between hIL-33 and hST2. However, the anti-hIL-33 chimeric antibody 10998 strongly inhibited the hIL-33 and hST2 interaction in a dose dependent manner, with an IC₅₀ at 1.5 nM.

The anti-hIL-33 antibody 10998 was humanized using CDRs grafting onto 5 different combinations of human VH and VL germlines. Appropriate back mutations were introduced into the human germline sequence to maintain correct folding of antibody. The humanization of mouse antibodies was well described by many researchers, see a review paper by Safdari Y et al. Biotechnol Genet Eng Rev. 2013; 29:175-186. The 5 initial humanized anti-hIL-33 antibodies were expressed by transient transection of ExpiCHO-S cells (ThermoFisher Scientific, Waltham MA cat. no. A29127) using Expifectamine CHO transfection reagent (ThermoFisher Scientific, cat. no. A29192) with a 12-day culture protocol. Recombinant antibodies were purified from clarified supernatants by Protein A affinity chromatography on an AKTA Avant HPLC using HiTrap MabSelect Sure resin (Cytiva, Marlborough MA cat. no. 29049104). The purified isotype control antibody 10825, anti-hIL-33 chimeric antibody 10998, or individual humanized anti-hIL-33 antibody (10999, 11000, 11001, 11002, 11004) at 1:3 series titration was pre-mixed with 10 nM hIL-33 and injected over the hST2-Fc immobilized CM5 surface as described above. As shown in FIG. 1C, the isotype control antibody 10825 did not inhibit the hIL-33 and hST2 interaction even at 100 nM, whereas the chimeric antibody 10998 and 4 humanized anti-hIL-33 antibodies (10999, 11001, 11002, 11004) comparably inhibited the hIL-33/hST2 interaction in a dose dependent manner, but the humanized antibody 11000 was less potent, with IC₅₀ at 2.269 nM.

The humanized anti-hIL-33 antibody 11004 (SEQ ID NOs: 9 and 11) was chosen for further engineering and affinity maturation because it has robust production, least back mutations, good blocking activity (IC₅₀=0.9271 nM). However, the humanized antibody 11004 showed an 89.8% main peak and 10.2% HMW (high molecular weight) peak in SEC analysis, suggesting that this antibody can aggregate during the production and purification process. To improve the purity by mitigating the aggregation issue, another round of engineering was carried out, several framework residues were mutated to the canonical residues of the VH3/VK2 germlines in antibody 11004. The antibody variant QB11061 (SEQ ID NOs: 13 and 15), with a Q41P substitution in VH-FW2, showed a 97.4% main peak and 2.6% HMW peak.

In silico analysis of the antibody QB11061 identified sequence motifs in both the VH and VL that may be susceptible to deamidation. The VL-CDR2 contains 2 potential deamidation liabilities at sequence motif ²⁸NG²⁹ and ³⁰NT³¹. The VH-CDR2 contains the ⁵⁶NT⁵⁷ potential deamidation liability. Accelerated deamidation experiments were performed to assess whether the susceptibility to deamidation is real. The anti-hIL-33 antibody QB11061 was denatured, reduced, and alkylated prior to trypsin digestion. Briefly, 1 mg of antibody was concentrated to ~10 mg/mL and then exchanged 3 times at 14,000×g at 15° C. for 5 min into 0.1 M triethylammonium bicarbonate buffer pH ~8.5 using a 10 kDa molecular weight cutoff (MWCO) microconcentrator. 350 μg (~35 μL) of protein was added to a 1.5 mL screw top microcentrifuge tube, followed with 315 μL of denaturing buffer containing 7.5 M Guanidine HC1 and 50 mM Tris pH 8.0. Then 8.5 μL of 0.1 M dithiothreitol (DTT) in 50 mM Tris pH 8.0 was added, the sample was briefly vortexed and incubated at 37° C. for 30 min. The reduced antibody was cooled to ambient temperature, then 25.5 μL of 0.1 M iodoacetamide (IAM) in water was added. The sample was briefly vortexed and incubated at ambient temperature in the dark for 30 min. 300 μL of the resulting reduced and alkylated sample was loaded onto a NAP5 desalting column that has been previously equilibrated with 10 ×1 mL portions of freshly prepared digest buffer containing 1M Urea and 50 mM Tris pH 8.0. Once loaded, the column was washed with 200 μL of digest buffer then eluted with 600 μL of digest buffer into a 1.5 mL screw top microcentrifuge tube. TEAB (Triethylammonium bicarbonate) was added at 0.1 M final concentration in one half of eluted sample whereas nothing was added in another half of eluted sample. Both samples were incubated at 45° C. for 36 hr. Sequencing Grade Modified Trypsin (Promega, cat. no. V5113) was added to the samples for digestion at 37° C. for 4 hr, finally 20 μL of 10% (v/v) formic acid was added to fully quench the reaction. The un-treated and treated antibody QB11061 were subjected to analysis by mass spectrometry to assess actual deamidation at the susceptible residues.

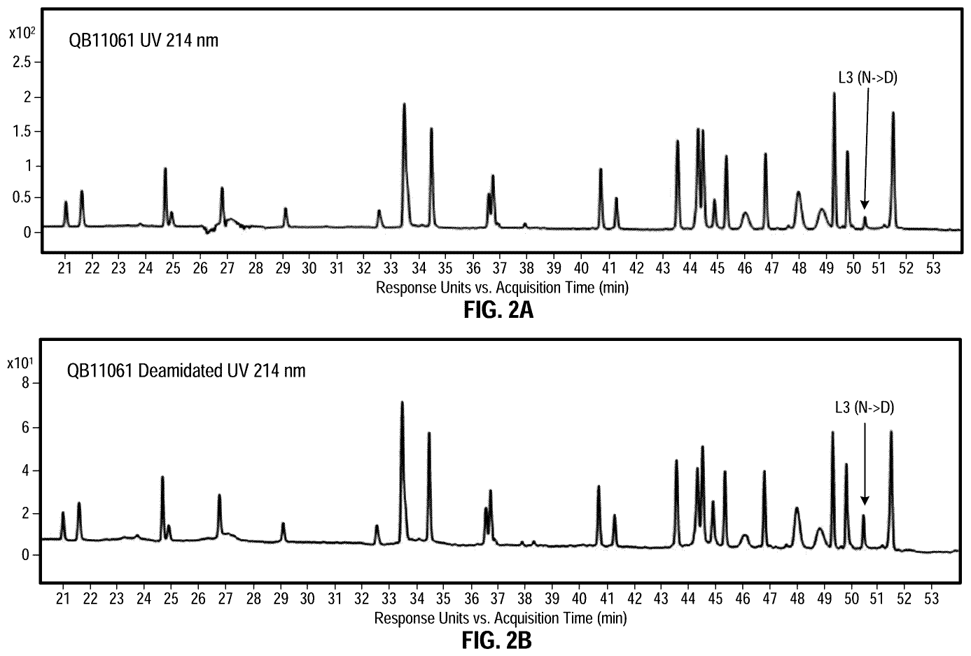

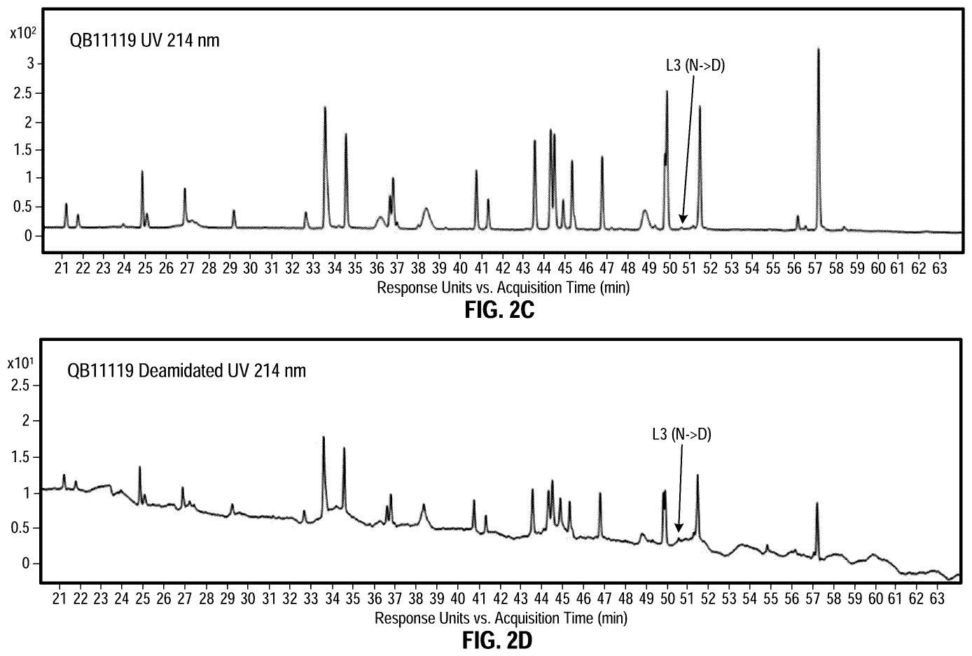

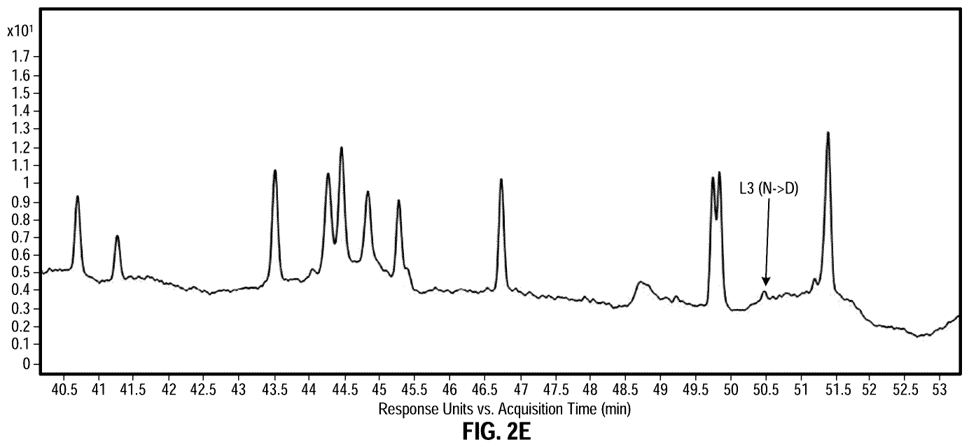

FIG. 2 shows a spectrum of trypsin digested peptides from un-treated anti-hIL-33 antibody QB11061 (A) and treated anti-hIL-33 antibody QB11061 (B); from un-treated ant-hIL-33 antibody QB11119 (C) and treated anti-hIL-33 antibody QB11119 under UV 215 nm scan. A peak representing the deamidation (L3 N→D) at ²⁸NG²⁹ dramatically goes up after treatment of accelerated deamidation, compare FIG. 2A versus FIG. 2B, indicating that the ²⁸NG²⁹ motif was susceptible to deamidation.

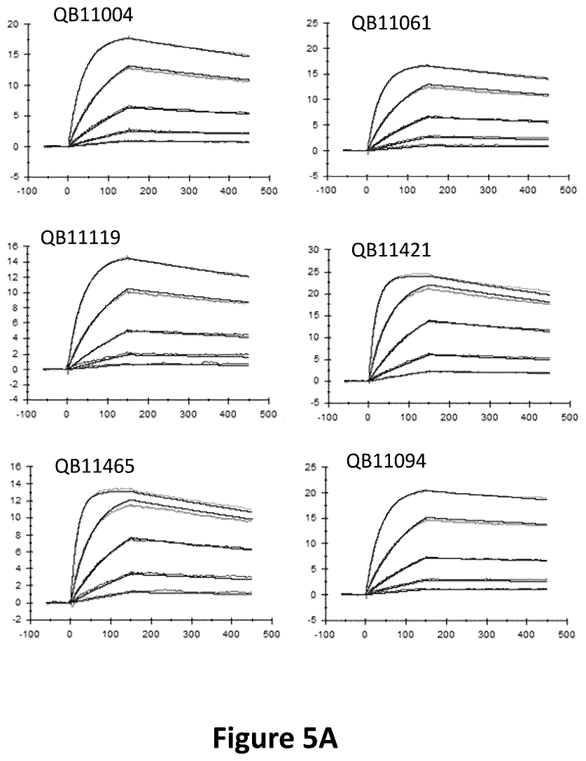

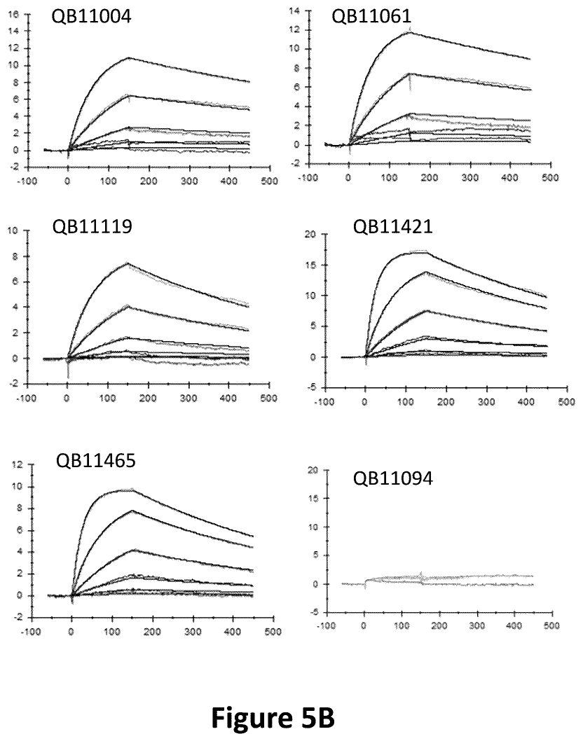

To mitigate the ²⁸NG²⁹ deamidation motif in VL-CDR2 of anti-hIL-33 antibody QB11061, the N28 residue was substituted to either glutamine, glutamate, histidine, lysine, or arginine. Additionally, an antibody variant QB11119 containing a substitution of G29A while retaining N28 (²⁸NA²⁹) was made simultaneously for assessment. The individual LC variants were co-expressed with the HC of QB11061 to produce hIgG1 with L234A+L235A+P329A substitutions which abolish the Fc effector functions, see Wang X. et al. Protein Cell. 2018; 9 (1): 63-73. These antibody variants were produced by transient transfection in ExpiCHO cells and purified by Protein A affinity chromatography. SPR analysis indicated that the anti-hIL-33 antibody QB11119 (SEQ ID NOs: 17 and 19), which contains the G29A substitution in VL-CDR2, maintained affinity for hIL-33 when compared to the humanized antibodies QB11004 and QB11061 (FIG. 5 and Table 7 below). After the ²⁸NG²⁹ motif was changed to ²⁸NA²⁹, the anti-hIL-33 antibody QB11119 was subject to the accelerated deamidation as described above. The peak representing the deamidated peptide L3 (N→D) was significantly decreased in either un-treated (FIG. 2C) or treated (FIG. 2D) anti-hIL-33 antibody QB11119, suggesting that the issue of deamidation at N28 was mitigated when a mutation of G29A was introduced. The spectrum of peptides was zoomed in to be shown in FIG. 2E, the peak representing the deamidated peptide was very minor. A modified product corresponding to the ³⁰NT³¹ deamidation in VL-CDR2 or ⁵⁶NT⁵⁷ deamidation in VH-CDR2 was not observed in either the untreated or the treated sample (data not shown), indicating no significant susceptibility to deamidation for these two sequence motifs. Therefore, after the deamidation mitigation the engineered anti-hIL-33 antibody variant QB11119 was chosen for further affinity maturation by yeast display.

### Example 2: Affinity Maturation of the Humanized Anti-hIL-33 Antibody QB11119 by Yeast Display

Generally, an antagonistic antibody with a high affinity has a high biological activity. To achieve high biological activity, Fab libraries of anti-hIL-33 antibody QB11119 for yeast display were generated to identify beneficial variants which could lead to a high binding affinity. The procedure of constructing Fab libraries for yeast display was briefly described in our patent U.S. Pat. No. 11,124,570.

The amino acid sequence of VL of the antibody QB11119 (SEQ ID NO: 16) was used to identify the position for all residues by Kabat numbering scheme (www.bioinf org.uk/abs/abnum/), the results are shown below. Five CDR residues (K27, M51, M89, L92 and E93) in the VL were chosen for randomization with NNK codons.

Six separate oligos covering the 5 randomized residues were synthesized by Integrated DNA Technologies, Inc. (Coralville IA, USA). The six overlapping oligos along with the short 5′ forward primer and 3′ reverse primer were added in the same tube to assemble and amplify the whole VL region by PCR reactions. The PCR band with expected size was cut out from 1.5% agarose gel then purified with a QIAquick Gel Extraction Kit (Qiagen, cat. no. 28704). The purified PCR product was used for 2^{nd} round of PCR amplification to obtain a large amount of material for library construction. A middle fragment containing the Kappa constant region, c-MYC-R6-pep2A sequence, and a signal peptide 2 (SP2), and a VH fragment of anti-hIL-33 antibody QB11119 were separately amplified by PCR reactions to obtain a large amount of material.

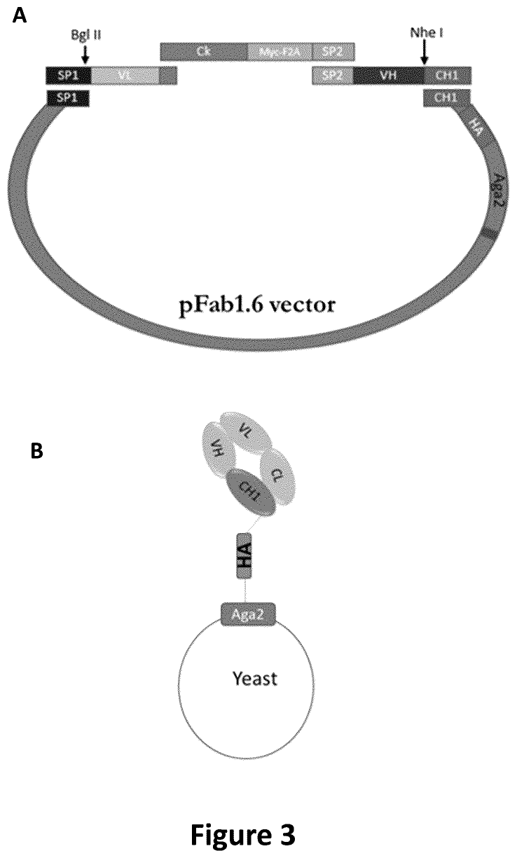

To display the library of randomized Fab fragments in yeast, Saccharomyces cerevisiae cells were electroporated with a linearized vector pFab1.6 having sequence encoding signal peptide (SP1) on one end (which is preceded by a galactose-inducible promoter) and sequence encoding a CH1 domain on the other end (followed by be sequence encoding agglutinin) plus the three purified PCR fragments described above encoding (1) SP1-VL-Ck with randomization at 5 residues, (2) middle fragment Ck-MYC-R6-Pep2A-SP2, and (3) SP2-VH-CH1 as wild type. PCR fragment (1) overlaps with the SPI end of the vector and the Ck end of PCR fragment 2. The signal peptide SP2 end of PCR fragment (2) overlaps with PCR fragment (3) on its SP2 end, and the CH1 end of PCR fragment 3 overlaps with the vector on its CH1 end. Since all of these overlaps are within the range of 30 to 60 base pairs, homologous recombination in yeast enables the fragments to assemble into a single vector containing an insert having the following order of sequence elements: SP1-VL-Ck-MYC-R6-Pep2A-SP2-VH—CH1-HA-agglutinin, where R6 encodes six consecutive arginine residues to serve as a furin cleavage site, and HA is an HA tag, i.e., a small peptide derived from human influenza hemagglutinin that has been extensively used as a protein tag, see FIG. 3A. Expression of these sequences is driven by a galactose inducible promoter upstream from SP1. Transformants were grown on selective agar plates made with yeast medium containing dextrose and lacking uracil. The vector contains a gene that complements the inability of the host yeast strain to make uracil. The library sizes, i.e., the total number of transformants, were in the range of about 1.36×10⁸ transformants, which is 10 times more than the calculated complexities of library. The estimated complexities of the library were less than about 107, meaning that less than about 107 different combinations of nucleotide sequences encoding Fab fragments existed within each library. These estimates were based on the number of possible combinations that could occur given the randomizations at the sites that were varied.

To assess the actual diversity and quality of the library, DNA segments encoding the Fab fragments from 50 randomly picked yeast clones were examined. The VL fragment of these clones was amplified by yeast colony PCR and sequenced by Genewiz Inc. DNA sequence analysis revealed that 60% of the colonies encoded in-frame VL and CL regions and in-frame with VH and CH1 regions, so the translated polypeptide can be processed to be displayed as Fab on yeast surface, see FIG. 3B. The VH-CH1-Aga2 polypeptide is anchored on yeast surface by forming 2 disulfide bonds with Aga1 protein while the processed VL-CL is captured since the VH-VL and CH1-CL interfaces have strong interactions, see Mei M. et al. Microbio. Res. 2017; 196:118-128. The DNA encoding the VL region contained the expected amino acid variations at the targeted positions.

Five CDR residues (S32, G98, R99, R100 and D100b in the VH of antibody QB11119 were chosen for randomization with NNK codons, see Table 8 below.

The VH library for yeast display was similarly constructed as above, with exception of using wild type VL fragment and randomized VH fragment after PCR amplification. The quality of VH library was similarly assessed by DNA sequencing of randomly picked 50 yeast colonies.

The biotinylated hIL-33 QB10975 (SEQ ID NO: 1) and cyIL-33 QB 10976 (SEQ ID NO: 3) proteins were reduced under 10 mM DTT from Sigma Aldrich (Milwaukee WI, cat. no. 3483-12-3) at 50° C. for 30 min to make active antigens. The reduced hIL-33 or cyIL-33 antigen was applied alternately during selection to enrich the antibody variants that can cross-species bind to both hIL-33 and cyIL-33 antigens.

The yeast cells of VL and VH libraries corresponding to >10 times of the library size were inoculated at 0.1 OD₆₀₀ yeast in CM glucose minus URA media (Teknova Inc., Hollister CA, cat. no. C18140) and grown overnight at 30° C. to an OD₆₀₀ of 3 ~5. The yeast cells with amount of 100 OD₆₀₀ were harvested by centrifugation then resuspended in 200 ml of 90% CM galactose+10% glucose media minus URA at 20° C. for 2 days to induce Fab expression on yeast surface. The induced libraries were enriched for antibody variants binding to biotinylated active antigen by streptavidin-conjugated magnetic bead sorting (MACS) as described by Chao et al. Nature Protocols, 2006 doi: 10.1038/nprot.2006.94. The 1^{st} round of MACS was performed using 500 nM biotinylated hIL-33 antigen (SEQ ID NO: 1) using an input of 3.0 ×10⁹ yeast. The 2^{nd} round of MACS was performed using 100 nM biotinylated cyIL-33 antigen (SEQ ID NO: 3).

The Fab yeast pools after MACS enrichment were further sorted by FACS to identify clones with very high binding affinity to hIL-33. The yeast VL pool was incubated with 125 nM bn-hIL-33 then stained with fluorescein isothiocyanate (FITC)-labeled anti-HA antibody from Thermo Fisher Scientific (Waltham MA, cat. no. A-21287) and allophycocyanin (APC)-labeled streptavidin from Thermo Fisher Scientific (cat. no. S868), and subject to FACS sorting. The enriched anti-hIL-33 VL pool was further sorted with 15 nM bn-cyIL-33 antigens consecutively. Similarly, the VH pool obtained after MACS enrichment was sorted against biotinylated 30 nM hIL-33 and 50 nM cyIL-33 antigens consecutively. After the final FACS sorting, yeast cells were plated on CM glucose agar plates minus Uracil (Teknova Inc., cat. no. C3080) and grown at 30° C. for 3 days.

A new combinatorial library containing the beneficial VL and VH variants was constructed to identify the very high affinity Fab binders. For each pool, plasmid DNA was extracted from one OD₆₀₀ of yeast representing >10-fold pool size. Specific primers were used to amplify the variable regions from the VL or VH pool by PCR. The gel purified VL and VH PCR fragments were amplified by a second round of PCR to obtain a large amount of material. The amplified VL fragment, middle fragment, and VH fragment were combined with the Bgl II/Nhe I digested pFab1.6 vector for electroporation into competent yeast cells. The electroporated yeast cells were plated on CM glucose minus URA agar plates, grown for 3 days and harvested. The combinatorial library was induced as described above and sorted by 3 rounds of FACS sorting using biotinylated 20 nM hIL-33, 4 nM cyIL-33, and 1 nM hIL-33 antigens consecutively.

96 yeast colonies were individually picked and induced, then ranked for their binding to hIL-33 antigen as determined by % Q2 double positive signal by FACS analysis, see FIG. 4. A total of 15 colonies with the strongest binding to hIL-33 antigen were selected for further analysis. Plasmid DNA was extracted from individual yeast colonies using a Zymoprep Yeast Plasmid Miniprep II kit (Zymo Research, Irvine CA, cat. no. D2004). The VL and VH regions were amplified by PCR using specific primers. The DNA encoding the VL and VH variable regions was revealed by Sanger DNA sequencing at Genewiz, Inc.

DNA gBlocks encoding the unique VL and VH sequences of the yeast clones with the highest affinity for hIL-33 antigen were synthesized by IDT Inc., the VL gBlocks were assembled with Kappa constant region in pSB01 expression vector by Gibson reaction; the VH gBlocks were assembled with IgG1 constant region in pSB01 expression vector by Gibson reaction. The IgGI HC contains L234A and L235A and P329A for the abolishment of antibody effector functions such as ADCC, CDC, and ADCP. After confirmation by DNA sequencing, the LC and HC plasmids were co-transfected in ExpiCHO cells and the secreted recombinant antibodies in supernatant were purified through Protein A affinity chromatography as described above. The SEC analysis showed that all antibodies had >98% main peak purity, and mass spectrometry analysis demonstrated that all antibodies had the expected LC and HC masses. Preliminary data by Octet analysis indicated that the antibody variant QB11421 (SEQ ID NOs: 21 and 23) had the highest binding affinity to both hIL-33 and cyIL-33 antigens (FIG. 5).

### Example 3: Biacore Measurements of the Top Anti-IL-33 Antibody QB11421 after Affinity Maturation by Yeast Display

The kinetic binding data was obtained using SPR on a Biacore T200 equipment. The anti-hIL-33 antibodies were individually captured to a CM4 chip on which the polyclonal goat-anti-human Fc-specific antibodies were immobilized. The analytes hIL-33 and cyIL-33 were diluted at 1:3 serially in the running buffer containing HBS-EP+0.05% BSA, then were injected across flow cells (fc) 1 through 4, where fc2, fc3, and fc4 had the captured different anti-hIL-33 antibody whereas fcl was flowed with the blank running buffer as a reference and were subtracted from the binding of fc2, fc3, and fc4 respectively to obtain the real antigen binding of the individual antibody. The analytes were injected at a flow rate of 50 μL/min and at a detection temperature of 37° C. for hIL-33 antigen and 25° C. for cyIL-33 antigen. The complex was allowed to associate for 3 min and dissociate for up to 1 hr. At the end of each cycle the CM4 chips were regenerated with 10 mM glycine pH1.5. The T200 Evaluation software V3.2 was used to align and double reference the data. The results were fit to a 1:1 binding model using the global data analysis option within the software.

TABLE 9
Summaries of anti-hIL-33 antibodies binding
to hIL-33 antigen on Biacore T200 equipment.

| | Ka (M⁻¹s⁻¹) | Kd (s⁻¹) | KD (M) | Rmax (RU) |
|---|---|---|---|---|
| QB11004 | 1.62 × 10⁶ | 6.28 × 10⁻⁴ | 3.87 × 10⁻¹⁰ | 17.5 |
| QB11061 | 1.85 × 10⁶ | 5.67 × 10⁻⁴ | 3.06 × 10⁻¹⁰ | 17.5 |
| QB11119 | 1.52 × 10⁶ | 6.04 × 10⁻⁴ | 3.97 × 10⁻¹⁰ | 15.0 |
| QB11421 | 3.23 × 10⁶ | 6.69 × 10⁻⁴ | 2.07 × 10⁻¹⁰ | 25.0 |
| QB11465 | 3.33 × 10⁶ | 6.95 × 10⁻⁴ | 2.09 × 10⁻¹⁰ | 13.5 |
| QB11094 | 1.58 × 10⁶ | 3.09 × 10⁻⁴ | 1.95 × 10⁻¹⁰ | 20.0 |

TABLE 10
Summaries of anti-hIL-33 antibodies binding
to cyIL-33 antigen on Biacore T200 equipment.

| | Ka (M⁻¹s⁻¹) | Kd (s⁻¹) | KD (M) | Rmax (RU) |
|---|---|---|---|---|
| QB11004 | 1.60 × 10⁵ | 9.92 × 10⁻⁴ | 6.22 × 10⁻⁹ | 11.0 |
| QB11061 | 1.88 × 10⁵ | 8.93 × 10⁻⁴ | 4.75 × 10⁻⁹ | 12.0 |
| QB11119 | 1.31 × 10⁵ | 2.06 × 10⁻³ | 1.58 × 10⁻⁸ | 7.5 |
| QB11421 | 3.84 × 10⁵ | 1.88 × 10⁻³ | 4.89 × 10⁻⁹ | 17.5 |
| QB11465 | 3.71 × 10⁵ | 1.91 × 10⁻³ | 5.14 × 10⁻⁹ | 10.0 |
| QB11094 | NA | NA | NA | NA |

As shown Table 9 above, SPR analysis of the top anti-IL-33 IgG1 antibody QB11421 after yeast display affinity maturation demonstrated a~2-fold increased affinity for hIL-33 compared to the parental antibody QB11119. The improvement is mainly derived from a faster association (Ka) whereas the dissociation (Kd) is kept almost the same. SPR analysis also demonstrated QB11421 had a very comparable binding affinity for hIL-33 as the comparator anti-IL-33 antibody QB11094, which has a slower association (Ka) and a slower dissociation (Kd). After the anti-hIL-33 IgG1 antibody QB11421 was converted, using methods well-known in the art, to an IgG4 antibody QB11465 (SEQ ID NOs: 25 and 27) for co-expression as a MabPair with the lead anti-TSLP IgGI antibody, the binding affinity was very similar since the VH and VL variable regions are not changed. The antibody described here was intended to bind to cyIL-33. SPR analysis was performed as described above using cyIL-33 protein as the analyte (see Table 10) and demonstrated that the top clone as IgGI (QB11421) and IgG4 (QB11465) after yeast display had similar binding affinity for cyIL-33 antigen, they both had a ~3-fold improved binding affinity over the parental antibody QB11119. However, the anti-hIL-33 comparator-3 antibody QB11094 had negligible binding to cyIL-33 antigen derived from mammalian cells.

### Example 4: Raising Anti-TSLP Antibodies with Rabbit Hybridomas and Identification of Blocking Antibodies

Three versions of recombinant hTSLP proteins with Avitag, c-MYC, His6 tags for biotinylation, detection and purification respectively at N-terminus were separately made by transient transfection of Expi293 cells and purified through standard Nickel column: (1) wild type TSLP, SEQ ID NO: 32, but it was produced in the presence of furin inhibitor II (Sigma cat no. SCP0148). (2) deletion of the furin cleavage motif of five consecutive residues ¹²⁶KRRKR¹³⁰, SEQ ID NO: 33. (3) introduction of two substitutions (R127A+R130S) to abolish the furin cleavage site, SEQ ID NO: 34. Similarly, three versions of recombinant cyTSLP proteins with Avitag, FLAG, His6 tags at N-terminus were separately made by transient transfection of Expi293 cells and purified through standard Nickel column, SEQ ID NO: 35 for wild type TSLP produced in the presence of furin inhibitor II; SEQ ID NO: 36 for the deletion of furin cleavage motif; SEQ ID NO: 37 for mutein with R127A+R130S. The in-house antigens were used for Biacore measurement, yeast display, and cell-based functional assays as described below.

Rabbit monoclonal antibodies can recognize diverse epitopes, including those poorly immunogenic in mice and humans. Because the rabbit's natural antibody repertoire is more diverse than that of mice, rabbit antibodies exhibit higher affinity for the antigen than mice antibodies. Rabbits have a larger spleen, a greater blood volume, and more bone marrow tissue than mice, providing convenience for operation. Four New Zealand White rabbits were immunized at Genscript (Piscataway, NJ) with hTSLP QB11033 antigen which has a deletion of ¹²⁶KRRKR¹³⁰. The serum from rabbits was drawn 8-10 weeks after immunization, and the polyclonal antibodies in the serum were purified at small scale by Protein A column. The purified polyclonal antibodies were normalized and used for the competition Biacore testing. The His6-tagged hTSLP antigen at various concentrations was captured by the CM5 chip on which the anti-His6 antibody was immobilized, hTSLPR-Fc (SEQ ID NO: 38) at 100 nM was flowed through the surface of CM5 chip. Since there is an interaction between hTSLP and hTSLPR, the binding signal at RU (Response Unit) was detected by Biacore T200. If an antibody which blocks the interaction between hTSLP and hTSLPR is present, the binding signal is decreased in this competition Biacore assay.

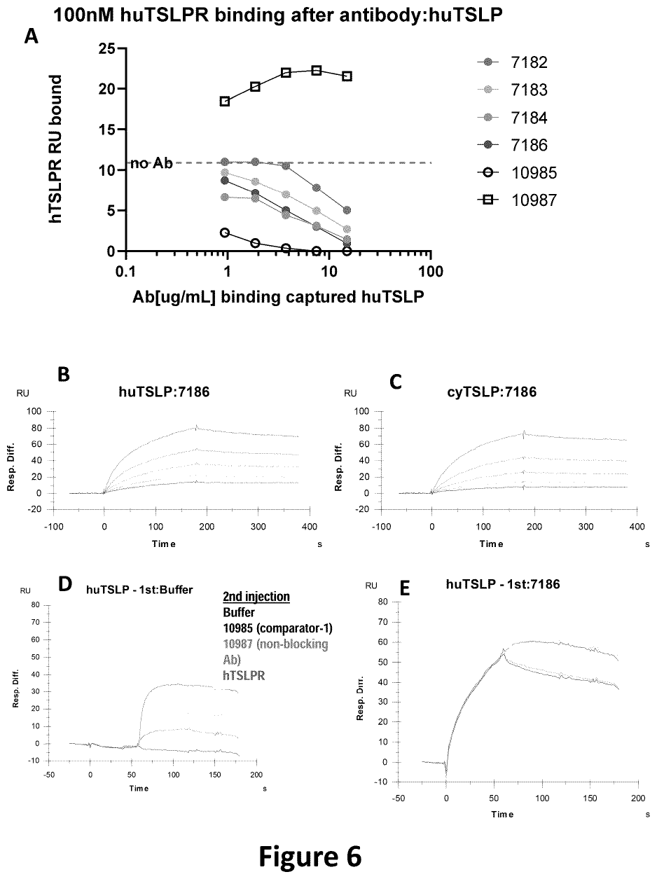

The anti-hTSLP comparator blocking antibody 10985 (open circle) showed very strong inhibition whereas a non-blocking anti-hTSLP antibody 10987 (open square) showed enhanced binding since 10987 binds to a different epitope which is not at the interface between hTSLP and hTSLPR. The polyclonal antibodies from rabbits 7184 and 7186 showed a strong inhibition whereas the polyclonal antibodies from rabbit 7183 showed a medium level of inhibition, and antibodies from rabbit 7182 showed a low level of inhibition, see FIG. 6A. The binding of polyclonal antibodies from rabbit 7184 and 7186 to hTSLP and cyTSLP was tested by Biacore, the polyclonal antibodies from rabbit 7186 showed similar RU binding level for both hTSLP and cyTSLP (FIGS. 6B and 6C) whereas the polyclonal antibodies from rabbit 7184 had half the binding level for cyTSLP compared to that for hTSLP (data not shown).

The His6-tagged hTSLP antigen at 5 μg/ml was captured at 20 ul/min by the CM5 chip on which the anti-His6 antibody was immobilized. Blank buffer was injected for 1 min followed by a second injection of blank buffer, hTSLPR-Fc at 100 nM, anti-hTSLP comparator blocking antibody 10985, or an anti-hTSLP non-blocking antibody 10987 over the surface of CM5 chip for 3 min. As shown in FIG. 6D, after the His6-tagged hTSLP antigen is captured on CM5 chip, the first injection of blank buffer did not interrupt the binding of hTSLPR, anti-hTSLP non-blocking antibody 10987, or anti-hTSLP comparator blocking antibody 10985 since they all can bind to hTSLP whereas there is no additional binding when the second injection of blank buffer is flowed through the CM5 surface. The results demonstrated that interaction between the hTSLP ligand and hTSLPR receptor; hTSLP antigen and anti-hTSLP antibody was observed by Biacore testing.

After the His6-taged hTSLP antigen is captured on CM5 chip, the first injection of purified rabbit polyclonal antibodies from rabbit 7186 at 30 μg/ml was flowed over CM5 surface. If any blocking antibody which binds to hTSLP and inhibits the interaction between hTSLP and hTSLPR is present in the rabbit polyclonal antibodies, the binding signal from the second injection of hTSLPR should be decreased. As shown in FIG. 6E, the second injection of blank buffer did not show any additional binding signal, instead the binding signal gradually drifted to decrease because there is disassociation between the captured hTSLP and flowed anti-hTSLP antibody present in the 7186 rabbit polyclonal antibodies. The second injection of hTSLPR did not generate any additional binding, instead, the binding signal between hTSLP and the purified polyclonal antibodies from rabbit 7186 was decreased, indicating that some blocking antibody in rabbit 7186 polyclonal antibodies existed to prevent the interaction between hTSLP and hTSLPR. Both non-blocking antibody 10987 and the anti-TSLP comparator blocking antibody 10985 showed additional binding signal from 60-180 seconds as expected because only a small amount of blocking antibody is present in the polyclonal antibodies of rabbit 7186. Based on all above data, the splenocytes from the rabbit 7186 were fused with the rabbit plasmacytoma cell line 240E-1 and cultured in HAT (hypoxanthine, aminopterin, and thymidine) containing medium for production of rabbit anti-hTSLP monoclonal antibodies.

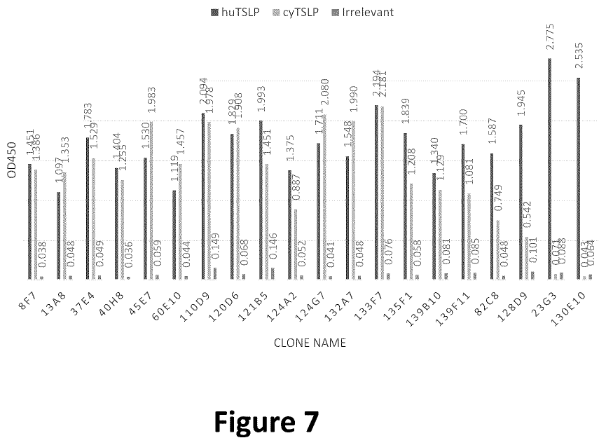

Supernatant from 14,100 wells from 150 96-w plates (in which a well for positive control and a well for negative control were used) were screened by ELISA for the binding to hTSLP, cyTSLP, or irrelevant antigen. A total of 177 hTSLP binders with signal/noise >3 were picked for secondary ELISA assay confirmation. Sixteen clones showed strong binding to both hTSLP and cyTSLP but not to irrelevant antigen, they are clones 8F7, 13A8, 37E4, 40H8, 45E7, 60E10, 110D9, 120D6, 121B5, 124A2, 124G7, 132A7, 133F7, 135F1, 139B10, 139F11. Two clones showed much stronger binding to hTSLP but weak binding to cyTSLP, they are clones 82C8 and 128D9. Two clones showed strong binding to hTSLP without any binding to cyTSLP, they are clones 23G3 and 130E10. See FIG. 7.

The 16 anti-hTSLP antibodies were produced by transient transfection of plasmid DNAs encoding the HC and LC of each antibody in ExpiCHO cells, then purified through Protein A affinity chromatography as described above. The His6-tagged hTSLP antigen at 2 μg/ml in 1×PBS buffer was coated in the 96-w Maxisorp plates overnight at 4° C., the plates were washed 3 times with 1×PBST, blocked with 2% BSA in 1×PBST, shaken at 200 rpm for 1 hr at RT. The individual anti-hTSLP rabbit antibodies or a rabbit isotype control antibody as duplicates were added at 5 μg/ml final concentration followed by the addition of the biotinylated hTSLPR at 2 μg/ml final concentration. The plates were shaken at 200 rpm at RT for 1 hr, washed 3 times with 1×PBST. The HRP-conjugated streptavidin was added to each well, the plates were washed for 3 times, the HRP substrate for color development was added in each well, the plates were shaken for 15 min before the H₂SO₄ solution was added to stop the reaction. The plates were read in Perkin-Elmer plate reader. The percentage of inhibition was plotted against blank 1×PBST buffer in which no antibody was included.

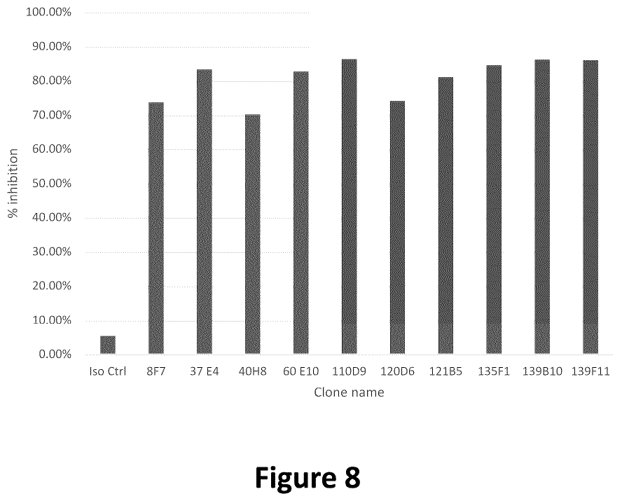

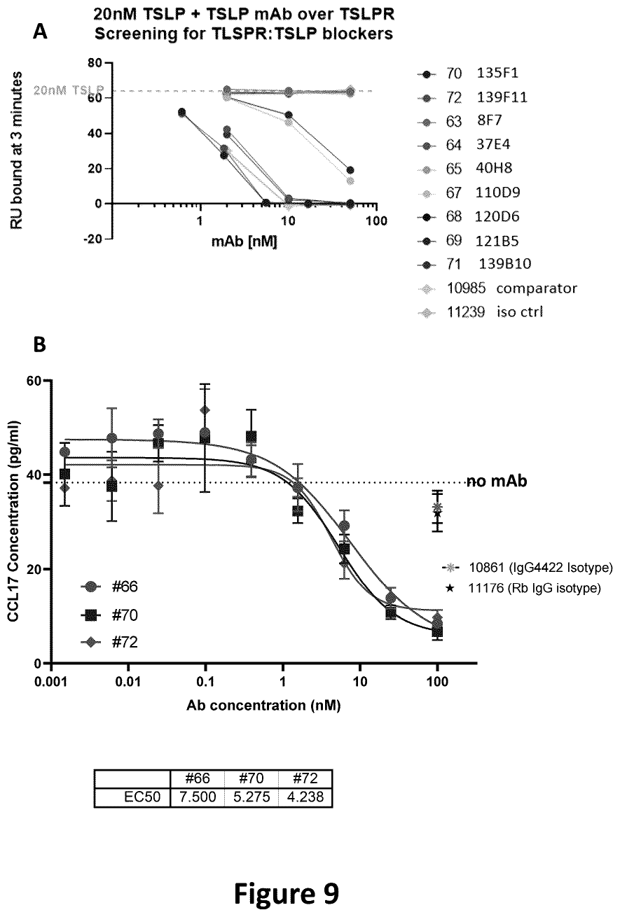

As shown in FIG. 8, the rabbit isotype control antibody had 5% basal level of inhibition, however, 10 out of 16 clones strongly inhibited the hTSLP's binding to hTSLPR (>70%) in the competition ELISA assay, they are clones 8F7, 37E4, 40H8, 60E10, 110D9, 120D6, 121B5, 135F1, 139B10, 139F11. The other 6 clones (13A8, 45E7, 124A2, 124G7, 132A7, 133F7) had <70% inhibition (data not shown). These top 10 purified rabbit IgGs were further screened by competition Biacore testing as described above. As seen from FIG. 9A, clone 135F1 rabbit IgG antibody has the highest activity in inhibiting the hTSLP-hTSLPR interaction followed in order by 139F11, the comparator antibody 10985, 139B10, 37E4, 110D9, 121B5. The top clone 135F1 at 5.5 nM can fully prevent the interaction of hTSLP-hTSLPR. The isotype control antibody 11239, 8F7, 40H8, and 120D6 did not show inhibition in this assay.

Primary human monocytes were purified from PBMCs using a EasySep™ Human Monocyte Isolation Kit from Stemcell Technologies (cat. no. 19359). On day of the assay, monocytes were seeded in 96-w plates and treated for 24 hrs with a constant amount of hTSLP (at concentrations close to EC₅₀ values) plus a titration of top anti-TSLP rabbit antibody 135F1, 139F11, or 139B10. CCL-17 chemokine levels in supernatants were determined using a human CCL-17 ELISA kit (R&D cat. no. DY364). As shown in FIG. 9B, human isotype control antibody 10861 or rabbit isotype control antibody 11176 did not inhibit the CCL17 release at the highest concentration 100 nM when compared to blank buffer. However, all three rabbit monoclonal antibodies 135F1 (#70), 139F11 (#72), and 139B10 (#66) can inhibit the CCL17 release with IC₅₀ at 4.238 nM, 5.275 nM, and 7.500 nM respectively. Clearly, the clone 135F1 showed the highest potency in this biological cell-based assay. Taken together, the lead rabbit antibody clone 135F1 (SEQ ID NOs: 39-42) was chosen for humanization by CDR grafting and yeast display.

### Example 5: Humanization of Rabbit Anti-hTSLP Antibody Clone 135F1 by CDRs Grafting and Yeast Display

To enable the successful development of rabbit monoclonal antibodies as therapeutics, the rabbit antibodies should be humanized, to reduce the chance that patients will produce neutralizing antibodies to the non-human molecules. The humanization of rabbit antibody was based on the pioneering work by Zhang Y et al. mAbs 2017; 9 (3): 419-429. The amino acid sequence of the V_{L} of the lead rabbit antibody clone 135F1 (SEQ ID NO: 41) was used to identify the position for all residues by Kabat numbering scheme (www.bioinf.org. uk/abs/abnum/), the results are shown below.

The amino acid sequence of the VH of the lead rabbit antibody 135F1 (SEQ ID NO: 39) was also used to identify the position for all residues by Kabat numbering scheme (www.bioinf org.uk/abs/abnam/), the results are shown below.

In all rabbit antibodies, the Cys residue at position 80 of VL-FW3 forms a covalent disulfide bond with the Cys residue at position 50 of VH-CDR2. However, in all human antibodies, there is no such extra disulfide bond. To start the humanization of clone 135F1, the Cys residue at position 80 of VL-FW3 is changed to Pro since this Cys80 residue is located at a turn of the VL structure; the Cys residue at position 50 of VH-CDR2 is changed to Ser since Cys residue has a similar size as Ser residue, see Zhang Y et al. mAbs 2017; 9 (3): 419-429. The new VL (C80P) and VH (C50S) sequences derived from clone 135F1 were used to search for the homologous human VL and VH sequences separately by running the IgBlast program (www.nebi nlm nih gov/igblast/). The human IGKV1-13*02 germline has the highest 67.8% identity and the human IGHV3-74*01 germline has the highest 60.4% identity with 135F1 VL sequence and VH sequence, respectively. The Kabat CDRs of VL and VH of clone 135F1 were grafted into human homologous IGKV1-13*02 and IGHV3.74*01 germlines separately.

Two β-sheets formed with β-strands ↓C″↑C′ ↓C ↑F ↓G of VL and VH regions pack together, forming a barrel-like structure that aligns the connecting CDR loops to confer the antigen-binding site. Therefore, the β-strands ↓C″ ↑C′ ↓C ↑F ↓G between VL and VH are very important to the correct folding and stability of antibodies, see Chiu M. L et al. Antibodies (Basel) 2019; 8 (4): 55. Amino acid sequences of original rabbit VL of clone 135F1 and CDRs-grafted VL on top of human IGKV1-13*02 germline were aligned using the software CLUSTALW (www.genome.jp/tools-bin/clustalw). Similarly, the amino acid sequences of original rabbit VH of clone 135F1 and CDRs grafted VH on top of human IGHV3-74*01 germline were aligned using the software CLUSTALW. The interface residues at Kabat positions 38, 42, 43, 83 are quite different between the rabbit VL and human germline IGKV1-13*02, the residue at Kabat position 70 (Q/D) is also quite different, although this position is located in the loop 4 of VL, it may contribute to the antigen binding, see Zhang Y et al. mAbs 2017; 9 (3): 419-429. Similarly, the interface residues at Kabat positions 44, 45, 89, 91 are quite different between the rabbit VH and human germline IGHV3-74*01. The ⁵⁵DS⁵⁶ motif in VH-CDR2 is a potential isomerization hot spot which could cause some instability issue, it is preferred to eliminate the potential instability issue at early stage.

TABLE 13
Degenerate codons for making yeast display library
to humanize the anti-hTSLP clone 135F1

| Kabat position | 38 | 42 | 43 | 70 | 83 |
|---|---|---|---|---|---|
| beta-strand | ↓C | ↑ C′ | ↑ C′ | ↓E | ↑F |
| rabbit 135F1-VL | H | Q | R | Q | A |
| CDRs grafted | Q | K | V | D | V |
| human VL | | | | | |
| Degenerate | C A | (A/C) A | (A/G) (G/T) A | (G/C) A | G (C/T) T |
| codons | (A/T) | A | | (A/T) | |
| Coded residues | H, Q | Q, K | R, I, G, V | E, D, Q, H | A, V |

| Kabat position | 44 | 45 | 55 | 56 | 89 | 91 |
|---|---|---|---|---|---|---|
| beta-strand | ↑ C′ | ↑ C′ | CDR2 | CDR2 | ↑F | ↑F |
| rabbit 135F1-VH | R | P | D | S | T | F |
| CDRs grafted | G | L | D | S | V | Y |
| human VH | | | | | | |
| Degenerate | (A/G) | C (C/T) | (A/G/C/T) | (A/G/C/T) | (A/G) | T |
| codons | G A | A | (A/G/C/T) | (A/G/C/T) | (C/T) | (A/T) |
| | | | (G/T) | (G/T) | T | T |
| Coded residues | R, G | P, L | all 20 | all 20 | T, I, A, V | F, Y |
| | | | residues | residues | | |

The key residues at the VL-VH interface were alternated between the rabbit residue and human germline residue using degenerate codons, the potential VH-CDR2 hot spot of isomerization motif ⁵⁵DS⁵⁶ was fully randomized using NNK codons. Due to the degeneration of codons, some extra amino acid residues may be introduced at certain positions. For example, the degenerate codon (A/G) (G/T) A at position 43 of VL encodes Arg(R), Ile(I), Gly(G), Val(V), more than the R and V residues alternated between the rabbit 135F1 VL and human IGKV1-13*02.

To quickly humanize the rabbit 135F1 antibody, 6 long oligos that cover the 5 degenerate codons in VL (shown in the Table 13 above) at a length of around 100 nt were used to assemble the full-length VL by PCR reactions with a short forward oligo and a reverse oligo. Similarly, 6 long oligos that cover the 4 degenerate codons and 2 randomized codons in VH at a length of around 100 nt were used to assemble the full-length VH by PCR reactions with another short forward oligo and another reverse oligo. A Fab library for yeast display as described in Example 2 above was constructed to select the humanized Fab variants through MACS and FACS approaches.

The induced yeast library was processed with 500 nM biotinylated hTSLP (bn-hTSLP) for the 1^{st} round of MACS, and with 100 nM biotinylated cyTSLP (bn-cyTSLP) for the 2^{nd} round of MACS to enrich the positive binders. The collected yeast pools were further sorted by FACS for 3 rounds with consecutively decreased concentrations of antigen: 10 nM bn-hTSLP antigen; 3 nM bn-cyTSLP antigen; 0.5 nM bn-hTSLP antigen. After the final FACS sorting, 94 individual yeast colonies were randomly picked with irrelevant yeast colony in well A01 and a positive yeast colony in well B01. Yeast cells were induced and subjected to FACS analysis using the complex of streptavidin-APC and bn-hTSLP (or bn-cyTSLP) and the ALEXA FLUOR®-labeled antibody specific for the HA tag. Of 94 individual colonies, 10 had strong antigen binding (Q2>30%), 17 had medium strong antigen binding (Q2=20~30%), and 21 had weak antigen binding (Q2=10~20%), the rest of yeast colonies showed negligible antigen binding. All 48 binders were subject to PCR reactions to amplify the VL and VH fragments then DNA sequencing. After sequence alignments, 6 unique VHs and 6 unique VLs can make up the combination of all 48 binders. Redundancy existed, for example, 20 binders share the same VL whereas other 28 binders share other 5 unique VLs.

The DNA gBlocks encoding these 6 VHs and 6 VLs were synthesized and converted to human IgGI as described in Example 2. The purified antibodies were subject to Biacore analysis as described above. The Table 14 below showed that the top clone QB1341 (SEQ ID NOs: 43 and 44 for VH and HC respectively; SEQ ID NO: 45 and 46 for VL and LC respectively) has 1.76 nM binding affinity to hTSLP (R127A+R130S) antigen (SEQ ID NO: 34), which is of around 10-fold lower binding affinity than that of the comparator antibody QB10985 with 0.184 nM binding affinity.

TABLE 14
Biacore kinetics of the lead humanized
anti-hTSLP antibody QB11341

| | Ka | Kd | KD | Rmax | |
|---|---|---|---|---|---|
| | (M⁻¹s⁻¹) | (s⁻¹) | (10⁻⁹ M) | (RU) | SD |
| QB11341 | 8.42 × 10⁵ | 1.48 × 10⁻³ | 1.76 | 42.65 | 0.413 |
| (top clone) | | | | | |
| QB10985 | 1.16 × 10⁶ | 2.13 × 10⁻⁴ | 0.184 | 45.37 | 0.413 |
| (comparator) | | | | | |

Human monocytes were purified from a healthy donor using a EasySep™ Human Monocyte Isolation Kit from Stemcell Technologies (cat. no.19059). On day of the assay, monocytes were seeded in 96-w plates and treated for 24 hrs with a constant amount of different flavor of hTSLP at concentrations close to EC₅₀ plus a titration of anti-hTSLP antibodies. The hTSLP or cyTSLP-induced CCL-17 chemokine levels in supernatants were determined using human CCL-17 ELISA kit (R&D Systems, cat. no. DY364).

TABLE 15
Inhibition of anti-hTSLP antibody on the hTSLP-induced CCL-17
secretion of human PBMCsN.A. means “not available)

| | | IC50 (nM) for inhibition of CCL17 secretion | | |
|---|---|---|---|---|
| | | hTSLP | hTSLP | hTSLP |
| Testing | | (del | (R127A + | (WT) + |
| Sample | Description | KRRKR) | R130S) | furin inhibitor |
| QB11571 | Isotype Control | N.A. | N.A. | N.A. |
| QB11341 | top clone | 0.3614 | 0.6265 | 0.3332 |
| QB10985 | comparator | 0.009714 | 0.004307 | 0.01047 |
| | analogue | | | |

The results in Table 15 showed that the anti-hTSLP top clone QB11341 has a 30~50 fold lower potency than the comparator analogue QB10985 in the hTSLP-induced CCL-17 secretion whereas the antibody isotype control has no inhibition.

TABLE 16
Inhibition of anti-hTSLP antibody on the cyTSLP-induced CCL-17
secretion of human PBMCsN.A. means “not available”

| | | IC50 (nM) for inhibition of CCL17 secretion | | |
|---|---|---|---|---|
| | | cyTSLP | cyTSLP | cyTSLP |
| Testing | | (del | (R127A + | (WT) + |
| Sample | Description | KRRKR) | R130S) | furin inhibitor |
| QB11571 | Isotype Control | N.A. | N.A. | N.A. |
| QB11341 | top clone | 0.7116 | 0.9297 | 0.8882 |
| QB10985 | comparator | 0.03527 | 0.06037 | 0.0212 |
| | analogue | | | |

The results in Table 16 showed that the anti-hTSLP top clone QB11341 has a 15~40 fold lower potency than the comparator analogue QB10985 in the cyTSLP-induced CCL-17 secretion whereas the antibody isotype control has no inhibition.

### Example 6: Affinity Maturation of the Top Anti-hTSLP Antibody QB11341 by Yeast Display for Activity Improvement

The top humanized anti-hTSLP clone QB11341 has a binding affinity of 1.76 nM to hTSLP (R127A+R130S) antigen, which is around 10-fold lower than that of anti-hTSLP comparator analogue QB10985, see Table 14. Generally, a higher binding affinity of an antagonistic antibody could lead to a higher blocking activity. The clone QB11341 was further engineered in its VL region by yeast display technology to improve the binding affinity to hTSLP and cyTSLP antigens for potential improved blocking potency.

A total of 5 residues in VL-CDR1 (position 34), VL-CDR2 (position 53), and VL-CDR3 (positions 91, 92, 93) were fully randomized using the degenerate codon NNK in the designed oligos, see Table 17 below.

A new VL library linked with the VH region from the parental clone QB11341 was constructed to display the Fab fragments on yeast surface. Two rounds of MACS selection and 3 rounds of FACS sorting were conducted as described in Example 2 to fish out the beneficial VL variants which would lead to a higher binding affinity. To find the Fab fragments which have cross-species binding to human and cynomolgus TSLP antigens, bn-hTSLP and bn-cyTSLP antigens were alternatively applied during the process of yeast display. After screening 94 individual colonies by FACS analysis, the VL fragment from top 20 binders were amplified by PCR and the sequences were revealed by DNA sequencing. The results showed that there were 8 different VL among the top 20 binders. The amino acid sequences of all 8 VL variants were used to design gBlocks of DNA sequences and converted to full-length LC as described in Example 2. Finally, 8 new recombinant antibodies were made by transient transfection of ExpiCHO cells and purified with Protein A column and subjected to Biacore analysis. The results showed that the new antibody QB11548 (SEQ ID NOs: 43, 44, 47, 48) has a higher binding affinity to both hTSLP (R127A+R130S) and cyTSLP (R127A+R130S) antigen than the parental clone QB11341. The substitutions K53R in VL-CDR2 and F92R in VL-CDR3 were introduced in QB11548 for the improved binding affinity. The inhibition of TSLP-induced CCL17 secretion from PBMCs also suggested that the clone QB11548 has more potent blocking activity than the parental clone QB11341.

TABLE 18
Inhibition of anti-hTSLP antibody on the hTSLP-induced CCL-17
secretion of human PBMCsN.A. means “not available”

| | | IC50 (nM) for inhibition of CCL17 secretion | | |
|---|---|---|---|---|
| | | hTSLP | hTSLP | hTSLP |
| Testing | | (del | (R127A + | (WT) + |
| Sample | Description | KRRKR) | R130S) | inhibitor |
| QB11571 | Isotype Control | N.A. | N.A. | N.A. |
| QB11548 | top clone | 0.09205 | 0.2859 | 0.07574 |
| QB10985 | comparator | 0.009714 | 0.004307 | 0.01047 |
| | analogue | | | |

The results in Table 18 showed that the anti-hTSLP top clone QB11548 has a 5~10 fold lower potency than the comparator analogue QB10985 in the hTSLP-induced CCL-17 secretion whereas the antibody isotype control has no inhibition.

TABLE 19
Inhibition of anti-hTSLP antibody on the cyTSLP-induced CCL-17
secretion of human PBMCsN.A. means “not available”

| | | IC50 (nM) for inhibition of CCL 17 secretion | | |
|---|---|---|---|---|
| | | cyTSLP | cyTSLP | cyTSLP |
| Testing | | (del | (R127A + | (WT) + |
| Sample | Description | KRRKR) | R130S) | furin inhibitor |
| QB11571 | Isotype | N.A. | N.A. | N.A. |
| | Control | | | |
| QB11548 | top clone | 0.2509 | 0.4639 | 0.2544 |
| QB10985 | comparator | 0.03527 | 0.06037 | 0.0212 |
| | analogue | | | |

The results in Table 19 showed that the anti-hTSLP top clone QB11548 has a 7~12 fold lower potency than the comparator analogue QB10985 in the cyTSLP-induced CCL-17 secretion whereas the antibody isotype control has no inhibition.

### Example 7: Humanization of a Rat-Anti-hTSLP Antibody and Affinity Maturation by Yeast Display

A rat-anti-hTSLP antibody clone 23B12 from a hybridoma was deposited at the American Type Culture Collection (10801 University Blvd., Manassas, VA 20110-2209, USA) with the patent deposit designation “PTA-7951”. The hybridoma was deposited on Oct. 26, 2006 under the conditions of the Budapest Treaty, and received accession number PTA-79-51. The amino acid sequences of VH and VL of clone 23B12 were extracted from patent U.S. Pat. No. 8,232,372B2, SEQ NOs: 57 and 59. The CDRs in VH of clone 23B12 were grafted into human germline IGHV1-69:06 and IGHJ101; the CDRs in VL of clone 23B12 were grafted into human germline IGKV3-20*01 and IGKJ4*01. Appropriate back mutations were introduced in the humanized antibody to maintain correct folding and structure. Four humanized antibody variants were made by transient transfection in ExpiCHO cells and purified by Protein A chromatography as described in Example 2 above. One variant hz-3C QB10990, SEQ ID NOs: 61-64) showed good production and comparable binding to the hTSLP and cyTSLP antigens, however, this antibody had only 90.2% main peak and 9.2% HMW peak in SEC analysis. To mitigate the aggregation issue, a new round of antibody engineering was conducted by testing the effect of different germline residues in the original rat antibody 23B12. One antibody variant hz-3C-V6 (QB11060, SEQ NOs: 63-66) which has D9P and S108L back mutations in FW1 and FW4 of VH region respectively showed a high yield; high SEC profile (97.9% main peak); quick Kon and high RU (reactive unit) by Biacore analysis; <10-fold binding difference when comparing with cyTSLP antigen. However, the binding affinity to hTSLP (R127A+R130S) is at around 1.62 nM, which is 8.8-fold lower than the comparator QB10985, see Table 20 below.

TABLE 20
Biacore kinetics of the humanized anti-hTSLP antibody clone
QB11060 (hz-3C-V6) and the comparator antibody QB10985.

| | Ka | Kd | KD | Rmax | |
|---|---|---|---|---|---|
| | (M⁻¹s⁻¹) | (s⁻¹) | (10⁻⁹M) | (RU) | SD |
| QB11060 | 5.27 × 10⁵ | 8.52 × 10⁻⁴ | 1.62 | 46.83 | 0.398 |
| (hz-3C-V6) | | | | | |
| QB10985 | 1.16 × 10⁶ | 2.13 × 10⁻⁴ | 0.184 | 45.37 | 0.413 |
| (comparator) | | | | | |

To achieve a higher blocking activity, 5 amino acid residues in VL of the anti-hTSLP clone QB11060 were fully randomized by NNK codons for yeast display as described in our patent U.S. Pat. No. 11,124,570 and Example 2 above to quickly find the beneficial variants which confer higher binding affinity with potentially higher biological activity, see Table 21 below.

Two rounds of MACS selection and three rounds of FACS sorting were carried out to enrich the high-affinity binders using alternative bn-hTSLP and bn-cyTSLP antigens during selection process to find cross-species binding clones. The VL fragments from top 20 binders were amplified by PCR reactions for DNA sequencing, then 6 unique clones with new VL sequence were converted to human IgG4 from production in ExpiCHO cells. Characterizations including Biacore, SEC analysis, cell-based CCL17 release assay were performed to identify the lead clone QB11237 IgG4 which has 5 new amino acid residues in VL-CDRs comparing to the original clone 23B12, the changed residues are P28H, I31R, T91S, F92Y, and L94F.

### Example 8: Inhibition Mechanism of the Anti-hTSLP Antibodies QB11341 and QB11237 by Biacore Analysis

To explore how the anti-hTSLP antibodies QB11341 and QB11237 inhibit the hTSLP-induced biological effects, Biacore assays were conducted to assess (1). the ligand-receptor interaction between hTSLP and hTSLPR; (2). the direct blocking of hTSLP-hTSLPR axis by antibody QB11341 and the blocking of hTSLP-hIL7Rα interaction by antibody QB11237; (3). the inhibition level of hTSLP-hIL7Rα interaction by antibody QB11237.

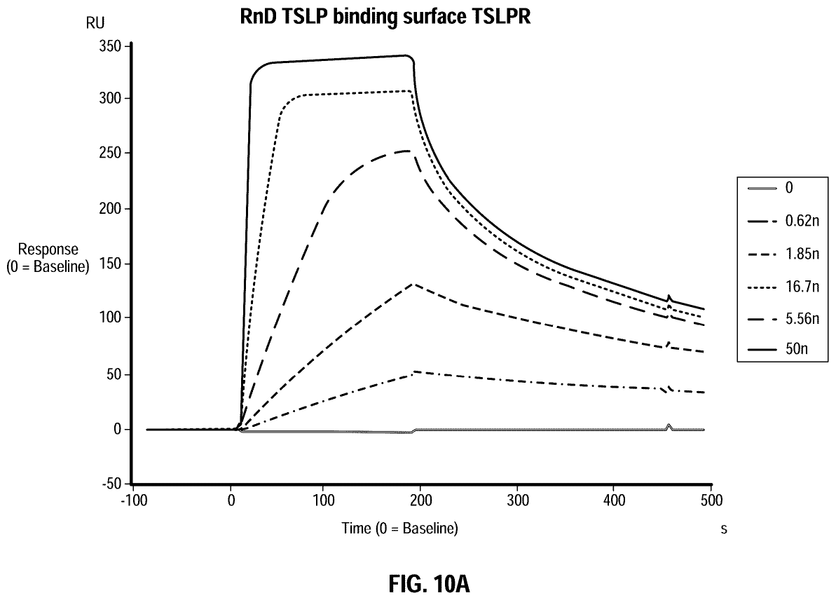

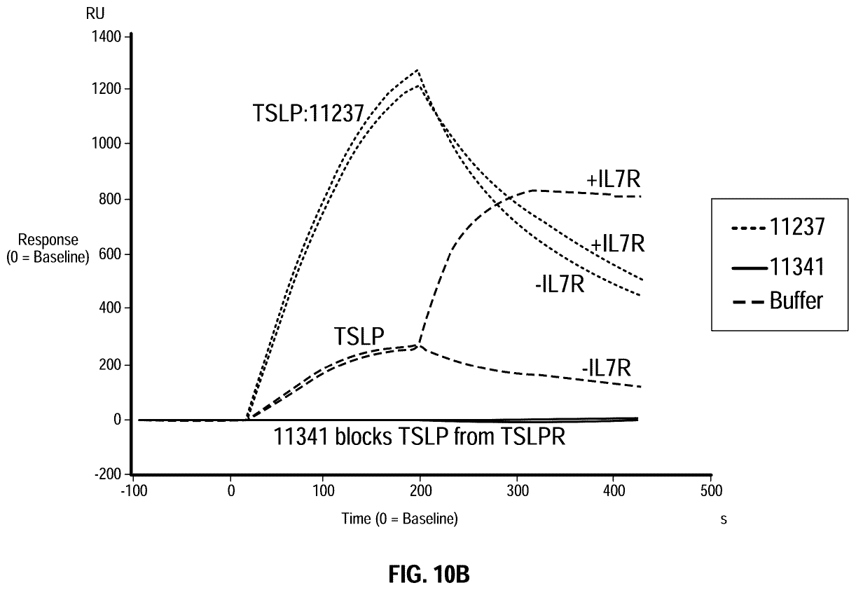

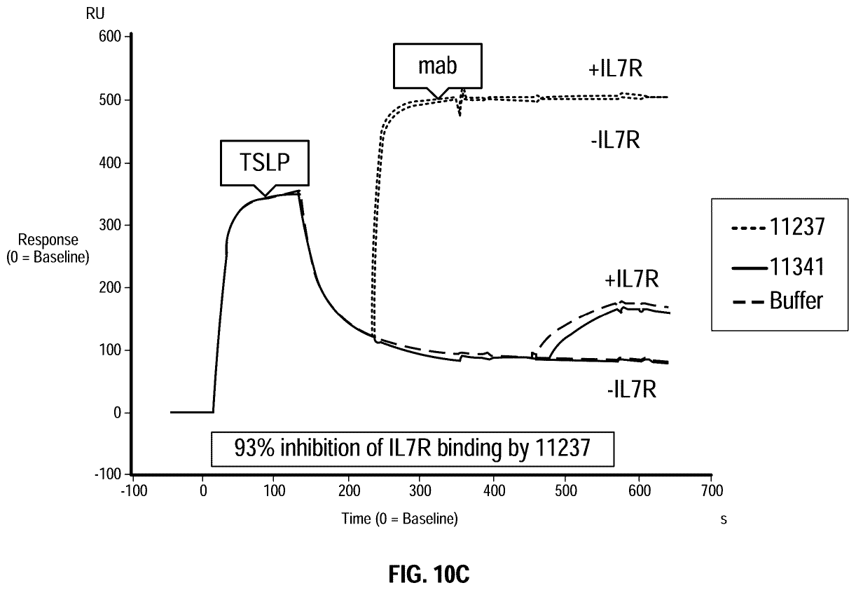

The hTSLPR-Fc dimer molecules from R&D Systems (cat. no. 981-TR) were directly immobilized on the CM5 surface by amine coupling method at high, medium, low density in flow cell 2, 3, 4 whereas no hTSLPR-Fc was immobilized in flow cell 1. The running buffer was flowed through flow cell 1, therefore, the background binding from flow cell 1 was a reference which was subtracted from the results of flow cell 2, 3, or 4 to obtain the real binding. The ligand hTSLP monomer antigen was injected over the CM5 surface at 0, 0.62, 1.85, 5.56, 16.7, and 50 nM, the association time was 3 min, the dissociation time was 5 min. The kinetic sensorgram was analyzed with BIAevaluation 4.1.1. As shown in FIG. 10A, the hTSLP ligand binds to immobilized hTSLPR-Fc in 1:1 mode with a dose dependent manner, the estimated binding affinity between hTSLP and hTSLPR is around 6 nM.

After the demonstration of direct hTSLP-hTSLPR interaction on CM5 surface, the same hTSLPR-Fc immobilized CM5 chip was injected with hTSLP alone or a premixed hTSLP-antibody complex with either antibody QB11341 or QB11237 for 3 min, finally 50 nM of hIL7Rα (R&D Systems, cat. no. 206-IR) or blank buffer was injected over the CM5 surface with an association time for 2 min and a dissociation time for 5 min. As shown in the middle part of FIG. 10B, when only hTSLP was injected, a peak from 0-300 RU was observed from time point 20-200 seconds, after that, when hIL7Rα was continuously injected, an additional peak from 300-900 RU was observed whereas the peak gradually drifted down when hIL7Rα was not injected because of the slow dissociation of hTSLP-hTSLPR complex. These results reflected the fact that hTSLP can bind to hTSLPR on CM5 chip surface and hIL7Rα further binds to hTSLP-hTSLPR to form a tripartite complex. As shown in the top part of FIG. 10B, when mixed hTSLP-QB11237 was injected, a peak from 0-1200 RU was observed, suggesting that the hTSLP in hTSLP-QB11237 complex can still bind to hTSLPR on CM5 surface. From time point 200-450 seconds, whether or not the hIL7Rα was injected, no additional peak was observed, instead, the sensorgram started to decrease from time point 200 seconds. The results indicated that hIL7Rα can no longer bind to the hTSLPR-hTSLP-QB11237 complex. As shown in the bottom part of FIG. 10B, there is not additional sensorgram peak when the premixed hTSLP-QB11341 complex was firstly injected to the CM5 surface where the hTSLPR-Fc was immobilized, indicating that QB11341 fully prevents the hTSLP's binding to hTSLPR-Fc.

The hTSLPR-Fc immobilized CM5 chip was injected with 20 nM of hTSLP with an association time of 2 min, and a dissociation time of 100 sec, anti-hTSLP antibody QB11341 or QB11237 at 20 nM was subsequently injected over the CM5 surface with an association time of 3 min and a quick dissociation time of 1 min, finally 50 nM of hIL7Rα or the blank running buffer was injected with an association time of 2 min and a dissociation time of 2 min. As shown in FIG. 10C, from time point 20-140 seconds, a peak from 0-300 RU was observed, indicating that the ligand hTSLP binds to the immobilized hTSLPR on CM5 surface. From time point 140-240 seconds, the peak gradually went down, indicating that the ligand hTSLP starts to dissociate from the complex of hTSLP-hTSLPR. At the time point 240 seconds, when the anti-hTSLP antibody QB11237 or QB11341 was injected, a sharp peak to ~1000 RU for QB11237 was observed whereas the sensorgram continued to drifted down for antibody QB11341, the results suggested that antibody QB11237 can still bind to the hTSLP-hTSLPR complex whereas the antibody QB11341 can't bind to hTSLP once the hTSLP and hTSLPR forms a complex, that means, QB11237 is not a direct blocking antibody of hTSLP whereas QB11341 is a direct blocker of hTSLP. At time point 440 seconds, either hIL7Rα or blank buffer was injected, a peak from 100 RU to 200 RU was observed for antibody QB11341, indicating that hIL7Rα can bind to the formed hTSLPR-hTSLP-QB11341 tripartite complex since the hTSLP still has an available interface for hIL7Rα binding. However, whether hIL7Rα was injected or not, no significant peak was observed for antibody QB11237. By calculating the peak height difference in the presence and absence of the antibody QB11237, the inhibition was estimated to be ~93%. The result suggested that antibody QB11237 blocks the interaction between hTSLP and hIL7Rα.

In summary, two types of hTSLP inhibitor were identified. The anti-hTSLP antibody QB11341 (and derived variants QB11548 and QB11764 as described in Examples 9-12 below) is a direct blocker of hTSLP, preventing the interaction between hTSLP and hTSLPR; the anti-hTSLP antibody QB11237 (and derived variant QB11718 as described in Example 9-12 below) doesn't directly block the interaction of hTSLP-hTSLPR, instead, antibody QB11237 prevents the hTSLP's binding to hIL7Rα. As explained in background, the receptor for hTSLP consists of two subunits, hTSLPR and hIL7Rα, the intracellular domain (ICD) of both receptor units can be phosphorylated for signal transduction, therefore, inhibiting the formation of either hTSLP-hTSLPR complex or hTSLP-hIL7Rα complex can prevent the hTSLP-mediated biological effects.

### Example 9: Generation of MabPair Consisting of Anti-IL-33 IgG4 and Different Anti-TSLP IgG1-D265A Antibodies

As described in our patent application WO2021041678A1, chain drop-out experiments were done to assess the natural pairing capability of matched and mismatched HCs and LCs. The plasmid DNAs encoding the HC and LC of anti-hIL-33 antibody QB11465, anti-hTSLP antibody QB11548, and anti-hTSLP antibody QB11237 were individually purified using a Qiagen® Midi-prep kit (Qiagen N.V., the Netherlands). The resulting DNAs were diluted in water and mixed in EPPENDORF TUBES®. A set of 4 tubes of mixed DNAs were transiently transfected into EXPICHO™ cells to assess whether non-cognate HC/LC pairings would naturally occur. Tube 1 contained DNAs encoding anti-hTSLP antibody HC (HC1) and its cognate LC (LC1). Tube 2 contained DNAs encoding a non-cognate HC/LC pair consisting of HC1 from anti-hTSLP and the LC2 from anti-hIL-33 antibody. Tube 3 contained the DNAs encoding a cognate HC/LC pair consisting of HC2 and LC2 of the anti-hIL-33 antibody. Tube 4 contained DNAs encoding a non-cognate HC2 of anti-hIL-33 and LC1 of anti-hTSLP antibody.

In more detail, the EXPICHO™ cells were transfected with the plasmid DNAs encoding the test antibody with LIPOFECTAMINE® 2000 in 24-well deep well blocks. Cells were continuously shaken at 150 rpm at 37° C. for 12 days. The supernatants were harvested by spinning down cells at 1,200 rpm for 15 minutes. For all samples (all of which were not reduced), 5 microliters (ul) of supernatant and 5 μl of 2×Laemmli Sample Buffer (65.8 mM Tris-HC1, pH 6.8, 2.1% sodium lauryl sulfate (SDS), 26.3% (w/v) glycerol, 0.01% bromophenol blue) were heated at 70° C. for 10 minutes. The treated samples were loaded into the wells of 4-15% CRITERION™ TGX STAIN-FREE™ Precast SDS-PAGE gels (Bio-Rad Laboratories, Inc., Hercules, CA, cat no. 567-8085). Electrophoresis was run for 45 min at 200 V. The image was visualized with a CHEMIDOC™ XRS+ imager from Bio-Rad Laboratories, Inc.

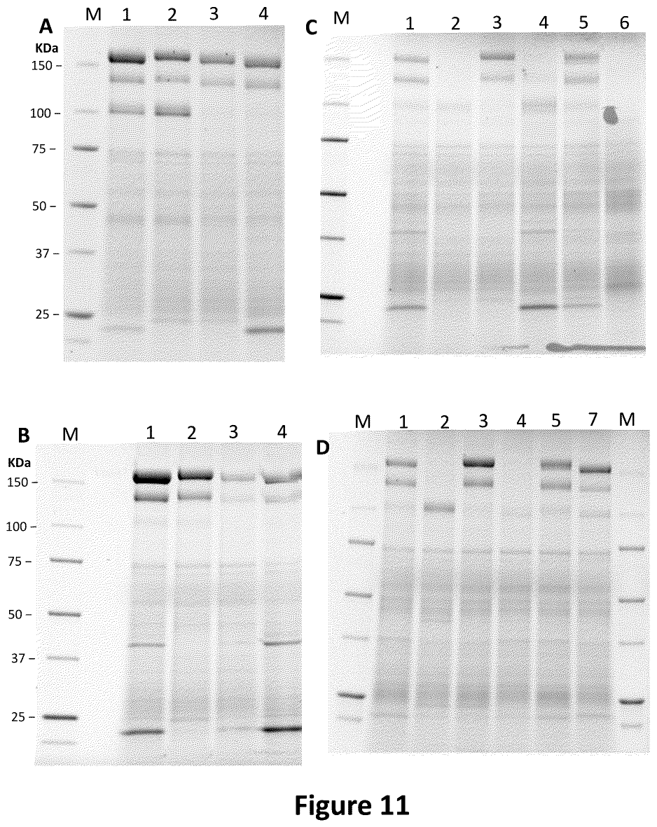

As shown in FIG. 11A, the anti-hTSLP QB11548 (lane 1) and anti-hIL-33 QB11465 (lane 3) antibodies are expressed well, since the band intensity of full-length antibody at ~150 KDa is strong. The antibody resulting from the cognate HC1/LC1 pair of anti-hTSLP QB11548 (lane 1) was expressed at a slightly higher level than the antibody resulting from the non-cognate pair of HC1 (from anti-hTSLP) and LC2 (from anti-hIL-33) in lane 2. This suggests that the HC1 of anti-hTSLP QB11548 can express better with its own LC1 than the non-cognate LC2 from anti-hIL-33 antibody QB11465. Interestingly, the non-cognate pairing of LC1 (from anti-hTSLP) and HC2 (from anti-hIL-33) is expressed at a slightly higher level than the cognate HC2/LC2 pair, FIG. 11A, compare lane 3 to lane 4. These results suggest that the HC2 of anti-hIL-33 QB11465 can work very well with non-cognate LC1 of anti-hTSLP QB11548 for expression over the cognate LC2. Therefore, the main issue to address in engineering these antibodies for making MabPair is the mispairing of HC2 from anti-hIL-33 QB11465 and LC1 from anti-hTSLP antibody QB11548, which is a direct blocker of hTSLP-hTSLPR axis.

The chain drop-out experiments for anti-hTSLP antibody QB11237 and anti-IL-33 antibody QB11465 were carried out similarly as described above. As shown in FIG. 11B, the co-expression of the matched HC1 and LC1 from anti-hTSLP QB11237 (lane 1) produced more full-length antibody from the co-expression of mis-matched HC1 from anti-hTSLP antibody QB11237 and LC2 from anti-IL-33 antibody QB11465 (lane 2). However, the matched HC2 and LC2 from anti-IL-33 antibody QB11237 produced less full-length antibody (lane 3) than the mis-matched HC2 from anti-hIL-33 antibody QB11237 and LC1 from anti-hTSLP antibody QB11237 (lane 4), suggesting that the main issue for making MabPair of these two antibodies in single cell line is to address the mispairing of HC2 from anti-hIL-33 antibody QB11465 and LC1 from anti-hTSLP antibody QB11237.

In the following experiment, the antibodies were altered to strengthen cognate HC/LC pairs, prevent non-cognate HC/LC pairs, and prevent HC/HC heterodimers. As described in Examples 2 and 3 of U.S. Pat. No. 11,130,808 (which is incorporated herein by reference), MabPair antibody consisting of altered version of anti-hTSLP antibody and unaltered anti-hIL-33 antibody were made as follows. Substitutions K147D, V173C, C220G for strengthening the cognate chain pairing in CH1; substitution D265A for the attenuated effector function (ADCC, ADCP, CDC) in CH2; substitutions D399R and K409E for preventing the formation of heterodimeric HCs in CH3 were introduced into the HC of anti-hTSLP antibody QB11548. The DNA fragment encoding the altered HC of anti-hTSLP antibody QB11548 was synthesized by IDT followed by a Gibson reaction with expression vector pSB01 to assemble the DNA encoding a full-length HC. Accordingly, substitutions S131K, S162C, C214S were introduced into the LC of anti-hTSLP antibody QB11548. The altered anti-hTSLP antibody is renamed as clone QB11764. SEQ ID NOs: 52 and 51 show the amino acid sequence of anti-hTSLP QB11764 HC and the nucleic acid sequence encoding it, respectively. SEQ ID NOs: 56 and 55 show the amino acid sequence of anti-hTSLP QB11764 LC and the nucleic acid sequence encoding it, respectively.

Since the same main issue for making MabPair of anti-hIL-33 QB11465 with either anti-hTSLP QB11548 or QB11237 is to prevent the mispairing of HC2 from anti-hIL-33 QB11465 and LC1 from anti-hTSLP antibody QB11548 or QB11237, the same set of substitutions K147D, V173C, C220G, D265A, D399R, and K409E are introduced in the HC of anti-hTSLP antibody QB11237; S131K, S162C, C214S are introduced in the LC of anti-hTSLP antibody QB11237, the new anti-hTSLP antibody with such substitutions is renamed as clone QB11718. SEQ ID NOs: 72 and 71 show the amino acid sequence of anti-hTSLP QB11718 HC and the nucleic acid sequence encoding it, respectively. SEQ ID NOs: 76 and 75 show the amino acid sequence of anti-hTSLP QB11718 LC and the nucleic acid sequence encoding it, respectively.

Plasmid DNAs encoding HCs and LCs, which made up one antibody or two different antibodies, were put into a series of EPPENDORF test tubes. The tubes contained DNAs encoding the following antibodies: Tube 1 contained DNAs encoding anti-hTSLP antibody QB11764 HC (HC1) and its cognate LC (LC1). Tube 2 contained DNAs encoding a non-cognate HC/LC pair consisting of HC1 from anti-hTSLP QB11764 and the LC2 from anti-hIL-33 antibody QB11465. Tube 3 contained the DNAs encoding a cognate HC/LC pair consisting of HC2 and LC2 of the anti-hIL-33 antibody QB11465. Tube 4 contained DNAs encoding a non-cognate HC2 of anti-hIL-33 QB11465 and LC1 of anti-hTSLP antibody QB11764. Tube 5 contained all four DNAs encoding HC1 and LC1 of the anti-hTSLP QB11764, HC2 and LC2 of the anti-hIL-33 antibody QB11465. Tube 6 did not contain any plasmid DNA to serve as a mock control. Tube 7 contained the DNAs encoding a HC and a LC of anti-HER2 trastuzumab IgGI to assess transfection efficiency. The mixed plasmid DNAs were used to transfect 3 mL of EXPICHO™ cells in 24-well plate. The plate containing the transfected EXPICHO™ cells was shaken at 37° C. at 10% CO₂ for 12 days. A volume of 10 μl harvested supernatant was loaded in each lane of a 4-15% CRITERION™ TGX STAIN-FREE™ Precast SDS-PAGE gel as described above.

As shown in FIG. 11C, a full-length antibody at a size of 150 KDa was observed for the cognate HC1 and LC1 of anti-hTSLP antibody QB11764 (lane 1) and for the cognate HC2 and LC2 of anti-hIL-33 antibody QB11465 (lane 3). However, no obvious band at a size of 150 KDa was observed for the non-cognate HC1 of anti-hTSLP antibody QB11764 and LC2 of anti-hIL-33 antibody QB11465 (lane 2); HC2 of anti-hIL-33 antibody QB11465 and LC1 of anti-hTSLP antibody QB11764. The mixture of all four chains for both anti-hTSLP QB11764 and anti-hIL-33 antibody QB11465 produced antibody mixtures (lane 5) at around 150 KDa whereas mock transfection did not yield any antibody in lane 6. The results suggested that only the matched HC and LC can produce the full-length antibody whereas the mis-matched HC and LC are prevented from producing the full-length antibody, when making MabPair in the same host cell.

Similar results were obtained for making the MabPair which consists of anti-hTSLP antibody QB11718 and anti-hIL-33 antibody QB11465. As shown in FIG. 11D, only the matched HC and LC from anti-hTSLP antibody QB11718 produced the full-length antibody (lane 1); matched HC and LC of anti-hIL-33 antibody QB11465 produced the full-length antibody (lane 3). The mis-matched HC1 and LC2 in lane 2, or mis-matched HC2 and LC1 in lane 4 did not produce any full-length antibody. The mixture of all four chains for both anti-hTSLP QB11764 and anti-hIL-33 antibody QB11465 produced antibody mixtures (lane 5) at around 150 KDa, the HC and LC of anti-HER2 trastuzumab produced a full-length antibody (lane 7), indicating the good transfection efficiency in the experiments. The results suggested that after appropriate mutations are introduced in the anti-hTSLP antibody QB11718, only the matched HC and LC can produce the full-length antibody whereas the mis-matched HC and LC are prevented to produce the full-length antibody when making MabPair product.

### Example 10: Confirmation of MabPair QB11750 Consisting of Anti-IL-33 IgG4 Antibody QB11465 and Anti-TSLP IgG1-D265A Antibody QB11718 by Mass Spectrometry

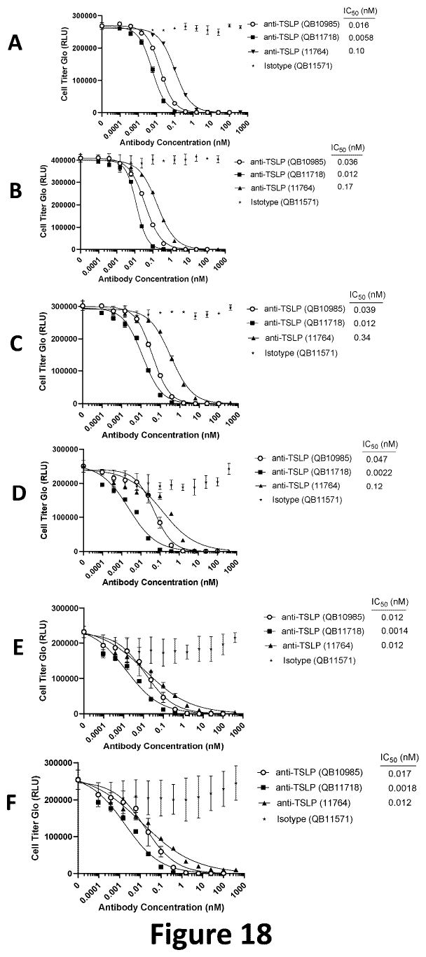

The recombinant monoclonal anti-hIL33 IgG4 antibody QB11465 and anti-hTSLP IgG1-D265A antibody QB11718 were produced by transient transfection with 4 plasmid DNAs encoding the respective HC and LC in EXPICHO™ cells. After 12 days of incubation by shaking at 37° C. at 10% CO₂, the supernatant was harvested and purified through the Protein A column by a standard protocol to generate the MabPair product QB11750. Mass spectrometry was conducted to demonstrate absence of any mispairing for HCs and LCs. Mass spectrometry methods used are described by Thompson et al. (2014), mAbs 6:1: 197-203, which is incorporated herein in its entirety, and in WO 2017/205014, page 92, line 31 to page 94, line 10 and FIG. 18, which portions of WO 2017/205014 are incorporated herein by reference.

To do this analysis, 20 μg of the purified MabPair QB11750 which consists of anti-hIL-33 IgG4 antibody QB11465 and anti-hTSLP IgG1-D265A antibody QB11718 was incubated at 37° C. with 2 μl of PNGase F endopeptidase (New England Biolabs, cat. No. P0704S) in 20 μl of 50 mM Tris pH 7.5 for 16 hr. PNGase F is the most effective enzyme for removing almost all N-linked oligosaccharides from glycoproteins. PNGase F is an amidase, which cleaves between the innermost GlcNAc and asparagine residues of high mannose, hybrid, and complex oligosaccharides. After deglycosylation, half of the sample was reduced by incubation at 55° C. in a buffer containing 4 M Guanidine Hydrochloride, 50 mM Tris pH8.0 with 50 mM DTT for 30 minutes. HPLC-MS analysis of the non-reduced or reduced samples was performed using an Agilent 6224 accurate-mass TOF mass spectrometer equipped with an ESI source and coupled to an Agilent 1200 HPLC. An Agilent Pursuit Diphenyl column (2.0×150 mm, 3 μm) was used with a column temperature of 80° C. and a flow rate of 0.4 μl/min. Mobile phase A consisted of water with 0.1% trifluoroacetic acid (TFA), and mobile phase B consisted of isopropyl alcohol (IPA): acetonitrile (ACN): water (70:30:10) with 0.9% TFA. Mobile phase B was held initially at 10%, then raised to 32% B over 5 minutes, and then increased to 42% over 35 minutes. The solvent was then changed to 90% B and held for 4 minutes to clean up the column. Finally, the solvent was reverted to 10% B and held for 4 minutes for re-equilibration of the column. MS instrumental parameters were as follows: the drying gas temperature, drying gas flow and nebulizer were set at 300° C., 12 L/min and 40 psig, respectively. The capillary, fragmentor, skimmer1 and Oct RF Vpp were set at 4500V, 250V, 60V and 750V, individually. The instrument was calibrated in m/z range of 100 to 3000 at 4 GHz high resolution. Data from HPLC-MS were analyzed using Agilent MassHunter Qualitative and BioConfirm software. Theoretical sizes of all deglyosylated antibody species that could potentially form in cells containing DNAs encoding anti-hIL-33 IgG4 antibody QB11465 and anti-hTSLP IgG1-D265A antibody QB11718 are shown in Table 22 below.

TABLE 22
Theoretical masses for deglycosylated
antibodies in MabPair QB11750

| | Theoretical mass (Daltons) |
|---|---|
| | anti-hTSLP IgG1-D265A antibody |
| Combination | QB11718 LC1 and HC1 |
| of HCs and LCs | anti-hIL-33 IgG4 antibody QB11465 LC2 and HC2 |
| LC1/HC1/HC1/LC1 | 145670.02 |
| LC2/HC2/HC2/LC2 | 145995.66 |
| LC1/HC2/HC2/LC1 | 145270.82 |
| LC2/HC1/HC1/LC2 | 146394.86 |
| LC1/HC2/HC1/LC2 | 145832.84 |
| LC1/HC1/HC2/LC2 | 145832.84 |
| LC1/HC1/HC1/LC2 | 146032.44 |
| LC1/HC2/HC2/LC2 | 145633.24 |
| LC1/HC1/HC2/LC1 | 145470.42 |
| LC2/HC1/HC2/LC2 | 146195.26 |

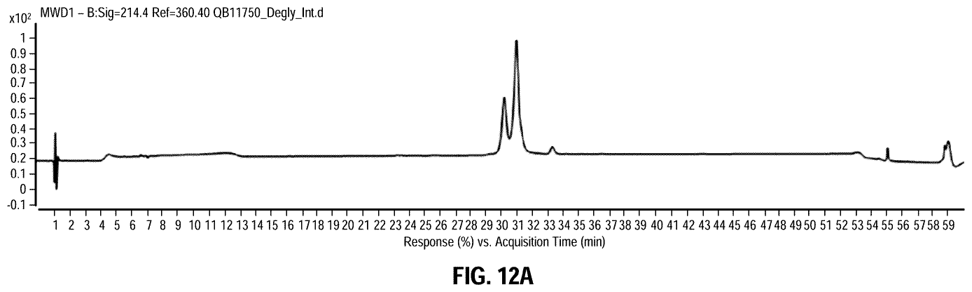

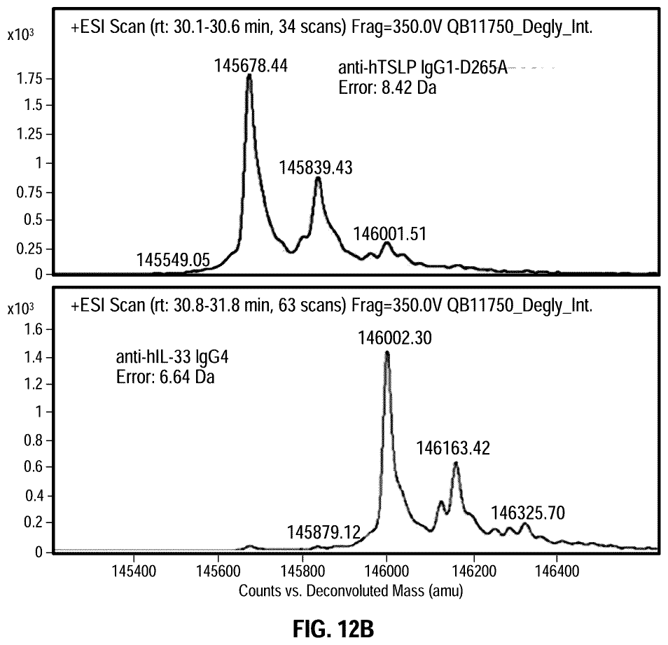

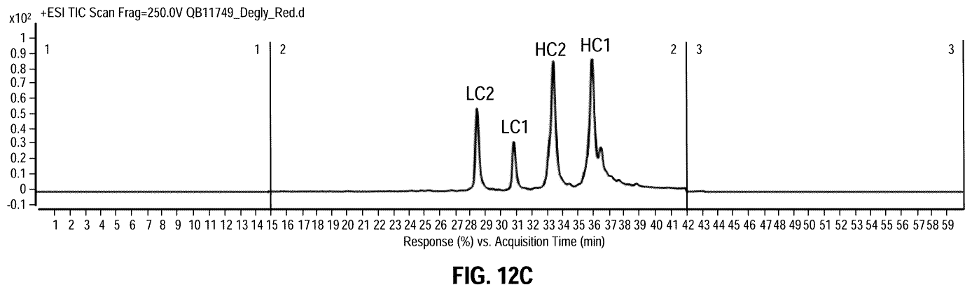

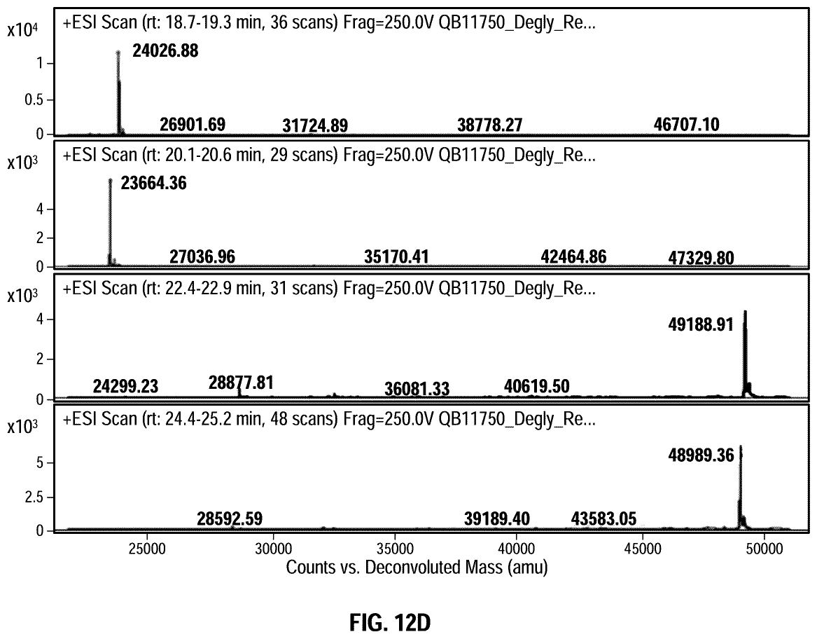

FIG. 12, panel A shows under UV detection the two main peaks of deglycosylated antibody produced by host cells containing DNAs encoding MabPair QB11750 which consists of anti-hIL-33 IgG4 antibody QB11465 and anti-hTSLP IgG1-D265A antibody QB11718. Panel B shows the intact mass of anti-hTSLP IgG1-D265A antibody QB11718 (top) and anti-hIL-33 IgG4 antibody QB11465 (bottom). As indicated, the observed mass of the major peak of anti-hTSLP IgG1-D265A antibody QB11718 was 145678.44 daltons (Da), which is 8.42 Da difference at 57.8 parts per million (ppm) away from the theoretical mass of 145670.02 Da. The minor peak at 145839.43 Da is a glycated anti-hTSLP IgG1-D265A antibody QB11718 since the observed difference is 160.99 Da (145839.43-145678.44), an increase of 162 Da mass indicates a glycated lysine, see Wei B. et al. MABS 2017; 9 (4): 586-594 and Lapolla A. et al. J Am Soc Mass Spectrom 2004; 15:496-509. The other minor peak is double glycated since the observed difference between the two minor peaks (146001.51-145839.43) is 162.08 Da. Similarly, the major peak of anti-hIL-33 IgG4 antibody QB11465 shows an observed mass at 146002.30 Da, which is 45.48 ppm (6.64 Da) from theoretical mass at 145995.66 Da. The minor peak at 146163.42 Da is a glycated version and the minor peak at 146325.70 Da is a double glycated version of anti-hIL-33 IgG4 antibody QB11465. Since both mass variations from the theoretical mass are less than 100 ppm, these experimentally determined masses suggested that the major peaks of two individual antibodies could result from antibodies with cognate HC/LC pairs.

Under reducing condition, MabPair QB11750 contains 4 different peaks under UV detection (FIG. 12C), an anti-hIL-33 LC (FIG. 12D, top) with a mass of 24026.88 Da, which is 14.6 ppm different from the theoretical mass of 24026.53 Da; an anti-hTSLP LC (FIG. 12D, second from the top) with a mass of 23664.36 Da, which is 2.1 ppm away from the theoretical mass of 23664.31 Da; an anti-hTSLP HC (FIG. 12C, third from the top) with a mass of 49188.91 Da, which is 38.2 ppm away from the theoretical mass of 49187.03 Da; an anti-hIL-33 HC (FIG. 12D, bottom) with a mass of 48989.36 Da, which is 39.4 ppm away from the theoretical mass of 48987.43 Da. All experimental errors are far below the allowable error of 100 ppm. These results clearly demonstrate that the MabPair QB11750 consisting of anti-hIL-33 IgG4 antibody QB11465 and anti-hTSLP IgG1-D265A antibody QB11718 has four different chains with the expected masses.

MabPair product QB11750 was digested by IdeS protease to generate F(ab′) 2 fragment since IdeS protease preferably cut the sequence below the hinge region of IgGI (CPPCPAPELLG↓GPSVFLFPP) and IgG4 (CPPCPAPEFLG↓GPSVFLFPP). A partial reduction using 2-MEA was followed to generate Fab fragments since 2-MEA preferably reduces the interchain disulfide bonds at the hinge region whereas keeping the intrachain disulfide bonds in the V_{H} and V_{L} intact. Therefore, the Fab fragments in MabPair can be easily analyzed by mass spectrometry to assess whether chain mispairings exist in MabPair QB11750.

400 μg of sample buffer exchanged 3 times by centrifugation at 15° C. at 14,000×g into 50 mM sodium phosphate (NaP) and 150 mM NaCl pH 6.6. Samples were digested with 100 units of IdeS at 37° C. overnight. For reduced samples, the nonreduced digest was diluted at 1:2 in 100 mM NaP, 150 mM NaCl, 5 mM EDTA pH 7.3 and 2.2 mM 2-MEA. Samples were thoroughly mixed and heated at 37° C. for up to 1 hr before quenched with 1/10 volume of 10% (v/v) formic acid. The non-reduced and reduced samples were separately injected into Agilent 6224 accurate-mass TOF mass spectrometer for analysis as described above.

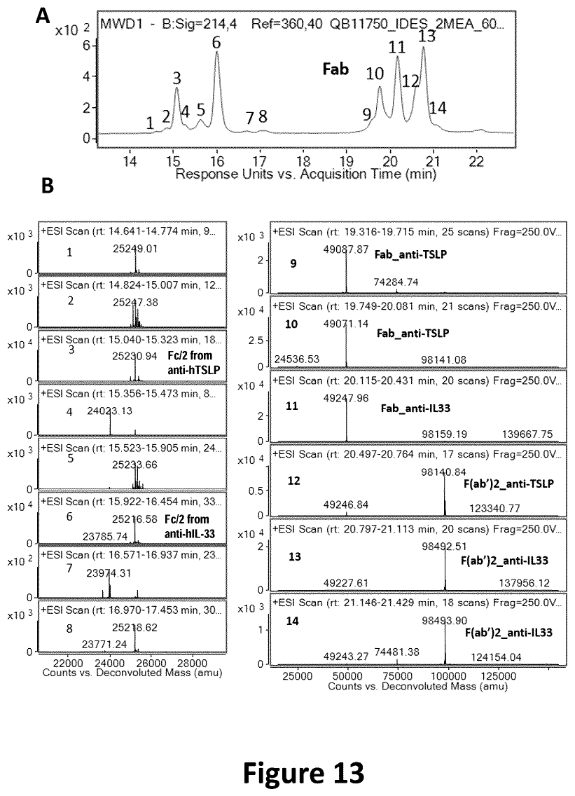

As shown in FIG. 13A, the IdeS and 2-MEA treated MabPair QB11750 showed 14 peaks under UV detection with peaks 3, 6, 10, 11, 13 as the major peaks. The expected theoretical masses of all possible combinations for F(ab′) 2 and Fab fragments are listed in Table 23 and Table 24 below, respectively. The major peak 3 at 25230.94 Da is the half Fc of anti-hTSLP antibody QB11718, it is 15 ppm away from the theoretical mass of 25230.55. The major peak 6 at 25216.28 Da is the half Fc of anti-hIL-33 antibody QB11465, it is 19 ppm away from the theoretical mass of 25216.76 Da. The major peak 10 at 49071.14 Da is the Fab fragment from anti-hTSLP antibody QB11718 with 1 ppm experimental error away from the theoretical mass at 49071.05 Da, the shoulder peak 9 with 49087.87 Da is an oxidated Fab of anti-hTSLP antibody QB11718 since the mass difference is 16.73 Da between shoulder peak 9 and major peak 10. The major peak 11 at 49247.96 Da is the Fab fragment from anti-hIL-33 antibody QB11465 with 0.8 ppm experimental error away from the theoretical mass at 49248.00 Da. The shoulder peak 12 with 98140.84 Da is the F(ab′) 2 of anti-hTSLP antibody QB11718 and the major peak 13 with 98492.51 Da is the F(ab′) 2 of anti-hIL-33 antibody QB11465 with an experimental error of 27.9 ppm and 5.2 ppm, respectively. A minor peak 14 with 98493.90 Da could be the F(ab′) 2 of anti-hIL-33 with a reduced disulfide bond since the mass difference between main peak 13 and the minor peak 14 is 1.39 Da. The presence of F(ab′) 2 fragments indicates that the 2-MEA did not fully reduce all F(ab′) 2 fragments into Fab fragment. Other minor peaks (1, 2, 4, 5, 7, 8) surrounding the major Fc/2 peaks 3 and 6 are the modified Fc/2 or LC of either anti-hTSLP antibody or anti-hIL-33 antibody. All observed masses are far below the allowable experimental error of 100 ppm; there is no peak with a mass near the non-cognate 8 fragments for F(ab′) 2; and there is no peak with a mass near the non-cognate 2 fragments for Fab, see the Tables 23 and 24 below. All these results clearly demonstrated that all HC-HC and LC-HC are correctly paired to produce MabPair antibody mixture as designed.

TABLE 23
Theoretical masses for F(ab′)2 fragment in MabPair QB11750

| | Theoretical mass (Daltons) |
|---|---|
| | anti-hTSLP IgG1-D265A antibody |
| | QB11718 LC1 and HC1 |
| Combination | anti-hIL-33 IgG4 antibody |
| of Fd and LC | QB11465 LC2 and HC2 |
| F(ab′)2 of anti-hTSLP | 98138.10 |
| F(ab′)2 of anti-hIL-33 | 98492.00 |
| LC1/Fd2/Fd2/LC1 | 97767.18 |
| LC2/Fd1/Fd1/LC2 | 98862.92 |
| LC1/Fd2/Fd1/LC2 | 98315.05 |
| LC1/Fd1/Fd2/LC2 | 98315.05 |
| LC1/Fd1/Fd1/LC2 | 98500.51 |
| LC1/Fd2/Fd2/LC2 | 98129.59 |
| LC1/Fd1/Fd2/LC1 | 97952.64 |
| LC2/Fd1/Fd2/LC2 | 98677.46 |

TABLE 24
Theoretical masses for Fab fragment in MabPair QB11750

| | Theoretical mass (Daltons) |
|---|---|
| Fd/LC | anti-hTSLP IgG1-D265A antibody QB11718 LC1 and HC1 |
| combination | anti-hIL-33 IgG4 antibody QB11465 LC2 and HC2 |
| Fd1/LC1 | 49071.05 |
| Fd2/LC2 | 49248.00 |
| Fd1/LC2 | 49433.46 |
| Fd2/LC1 | 48885.59 |

### Example 11: Confirmation of MabPair QB11823 Consisting of Anti-IL-33 IgG4 Antibody QB11465 and Anti-TSLP IgG1-D265A Antibody QB11764 by Mass Spectrometry

The MabPair QB11823 consisting of anti-hIL33 IgG4 antibody QB11465 and anti-hTSLP IgG1-D265A antibody QB11764 was produced by transient transfection and analyzed by mass spectrometry in the same way as described in Example 10. The anti-hTSLP antibody QB11718 blocks the interaction between hTSLP and hIL7Rα whereas the anti-hTSLP antibody QB11764 directly blocks the interaction between hTSLP and hTSLPR, see Example 8 above. FIG. 14, panel A shows under UV detection the two main peaks after deglycosylation by PNGase F treatment. As indicated, the observed mass of the major peak at 144,022.65 Da is the intact anti-hTSLP IgG1-D265A antibody QB11764, which is 4.66 Da difference at 32.36 ppm away from the theoretical mass of 144,018.08 Da. The minor peak at 144184.95 Da is a glycated anti-hTSLP IgG1-D265A antibody QB11764 since the observed difference is 162.3 Da. Similarly, the major peak of anti-hIL-33 IgG4 antibody QB11465 shows an observed mass at 146000.75 Da, which is 35.28 ppm (5.15 Da) from theoretical mass at 145995.66 Da. The minor peak at 146163.7 Da is a glycated version of anti-hIL-33 IgG4 antibody QB11465 with a difference of 162.95 Da. Since the mass variations from the theoretical mass are less than 100 ppm, and other antibody species derived from mismatched HC/LC pairings were not observed (see Table 25 below), these experimentally determined masses suggested that the major peaks of two individual antibodies could result from antibodies with cognate HC/LC pairs only.

TABLE 25
Theoretical masses for deglycosylated
antibodies in MabPair QB11823

| | Theoretical mass (Daltons) |
|---|---|
| | anti-hTSLP IgG1-D265A antibody |
| Combination | QB11764 LC1 and HC1 |
| of HCs and LCs | anti-hIL-33 IgG4 antibody QB11465 LC2 and HC2 |
| LC1/HC1/HC1/LC1 | 144018.08 |
| LC2/HC2/HC2/LC2 | 145995.66 |
| LC1/HC2/HC2/LC1 | 145400.92 |
| LC2/HC1/HC1/LC2 | 144612.82 |
| LC1/HC2/HC1/LC2 | 145006.87 |
| LC1/HC1/HC2/LC2 | 145006.87 |
| LC1/HC1/HC1/LC2 | 144315.45 |
| LC1/HC2/HC2/LC2 | 145698.29 |
| LC1/HC1/HC2/LC1 | 144709.50 |
| LC2/HC1/HC2/LC2 | 145304.24 |

As shown in FIG. 14B, the IdeS treated MabPair QB11823 showed 4 major peaks under UV detection. As shown in FIG. 14C, the major peak 1 at 25230.30 Da is the half Fc (Fc/2) anti-hTSLP antibody QB11764, it is 9.9 ppm away from the theoretical mass of 25230.55. The major peak 2 at 25216.15 Da is the half Fc of anti-hIL-33 antibody QB11465, it is 24.2 ppm away from the theoretical mass of 25216.76 Da. The major peak 3 at 98492.43 Da is the F(ab′) 2 fragment from anti-hIL-33 antibody QB11465 with 17.4 ppm experimental error away from the theoretical mass at 98490.72 Da. The major peak 4 at 98486.25 Da is the F(ab′) 2 fragment from anti-hTSLP antibody QB11764 with 14.6 ppm experimental error away from the theoretical mass at 98484.81 Da. All observed masses are far below the allowable experimental error of 100 ppm; there is no peak with a mass near the 8 non-cognate F(ab′) 2 fragments, see Table 26 below.

TABLE 26
Theoretical masses for F(ab′)2 fragment in MabPair QB11823

| | Theoretical mass (Daltons) |
|---|---|
| | anti-hTSLP IgG1-D265A antibody |
| | QB11764 LC1 and HC1 |
| Combination | anti-hIL-33 IgG4 antibody |
| of Fd and LC | QB11465 LC2 and HC2 |
| F(ab′)2 of anti-hTSLP | 96484.81 |
| F(ab′)2 of anti-hIL-33 | 98490.72 |
| LC1/Fd2/Fd2/LC1 | 97895.97 |
| LC2/Fd1/Fd1/LC2 | 97079.56 |
| LC1/Fd2/Fd1/LC2 | 97487.77 |
| LC1/Fd1/Fd2/LC2 | 97487.77 |
| LC1/Fd1/Fd1/LC2 | 96782.19 |
| LC1/Fd2/Fd2/LC2 | 98193.35 |
| LC1/Fd1/Fd2/LC1 | 97190.39 |
| LC2/Fd1/Fd2/LC2 | 97785.14 |

As shown in FIG. 15A, the IdeS and 2-MEA treated MabPair QB11823 showed 4 major peaks under UV detection. The major peaks 1 and 2 are the F(ab′) 2 of anti-hTSLP antibody QB11764 and anti-hIL-33 antibody QB11465, respectively. The presence of F(ab′) 2 fragments indicates that the 2-MEA did not fully reduce all F(ab′) 2 fragments into Fab fragment. The major peak B with a mass of 49247.72 Da is the Fab fragment of anti-hIL-33 antibody QB11465 with an experimental error of 6.9 ppm away from the theoretical mass of 49247.38 Da. The major peak D with a mass of 48246.10 Da is the Fab fragment of anti-hTSLP antibody QB11764 with an experimental error of 34.8 ppm away from the theoretical mass of 48244.42 Da. Other minor peaks A and C are the 2-MEA adducted Fab fragments. All observed masses are far below the allowable experimental error of 100 ppm; there is no peak with a mass near the 2 non-cognate Fab fragments (48950.00 Da and 48541.80 Da), see Table 27 below. All above results clearly demonstrated that all HC-HC and LC-HC pairings are correctly matched to produce MabPair antibody mixture as designed.

TABLE 27
Theoretical masses for Fab fragment in MabPair QB11823

| | Theoretical mass (Daltons) |
|---|---|
| Fd/LC | anti-hTSLP IgG1-D265A antibody QB11764 LC1 and HC1 |
| combination | anti-hIL-33 IgG4 antibody QB11465 LC2 and HC2 |
| Fd1/LC1 | 48244.42 |
| Fd2/LC2 | 49247.38 |
| Fd1/LC2 | 48950.00 |
| Fd2/LC1 | 48541.80 |

### Example 12: Assessment of Binding Features of Anti-TSLP IgG1-D265A Antibodies by Biacore Analysis

To fully characterize the binding properties of lead anti-hTSLP antibody QB11764 and QB11718 which have different mechanisms for inhibiting the hTSLP-induced biological effects (Examples 4 and 6), multiple commercial antigens were purchased, in-house antigens were purified to provide a full spectrum of binding profiles.

TABLE 28
TSLP antigens from different sources for Biacore measurements

| | | Catalog | | Production | | |
|---|---|---|---|---|---|---|
| Molecule | Vendor | number | Tags | Cell | Amino Acids | Abbreviation |
| hTSLP | ACRO | TSP-H52Hb | c-terminal poly- | HEK293 | Tyr29-Gln159 | Acro-WT |
| | Biosystems | | his | | | |
| hTSLP | Sino | 16135- | c-terminal poly- | HEK293 | Met1-Gln159 | Sino-RA-RS |
| | Biological | H08H | his | | (R127A, R130S) | |
| hTSLP | R&D | 1398-TS- | none | E. coli | Tyr29-Gln159 | R&D |
| | Systems | 010 | | | | |
| hTSLP | Sound | QB11630 | n-terminal avi- | Expi293 + | Tyr29-Gln159 | QB11630 |
| | Biologics | | tag, c-myc, His6 | furin | | |
| | | | | inhibitor | | |
| hTSLP | Sound | QB11631 | n-terminal avi- | Expi293 | Tyr29-Gln159 | QB11631 |
| | Biologics | | tag, c-myc, His6 | | (R127A, R130S) | |
| cyTSLP | ACRO | TSP-C52H8 | c-terminal poly- | HEK293 | Tyr29-Gln159 | Acro-WT |
| | Biosystems | | his | | | |
| cyTSLP | ACRO | TSP-C52H4 | c-terminal poly- | HEK293 | Tyr29-Gln159 | Acro-RA-RS |
| | Biosystems | | his | | (R127A, R130S) | |
| cyTSLP | Sino | 90911- | c-terminal poly- | HEK293 | Met1-Gln159 | Sino-RA-RS |
| | Biological | C08H-100 | his | | (R127A, R130S) | |
| cyTSLP | Sound | QB11632 | n-terminal avi- | Expi293 + | Tyr29-Gln159 | QB11632 |
| | Biologics | | tag, c-myc, His6 | furin | | |
| | | | | inhibitor | | |
| cyTSLP | Sound | QB11633 | n-terminal avi- | Expi293 | Tyr29-Gln159 | QB11633 |
| | Biologics | | tag, c-myc, His6 | | (R127A, R130S) | |

Goat anti-human IgG capture antibody was immobilized to flow cells of the CM4 sensor chip using the Amine Coupling Kit and HBS-EP as running buffer. The Goat-anti-human IgG reagent was prepared in Acetate 5.5 buffer at 30 μg/mL for the immobilization procedure. Each flow cell had ~8000RU immobilized to the surface.

TABLE 29
Conditions used for Biacore measurements to assess
the binding properties of anti-hTSLP antibodies

| | Abbreviation | Association | Dissociation | Analyte Concentrations |
|---|---|---|---|---|
| Molecule | used in results | [s] | [s] | [nM] |
| cyTSLP | Acro - WT | 180 | 500 | 100; 33.3; 11.1; 3.7; 1.24; 0.412 |
| cyTSLP | Acro - RA-RS | 180 | 720 | 8; 4; 2; 1; 0.5; 0.25; 0.13 |
| cyTSLP | Sino - RA-RA | 180 | 500 | 100; 33.3; 11.1; 3.7; 1.24; 0.412 |
| cyTSLP | QB11632 | 180 | 500 | 200; 66.7; 22.2; 7.4; 2.47; 0.823 |
| cyTSLP | QB11633 | 180 | 500 | 50; 16.7; 5.56; 1.85; 0.617; 0.206 |
| hTSLP | Acro | 180 | 720 | 8; 4; 2; 1; 0.5; 0.25; 0.13 |
| hTSLP | Sino | 180 | 720 | 8; 4; 2; 1; 0.5; 0.25; 0.13 |
| hTSLP | R&D | 180 | 500 | 1; 0.5; 0.25; 0.13; 0.063; 0.031 |
| hTSLP | QB11630 | 180 | 720 | 10; 4; 1.6; 0.64; 0.256; 0.1024 |
| hTSLP | QB11631 | 300 | 720 | 8; 4; 2; 1; 0.5; 0.25; 0.13 |

To collect kinetic binding data, the analytes as listed in the above table 29 were diluted in running buffer, which consists of HBS-EP+0.05% BSA, were injected over flow cells (fc) 1 through 4, where fc2 had captured QB11718, fc3 had captured QB11764, and fc4 had captured QB10985, all at approximately 100 RU's. Fcl was used as the reference flow cell. The analytes were injected at the concentrations listed in the table 29 at a flow rate of 30 μL/min and at a detection temperature of 25° C. The complex was allowed to associate and dissociate according to the times within the table 29. At the end of each cycle the surfaces were regenerated with a 20 second injection of 10 mM glycine-HC1, pH 1.5. Duplicate injections of one analyte concentration per analyte and buffer blanks were flowed over the reference and captured ligand surface. The T200 Evaluation software was used to align and double reference the data. The data were fit to a simple 1:1 Langmuir interaction model using the global data analysis option within the software.

TABLE 30
Summaries of binding properties of anti-hTSLP antibodies to monomeric hTSLP antigen

| | R&D | | | Acro | | | Sino | | |
|---|---|---|---|---|---|---|---|---|---|
| | 1398-TS-010 | | | TSP-H52Hb | | | 16135-H08H | | |
| | ka[1/Ms] | kd[1/s] | KD[M] | ka[1/Ms] | kd[1/s] | KD[M] | ka[1/Ms] | kd[1/s] | KD[M] |
| QB10985 | 4.32E+07 | 1.53E−04 | 3.53E−12 | 5.64E+06 | 5.54E−05 | 9.82E−12 | 5.86E+06 | 6.50E−05 | 1.11E−11 |
| QB11718 | 2.30E+07 | 1.15E−04 | 5.02E−12 | 7.91E+06 | 9.35E−05 | 1.18E−11 | 8.61E+06 | 1.15E−04 | 1.33E−11 |

| | | | | | |
|---|---|---|---|---|---|
| QB11764 | 3.56E+07 | 1.42E−04 | 4.00E−12 | No measurable binding | No measurable binding |
| | | | | observed | observed |

| | QB11630 | | | QB11631 | | |
|---|---|---|---|---|---|---|
| | In-house hTSLP | | | In-house hTSLP | | |
| | ka[1/Ms] | kd[1/s] | KD[M] | ka[1/Ms] | kd[1/s] | KD[M] |
| QB10985 | 1.13E+06 | 5.74E−05 | 5.07E−11 | 2.45E+06 | 5.85E−05 | 2.39E−11 |
| QB11718 | 1.70E+06 | 1.32E−04 | 7.78E−11 | 5.42E+06 | 8.82E−05 | 1.63E−11 |
| QB11764 | 1.27E+06 | 1.96E−04 | 1.54E−10 | 3.23E+06 | 1.92E−04 | 5.95E−11 |

TABLE 31
Summaries of binding properties of anti-hTSLP antibodies to monomeric cyTSLP antigen

| | Acro WT | | | Acro-RA-RS | | | Sino-RA-RS | | |
|---|---|---|---|---|---|---|---|---|---|
| | TSP-C52H8 | | | TSP-C52H4 | | | 90911-C08H-100 | | |
| | ka[1/Ms] | kd[1/s] | KD[M] | ka[1/Ms] | kd[1/s] | KD[M] | ka[1/Ms] | kd[1/s] | KD[M] |
| QB10985 | 5.12E+06 | 1.13E−04 | 2.20E−11 | 4.88E+06 | 8.90E−05 | 1.82E−11 | 7.58E+06 | 6.18E−05 | 8.15E−12 |
| QB11718 | 3.56E+06 | 1.05E−04 | 2.95E−11 | 4.08E+06 | 1.07E−04 | 2.63E−11 | 4.15E+06 | 7.76E−05 | 1.87E−11 |

| | | | |
|---|---|---|---|
| QB11764 | No measurable binding | No measurable binding | No measurable binding |
| | observed | observed | observed |

| | QB11632 | | | QB11633 | | |
|---|---|---|---|---|---|---|
| | in-house cyTSLP | | | in-house cyTSLP | | |
| | ka[1/Ms] | kd[1/s] | KD[M] | ka[1/Ms] | kd[1/s] | KD[M] |
| QB10985 | 2.15E+05 | 8.17E−05 | 3.80E−10 | 1.52E+06 | 7.20E−05 | 4.74E−11 |
| QB11718 | 2.90E+05 | 2.41E−04 | 8.31E−10 | 1.51E+06 | 1.13E−04 | 7.50E−11 |
| QB11764 | 1.37E+05 | 1.74E−04 | 1.27E−09 | 1.01E+06 | 1.32E−04 | 1.31E−10 |

When the hTSLP antigen produced from E. coli (R&D systems, 1398-TS-010) was used for Biacore measurement, the lead anti-hTSLP antibodies QB11718 and QB11764 have very comparable binding profiles with similar Ka, Kd and single digit pM binding affinity (KD), see Table 30. When commercial hTSLP antigens (Acro, TSP-H52Hb; Sino 16135-H08H) produced from mammalian cells were used for Biacore measurement, the lead anti-hTSLP antibody QB11718 has very comparable binding affinity as the comparator antibody QB10985 whereas no significant binding was observed for another anti-hTSLP lead antibody QB11764. When the in-house hTSLP native antigen QB11630 which was produced in the presence of furin inhibitor was used in Biacore, the lead anti-hTSLP antibody QB11718 has very comparable binding affinity as comparator antibody QB10985 whereas another anti-hTSLP lead antibody QB11764 has ~3-fold lower binding affinity (KD=1.54×10⁻¹⁰ M) comparing to the QB10985 (KD=5.07×10⁻¹¹ M); very similar results were obtained when the in-house hTSLP antigen QB11631 which has two substitutions R127A+R130S was applied in Biacore measurement. These results indicate that antigens from different sources (prokaryotic versus eukaryotic) can have different binding profiles because of different post-translation modifications of the produced antigens; the lead anti-hTSLP antibody QB11718 has very comparable binding properties as the comparator QB10985 across the board of different antigens whereas another anti-hTSLP lead antibody QB11764 showed decreased or negligible binding when different antigens were used, which could be due to different epitope (that QB11764 binds to) impacted by post-translational modifications. As summarized in Table 31, the lead anti-hTSLP antibody QB11718 has slightly lower affinity for cyTSLP than comparator QB10985 and ~3 times higher than another lead anti-hTSLP antibody QB11764.

### Example 13: Assessment of Blocking Activities of the Anti-hIL-33 IgG4 Antibody QB11465 by Cell-Based Assays

Multiple cell-based assays including the measurement of IFNγ cytokine secretion from primary human NK cells (FIG. 17A), IL-5 secretion from primary human ILC2 cells (FIG. 17B), and the level of p38 MAPK phosphorylation of primary human ILC2 cells (FIGS. 17C, 17D) were conducted to assess the biological activity of the lead anti-hIL-33 IgG4 blocking antibody QB11465.

Primary human NK cells were purified using a EasySep™ Human NK Cell Isolation Kit from Stemcell Technologies (cat. no. 17955). The purified human NK cells were seeded at 30,000 cells/well in assay media of RPMI1640 Medium containing 2 mM L-Glutamine, 10% Heat-Inactivated Fetal Bovine serum, Penicillin-Streptomycin 100 Units/mL and treated for 24 hr with a constant amount of hIL-33 at concentrations close to EC₅₀ values and IL-12 at 1 ng/mL in combination with a 1:3 series titration of anti-hIL-33 antibody. IFNγ cytokine levels in the supernatants were determined using human IFNγ ELISA kit from R&D Systems (cat. no. DY285B) as per the manufacturer's instructions.

As shown in FIG. 17A, the isotype control antibody QB11827 at 1,000 nM concentration didn't inhibit the IFNγ secretion from primary human NK cells, IFNγ was determined at ~1,200 pg/mL. However, the comparator-3 anti-hIL-33 IgG4 antibody QB11094 potently inhibited the IFNγ secretion with an IC₅₀=0.05 nM. The lead anti-hIL-33 IgG4 antibody QB11465 strongly inhibited the IFNγ secretion with an IC₅₀=1.0 nM, which was 20-fold less potent than the comparator-3 antibody QB11094.

Primary human innate lymphoid cells type 2 (ILC2) cells were purified from PBMCs using a EasySep™ Human ILC2 Enrichment Kit from Stemcell Technologies (cat. no. 17972). The purified ILC2s were cultured in the assay media RPMI 1640 containing 2 mM L-Glutamine, 10% Heat-Inactivated Fetal Bovine serum, 1 mM Sodium Pyruvate, 0.075% Sodium Bicarbonate, 100 mM HEPES, Penicillin-Streptomycin 100 Units/mL, and cytokines human IL-2, IL-25, TSLP and IL-33 cytokines all at 10 ng/ml were added biweekly for replenishment to expand for a minimum of 3-4 weeks (~40 fold expansion) or up to 6-8 weeks (>100 fold expansion). Prior to the day of the assay, ILC2 cells were washed three times with assay media before being rested overnight in Tissue Culture incubator to reduce background signal. On the day of the assay, ILC2 cells were seeded at 30,000 cells/well and treated for 48 hr with an amount of hIL-33 (Biolegend, cat. no. 581804) at concentrations close to EC₉₀ values in combination with a 1:3 series titrated anti-hIL-33 antibody, isotype control IgG4 antibody was added at the highest concentration of 1,000 nM. The levels of hIL-5 in supernatants were determined using human IL-5 ELISA kit (R&D Systems, cat. no. DY205) as per the manufacturer's instructions.

As shown in FIG. 17B, the isotype control antibody QB11827 at 1,000 nM concentration didn't inhibit the IFNγ secretion from primary human NK cells, hIL-5 was determined at ~300 μg/mL. However, the comparator-3 anti-hIL-33 IgG4 antibody QB11094 potently inhibited the hIL-5 secretion with an IC₅₀=0.228 nM. The lead anti-hIL-33 IgG4 antibody QB11465 strongly inhibited the hIL-5 secretion with an IC₅₀=2.39 nM, which was around 10-fold less potent than the comparator-3 antibody QB11094.

Prior to the day of the assay, isolated ILC2s were washed three times with assay media before being rested overnight in Tissue Culture incubator to reduce background signal. On the day of the assay, ILC2s were seeded at 100,000 cells/well and treated for 15 min with an amount of hIL-33 or cyIL-33 at concentrations close to EC₉₀ values plus a 1:3 serially titrated anti-IL33 antibody to determine potency. p38 MAPK phosphorylation levels were determined using an AlphaLISA SureFire Ultra p-38 MAPK HV Assay Kit from Perkin Elmer (Shelton, CT) as per the manufacturer's instructions. The signal in each well was read in an Alpha Signal-enabled plate reader.

As shown in FIG. 17C, the isotype control antibody QB11827 didn't inhibit the p38 MAPK phosphorylation of ILC2s. However, the comparator-3 anti-hIL-33 IgG4 antibody QB11094 strongly inhibited the p38 MAPK phosphorylation with an IC₅₀=0.87 nM. The lead anti-hIL-33 IgG4 antibody QB11465 potently inhibited the p38 MAPK phosphorylation with an IC₅₀=0.33 nM, which was around 2.5-fold more potent than the comparator-3 antibody QB11094. To test how well the anti-hIL-33 antibodies could block the cyIL-33 induced p38 MAPK phosphorylation in ILC2s, hIL-33 and cyIL-33 were tested in this assay simultaneously (FIG. 17D). The isotype control antibody QB11827 didn't inhibit the p38 MAPK phosphorylation induced by either hIL-33 or cyIL-33 at the highest concentration of 2,000 nM. The lead anti-hIL-33 IgG4 antibody QB11465 potently inhibited the p38 MAPK phosphorylation with an IC₅₀ =0.82 nM when hIL-33 was used and with an IC₅₀=4.4 nM when cyIL-33 was used to induce the p38 MAPK phosphorylation.

Collectively, our lead anti-hIL-33 IgG4 antibody QB11465 strongly inhibited the hIL-33 induced biological functions, the cross-species binding to cyIL-33 leads to the inhibition of cyIL-33 induced biological activities. The anti-hIL-33 comparator-3 antibody QB11094 is generally more potent than our lead antibody QB11465 in the long-term cell-based assays (24 hr or 48 hr), most likely due to its slower dissociation rate (Kd), but less potent than our lead antibody QB11465 in the short-term p38 MAPK cell-based assay (15 min).

### Example 14: The Anti-TSLP IgG1-D265A Antibodies QB11718 and QB11764 Strongly Inhibit hTSLP-Induced Proliferation of BaF3 Cells Stably Expressing h TSLPR/hIL7Rα

BaF3 cells stably expressing hTSLPR/hIL-7Rα were seeded at 10,000 cells/well and treated for 72 hrs with an amount of hTSLP at concentrations close to EC₅₀ values plus a titrated anti-hTSLP antibodies to determine potency. Proliferation was measured using a CellTiter-Glo Luminescent Cell Viability Assay kit from Promega (Madison, WI) according to the manufacturer's instructions. Several forms of TSLP were used in this assay, hTSLP QB11630 (SEQ ID NO: 32) in FIG. 18A; hTSLP QB11631 (SEQ ID NO: 34) in FIG. 18B; cyTSLP QB11632 (SEQ ID NO: 35) in FIG. 18C; cyTSLP QB11633 (SEQ ID NO: 37) in FIG. 18D; a native hTSLP generated from healthy donor-1 in FIG. 18E; a native hTSLP generated from healthy donor-2. The native hTSLP was derived from human Small Airway Epithelial Cells (SAECs) purchased from Lonza (cat. no. CC-2547). SAECs were grown to sub-confluency according to the manufacturer's instructions then treated with human TNFα at 25 ng/mL, both IL-la and IL-1ß at 10 ng/ml in growth media for 48 hrs at 37° C. After treatment supernatants were collected, one half was frozen at −80° C. as neat and the other half was concentrated 20-fold with spin columns before freezing at −80° C.

Collectively, the isotype control antibody QB11827 didn't inhibit the proliferation of BaF3 cells stably expressing hTSLPR/hIL-7Rα at all, the lead anti-hTSLP antibody QB11718 is the most potent blocker which inhibit the proliferation of BaF3 cells stably expressing hTSLPR/hIL-7Rα, with 3~10 folder higher potency than the anti-hTSLP comparator antibody QB10985. Another lead anti-hTSLP antibody QB11764 can inhibit the proliferation of BsF3 cells induced by different forms of hTSLP and cyTSLP, but it is generally less potent (>10-fold) than the anti-hTSLP comparator antibody QB10985.

### Example 15: The Anti-TSLP IgG1-D265A Antibody QB11718 Strongly Inhibits hTSLP-Induced pSTAT5 Phosphorylation in BaF3 Cells Stably Expressing hTSLPR/hIL-7Rα

TSLP T mediates signaling by establishing a heteromeric complex involving TSLPR and IL-7Rα. In the absence of ligand TSLP, the interaction between TSLPR and IL-7Rα is very weak with an affinity at 20 μM, such a weak interaction can't trigger the activation of downstream pathways. Once the TSLP binds to TSLPR, a conformational change allows the TSLP/TSLPR complex to quickly bind to IL-7Rα, making this binary assembly a mechanistic prerequisite for effective signal transduction. The dimerization of both receptor chains upon TSLP binding results in activation of Janus kinases (JAKs) and signal transducers and activators of transcription (STATs) leading to transcription of target genes and subsequent tightly coordinated immune responses. STAT5 is the major substrate for phosphorylation shortly after the tertiary TSLP/TSLPR/IL-7Rα complex forms.

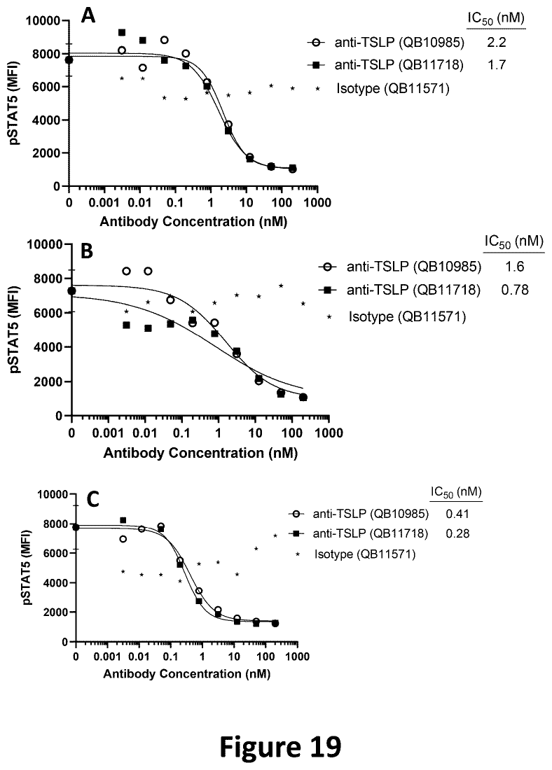

BaF3 cells stably expressing hTSLPR/hIL-7Rα were seeded at 10,000 cells/well and treated with different forms of TSLP stimulator in combination with the titrated antibodies for 15 min. The stimulator TSLPs include hTSLP QB11630 (SEQ ID NO: 32) in FIG. 19A; hTSLP QB11631 (SEQ ID NO: 34) in FIG. 19B; cyTSLP QB11632 (SEQ ID NO: 35) in FIG. 19C. The pSTAT5 phosphorylation level was measured using an AlphaLISA SureFire Ultra p-STAT5 HV Assay Kit from Perkin Elmer (cat. no. ALSU-PST5-B-HV) as per the manufacturer's instructions. The plates were read in an Alpha Signal-enabled plate reader, IC₅₀ values were calculated using GraphPad Prism software.

Collectively, the isotype control antibody QB11827 didn't inhibit the pSTAT5 phosphorylation of BaF3 cells stably expressing hTSLPR/hIL-7Rα, the lead anti-hTSLP antibody QB11718 is a very potent blocker, which has comparable potency (FIG. 19A) or slightly higher potency (FIGS. 19B and 19C) when compared to the anti-hTSLP comparator antibody QB10985.

### Example 16: The Anti-TSLP IgG1-D265A Antibody QB11718 Strongly Inhibits TSLP-Induced CCL17 Secretion from Human Monocytes

Once the TSLP binds to TSLPR and forms a tertiary complex with IL7Rα, the myeloid dendritic cells (mDCs) upregulate the production of CCL17 and CCL22, chemokines for the receptor CCR4 on Th2 cells. OX40L is also induced, which in turn contributes to Th2-type immune responses such as secretion of IL-4, IL-5, IL-13. See Gu C. et al. Front Immunol. 2021; 12:678036.

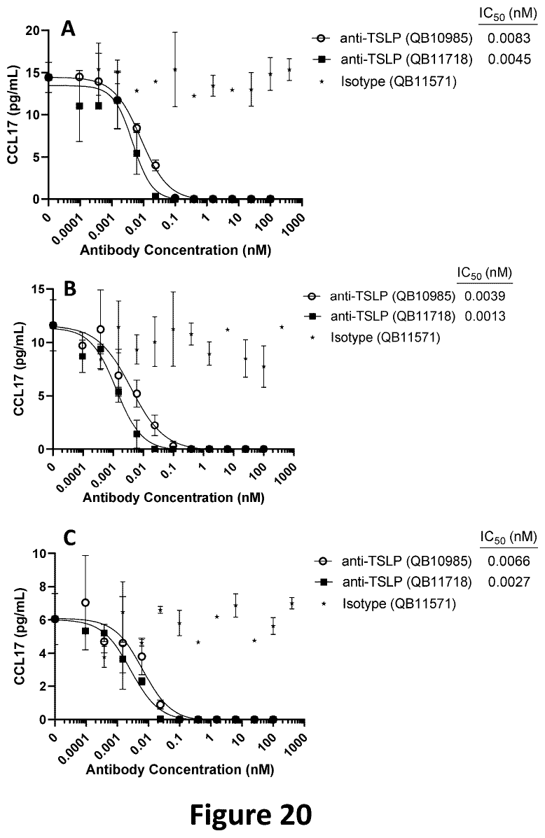

Primary human monocytes were purified from PBMCs using a EasySep™ Human Monocyte Isolation Kit from Stemcell Technologies (cat. no. 19359). The purified human monocytes were seeded at 150,000 cells/well and treated for 24 hr with a constant amount of TSLP at concentrations close to EC₅₀ values plus the titrated anti-TSLP antibodies. CCL17 chemokine levels in supernatants were determined using a Human CCL17/TARC DuoSet ELISA kit from R&D Systems (cat. no. DY364) as per the manufacturer's instructions. Several forms of recombinant TSLP, hTSLP QB11630 (SEQ ID NO:32) which was generated in presence of furin inhibitor (FIG. 20A), hTSLP QB11631 (SEQ ID NO:34) which has two substitutions (R127A+R130S) at furin cleavage site (FIG. 20B), and cyTSLP QB11632 (SEQ ID NO:35) which was generated in presence of furin inhibitor (FIG. 20C), were used in this assay. The IC₅₀ values were calculated using GraphPad Prism software.

Collectively, the isotype control antibody QB11827 didn't inhibit the CCL17 secretion from human monocytes, the lead anti-hTSLP antibody QB11718 is a very potent blocker, which has 2~3 folder higher potency when compared to the anti-hTSLP comparator antibody QB10985.

### Example 17: The Anti-TSLP IgG1-D265A Antibody QB11718 Strongly Inhibits TSLP-Induced IL-5 Secretion from Human ILC2s

Type-2 innate lymphoid cells (ILC2s) belong to an expanding family of innate lymphocytes that provide a potent source of immune effector cytokines at the initiation of immune responses. Human ILC2s were reported in human lung parenchyma and bronchoalveolar lavage (BAL) fluid, as lineage-negative cells expressing TSLPR, IL-7Rα, and ST2 receptor for IL-33, see Monticelli L. A. et al. Nat Immunol. 2011; 12:1045-1054. ILC2s arise, under the control of the transcription factors RORa and GATA3, from lymphoid progenitors in the bone marrow, to secrete type-2 cytokines including IL-5 and IL-13.

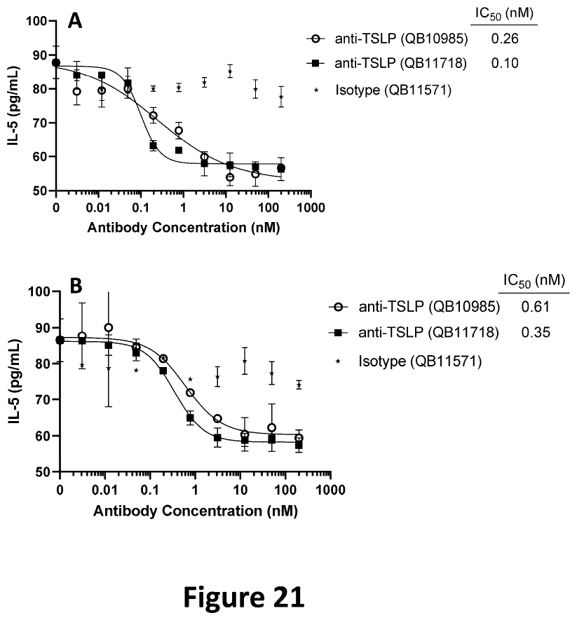

Human ILCs were isolated from PBMCs as described in Example 13. The purified ILC2s were cultured in the assay media RPMI 1640 containing 2 mM L-Glutamine, 10% Heat-Inactivated Fetal Bovine serum, 1 mM Sodium Pyruvate, 0.075% Sodium Bicarbonate, 100 mM HEPES, Penicillin-Streptomycin 100 Units/mL, and cytokines human IL-2, IL-25, TSLP and IL-33 each at 10 ng/ml were added biweekly for replenishment to expand for a minimum of 3-4 weeks (~40 fold expansion) or up to 6-8 weeks (>100 fold expansion). Prior to the day of the assay, ILC2 cells were washed three times with assay media before being rested overnight in Tissue Culture incubator to reduce background signal. On the day of the assay, ILC2 cells were seeded at 30,000 cells/well and treated for 48 hr with an amount of hTSLP QB11630 (SEQ ID NO: 32, in FIG. 21A) and hTSLP QB11631 (SEQ ID NO: 34, in FIG. 21B) at concentrations close to EC₉₀ values in combination with a 1:4 series titrated antibody. The levels of hIL-5 in supernatants were determined using human IL-5 ELISA kit (R&D Systems, cat. no. DY205) as per the manufacturer's instructions. IC₅₀ values were calculated using GraphPad Prism software.

As shown in FIG. 21, hTSLP-induced IL-5 secretion was strongly inhibited by anti-TSLP antibodies but not by isotype control antibody QB11571. The lead anti-hTSLP antibody QB11718 showed an IC₅₀ of 0.10 nM and 0.35 nM when the native hTSLP QB11630 and hTSLP (R127A+R130S) QB11631 were used, respectively. The anti-hTSLP comparator antibody QB10985 is less potent, with an IC₅₀ of 0.26 nM and 0.61 nM when the native hTSLP QB11630 and hTSLP (R127A+R130S) QB11631 were used, respectively.

### Example 18: The Anti-TSLP IgG1-D265A Antibody QB11718 Strongly Inhibits hTSLP-Induced pSTAT5 Phosphorylation in ILC2s

Whereas the lead anti-hTSLP antibody QB11718 inhibited the TSLP-induced pSTAT5 phosphorylation in BaF3 cells stably expressing TSLPR/IL-7Rα, see Example 15 above. It's not known whether the antibody also inhibits the TSLP-induced pSTAT5 phosphorylation in human primary cells since the BaF3/TSLPR/IL-7Rα cells are artificial.

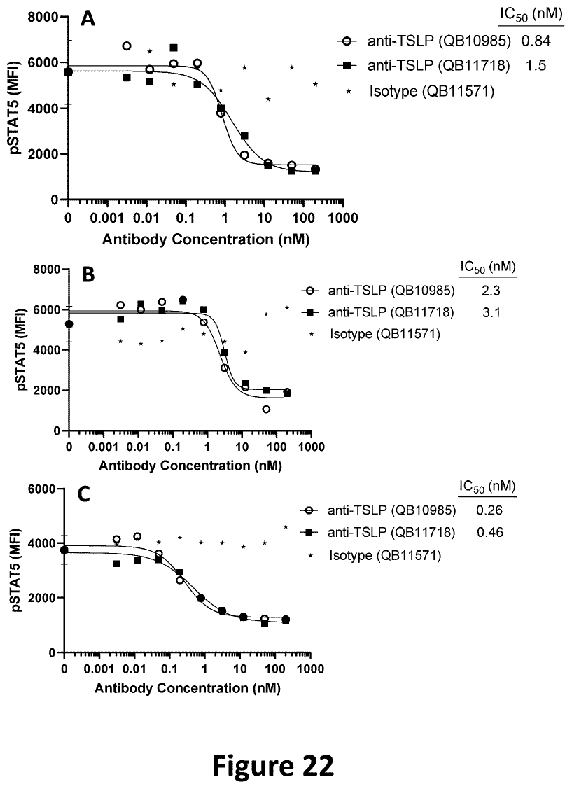

The human ILC2s were isolated and cultured in the same way as described in Example 17 above. ILC2s were treated with an amount of hTSLP QB11630 (SEQ ID NO: 32, in FIG. 22A), hTSLP QB11631 (SEQ ID NO: 34, in FIG. 22B), or cyTSLP QB11632 (SEQ ID NO: 35, in FIG. 22C) at concentrations close to EC₉₀ values in combination with a 1:4 serially titrated antibody. The pSTAT5 phosphorylation level was determined using an AlphaLISA SureFire Ultra p-STAT5 HV Assay Kit from Perkin Elmer (cat. no. ALSU-PST5-B-HV) as per the manufacturer's instructions. The plates were read in an Alpha Signal-enabled plate reader, IC₅₀ values were calculated using GraphPad Prism software.

As shown in FIG. 22, the isotype control antibody QB11571 didn't inhibit the pSTAT5 phosphorylation of cultured human ILC2s. However, the lead anti-hTSLP antibody QB11718 and the anti-hTSLP comparator antibody QB10985 inhibit the pSTAT5 phosphorylation of cultured human ILC2s comparably, with IC₅₀ values at sub-digit or low single digit nM.

### Example 19: Combination of the lead anti-hIL-33 IgG4 antibody QB11465 and anti-TSLP IgG1-D265A antibody QB11718 synergistically inhibits hTSLP-induced IL-5 secretion from human ILC2s

Human ILC2s express IL-7Rα (CD127), IL-33 receptor (ST2), IL-2 receptor (CD25), inducible T cell costimulator (ICOS), thymocyte marker (CD90), hematopoietic progenitor marker c-kit (CD117), stem cells antigen 1 (Sca-1; Ly6A/E), and a hematopoietic marker (CD45). The receptors for IL-2, IL-7, IL-25, and IL-33 are all important for ILC2 development and activation. See Moro K. et al. Nature 2010; 463:540-544, Neill D. R. et al. Nature 2010; 464:1367-1370, and Walker J A et al. Nat Rev Immunol. 2013; 13:75-87. Whereas the lead anti-hIL-33 IgG4 antibody inhibited the hIL-33 induced IL-5 secretion in ILC2s (Example 13) and the lead anti-hTSLP IgG1-D265A antibody QB11718 also inhibited the hTSLP induced IL-5 secretion in ILC2s (Example 17), there was no publication about the combined effect of anti-hTSLP and anti-hIL-33 blocking antibodies.

Human ILC2s were isolated and cultured in the same way as described in Example 17 above. Prior to the day of the assay, ILC2s were washed three times with assay media before being rested overnight in Tissue Culture incubator to reduce background signal. On the day of the assay, ILC2 cells were seeded at 30,000 cells/well and treated for 48 hrs with in-house hTSLP QB11630 and hIL-33 from BioLegend (cat. no. 581814) each at 5 ng/ml in combination with 4×, 2×, 1×, 0.5×or 0.25×of IC₅₀ concentrations of (1). the lead anti-hTSLP IgG1-D265A antibody QB11718 alone; (2). the anti-hTSLP comparator antibody QB10985 alone; (3). the lead anti-hIL-33 IgG4 antibody QB11465 alone; (4). the anti-hIL-33 comparator-3 antibody QB11094 alone; (5). the combination of the lead anti-hTSLP IgG1-D265A antibody QB11718 and the lead anti-hIL-33 IgG4 antibody QB11465; (6). the combination of the lead anti-hTSLP IgG1-D265A antibody QB11718 and the anti-hIL-33 comparator-3 antibody QB11094; (7). the combination of the anti-hTSLP comparator antibody QB10985 and the lead anti-hIL-33 IgG4 antibody QB11465. The hIL-5 cytokine levels in supernatants were determined using a Human IL-5 DuoSet ELISA kit from R&D Systems (cat. no. DY205). The results were processed using GraphPad Prism software. The responses of inhibition were plotted as percent response to no antibody treatment against IC₅₀ fold. The Combination Index (C.I.) was calculated according to Chou-Talalay method, see Chou T. C. Pharmacol Rev. 2006; 58 (3): 621-681 and Cancer Res. 2010; 70 (2): 440-446.

The Chou-Talalay method for drug combination is based on the median-effect equation, derived from the mass-action law principle, which is the unified theory that provides the common link between single entity and multiple entities, and first order and higher order dynamics. This general equation encompasses the Michaelis-Menten, Hill, Henderson-Hasselbalch, and Scatchard equations in biochemistry and biophysics. The resulting combination index (CI) theorem of Chou-Talalay offers quantitative definition for additive effect (CI=1), synergism (CI<1), and antagonism (CI>1) in drug combinations. This theory also provides algorithms for automated computer simulation for synergism and/or antagonism at any effect and dose level, as shown in the CI plot and isobologram, respectively.

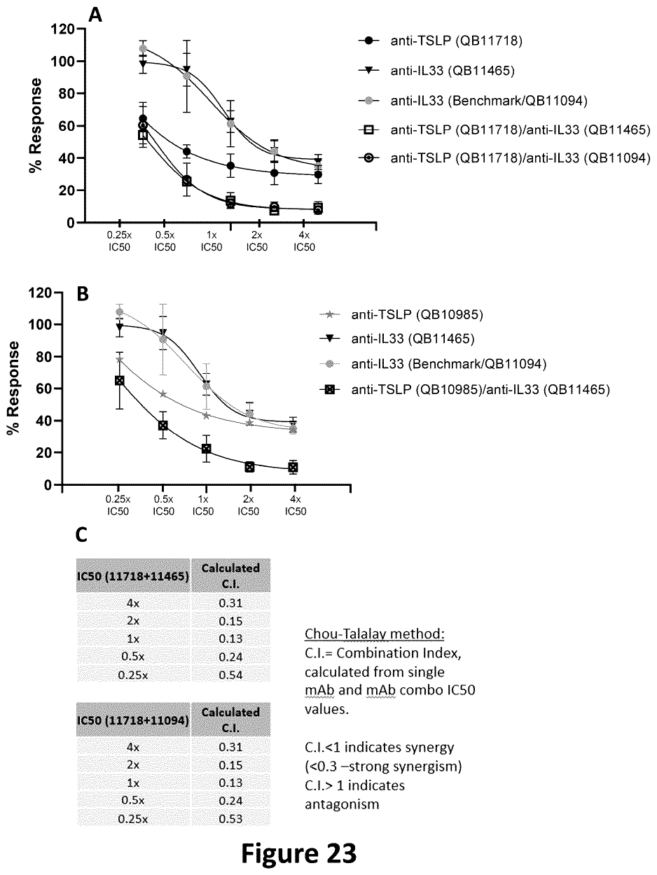

As shown in FIG. 23A, the lead anti-hTSLP IgG1-D265A antibody QB11718 potently inhibited the hIL-5 secretion from ILC2s. The anti-hIL-33 antibody IgG4 antibody QB11465 and the anti-hIL-33 comparator-3 antibody QB11094 comparably inhibited the hIL-5 secretion, but they both are less potent than the anti-hTSLP antibody QB11718. However, the combination of (5) the lead anti-hTSLP IgG1-D265A antibody QB11718 and the lead anti-hIL-33 IgG4 antibody QB11465, and (6) the combination of the lead anti-hTSLP IgG1-D265A antibody QB11718 and the anti-hIL-33 comparator antibody QB11094 showed much higher inhibition for hIL-5 secretion, as the curves shift to the left. The calculated CI values are all below 1.0 (CI<1), see the table in FIG. 23C, indicating that the anti-hTSLP and anti-hIL-33 antibody combinations are more potent than the sum of individual antibodies, and that an anti-hTSLP/anti-hIL-33 MabPair product has a potential to achieve synergistic effect for treating asthma and COPD patients. When the lead anti-hTSLP IgG1-D265A antibody QB11718 was replaced with the anti-hTSLP comparator antibody QB10985 for testing the combined effect with anti-hIL-33 antibody IgG4 antibody QB11465, similar results were obtained. To our knowledge, this is the first time that the combination of anti-hTSLP blocking antibody and anti-hIL-33 blocking antibody can accomplish a synergistic biological effect on human primary cells.

### Example 20: The Anti-hIL-33 IgG4 Antibody QB11465 Significantly Inhibited Allergen-Induced Lung Eosinophilic Inflammation and IL-4 Production

As demonstrated in Example 13 above, the lead anti-hIL-33 IgG4 antibody QB11465 has strong biological activity in inhibiting the IFNγ cytokine secretion from primary human NK cells (FIG. 17A), IL-5 secretion from primary human ILC2s (FIG. 17B), and the level of p38 MAPK phosphorylation of primary human ILC2s (FIGS. 17C, 17D). It is important to test whether this antibody could have biological efficacy in mice asthma model.

Female C57BL/6 knock-in mice with hIL-33 (C57BL/6-IL33tml (hIL33)/Bcgen) with an age of 5~7 weeks were housed in a controlled environment at Biocytogen Inc. (Beijing, China). Mice were provided free access to autoclaved pellet food and tap water.

Purified House Dust Mite (HDM) extract purchased from Greer Laboratories (Lenoir, NC) was re-suspended in PBS to a final concentration of 1.42 mg/ml. Purified HDM extract (17.5 μl/mouse/day) containing 25 μg protein was administered intranasally to female C57BL/6 hIL-33 knock-in mice, once a day, for 10 consecutive days under isoflurane anesthesia, to develop airway inflammation. Five mice for each group: (1). No antibody treatment (HDM only), (2). the lead anti-hIL-33 IgG4 antibody QB11465 at 5 mg/kg, (3). the anti-hIL-33 comparator-3 antibody QB11094 at 5 mg/kg, were dosed intraperitoneally twice a week, for 3 consecutive weeks (Day 22, 25, 29, 32, 36, and 39). Five hIL-33 knock-in mice without HDM treatment were used as a reference. The animals were sacrificed on day 48 after the HDM administration. At the study endpoint, each animal was anesthetized by isoflurane inhalation followed by collection of Bronchoalveolar Lavage Fluid (BALF) as described by Hoecke L. V. et al. J Vis Exp. 2017; (123): 55398. Mouse cytokines IL-4, IL-5, IL-13, TSLP, and IL-33 in BALF were determined using a LEGENDplex™ 5-plex Panel Kit (Biolegend), human IL-33 in BALF was determined using a Human IL-33 Pre-coated ELISA Kit (Biolegend), all cytokines were determined in duplicate on the same day as BALF collection. The immune cells in BALF were collected and spun down at 800×g for 5 min, washed once with 200 μL DPBS. 100 μL DPBS containing fixable viability stain Zombie Aqua was added, cells were incubated at RT for 20 min. Immune cells were washed once again with DPBS, resuspended in 100 μL of eBioscience™ Flow Cytometry Staining Buffer (Thermo Fisher, Cat #00-4222-26). Immune cells were blocked with Mouse TruStain FcX™ Plus for 15 mins at 4° C., then stained with fluorescent antibodies shown in Table 32 below for 45 min at 4° C. in the dark, washed twice with flow staining buffer. Immune cells were resuspended in 100 μL DPBS+100 μL of IC Fix solution (Thermo Fisher, Cat #00-8222-49) and analyzed in BDR LSR II Flow Cytometer. After exclusion of red blood cells, debris, and doublets, the eosinophils were identified as CD45+, SiglecF+, CD3−, CD19−, NKp46− and are reported as % of live cells. FlowJo software was used for data analysis, One-way ANOVA test was employed for statistical analysis.

TABLE 32
Fluorescence labeled antibodies used for analysis of the
different cell types in the BAL fluid by flow cytometry.

| | Species | | | | | | |
|---|---|---|---|---|---|---|---|
| Channel | Reactivity | Marker | Fluorochrome | Clone | Dilution | Vendor | Cat # |
| VL2 | m/h | Zombie Aqua | Zombie Aqua | — | 1:500 | Biolegend | 423102 |
| VL3 | m | CD19 | BV605 | 6D5 | 1:100 | Blolegend | 115540 |
| VL6 | m | NKp46 | BV785 | 29A1.4 | 1:100 | Biolegend | 137637 |
| | | (CD335) | | | | | |
| BL1 | m | CD3 | AF488 | 17A2 | 1:100 | Biolegend | 100210 |
| BL3 | m | Siglec-F | PerCP-Cy5.5 | S17007L | 1:100 | Blolegend | 155526 |
| RL3 | m | CD45 | APC-Fire 750 | 30-F11 | 1:100 | Biolegend | 103154 |

As shown in FIG. 24A, the hIL-33 knock-in C57BL/6 mice had ~6% eosinophils at base level (labeled as “Control”), HDM stimulation increased the eosinophils to ~27%, indicating that HDM stimulation caused an inflammatory response in lung. The lead anti-hIL-33 IgG4 antibody QB11465 decreased the level of eosinophils from ~27% to ~15%, suggesting that anti-hIL-33 antibody QB11465 can significantly inhibit the HDM-induced airway inflammation (p=0.0002). The anti-hIL-33 comparator-3 antibody QB11094 decreased to eosinophils to ~17%, suggesting that this comparator antibody also inhibits the HDM-induced airway inflammation significantly (p=0.0024). The mouse cytokines IL-5, IL-13, TSLP and human IL-33 in BALF were all below the detection limit, however, mouse IL-4 production was increased upon HDM stimulation. The treatment of the lead anti-hIL-33 IgG4 antibody QB11465 and the anti-hIL-33 comparator-3 antibody QB11094 significantly decreased the mouse IL-4 production with p=0.0462 and p=0.0046, respectively. Since IL-4 is a major TH2 cytokine, the decrease of IL-4 production suggests an inhibition of lung inflammation.

Sequence Listing (using the Kabat Numbering System):
Amino acid sequence of Avitag_c-MYC_His6-tagged hIL-33 (Ser112-Thr270),
QB10975
SEQ ID NO: 1
GLNDIFEAQKIEWHEGGGGSEQKLISEEDLGSSHHHHHHSSGSITGISPITEYLASLSTYNDQSITFALE
DESYEIYVEDLKKDEKKDKVLLSYYESQHPSNESGDGVDGKMLMVTLSPTKDFWLHANNKEHSVELHKCE
KPLPDQAFFVLHNMHSNCVSFECKTDPGVFIGVKDNHLALIKVDSSENLCTENILFKLSET
Amino acid sequence of His6-tagged hIL-33 (Ser112-Thr270) with C208S + C232S,
QB11921
SEQ ID NO: 2
HHHHHHSSGSITGISPITEYLASLSTYNDQSITFALEDESYEIYVEDLKKDEKKDKVLLSYYESQHPSNE
SGDGVDGKMLMVTLSPTKDFWLHANNKEHSVELHKSEKPLPDQAFFVLHNMHSNCVSFESKTDPGVFIGV
KDNHLALIKVDSSENLCTENILFKLSET
Amino acid sequence of His6-tagged cyIL-33 (Ser112-Ile270), QB10976
SEQ ID NO: 3
GLNDIFEAQKIEWHEGGGGSDYKDDDDKGSSHHHHHHSSGSITGISPITESLASLSTYNDQSITFALEDE
SYEIYVEDLKKDKKKDKVLLSYYESQHPSSESGDGVDGKMLMVTLSPTKDFWLQANNKEHSVELHKCEKP
LPDQAFFVLHNRSFNCVSFECKTDPGVFIGVKDNHLALIKVDYSENLGSENILFKLSEI
Amino acid sequence of murine anti-hIL-33 QB10998 VL
SEQ ID NO: 4
DIVMTQAAPSIPVTPGESVSISCKSSKSLLHSNGNTYLYWFLQRPGQSPQLLIYRMSNLASGVPDRFSGS
GSGTAFTLRISRVEAEDVGVYYCMQHLENPYTFGGGTKLELN
Amino acid sequence of chimeric anti-hIL-33 QB10998 LC
SEQ ID NO: 5
DIVMTQAAPSIPVTPGESVSISCKSSKSLLHSNGNTYLYWFLQRPGQSPQLLIYRMSNLASGVPDRFSGS
GSGTAFTLRISRVEAEDVGVYYCMQHLENPYTFGGGTKLELNRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC
Amino acid sequence of murine anti-hIL-33 QB10998 VH
SEQ ID NO: 6
EVMLVESGGGLVKPGGSLKLSCAASGFTFYSSAMSWVRQTPEKRLEWVATISSGGSNTYYPDSVKGRFTI
SRDNAKNTLYLQMSSLGSEDTAMYYCASAYYGRRYDAMDYWGQGTSVTVSS
Amino acid sequence of chimeric anti-hIL-33 QB10998 HC
SEQ ID NO: 7
EVMLVESGGGLVKPGGSLKLSCAASGFTFYSSAMSWVRQTPEKRLEWVATISSGGSNTYYPDSVKGRFTI
SRDNAKNTLYLQMSSLGSEDTAMYYCASAYYGRRYDAMDYWGQGTSVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
Amino acid sequence of anti-hIL-33 QB11004 VL
SEQ ID NO: 8
DVVMTQSPPSLPVTLGQSASISCKSSKSLLHSNGNTYLYWFQQRPGQSPQLLIYRMSNLASGVPDRFSGS
GSGTDFTLKISRVEAEDVGVYYCMQHLENPYTFGQGTKVEIK
Amino acid sequence of anti-hIL-33 QB11004 LC
SEQ ID NO: 9
DVVMTQSPPSLPVTLGQSASISCKSSKSLLHSNGNTYLYWFQQRPGQSPQLLIYRMSNLASGVPDRFSGS
GSGTDFTLKISRVEAEDVGVYYCMQHLENPYTFGQGTKVEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC
Amino acid sequence of anti-hIL-33 QB11004 VH
SEQ ID NO: 10
EVQLLESGGGLVQPGGSLRLSCAASGFTFYSSAMSWVRQTQGKGLEWVSTISSGGSNTYYPDSVKGRFTI
SRDNSKNTLYLQMNSLGAEDTAVYYCASAYYGRRYDAMDYWGQGTLVTVSS
Amino acid sequence of anti-hIL-33 QB11004 HC
SEQ ID NO: 11
EVQLLESGGGLVQPGGSLRLSCAASGFTFYSSAMSWVRQTQGKGLEWVSTISSGGSNTYYPDSVKGRFTI
SRDNSKNTLYLQMNSLGAEDTAVYYCASAYYGRRYDAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
Amino acid sequence of anti-hIL-33 QB11061 VL
SEQ ID NO: 12
DVVMTQSPPSLPVTLGQSASISCKSSKSLLHSNGNTYLYWFQQRPGQSPQLLIYRMSNLASGVPDRFSGS
GSGTDFTLKISRVEAEDVGVYYCMQHLENPYTFGQGTKVEIK
Amino acid sequence of anti-hIL-33 QB11061 LC
SEQ ID NO: 13
DVVMTQSPPSLPVTLGQSASISCKSSKSLLHSNGNTYLYWFQQRPGQSPQLLIYRMSNLASGVPDRFSGS
GSGTDFTLKISRVEAEDVGVYYCMQHLENPYTFGQGTKVEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC
Amino acid sequence of anti-hIL-33 QB11061 VH
SEQ ID NO: 14
EVQLLESGGGLVQPGGSLRLSCAASGFTFYSSAMSWVRQTPGKGLEWVSTISSGGSNTYYPDSVKGRFTI
SRDNSKNTLYLQMNSLGAEDTAVYYCASAYYGRRYDAMDYWGQGTLVTVSS
Amino acid sequence of anti-hIL-33 QB11061 HC
SEQ ID NO: 15
EVQLLESGGGLVQPGGSLRLSCAASGFTFYSSAMSWVRQTPGKGLEWVSTISSGGSNTYYPDSVKGRFTI
SRDNSKNTLYLQMNSLGAEDTAVYYCASAYYGRRYDAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
Amino acid sequence of anti-hIL-33 QB11119 VL
SEQ ID NO: 16
DVVMTQSPPSLPVTLGQSASISCKSSKSLLHSNANTYLYWFQQRPGQSPQLLIYRMSNLASGVPDRFSGS
GSGTDFTLKISRVEAEDVGVYYCMQHLENPYTFGQGTKVEIK
Amino acid sequence of anti-hIL-33 QB11119 LC
SEQ ID NO: 17
DVVMTQSPPSLPVTLGQSASISCKSSKSLLHSNANTYLYWFQQRPGQSPQLLIYRMSNLASGVPDRFSGS
GSGTDFTLKISRVEAEDVGVYYCMQHLENPYTFGQGTKVEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC
Amino acid sequence of anti-hIL-33 QB11119 VH
SEQ ID NO: 18
EVQLLESGGGLVQPGGSLRLSCAASGFTFYSSAMSWVRQTQGKGLEWVSTISSGGSNTYYPDSVKGRFTI
SRDNSKNTLYLQMNSLGAEDTAVYYCASAYYGRRYDAMDYWGQGTLVTVSS
Amino acid sequence of anti-hIL-33 QB11119 HC
EVQLLESGGGLVQPGGSLRLSCAASGFTFYSSAMSWVRQTQGKGLEWVSTISSGGSNTYYPDSVKGRFTI
SEQ ID NO: 19
SRDNSKNTLYLQMNSLGAEDTAVYYCASAYYGRRYDAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPEAAGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALAAPIEKTISKAKGQPREP
QVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
Amino acid sequence of anti-hIL-33 QB11421 VL
SEQ ID NO: 20
DVVMTQSPPSLPVTLGQSASISCKSSESLLHSNANTYLYWFQQRPGQSPQLLIYRGSNLASGVPDRFSGS
GSGTDFTLKISRVEAEDVGVYYCMQHLRNPYTFGQGTKVEIK
Amino acid sequence of anti-hIL-33 QB11421 LC
SEQ ID NO: 21
DVVMTQSPPSLPVTLGQSASISCKSSESLLHSNANTYLYWFQQRPGQSPQLLIYRGSNLASGVPDRFSGS
GSGTDFTLKISRVEAEDVGVYYCMQHLRNPYTFGQGTKVEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC
Amino acid sequence of anti-hIL-33 QB11421 VH
SEQ ID NO: 22
EVQLLESGGGLVQPGGSLRLSCAASGFTFYSSAMSWFRQTQGKGLEWVSTISSGGSNTYYPDSVKGRFTI
SRDNSKNTLYLQMNSLGAEDTAVYYCASAYYGRRYDAMDYWGQGTLVTVSS
Amino acid sequence of anti-hIL-33 QB11421 HC
SEQ ID NO: 23
EVQLLESGGGLVQPGGSLRLSCAASGFTFYSSAMSWFRQTQGKGLEWVSTISSGGSNTYYPDSVKGRFTI
SRDNSKNTLYLQMNSLGAEDTAVYYCASAYYGRRYDAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPEAAGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALAAPIEKTISKAKGQPREP
QVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
Nucleotide sequence encoding anti-hIL-33 QB11465 VL
SEQ ID NO: 24
GACGTAGTGATGACTCAGAGTCCTCCAAGCCTCCCTGTTACTCTCGGTCAGTCCGCCAGCATAAGCTGTA
AGTCCTCCGAATCACTTCTGCATTCAAATGCTAACACTTATCTCTACTGGTTTCAGCAAAGACCTGGCCA
ATCACCCCAGTTGCTCATTTATAGGGGGAGCAATTTGGCTAGTGGGGTTCCAGATCGCTTTTCAGGAAGC
GGCTCTGGTACCGACTTTACCCTCAAAATCAGTCGAGTAGAAGCTGAGGACGTTGGAGTTTACTATTGTA
TGCAGCACCTCCGAAATCCATACACTTTTGGGCAGGGGACAAAGGTCGAAATAAAG
Amino acid sequence of anti-hIL-33 QB11465 VL
SEQ ID NO: 25
DVVMTQSPPSLPVTLGQSASISCKSSESLLHSNANTYLYWFQQRPGQSPQLLIYRGSNLASGVPDRFSGS
GSGTDFTLKISRVEAEDVGVYYCMQHLRNPYTFGQGTKVEIK
Nucleotide sequence encoding anti-hIL-33 QB11465 LC
SEQ ID NO: 26
GACGTAGTGATGACTCAGAGTCCTCCAAGCCTCCCTGTTACTCTCGGTCAGTCCGCCAGCATAAGCTGTA
AGTCCTCCGAATCACTTCTGCATTCAAATGCTAACACTTATCTCTACTGGTTTCAGCAAAGACCTGGCCA
ATCACCCCAGTTGCTCATTTATAGGGGGAGCAATTTGGCTAGTGGGGTTCCAGATCGCTTTTCAGGAAGC
GGCTCTGGTACCGACTTTACCCTCAAAATCAGTCGAGTAGAAGCTGAGGACGTTGGAGTTTACTATTGTA
TGCAGCACCTCCGAAATCCATACACTTTTGGGCAGGGGACAAAGGTCGAAATAAAGCGTACGGTGGCTGC
ACCATCTGTCTTCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCCAATCGGGTAACT
CCCAGGAGAGTGTCACAGAGCAGGACAGCAAGGACAGCACCTACAGCCTCAGCAGCACCCTGACGCTGAG
CAAAGCAGACTACGAGAAACACAAAGTCTACGCCTGCGAAGTCACCCATCAGGGCCTGAGCTCGCCCGTC
ACAAAGAGCTTCAACAGGGGAGAGTGTTGATAA
Amino acid sequence of anti-hIL-33 QB11465 LC
SEQ ID NO: 27
DVVMTQSPPSLPVTLGQSASISCKSSESLLHSNANTYLYWFQQRPGQSPQLLIYRGSNLASGVPDRFSGS
GSGTDFTLKISRVEAEDVGVYYCMQHLRNPYTFGQGTKVEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC
Nucleotide sequence encoding anti-hIL-33 QB11465 VH
SEQ ID NO: 28
GAGGTTCAACTTTTGGAGTCTGGCGGTGGTCTGGTGCAACCCGGTGGGAGTTTGCGACTTAGCTGTGCAG
CCAGTGGATTCACTTTTTATAGCTCTGCCATGTCATGGTTTCGGCAGACTCAAGGAAAGGGACTGGAATG
GGTTTCCACCATATCCTCAGGGGGCTCAAATACATACTACCCAGACTCCGTGAAAGGCCGGTTTACCATT
TCACGAGACAACTCAAAGAACACACTTTACCTGCAAATGAATAGTCTGGGCGCTGAAGATACAGCCGTAT
ATTATTGTGCATCAGCATACTACGGACGCCGATACGACGCAATGGATTATTGGGGACAGGGCACCCTGGT
CACTGTCAGCTCT
Amino acid sequence of anti-hIL-33 QB11465 VH
SEQ ID NO: 29
EVQLLESGGGLVQPGGSLRLSCAASGFTFYSSAMSWFRQTQGKGLEWVSTISSGGSNTYYPDSVKGRFTI
SRDNSKNTLYLQMNSLGAEDTAVYYCASAYYGRRYDAMDYWGQGTLVTVSS
Nucleotide sequence encoding anti-hIL-33 QB11465 IgG4 HC
SEQ ID NO: 30
GAGGTTCAACTTTTGGAGTCTGGCGGTGGTCTGGTGCAACCCGGTGGGAGTTTGCGACTTAGCTGTGCAG
CCAGTGGATTCACTTTTTATAGCTCTGCCATGTCATGGTTTCGGCAGACTCAAGGAAAGGGACTGGAATG
GGTTTCCACCATATCCTCAGGGGGCTCAAATACATACTACCCAGACTCCGTGAAAGGCCGGTTTACCATT
TCACGAGACAACTCAAAGAACACACTTTACCTGCAAATGAATAGTCTGGGCGCTGAAGATACAGCCGTAT
ATTATTGTGCATCAGCATACTACGGACGCCGATACGACGCAATGGATTATTGGGGACAGGGCACCCTGGT
CACTGTCAGCTCTGCTAGCACCAAGGGGCCATCCGTCTTCCCCCTGGCGCCCTGCTCCAGGAGCACCTCC
GAGAGCACAGCCGCCCTGGGCTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCC
AGCAACACCAAGGTGGACAAGAGAGTTGAGTCCAAATATGGCCCCCCATGCCCACCATGCCCAGCACCTG
AGTTCCTGGGGGGACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTTCAACTGGTACGTG
GATGGCGTGGAGGTGCATAATGCCAAGACAAAGCCGCGGGAGGAGCAGTTCAACAGCACGTACCGTGTGG
TCAGCGTCCTCACCGTCCTGCACCAGGACTGGCTGAACGGCAAGGAGTACAAGTGCAAGGTCTCCAACAA
AGGCCTCCCGTCCTCCATCGAGAAAACCATCTCCAAAGCCAAAGGGCAGCCCCGAGAGCCACAGGTGTAC
ACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTACAGCAGGCTAACCGTGGACAAGAGCAGGTGGCAGGAG
GGGAATGTCTTCTCATGCTCCGTGATGCATGAGGCTCTGCACAACCACTACACACAGAAGAGCCTCTCCC
TGTCTCTGGGTAAATGATAA
Amino acid sequence of anti-hIL-33 QB11465 IgG4 HC
SEQ ID NO: 31
EVQLLESGGGLVQPGGSLRLSCAASGFTFYSSAMSWFRQTQGKGLEWVSTISSGGSNTYYPDSVKGRFTI
SRDNSKNTLYLQMNSLGAEDTAVYYCASAYYGRRYDAMDYWGQGTLVTVSSASTKGPSVFPLAPCSRSTS
ESTAALGCLVKDYFPEPVTVSWNSGALTSGVHTFPAVLQSSGLYSLSSVVTVPSSSLGTKTYTCNVDHKP
SNTKVDKRVESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFLYSRLTVDKSRWQE
GNVFSCSVMHEALHNHYTQKSLSLSLGK
Amino acid sequence of Avitag_c-MYC_His6_hTSLP(wt) QB11630, which is a wild
type hTSLP produced in the presence of 25 uM furin inhibitor II from
Sigma (cat no. SCP0148).
SEQ ID NO: 32
GLNDIFEAQKIEWHEGGGGSEQKLISEEDLGSSHHHHHHSSGLVPRGSHMYDFTNCDFEKIKAAYLSTIS
KDLITYMSGTKSTEFNNTVSCSNRPHCLTEIQSLTFNPTAGCASLAKEMFAMKTKAALAIWCPGYSETQI
NATQAMKKRRKRKVTTNKCLEQVSQLQGLWRRFNRPLLKQQ
Amino acid sequence of Avitag_c-MYC_His6_hTSLP QB11033 with deletion of
126KRRKR130
SEQ ID NO: 33
GLNDIFEAQKIEWHEGGGGSEQKLISEEDLGSSHHHHHHSSGLVPRGSHMYDFTNCDFEKIKAAYLSTIS
KDLITYMSGTKSTEFNNTVSCSNRPHCLTEIQSLTFNPTAGCASLAKEMFAMKTKAALAIWCPGYSETQI
NATQAMKKVTTNKCLEQVSQLQGLWRRFNRPLLKQQ
Amino acid sequence of Avitag_c-MYC_His6_hTSLP QB11631 with mutations of
R127A+R130S
SEQ ID NO: 34
GLNDIFEAQKIEWHEGGGGSEQKLISEEDLGSSHHHHHHSSGLVPRGSHMYDFTNCDFEKIKAAYLSTIS
KDLITYMSGTKSTEFNNTVSCSNRPHCLTEIQSLTFNPTAGCASLAKEMFAMKTKAALAIWCPGYSETQI
NATQAMKKARKSKVTTNKCLEQVSQLQGLWRRFNRPLLKQQ
Amino acid sequence of Avitag_FLAG_His6_cyTSLP QB11632, which is a wild type
cyTSLP in the presence of 25 uM furin inhibitor II from Sigma (cat no. SCP0148)
SEQ ID NO: 35
GLNDIFEAQKIEWHEGGGGSDYKDDDDKGSSHHHHHHSSGLVPRGSHMYDFTNCDFQKIEADYLRTISKD
LITYMSGTKSTDFNNTVSCSNRPHCLTEIQSLTFNPTPRCASLAKEMFARKTKATLALWCPGYSETQINA
TQAMKKRRKRKVTTNKCLEQVSQLLGLWRRFIRTLLKKQ
Amino acid sequence of Avitag_FLAG_His6_cyTSLP QB10974 with deletion of
126KRRKR130
SEQ ID NO: 36
GLNDIFEAQKIEWHEGGGGSDYKDDDDKGSSHHHHHHSSGLVPRGSHMYDFTNCDFQKIEADYLRTISKD
LITYMSGTKSTDFNNTVSCSNRPHCLTEIQSLTFNPTPRCASLAKEMFARKTKATLALWCPGYSETQINA
TQAMKKVTTNKCLEQVSQLLGLWRRFIRTLLKKQ
Amino acid sequence of Avitag_FLAG_His6_cyTSLP QB11633 with mutations of
R127A+R130S
SEQ ID NO: 37
GLNDIFEAQKIEWHEGGGGSDYKDDDDKGSSHHHHHHSSGLVPRGSHMYDFTNCDFQKIEADYLRTISKD
LITYMSGTKSTDFNNTVSCSNRPHCLTEIQSLTFNPTPRCASLAKEMFARKTKATLALWCPGYSETQINA
TQAMKKARKSKVTTNKCLEQVSQLLGLWRRFIRTLLKKQ
Amino acid sequence of hTSLPR-Fc fusion protein QB11034
SEQ ID NO: 38
GAAEGVQIQIIYFNLETVQVTWNASKYSRTNLTFHYRFNGDEAYDQCTNYLLQEGHTSGCLLDAEQRDDI
LYFSIRNGTHPVFTASRWMVYYLKPSSPKHVRFSWHQDAVTVTCSDLSYGDLLYEVQYRSPFDTEWQSKQ
ENTCNVTIEGLDAEKCYSFWVRVKAMEDVYGPDTYPSDWSEVTCWQRGEIRDACAETPTPPKPKLSKAAA
EPKDKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRWQQGNVFSCSV
MHEALHNHYTQKSLSLSPGK
Amino acid sequence of anti-hTSLP VH of clone 135F1 from rabbit hybridoma
SEQ ID NO: 39
QEQLEESGGDLVKPEGSLTLTCKASGFDFSSYWISWVRQAPGKRPEWIACIDSDIDSADYASWAKGRFTM
SRTSSTTVTLQMTSLTAADTATYFCVRNLGLWGPGTLVTVSS
Amino acid sequence of anti-hTSLP HC of clone 135F1 from rabbit hybridoma
SEQ ID NO: 40
QEQLEESGGDLVKPEGSLTLTCKASGFDFSSYWISWVRQAPGKRPEWIACIDSDIDSADYASWAKGRFTM
SRTSSTTVTLQMTSLTAADTATYFCVRNLGLWGPGTLVTVSSGQPKAPSVFPLAPCCGDTPSSTVTLGCL
VKGYLPEPVTVTWNSGTLTNGVRTFPSVRQSSGLYSLSSVVSVTSSSQPVTCNVAHPATNTKVDKTVAPS
TCSKPMCPPPELLGGPSVFIFPPKPKDTLMISRTPEVTCVVVDVSQDDPEVQFTWYINNEQVRTARPPLR
EQQFNSTIRVVSTLPIAHQDWLRGKEFKCKVHNKALPAPIEKTISKARGQPLEPKVYTMGPPREELSSRS
VSLTCMINGFYPSDISVEWEKNGKAEDNYKTTPTVLDSDGSYFLYSKLSVPTSEWQRGDVFTCSVMHEAL
HNHYTQKSISRSPGK
Amino acid sequence of anti-hTSLP VL of clone 135F1 from rabbit hybridoma
SEQ ID NO: 41
AQVLTQTASPVSAAVGGTVTINCQSSQNVYDKDALAWYQHKPGQRPKLLIYEASKLASGVPSRFSGSGAG
TQFTLTISGVQCDDAATYYCAGTFIDNIYTFGGGTEVVVK
Amino acid sequence of anti-hTSLP LC of clone 135F1 from rabbit hybridoma
SEQ ID NO: 42
AQVLTQTASPVSAAVGGTVTINCQSSQNVYDKDALAWYQHKPGQRPKLLIYEASKLASGVPSRFSGSGAG
TQFTLTISGVQCDDAATYYCAGTFIDNIYTFGGGTEVVVKGDPVAPTVLIFPPAADQVATGTVTIVCVAN
KYFPDVTVTWEVDGTTQTTGIENSKTPQNSADCTYNLSSTLTLTSTQYNSHKEYTCKVTQGTTSVVQSFN
RGDC
Amino acid sequence of anti-hTSLP QB11341 and QB11548 VH of humanized clone
135F1
SEQ ID NO: 43
EVQLVESGGGLVQPGGSLRLSCAASGFDFSSYWISWVRQAPGKGPEWIASIDIDIDIADYASWAKGRFTI
SRDNSKNTLYLQMNSLRAEDTAVYYCVRNLGLWGPGTLVTVSS
Amino acid sequence of anti-hTSLP QB11341 and QB11548 HC of humanized clone
135F1
SEQ ID NO: 44
EVQLVESGGGLVQPGGSLRLSCAASGFDFSSYWISWVRQAPGKGPEWIASIDIDIDIADYASWAKGRFTI
SRDNSKNTLYLQMNSLRAEDTAVYYCVRNLGLWGPGTLVTVSSASTKGPSVFPLAPCSRSTSESTAALGC
LVKDYFPEPVTVSWNSGALTSGVHTFPAVLQSSGLYSLSSVVTVPSSSLGTKTYTCNVDHKPSNTKVDKR
VESKYGPPCPPCPAPPVAGPSVFLFPPKPKDTLMISRTPEVTCVVVDVSHEDPEVQFNWYVDGVEVHNAK
TKPREEQFNSTFRVVSVLTVVHQDWLNGKEYKCKVSNKGLAAPIEKTISKTKGQPREPQVYTLPPSREEM
TKNQVSLTCLVKGFYPSDIAVEWESNGQPENNYKTTPPMLDSDGSFFLYSKLTVDKSRWQQGNVFSCSVM
HEALHNHYTQKSLSLSPGK
Amino acid sequence of anti-hTSLP QB11341 VL of humanized clone 135F1
SEQ ID NO: 45
DIQMTQSPSSLSASVGDRVTITCQSSQNVYDKDALAWYQHKPGKRPKLLIYEASKLASGVPSRFSGSGSG
THFTLTISSLQPEDAATYYCAGTFIDNIYTFGGGTKVEIK
Amino acid sequence of anti-hTSLP QB11341 LC of humanized clone 135F1
SEQ ID NO: 46
DIQMTQSPSSLSASVGDRVTITCQSSQNVYDKDALAWYQHKPGKRPKLLIYEASKLASGVPSRFSGSGSG
THFTLTISSLQPEDAATYYCAGTFIDNIYTFGGGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVTK
SFNRGEC
Amino acid sequence of anti-hTSLP QB11548 VL of humanized clone 135F1 after yeast
display
SEQ ID NO: 47
DIQMTQSPSSLSASVGDRVTITCQSSQNVYDKDALAWYQHKPGKRPKLLIYEASRLASGVPSRFSGSGSG
THFTLTISSLQPEDAATYYCAGTRIDNIYTFGGGTKVEIK
Amino acid sequence of anti-hTSLP QB11548 LC of humanized clone 135F1 after yeast
display
SEQ ID NO: 48
DIQMTQSPSSLSASVGDRVTITCQSSQNVYDKDALAWYQHKPGKRPKLLIYEASRLASGVPSRFSGSGSG
THFTLTISSLQPEDAATYYCAGTRIDNIYTFGGGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVTK
SFNRGEC
Nucleotide sequence encoding anti-hTSLP QB11764 VH of the final clone
SEQ ID NO: 49
GAGGTGCAATTGGTGGAGTCTGGTGGGGGACTTGTGCAACCCGGCGGATCACTGCGGCTTAGCTGCGCAG
CATCAGGATTTGATTTTAGTAGCTATTGGATCAGTTGGGTTAGGCAGGCACCAGGGAAACGCCCCGAGTG
GATAGCAAGTATTGACATCGATATTGATATTGCCGATTATGCTTCCTGGGCCAAAGGCCGATTTACAATA
AGTCGCGATAACTCAAAAAACACCCTTTATCTTCAAATGAATTCCCTCCGAGCAGAAGATACCGCTGTTT
ACTACTGTGTCCGAAATCTGGGATTGTGGGGCCCAGGTACCCTTGTTACAGTTTCTTCC
Amino acid sequence of anti-hTSLP QB11764 VH of the final clone
SEQ ID NO: 50
EVQLVESGGGLVQPGGSLRLSCAASGFDFSSYWISWVRQAPGKRPEWIASIDIDIDIADYASWAKGRFTI
SRDNSKNTLYLQMNSLRAEDTAVYYCVRNLGLWGPGTLVTVSS
Nucleotide sequence encoding anti-hTSLP QB11764 IgG1-D265A of the final clone for
MabPair production
SEQ ID NO: 51
GAGGTGCAATTGGTGGAGTCTGGTGGGGGACTTGTGCAACCCGGCGGATCACTGCGGCTTAGCTGCGCAG
CATCAGGATTTGATTTTAGTAGCTATTGGATCAGTTGGGTTAGGCAGGCACCAGGGAAACGCCCCGAGTG
GATAGCAAGTATTGACATCGATATTGATATTGCCGATTATGCTTCCTGGGCCAAAGGCCGATTTACAATA
AGTCGCGATAACTCAAAAAACACCCTTTATCTTCAAATGAATTCCCTCCGAGCAGAAGATACCGCTGTTT
ACTACTGTGTCCGAAATCTGGGATTGTGGGGCCCAGGTACCCTTGTTACAGTTTCTTCCGCCTCAACTAA
GGGACCAAGCGTCTTCCCCCTTGCTCCCTCAAGCAAGTCCACAAGCGGGGGGACCGCTGCTTTGGGCTGC
CTCGTTGATGACTACTTTCCCGAACCTGTCACCGTGTCATGGAATTCCGGAGCCTTGACTTCTGGGGTAC
ATACCTGCCCTGCTGTATTGCAGAGTTCTGGACTGTATTCTCTGAGTAGTGTAGTGACTGTTCCATCTAG
CTCCCTGGGTACTCAGACCTACATTTGTAATGTCAATCACAAGCCTAGTAACACCAAAGTGGATAAGAAA
GTGGAGCCTAAGTCTGGTGATAAGACACACACATGCCCTCCCTGTCCAGCACCAGAGCTCCTTGGGGGAC
CTTCCGTCTTTCTTTTCCCTCCCAAACCCAAGGATACACTTATGATTAGCCGGACACCAGAAGTTACTTG
CGTCGTCGTTGCAGTGAGCCATGAGGACCCAGAAGTTAAGTTCAATTGGTACGTGGATGGCGTCGAGGTA
CATAATGCCAAGACCAAGCCACGTGAGGAGCAGTACAACAGTACATATAGGGTCGTGTCCGTACTTACAG
TGCTCCACCAAGATTGGCTGAATGGTAAGGAATATAAGTGTAAGGTTAGTAATAAAGCACTGCCCGCCCC
TATCGAAAAGACCATATCTAAAGCCAAAGGCCAGCCCCGTGAACCCCAGGTATATACACTTCCACCATCC
CGTGAGGAAATGACTAAAAATCAGGTATCTCTTACCTGCCTCGTAAAAGGTTTCTACCCATCCGATATAG
CAGTAGAGTGGGAAAGCAATGGCCAACCCGAGAACAATTACAAAACCACCCCCCCTGTGCTGCGTAGCGA
TGGTTCTTTTTTTCTTTACTCCGAACTTACAGTGGATAAGTCCCGTTGGCAGCAAGGAAACGTATTCTCT
TGTTCTGTAATGCATGAAGCACTTCATAATCATTATACTCAAAAGTCCCTGTCTCTCTCCCCCGGCAAGT
GA
Amino acid sequence of anti-hTSLP QB11764 IgG1-D265A of the final clone for MabPair
production
SEQ ID NO: 52
EVQLVESGGGLVQPGGSLRLSCAASGFDFSSYWISWVRQAPGKRPEWIASIDIDIDIADYASWAKGRFTI
SRDNSKNTLYLQMNSLRAEDTAVYYCVRNLGLWGPGTLVTVSSASTKGPSVFPLAPSSKSTSGGTAALGC
LVDDYFPEPVTVSWNSGALTSGVHTCPAVLQSSGLYSLSSVVTVPSSSLGTQTYICNVNHKPSNTKVDKK
VEPKSGDKTHTCPPCPAPELLGGPSVFLFPPKPKDTLMISRTPEVTCVVVAVSHEDPEVKFNWYVDGVEV
HNAKTKPREEQYNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLRSDGSFFLYSELTVDKSRWQQGNVFS
CSVMHEALHNHYTQKSLSLSPGK
Nucleotide sequence encoding anti-hTSLP QB11764 VL of the final clone
SEQ ID NO: 53
GACATACAGATGACCCAGTCCCCCAGCTCTTTGTCAGCATCCGTAGGAGATCGGGTCACCATCACCTGTC
AATCATCTCAAAACGTTTATGATAAAGACGCATTGGCCTGGTACCAGCATAAGCCAGGTAAAAGGCCAAA
ATTGCTCATCTACGAAGCTAGTCGGCTGGCATCAGGGGTGCCATCTAGGTTTTCCGGTAGCGGAAGTGGT
ACACATTTTACACTTACCATTTCCTCTCTTCAACCAGAAGATGCCGCCACCTATTATTGTGCCGGTACAA
GAATCGACAATATATACACTTTCGGAGATGGGACAAAGGTAGAAATAAAG
Amino acid sequence of anti-hTSLP QB11764 VL of the final clone
SEQ ID NO: 54
DIQMTQSPSSLSASVGDRVTITCQSSQNVYDKDALAWYQHKPGKRPKLLIYEASRLASGVPSRFSGSGSG
THFTLTISSLQPEDAATYYCAGTRIDNIYTFGDGTKVEIK
Nucleotide sequence encoding anti-hTSLP QB11764 LC of the final clone for MabPair
production
SEQ ID NO: 55
GACATACAGATGACCCAGTCCCCCAGCTCTTTGTCAGCATCCGTAGGAGATCGGGTCACCATCACCTGTC
AATCATCTCAAAACGTTTATGATAAAGACGCATTGGCCTGGTACCAGCATAAGCCAGGTAAAAGGCCAAA
ATTGCTCATCTACGAAGCTAGTCGGCTGGCATCAGGGGTGCCATCTAGGTTTTCCGGTAGCGGAAGTGGT
ACACATTTTACACTTACCATTTCCTCTCTTCAACCAGAAGATGCCGCCACCTATTATTGTGCCGGTACAA
GAATCGACAATATATACACTTTCGGAGATGGGACAAAGGTAGAAATAAAGCGTACGGTGGCCGCTCCTAG
TGTATTCATCTTTCCCCCTTCTGATGAACAATTGAAGTCTGGGACTGCAAAGGTTGTATGTCTCCTTAAT
AACTTCTACCCACGAGAAGCCAAAGTACAGTGGAAGGTGGATAATGCCCTCCAAAGCGGAAACAGTCAGG
AATGCGTAACTGAACAAGACAGCAAGGACTCAACCTACTCTCTTTCCTCCACACTCACACTGTCAAAGGC
AGATTACGAGAAGCACAAAGTCTACGCTTGCGAGGTCACTCATCAGGGCCTCAGCTCACCCGTCACTAAG
TCTTTTAACAGAGGCGAATCTTGA
Amino acid sequence of anti-hTSLP QB11764 LC of the final clone for MabPair production
SEQ ID NO: 56
DIQMTQSPSSLSASVGDRVTITCQSSQNVYDKDALAWYQHKPGKRPKLLIYEASRLASGVPSRFSGSGSG
THFTLTISSLQPEDAATYYCAGTRIDNIYTFGDGTKVEIKRTVAAPSVFIFPPSDEQLKSGTAKVVCLLN
NFYPREAKVQWKVDNALQSGNSQECVTEQDSKDSTYSLSSTLTLSKADYEKHKVYACEVTHQGLSSPVTK
SFNRGES
Amino acid sequence of anti-hTSLP QB10987 VH of chimeric antibody clone 23B12
SEQ ID NO: 57
EEKLQQSGDDLVRPGAAVKMSCKASGYIFTDYAMHWVKQRPGQGLEWIGTFIPLLDTSDYNQNFKGRATL
TADKSSNTAYMELSRLTSEDSAVYYCARMGVTHSYVMDAWGQGASVTVSS
Amino acid sequence of anti-hTSLP QB10987 HC of chimeric antibody clone 23B12
SEQ ID NO: 58
EEKLQQSGDDLVRPGAAVKMSCKASGYIFTDYAMHWVKQRPGQGLEWIGTFIPLLDTSDYNQNFKGRATL
TADKSSNTAYMELSRLTSEDSAVYYCARMGVTHSYVMDAWGQGASVTVSSASTKGPSVFPLAPCSRSTSE
STAALGCLVKDYFPEPVTVSWNSGALTSGVHTFPAVLQSSGLYSLSSVVTVPSSSLGTKTYTCNVDHKPS
NTKVDKRVESKYGPPCPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLAAPIEKTISKTKGQPREPQVYTL
PPSREEMTKNQVSLTCLVKGFYPSDIAVEWESNGQPENNYKTTPPMLDSDGSFFLYSKLTVDKSRWQQGN
VFSCSVMHEALHNHYTQKSLSLSPGK
Amino acid sequence of anti-hTSLP QB10987 VL of chimeric antibody clone 23B12
SEQ ID NO: 59
DIVLTQSPATLSVTPGESVSLSCRASQPISISVHWFQQKSNESPRLLIKFASQSISGIPSRFSGSGSGTD
FTLNINRVESEDFSVYYCQQTFSLPYTFGTGTKLELK
Amino acid sequence of anti-hTSLP QB10987 LC of chimeric antibody clone 23B12
SEQ ID NO: 60
DIVLTQSPATLSVTPGESVSLSCRASQPISISVHWFQQKSNESPRLLIKFASQSISGIPSRFSGSGSGTD
FTLNINRVESEDFSVYYCQQTFSLPYTFGTGTKLELKRTVAAPSVFIFPPSDEQLKSGTASVVCLINNFY
PREAKVQWKVDNALQSGNSQESVTEQDSKDSTYSLSSTLTLSKADYEKHKVYACEVTHQGLSSPVTKSEN
RGEC
Amino acid sequence of anti-hTSLP QB10990 VH of humanized antibody clone hz-3C
SEQ ID NO: 61
QVQLVQSGDEVKKPGSSVKVSCKASGYIFTDYAMHWVRQRPGQGLEWMGTFIPLLDTSDYNQNFKGRATI
TADKSTSTAYMELSSLRSEDTAVYYCARMGVTHSYVMDAWGQGTSVTVSS
Amino acid sequence of anti-hTSLP QB10990 HC of humanized antibody clone hz-3C
SEQ ID NO: 62
QVQLVQSGDEVKKPGSSVKVSCKASGYIFTDYAMHWVRQRPGQGLEWMGTFIPLLDTSDYNQNFKGRATI
TADKSTSTAYMELSSLRSEDTAVYYCARMGVTHSYVMDAWGQGTSVTVSSASTKGPSVFPLAPCSRSTSE
STAALGCLVKDYFPEPVTVSWNSGALTSGVHTFPAVLQSSGLYSLSSVVTVPSSSLGTKTYTCNVDHKPS
NTKVDKRVESKYGPPCPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLAAPIEKTISKTKGQPREPQVYTL
PPSREEMTKNQVSLTCLVKGFYPSDIAVEWESNGQPENNYKTTPPMLDSDGSFFLYSKLTVDKSRWQQGN
VFSCSVMHEALHNHYTQKSLSLSPGK
Amino acid sequence of anti-hTSLP QB10990 VL of humanized antibody clone hz-3C,
identical to QB11060 VL
SEQ ID NO: 63
EIVLTQSPGTLSLSPGERATLSCRASQPISISVHWYQQKPGEAPRLLIYFASQSISGIPDRFSGSGSGTD
FTLTISRLESEDFAVYYCQQTFSLPYTFGTGTKVEIK
Amino acid sequence of anti-hTSLP QB10990 LC of humanized antibody clone hz-3C,
identical to QB11060 LC
SEQ ID NO: 64
EIVLTQSPGTLSLSPGERATLSCRASQPISISVHWYQQKPGEAPRLLIYFASQSISGIPDRFSGSGSGTD
FTLTISRLESEDFAVYYCQQTFSLPYTFGTGTKVEIKRTVAAPSVFIFPPSDEQLKSGTASVVCLINNFY
PREAKVQWKVDNALQSGNSQESVTEQDSKDSTYSLSSTLTLSKADYEKHKVYACEVTHQGLSSPVTKSFN
RGEC
Amino acid sequence of anti-hTSLP QB11060 VH of humanized antibody clone hz-3C,
with D9P+S108L substitutions, identical to QB11237 VH
SEQ ID NO: 65
QVQLVQSGPEVKKPGSSVKVSCKASGYIFTDYAMHWVRQRPGQGLEWMGTFIPLLDTSDYNQNFKGRATI
TADKSTSTAYMELSSLRSEDTAVYYCARMGVTHSYVMDAWGQGTLVTVSS
Amino acid sequence of anti-hTSLP QB11060 HC of humanized antibody clone hz-3C,
with D9P+S108L substitutions, identical to QB11237 HC
SEQ ID NO: 66
QVQLVQSGPEVKKPGSSVKVSCKASGYIFTDYAMHWVRQRPGQGLEWMGTFIPLLDTSDYNQNFKGRATI
TADKSTSTAYMELSSLRSEDTAVYYCARMGVTHSYVMDAWGQGTLVTVSSASTKGPSVFPLAPCSRSTSE
STAALGCLVKDYFPEPVTVSWNSGALTSGVHTFPAVLQSSGLYSLSSVVTVPSSSLGTKTYTCNVDHKPS
NTKVDKRVESKYGPPCPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLAAPIEKTISKTKGQPREPQVYTL
PPSREEMTKNQVSLTCLVKGFYPSDIAVEWESNGQPENNYKTTPPMLDSDGSFFLYSKLTVDKSRWQQGN
VFSCSVMHEALHNHYTQKSLSLSPGK
Amino acid sequence of anti-hTSLP QB11237 VL of humanized antibody clone hz-3C
after yeast display
SEQ ID NO: 67
EIVLTQSPGTLSLSPGERATLSCRASQHISRSVHWYQQKPGEAPRLLIYFASQSISGIPDRFSGSGSGTD
FTLTISRLESEDFAVYYCQQSYSFPYTFGTGTKVEIK
Amino acid sequence of anti-hTSLP QB11237 LC of humanized antibody clone hz-3C
after yeast display
SEQ ID NO: 68
EIVLTQSPGTLSLSPGERATLSCRASQHISRSVHWYQQKPGEAPRLLIYFASQSISGIPDRFSGSGSGTD
FTLTISRLESEDFAVYYCQQSYSFPYTFGTGTKVEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC
Nucleotide sequence encoding anti-hTSLP QB11718 VH of the final clone
SEQ ID NO: 69
CAGGTCCAACTTGTTCAGTCAGGCCCAGAAGTCAAAAAACCTGGTAGTTCCGTCAAGGTATCCTGCAAGG
CAAGTGGATACATCTTCACAGACTATGCTATGCACTGGGTGCGTCAGAGACCAGGTCAGGACCTCGAATG
GATGGGCACTTTCATTCCCCTTCTGGATACCTCAGACTACAATCAGAATTTCAAGGGAAGAGCAACCATT
ACTGCTGATAAGTCAACAAGTACTGCTTATATGGAACTGAGCTCCTTGCGCAGCGAAGACACAGCCGTTT
ATTATTGCGCCCGGATGGGTGTTACACATAGTTACGTGATGGACGCATGGGGACAGGGAACACTCGTAAC
AGTGTCTTCA
Amino acid sequence of anti-hTSLP QB11718 VH of the final clone
SEQ ID NO: 70
QVQLVQSGPEVKKPGSSVKVSCKASGYIFTDYAMHWVRQRPGQDLEWMGTFIPLLDTSDYNQNFKGRATI
TADKSTSTAYMELSSLRSEDTAVYYCARMGVTHSYVMDAWGQGTLVTVSS
Nucleotide sequence encoding anti-hTSLP QB11718 IgG1-D265A HC of the final clone
for MabPair production
SEQ ID NO: 71
CAGGTCCAACTTGTTCAGTCAGGCCCAGAAGTCAAAAAACCTGGTAGTTCCGTCAAGGTATCCTGCAAGG
CAAGTGGATACATCTTCACAGACTATGCTATGCACTGGGTGCGTCAGAGACCAGGTCAGGACCTCGAATG
GATGGGCACTTTCATTCCCCTTCTGGATACCTCAGACTACAATCAGAATTTCAAGGGAAGAGCAACCATT
ACTGCTGATAAGTCAACAAGTACTGCTTATATGGAACTGAGCTCCTTGCGCAGCGAAGACACAGCCGTTT
ATTATTGCGCCCGGATGGGTGTTACACATAGTTACGTGATGGACGCATGGGGACAGGGAACACTCGTAAC
AGTGTCTTCAGCTAGCACTAAAGGCCCTAGTGTGTTCCCACTCGCTCCAAGCTCCAAGTCAACCTCCGGC
GGCACCGCTGCCCTGGGTTGTCTCGTCGATGATTATTTTCCCGAACCAGTGACTGTGAGCTGGAATAGCG
GAGCTCTCACAAGTGGCGTGCACACTTGTCCTGCCGTATTGCAATCCTCCGGCCTGTATTCACTCAGTTC
TGTCGTTACCGTCCCCTCATCATCCCTCGGGACACAAACATATATATGCAATGTCAATCACAAGCCTAGT
AACACAAAAGTAGATAAAAAAGTCGAGCCAAAGTCCGGCGATAAAACCCATACTTGCCCACCCTGTCCAG
CTCCCGAGCTTCTGGGGGGACCCAGTGTATTTCTCTTTCCACCAAAGCCTAAAGACACACTGATGATTAG
CAGAACCCCCGAAGTTACCTGTGTCGTGGTAGCAGTTTCCCATGAAGATCCTGAAGTCAAGTTCAATTGG
TACGTAGATGGGGTGGAGGTCCATAACGCAAAAACAAAACCCCGCGAAGAGCAGTATAACTCTACTTACC
GGGTTGTGTCCGTGCTCACCGTGCTGCATCAAGATTGGCTGAATGGAAAGGAATACAAATGTAAGGTCAG
CAATAAGGCCCTTCCCGCACCTATTGAAAAAACAATCTCTAAAGCTAAGGGCCAACCACGAGAGCCTCAA
GTTTATACCCTCCCTCCATCTAGAGAAGAGATGACTAAAAACCAGGTGTCCCTCACTTGCCTCGTTAAGG
GCTTTTACCCAAGTGACATTGCTGTGGAGTGGGAATCAAATGGACAACCTGAAAACAATTACAAAACTAC
ACCCCCAGTCTTGAGGAGCGACGGCTCATTTTTTTTGTACTCTGAGCTTACCGTTGACAAAAGCCGCTGG
CAGCAAGGAAACGTTTTTTCATGCAGTGTAATGCATGAGGCACTTCACAATCATTACACACAGAAGTCAT
TGAGCCTCAGCCCTGGGAAGTGA
Amino acid sequence of anti-hTSLP QB11718 IgG1-D265A HC of the final clone for
MabPair production
SEQ ID NO: 72
QVQLVQSGPEVKKPGSSVKVSCKASGYIFTDYAMHWVRQRPGQDLEWMGTFIPLLDTSDYNQNFKGRATI
TADKSTSTAYMELSSLRSEDTAVYYCARMGVTHSYVMDAWGQGTLVTVSSASTKGPSVFPLAPSSKSTSG
GTAALGCLVDDYFPEPVTVSWNSGALTSGVHTCPAVLQSSGLYSLSSVVTVPSSSLGTQTYICNVNHKPS
NTKVDKKVEPKSGDKTHTCPPCPAPELLGGPSVFLFPPKPKDTLMISRTPEVTCVVVAVSHEDPEVKENW
YVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLRSDGSFFLYSELTVDKSRW
QQGNVFSCSVMHEALHNHYTQKSLSLSPGK
Nucleotide sequence encoding anti-hTSLP QB11718 VL of the final clone
SEQ ID NO: 73
GAAATAGTCCTCACACAGTCCCCAGGTACCCTCTCACTGTCACCCGGCGAAAGGGCCACTCTGTCCTGTC
GTGCAAGCCAGCACATCAGTCGCTCTGTCCATTGGTATCAACAGAAGCCAGGTGAGGCCCCACGTCTTCT
GATTTACTTCGCTAGTCAATCTATCTCCGGCATTCCTGACCGATTTAGCGGCAGCGGAAGCGGAACCGAC
TTCACTCTCACAATATCACGGCTTGAATCAGAGGATTTCGCCGTCTATTACTGCCAGCAGAGTTACAGCT
TCCCCTATACTTTCGGGCGCGGAACTAAGGTAGAAATTAAG
Amino acid sequence of anti-hTSLP QB11718 VL of the final clone
SEQ ID NO: 74
EIVLTQSPGTLSLSPGERATLSCRASQHISRSVHWYQQKPGEAPRLLIYFASQSISGIPDRFSGSGSGTD
FTLTISRLESEDFAVYYCQQSYSFPYTFGRGTKVEIK
Nucleotide sequence encoding anti-hTSLP QB11718 LC of the final clone for MabPair
production
SEQ ID NO: 75
GAAATAGTCCTCACACAGTCCCCAGGTACCCTCTCACTGTCACCCGGCGAAAGGGCCACTCTGTCCTGTC
GTGCAAGCCAGCACATCAGTCGCTCTGTCCATTGGTATCAACAGAAGCCAGGTGAGGCCCCACGTCTTCT
GATTTACTTCGCTAGTCAATCTATCTCCGGCATTCCTGACCGATTTAGCGGCAGCGGAAGCGGAACCGAC
TTCACTCTCACAATATCACGGCTTGAATCAGAGGATTTCGCCGTCTATTACTGCCAGCAGAGTTACAGCT
TCCCCTATACTTTCGGGCGCGGAACTAAGGTAGAAATTAAGCGTACGGTCGCCGCCCCCAGCGTATTCAT
ATTTCCTCCTAGCGATGAGCAGCTTAAATCAGGAACAGCAAAGGTTGTCTGTTTGTTGAACAATTTCTAT
CCTCGGGAAGCTAAGGTCCAATGGAAAGTGGACAACGCCTTGCAGAGCGGTAATTCACAAGAATGCGTGA
CAGAGCAAGATAGCAAAGACTCAACCTACTCACTGAGCTCTACTCTGACACTTTCTAAAGCTGATTATGA
GAAACACAAGGTATATGCATGTGAAGTGACTCACCAGGGGCTCAGCTCACCTGTGACTAAATCCTTTAAC
AGGGGCGAATCCTGA
Amino acid sequence of anti-hTSLP QB11718 LC of the final clone for MabPair production
SEQ ID NO: 76
EIVLTQSPGTLSLSPGERATLSCRASQHISRSVHWYQQKPGEAPRLLIYFASQSISGIPDRFSGSGSGTD
FTLTISRLESEDFAVYYCQQSYSFPYTFGRGTKVEIKRTVAAPSVFIFPPSDEQLKSGTAKVVCLLNNFY
PREAKVQWKVDNALQSGNSQECVTEQDSKDSTYSLSSTLTLSKADYEKHKVYACEVTHQGLSSPVTKSFN
RGES
Amino acid sequence of CDRI region of VL of anti-hIL-33 antibody QB11421 and
QB11465
SEQ ID NO: 77
KSSESLLHSNANTYLY
Amino acid sequence of CDR2 region of VL of anti-hIL-33 antibody QB11421 and
QB11465
SEQ ID NO: 78
RGSNLAS
Amino acid sequence of CDR3 region of VL of anti-hIL-33 antibody QB11421 and
QB11465
SEQ ID NO: 79
MQHLRNPYT
Amino acid sequence of CDRI region of VH of anti-hIL-33 antibody QB11421 and
QB11465
SEQ ID NO: 80
SSAMS
Amino acid sequence of CDR2 region of VH of anti-hIL-33 antibody QB11421 and
QB11465
SEQ ID NO: 81
TISSGGSNTYYPDSVKG
Amino acid sequence of CDR3 region of VH of anti-hIL-33 antibody QB11421 and
QB11465
SEQ ID NO: 82
AYYGRRYDAMDY
Amino acid sequence of CDRI region of VL of anti-hTSLP antibody QB11341,
QB11548, QB11764
SEQ ID NO: 83
QSSQNVYDKDALA
Amino acid sequence of CDR2 region of VL of anti-hTSLP antibody QB11341
SEQ ID NO: 84
EASKLAS
Amino acid sequence of CDR3 region of VL of anti-hTSLP antibody QB11341
SEQ ID NO: 85
AGTFIDNIYT
Amino acid sequence of CDRI region of VH of anti-hTSLP antibody QB11341,
QB11548, QB11764
SEQ ID NO: 86
SYWIS
Amino acid sequence of CDR2 region of VH of anti-hTSLP antibody QB11341,
QB11548, QB11764
SEQ ID NO: 87
SIDIDIDIADYASWAKG
Amino acid sequence of CDR3 region of VH of anti-hTSLP antibody QB11341,
QB11548, QB11764
SEQ ID NO: 88
NLGL
Amino acid sequence of CDR2 region of VL of anti-hTSLP antibody QB11548,
QB11764
SEQ ID NO: 89
EASRLAS
Amino acid sequence of CDR3 region of VL of anti-hTSLP antibody QB11548,
QB11764
SEQ ID NO: 90
AGTRIDNIYT
Amino acid sequence of CDR1 region of VL of anti-hTSLP antibody QB11237,
QB11718
SEQ ID NO: 91
RASQHISRSVH
Amino acid sequence of CDR2 region of VL of anti-hTSLP antibody QB11237,
QB11718
SEQ ID NO: 92
FASQSIS
Amino acid sequence of CDR3 region of VL of anti-hTSLP antibody QB11237,
QB11718
SEQ ID NO: 93
QQSYSFPYT
Amino acid sequence of CDRI region of VH of anti-hTSLP antibody QB11237,
QB11718
SEQ ID NO: 94
DYAMH
Amino acid sequence of CDR2 region of VH of anti-hTSLP antibody QB11237,
QB11718
SEQ ID NO: 95
TFIPLLDTSDYNQNFKG
Amino acid sequence of CDR3 region of VH of anti-hTSLP antibody QB11237,
QB11718
SEQ ID NO: 96
MGVTHSYVMDA

What is claimed is:

1. An anti-human IL33 (anti-hIL33) antibody comprising a heavy chain variable domain (VH) and a light chain variable region (VL), each comprising a complementarity determining region 1 (CDR1), a CDR2, and a CDR3,
wherein the anti-hIL33 antibody comprises a VH CDR1, VH CDR2, VH CDR3, VL CDR1, VL CDR 2, and VL CDR3 comprising, respectively, the following amino acid sequences: SEQ ID NOs: 80, 81, 82, 77, 78, and 79, and
wherein the anti-hIL33 antibody inhibits the interaction of human IL-33 and ST2/IL1AcP complex.

2. The anti-hIL33 antibody of claim 1, wherein the VH comprises an amino acid sequence which comprises no more than four alterations relative to an amino acid sequence selected from the group consisting of SEQ ID NOs: 22, and 29, and/or the VL comprises an amino acid sequence which comprises no more than four amino acid alterations relative to an amino acid sequence selected from the group consisting of SEQ ID Nos: 20, and 25.

3. The anti-hIL33 antibody of claim 2,
wherein the VH and the VL of the anti-hIL33 antibody each comprise an amino acid sequence, which, together, comprise two sequences,
wherein one of the two sequences comprises not more than four amino acid alterations relative to one sequence in a VH/VL pair of sequences, and the other of the two sequences comprises not more than four amino acid alterations relative to the other sequence in the VH/VL pair of sequences, and
wherein the VH/VL pair of sequences is selected from the group consisting of: SEQ ID NOs: 29 (VH) and 25 (VL); and SEQ ID NOs: 22 (VH) and 20 (VL).

4. The anti-hIL33 antibody of claim 1, wherein
the VH CDR1, VH CDR2, VH CDR3, VL CDR1, VL CDR 2, and VL CDR3 comprise, respectively, the amino acid sequences of SEQ ID NOs: 80, 81, 82, 77, 78, and 79,
the VH comprises an amino acid sequence which comprises no more than four alterations relative to the amino acid sequence of SEQ ID NO: 29, and
the VL comprises an amino acid sequence which comprises no more than four alterations relative to the amino acid sequence of SEQ ID NO: 25.

5. One or more polynucleotide(s) encoding an anti-hIL33 antibody of claim 1.

6. A host cell comprising one or more polynucleotide(s) of claim 5.

7. A method of treating a patient, in need thereof, having inflammation, an inflammatory disease, a chronic inflammatory airway disease, asthma, COPD, and/or Type 2 inflammation, said method comprising:
(a) administering to the patient an anti-hIL33 antibody of claim 1; or
(b) administering to the patient one or more polynucleotide(s) encoding the anti-hIL33 antibody of (a).

8. An anti-human TSLP (anti-hTSLP) antibody comprising a VH and a VL, each comprising a CDR1, a CDR2, and a CDR3,
wherein the anti-hTSLP antibody comprises a VH CDR1, VH CDR2, VH CDR3, VL CDR1, VL CDR 2, and VL CDR3 comprising, respectively, the following sequences: SEQ ID NOs: 86, 87, 88, 83, 84, and 85; SEQ ID NOs: 86, 87, 88, 83, 89, and 90; SEQ ID NOs: 94, 95, 96, 91, 92, and 93, and
wherein the anti-hTSLP antibody inhibits hTSLP binding to hTSLPR or hTSLP interacting with an hTSLPR/hIL7Rα complex.

9. The anti-hTSLP antibody of claim 8,
wherein the anti-hTSLP VH comprises an amino acid sequence which comprises no more than four alterations relative to an amino acid sequence selected from the group consisting of SEQ ID NOs: 43, 50, 65, and 70, and/or the anti-hTSLP VL comprises an amino acid sequence which comprises no more than four alterations relative to an amino acid sequence selected from the group consisting of SEQ ID NOs: 45, 47, 54, 67, and 74.

10. The anti-hTSLP antibody of claim 9,
wherein the VH and the VL of the anti-hTSLP antibody each comprise an amino acid sequence, which, together, comprise two sequences,
wherein one of the two sequences comprises not more than four amino acid alterations relative to one sequence in a VH/VL pair of sequences, and the other of the two sequences comprises not more than four amino acid alterations relative to the other sequence in the VH/VL pair of sequences, and
wherein the VH/VL pair of sequences is selected from the group consisting of: SEQ ID NOs: 43 (VH) and 45 (VL); SEQ ID NOs: 43 (VH) and 47 (VL); SEQ ID NOs: 50 (VH) and 54 (VL); SEQ ID NOs: 65 (VH) and 67 (VL); and SEQ ID NOs: 70 (VH) and 74 (VL).

11. The anti-hTSLP antibody of claim 8, wherein
the VH CDR1, VH CDR2, VH CDR3, VL CDR1, VL CDR 2, and VL CDR3 comprise, respectively, the amino acid sequences of SEQ ID NOs: 94, 95, 96, 91, 92, and 93,
the VH comprises an amino acid sequence which comprises no more than four alterations relative to the amino acid sequence of SEQ ID NO: 70, and
the VL comprises an amino acid sequence which comprises no more than four alterations relative to the amino acid sequence of SEQ ID NO: 74.

12. One or more polynucleotide(s) encoding anti-hTSLP antibody of claim 8.

13. A host cell comprising one or more polynucleotide(s) of claim 12.

14. A method of treating a patient, in need thereof, having inflammation, an inflammatory disease, a chronic inflammatory airway disease, asthma, COPD, and/or Type 2 inflammation, said method comprising:
(a) administering to the patient an anti-hTSLP antibody of claim 8; or
(b) administering to the patient one or more polynucleotide(s) encoding the anti-hTSLP antibody of (a).

15. A mixture comprising an anti-hIL33 antibody and an anti-hTSLP antibody, wherein:
(a) (1) the anti-hIL33 antibody comprises a VH and a VL, each comprising a CDR1, a CDR2, and a CDR3, (2) the anti-hIL33 antibody comprises a VH CDR1, VH CDR2, VH CDR3, VL CDR1, VL CDR 2, and VL CDR3 comprising, respectively, the following sequences: SEQ ID NOs: 80, 81, 82, 77, 78, and 79, and (3) the anti-hIL33 antibody inhibits the interaction of human IL-33 and ST2/IL1AcP complex; and
(b) (1) the anti-hTSLP antibody comprises a VH and a VL, each comprising a CDR1, a CDR2, and a CDR3, (2) anti-hTSLP antibody comprises a VH CDR1, VH CDR2, VH CDR3, VL CDR1, VL CDR 2, and VL CDR3 comprising, respectively, the following sequences: SEQ ID NOS: 86, 87, 88, 83, 84, and 85; SEQ ID NOs: 86, 87, 88, 83, 89, and 90; SEQ ID NOs: 94, 95, 96, 91, 92, and 93; and (3) the anti-hTSLP antibody inhibits hTSLP binding to hTSLPR or hTSLP interacting with an hTSLPR/hIL7Rα complex.

16. The mixture of claim 15, wherein
(a) (1) the VH CDR1, VH CDR2, VH CDR3, VL CDR1, VL CDR 2, and VL CDR3 of the anti-hIL33 antibody comprise, respectively, the amino acid sequences of SEQ ID NOs: 80, 81, 82, 77, 78, and 79, (2) the VH of the anti-hIL33 antibody comprises an amino acid sequence which comprises no more than four alterations relative to the amino acid sequence of SEQ ID NO: 29, and (3) the VL of the anti-hIL33 antibody comprises an amino acid sequence which comprises no more than four alterations relative to the amino acid sequence of SEQ ID NO: 25, and
(b) (1) the VH CDR1, VH CDR2, VH CDR3, VL CDR1, VL CDR 2, and VL CDR3 of the anti-hTSLP antibody comprise, respectively, the amino acid sequences of SEQ ID NOs: 94, 95, 96, 91, 92, and 93, (2) the VH comprises an amino acid sequence which comprises no more than four alterations relative to the amino acid sequence of SEQ ID NO: 70, and (3) the VL comprises an amino acid sequence which comprises no more than four alterations relative to the amino acid sequence of SEQ ID NO: 74.

17. One or more polynucleotide(s) encoding the mixture of claim 15.

18. A host cell comprising one or more polynucleotides of claim 17.

19. A method of treating a patient, in need thereof, having inflammation, an inflammatory disease, a chronic inflammatory airway disease, asthma, COPD, and/or Type 2 inflammation, said method comprising:
(a) administering to the patient the mixture of claim 15; or
(b) administering to the patient one or more polynucleotide(s) encoding the mixture of (a).

20. A mixture of antibodies comprising:
(a) an anti-hIL33 antibody comprising a heavy chain (HC) and a light chain (LC), wherein (1) the HC of the anti-hIL33 antibody is encoded by a nucleic acid sequence which encodes the amino acid sequence of SEQ ID NO: 31, and (2) the LC of the anti-hIL33 antibody is encoded by a nucleic acid sequence which encodes the amino acid sequence of SEQ ID NO: 27; and
(b) an anti-hTSLP antibody comprising an HC and an LC, wherein (1) the HC of the anti-hTSLP antibody is encoded by a nucleic acid sequence which encodes the amino acid sequence of SEQ ID NO: 72, and (2) the LC of the anti-hTSLP antibody is encoded by a nucleic acid sequence which encodes the amino acid sequence of SEQ ID NO: 76.

21. The mixture of claim 20, wherein:
the amino acid sequences of the HC and LC of the anti-hIL33 antibody are encoded by the nucleic acid sequences of SEQ ID NOs: 30 and 26, respectively; and
the amino acid sequences of the HC and LC of the anti-hTSLP antibody are encoded by the nucleic acid sequences of SEQ ID NOs: 71 and 75, respectively.


SEQUENCE LISTING (108 sequences)

SEQ ID NO: 1 - protein, 201 aa, synthetic construct
  source 1..201: note = Amino acid sequence of His6-tagged hIL-33 (Ser112 - Thr270), QB10975
GLNDIFEAQKIEWHEGGGGSEQKLISEEDLGSSHHHHHHSSGSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 2 - protein, 168 aa, synthetic construct
  source 1..168: note = Amino acid sequence of His6-tagged hIL-33 (Ser112 - Thr270) with C208S + C232S, QB11921
HHHHHHSSGSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNCVSFESKTDPGVFIGVKDNHLALIKVDSSENLCTENILFKLSET

SEQ ID NO: 3 - protein, 199 aa, synthetic construct
  source 1..199: note = Amino acid sequence of His6-tagged cyIL-33 (Ser112 - Ile270), QB10976
GLNDIFEAQKIEWHEGGGGSDYKDDDDKGSSHHHHHHSSGSITGISPITESLASLSTYNDQSITFALEDESYEIYVEDLKKDKKKDKVLLSYYESQHPSSESGDGVDGKMLMVTLSPTKDFWLQANNKEHSVELHKCEKPLPDQAFFVLHNRSFNCVSFECKTDPGVFIGVKDNHLALIKVDYSENLGSENILFKLSEI

SEQ ID NO: 4 - protein, 112 aa, synthetic construct
  source 1..112: note = Amino acid sequence of murine anti-hIL-33 QB10998 VL
DIVMTQAAPSIPVTPGESVSISCKSSKSLLHSNGNTYLYWFLQRPGQSPQLLIYRMSNLASGVPDRFSGSGSGTAFTLRISRVEAEDVGVYYCMQHLENPYTFGGGTKLELN

SEQ ID NO: 5 - protein, 219 aa, synthetic construct
  source 1..219: note = Amino acid sequence of chimeric anti-hIL-33 QB10998 LC
DIVMTQAAPSIPVTPGESVSISCKSSKSLLHSNGNTYLYWFLQRPGQSPQLLIYRMSNLASGVPDRFSGSGSGTAFTLRISRVEAEDVGVYYCMQHLENPYTFGGGTKLELNRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 121 aa, synthetic construct
  source 1..121: note = Amino acid sequence of murine anti-hIL-33 QB10998 VH
EVMLVESGGGLVKPGGSLKLSCAASGFTFYSSAMSWVRQTPEKRLEWVATISSGGSNTYYPDSVKGRFTISRDNAKNTLYLQMSSLGSEDTAMYYCASAYYGRRYDAMDYWGQGTSVTVSS

SEQ ID NO: 7 - protein, 451 aa, synthetic construct
  source 1..451: note = Amino acid sequence of chimeric anti-hIL-33 QB10998 HC
EVMLVESGGGLVKPGGSLKLSCAASGFTFYSSAMSWVRQTPEKRLEWVATISSGGSNTYYPDSVKGRFTISRDNAKNTLYLQMSSLGSEDTAMYYCASAYYGRRYDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 112 aa, synthetic construct
  source 1..112: note = Amino acid sequence of anti-hIL-33 QB11004 VL
DVVMTQSPPSLPVTLGQSASISCKSSKSLLHSNGNTYLYWFQQRPGQSPQLLIYRMSNLASGVPDRFSGSGSGTDFTLKISRVEAEDVGVYYCMQHLENPYTFGQGTKVEIK

SEQ ID NO: 9 - protein, 219 aa, synthetic construct
  source 1..219: note = Amino acid sequence of anti-hIL-33 QB11004 LC
DVVMTQSPPSLPVTLGQSASISCKSSKSLLHSNGNTYLYWFQQRPGQSPQLLIYRMSNLASGVPDRFSGSGSGTDFTLKISRVEAEDVGVYYCMQHLENPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 121 aa, synthetic construct
  source 1..121: note = Amino acid sequence of anti-hIL-33 QB11004 VH
EVQLLESGGGLVQPGGSLRLSCAASGFTFYSSAMSWVRQTQGKGLEWVSTISSGGSNTYYPDSVKGRFTISRDNSKNTLYLQMNSLGAEDTAVYYCASAYYGRRYDAMDYWGQGTLVTVSS

SEQ ID NO: 11 - protein, 451 aa, synthetic construct
  source 1..451: note = Amino acid sequence of anti-hIL-33 QB11004 HC
EVQLLESGGGLVQPGGSLRLSCAASGFTFYSSAMSWVRQTQGKGLEWVSTISSGGSNTYYPDSVKGRFTISRDNSKNTLYLQMNSLGAEDTAVYYCASAYYGRRYDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 112 aa, synthetic construct; identical to SEQ ID NO: 8
  source 1..112: note = Amino acid sequence of anti-hIL-33 QB11061 VL

SEQ ID NO: 13 - protein, 219 aa, synthetic construct; identical to SEQ ID NO: 9
  source 1..219: note = Amino acid sequence of anti-hIL-33 QB11061 LC

SEQ ID NO: 14 - protein, 121 aa, synthetic construct
  source 1..121: note = Amino acid sequence of anti-hIL-33 QB11061 VH
EVQLLESGGGLVQPGGSLRLSCAASGFTFYSSAMSWVRQTPGKGLEWVSTISSGGSNTYYPDSVKGRFTISRDNSKNTLYLQMNSLGAEDTAVYYCASAYYGRRYDAMDYWGQGTLVTVSS

SEQ ID NO: 15 - protein, 451 aa, synthetic construct
  source 1..451: note = Amino acid sequence of anti-hIL-33 QB11061 HC
EVQLLESGGGLVQPGGSLRLSCAASGFTFYSSAMSWVRQTPGKGLEWVSTISSGGSNTYYPDSVKGRFTISRDNSKNTLYLQMNSLGAEDTAVYYCASAYYGRRYDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 112 aa, synthetic construct
  source 1..112: note = Amino acid sequence of anti-hIL-33 QB11119 VL
DVVMTQSPPSLPVTLGQSASISCKSSKSLLHSNANTYLYWFQQRPGQSPQLLIYRMSNLASGVPDRFSGSGSGTDFTLKISRVEAEDVGVYYCMQHLENPYTFGQGTKVEIK

SEQ ID NO: 17 - protein, 219 aa, synthetic construct
  source 1..219: note = Amino acid sequence of anti-hIL-33 QB11119 LC
DVVMTQSPPSLPVTLGQSASISCKSSKSLLHSNANTYLYWFQQRPGQSPQLLIYRMSNLASGVPDRFSGSGSGTDFTLKISRVEAEDVGVYYCMQHLENPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 121 aa, synthetic construct; identical to SEQ ID NO: 10
  source 1..121: note = Amino acid sequence of anti-hIL-33 QB11119 VH

SEQ ID NO: 19 - protein, 451 aa, synthetic construct
  source 1..451: note = Amino acid sequence of anti-hIL-33 QB11119 HC
EVQLLESGGGLVQPGGSLRLSCAASGFTFYSSAMSWVRQTQGKGLEWVSTISSGGSNTYYPDSVKGRFTISRDNSKNTLYLQMNSLGAEDTAVYYCASAYYGRRYDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 112 aa, synthetic construct
  source 1..112: note = Amino acid sequence of anti-hIL-33 QB11421 VL
DVVMTQSPPSLPVTLGQSASISCKSSESLLHSNANTYLYWFQQRPGQSPQLLIYRGSNLASGVPDRFSGSGSGTDFTLKISRVEAEDVGVYYCMQHLRNPYTFGQGTKVEIK

SEQ ID NO: 21 - protein, 219 aa, synthetic construct
  source 1..219: note = Amino acid sequence of anti-hIL-33 QB11421 LC
DVVMTQSPPSLPVTLGQSASISCKSSESLLHSNANTYLYWFQQRPGQSPQLLIYRGSNLASGVPDRFSGSGSGTDFTLKISRVEAEDVGVYYCMQHLRNPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 121 aa, synthetic construct
  source 1..121: note = Amino acid sequence of anti-hIL-33 QB11421 VH
EVQLLESGGGLVQPGGSLRLSCAASGFTFYSSAMSWFRQTQGKGLEWVSTISSGGSNTYYPDSVKGRFTISRDNSKNTLYLQMNSLGAEDTAVYYCASAYYGRRYDAMDYWGQGTLVTVSS

SEQ ID NO: 23 - protein, 451 aa, synthetic construct
  source 1..451: note = Amino acid sequence of anti-hIL-33 QB11421 HC
EVQLLESGGGLVQPGGSLRLSCAASGFTFYSSAMSWFRQTQGKGLEWVSTISSGGSNTYYPDSVKGRFTISRDNSKNTLYLQMNSLGAEDTAVYYCASAYYGRRYDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 336 bp, synthetic construct
  source 1..336: mol_type = other DNA; note = Nucleotide sequence encoding anti-hIL-33 QB11465 VL
gacgtagtgatgactcagagtcctccaagcctccctgttactctcggtcagtccgccagcataagctgtaagtcctccgaatcacttctgcattcaaatgctaacacttatctctactggtttcagcaaagacctggccaatcaccccagttgctcatttatagggggagcaatttggctagtggggttccagatcgcttttcaggaagcggctctggtaccgactttaccctcaaaatcagtcgagtagaagctgaggacgttggagtttactattgtatgcagcacctccgaaatccatacacttttgggcaggggacaaaggtcgaaataaag

SEQ ID NO: 25 - protein, 112 aa, synthetic construct; identical to SEQ ID NO: 20
  source 1..112: note = Amino acid sequence of anti-hIL-33 QB11465 VL

SEQ ID NO: 26 - DNA, 663 bp, synthetic construct
  source 1..663: mol_type = other DNA; note = Nucleotide sequence encoding anti-hIL-33 QB11465 LC
gacgtagtgatgactcagagtcctccaagcctccctgttactctcggtcagtccgccagcataagctgtaagtcctccgaatcacttctgcattcaaatgctaacacttatctctactggtttcagcaaagacctggccaatcaccccagttgctcatttatagggggagcaatttggctagtggggttccagatcgcttttcaggaagcggctctggtaccgactttaccctcaaaatcagtcgagtagaagctgaggacgttggagtttactattgtatgcagcacctccgaaatccatacacttttgggcaggggacaaaggtcgaaataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgataa

SEQ ID NO: 27 - protein, 219 aa, synthetic construct; identical to SEQ ID NO: 21
  source 1..219: note = Amino acid sequence of anti-hIL-33 QB11465 LC

SEQ ID NO: 28 - DNA, 363 bp, synthetic construct
  source 1..363: mol_type = other DNA; note = Nucleotide sequence encoding anti-hIL-33 QB11465 VH
gaggttcaacttttggagtctggcggtggtctggtgcaacccggtgggagtttgcgacttagctgtgcagccagtggattcactttttatagctctgccatgtcatggtttcggcagactcaaggaaagggactggaatgggtttccaccatatcctcagggggctcaaatacatactacccagactccgtgaaaggccggtttaccatttcacgagacaactcaaagaacacactttacctgcaaatgaatagtctgggcgctgaagatacagccgtatattattgtgcatcagcatactacggacgccgatacgacgcaatggattattggggacagggcaccctggtcactgtcagctct

SEQ ID NO: 29 - protein, 121 aa, synthetic construct; identical to SEQ ID NO: 22
  source 1..121: note = Amino acid sequence of anti-hIL-33 QB11465 VH

SEQ ID NO: 30 - DNA, 1350 bp, synthetic construct
  source 1..1350: mol_type = other DNA; note = Nucleotide sequence encoding anti-hIL-33 QB11465 IgG4 HC
gaggttcaacttttggagtctggcggtggtctggtgcaacccggtgggagtttgcgacttagctgtgcagccagtggattcactttttatagctctgccatgtcatggtttcggcagactcaaggaaagggactggaatgggtttccaccatatcctcagggggctcaaatacatactacccagactccgtgaaaggccggtttaccatttcacgagacaactcaaagaacacactttacctgcaaatgaatagtctgggcgctgaagatacagccgtatattattgtgcatcagcatactacggacgccgatacgacgcaatggattattggggacagggcaccctggtcactgtcagctctgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgataa

SEQ ID NO: 31 - protein, 448 aa, synthetic construct
  source 1..448: note = Amino acid sequence of anti-hIL-33 QB11465 IgG4 HC
EVQLLESGGGLVQPGGSLRLSCAASGFTFYSSAMSWFRQTQGKGLEWVSTISSGGSNTYYPDSVKGRFTISRDNSKNTLYLQMNSLGAEDTAVYYCASAYYGRRYDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 181 aa, synthetic construct
  source 1..181: note = Amino acid sequence of Avitag_c-MYC_His6_hTSLP (wild type) QB11630
GLNDIFEAQKIEWHEGGGGSEQKLISEEDLGSSHHHHHHSSGLVPRGSHMYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 33 - protein, 176 aa, synthetic construct
  source 1..176: note = Amino acid sequence of Avitag_c-MYC_His6_hTSLP QB11033 with deletion of 126KRRKR130
GLNDIFEAQKIEWHEGGGGSEQKLISEEDLGSSHHHHHHSSGLVPRGSHMYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKVTTNKCLEQVSQLQGLWRRFNRPLLKQQ

SEQ ID NO: 34 - protein, 181 aa, synthetic construct
  source 1..181: note = Amino acid sequence of Avitag_c-MYC_His6_hTSLP QB11631 with mutations of R127A+R130S
GLNDIFEAQKIEWHEGGGGSEQKLISEEDLGSSHHHHHHSSGLVPRGSHMYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKARKSKVTTNKCLEQVSQLQGLWRRFNRPLLKQQ

SEQ ID NO: 35 - protein, 179 aa, synthetic construct
  source 1..179: note = Amino acid sequence of Avitag_FLAG_His6_cyTSLP QB11632 (wild type)
GLNDIFEAQKIEWHEGGGGSDYKDDDDKGSSHHHHHHSSGLVPRGSHMYDFTNCDFQKIEADYLRTISKDLITYMSGTKSTDFNNTVSCSNRPHCLTEIQSLTFNPTPRCASLAKEMFARKTKATLALWCPGYSETQINATQAMKKRRKRKVTTNKCLEQVSQLLGLWRRFIRTLLKKQ

SEQ ID NO: 36 - protein, 174 aa, synthetic construct
  source 1..174: note = Amino acid sequence of Avitag_FLAG_His6_cyTSLP QB10974 with deletion of 126KRRKR130
GLNDIFEAQKIEWHEGGGGSDYKDDDDKGSSHHHHHHSSGLVPRGSHMYDFTNCDFQKIEADYLRTISKDLITYMSGTKSTDFNNTVSCSNRPHCLTEIQSLTFNPTPRCASLAKEMFARKTKATLALWCPGYSETQINATQAMKKVTTNKCLEQVSQLLGLWRRFIRTLLKKQ

SEQ ID NO: 37 - protein, 179 aa, synthetic construct
  source 1..179: note = Amino acid sequence of Avitag_FLAG_His6_cyTSLP QB11633 with mutations of R127A+R130S
GLNDIFEAQKIEWHEGGGGSDYKDDDDKGSSHHHHHHSSGLVPRGSHMYDFTNCDFQKIEADYLRTISKDLITYMSGTKSTDFNNTVSCSNRPHCLTEIQSLTFNPTPRCASLAKEMFARKTKATLALWCPGYSETQINATQAMKKARKSKVTTNKCLEQVSQLLGLWRRFIRTLLKKQ

SEQ ID NO: 38 - protein, 440 aa, synthetic construct
  source 1..440: note = Amino acid sequence of hTSLPR-Fc fusion protein QB11034
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKAAAEPKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 112 aa, synthetic construct
  source 1..112: note = Amino acid sequence of anti-hTSLP VH of clone 135F1 from rabbit hybridoma
QEQLEESGGDLVKPEGSLTLTCKASGFDFSSYWISWVRQAPGKRPEWIACIDSDIDSADYASWAKGRFTMSRTSSTTVTLQMTSLTAADTATYFCVRNLGLWGPGTLVTVSS

SEQ ID NO: 40 - protein, 435 aa, synthetic construct
  source 1..435: note = Amino acid sequence of anti-hTSLP HC of clone 135F1 from rabbit hybridoma
QEQLEESGGDLVKPEGSLTLTCKASGFDFSSYWISWVRQAPGKRPEWIACIDSDIDSADYASWAKGRFTMSRTSSTTVTLQMTSLTAADTATYFCVRNLGLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 41 - protein, 110 aa, synthetic construct
  source 1..110: note = Amino acid sequence of anti-hTSLP VL of clone 135F1 from rabbit hybridoma
AQVLTQTASPVSAAVGGTVTINCQSSQNVYDKDALAWYQHKPGQRPKLLIYEASKLASGVPSRFSGSGAGTQFTLTISGVQCDDAATYYCAGTFIDNIYTFGGGTEVVVK

SEQ ID NO: 42 - protein, 214 aa, synthetic construct
  source 1..214: note = Amino acid sequence of anti-hTSLP LC of clone 135F1 from rabbit hybridoma
AQVLTQTASPVSAAVGGTVTINCQSSQNVYDKDALAWYQHKPGQRPKLLIYEASKLASGVPSRFSGSGAGTQFTLTISGVQCDDAATYYCAGTFIDNIYTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 43 - protein, 113 aa, synthetic construct
  source 1..113: note = Amino acid sequence of anti-hTSLP QB11341 and QB11548 VH of humanized clone 135F1
EVQLVESGGGLVQPGGSLRLSCAASGFDFSSYWISWVRQAPGKGPEWIASIDIDIDIADYASWAKGRFTISRDNSKNTLYLQMNSLRAEDTAVYYCVRNLGLWGPGTLVTVSS

SEQ ID NO: 44 - protein, 439 aa, synthetic construct
  source 1..439: note = Amino acid sequence of anti-hTSLP QB11341 and QB11548 HC of humanized clone 135F1
EVQLVESGGGLVQPGGSLRLSCAASGFDFSSYWISWVRQAPGKGPEWIASIDIDIDIADYASWAKGRFTISRDNSKNTLYLQMNSLRAEDTAVYYCVRNLGLWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLAAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 45 - protein, 110 aa, synthetic construct
  source 1..110: note = Amino acid sequence of anti-hTSLP QB11341 VL of humanized clone 135F1
DIQMTQSPSSLSASVGDRVTITCQSSQNVYDKDALAWYQHKPGKRPKLLIYEASKLASGVPSRFSGSGSGTHFTLTISSLQPEDAATYYCAGTFIDNIYTFGGGTKVEIK

SEQ ID NO: 46 - protein, 217 aa, synthetic construct
  source 1..217: note = Amino acid sequence of anti-hTSLP QB11341 LC of humanized clone 135F1
DIQMTQSPSSLSASVGDRVTITCQSSQNVYDKDALAWYQHKPGKRPKLLIYEASKLASGVPSRFSGSGSGTHFTLTISSLQPEDAATYYCAGTFIDNIYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 110 aa, synthetic construct
  source 1..110: note = Amino acid sequence of anti-hTSLP QB11548 VL of humanized clone 135F1 after yeast display
DIQMTQSPSSLSASVGDRVTITCQSSQNVYDKDALAWYQHKPGKRPKLLIYEASRLASGVPSRFSGSGSGTHFTLTISSLQPEDAATYYCAGTRIDNIYTFGGGTKVEIK

SEQ ID NO: 48 - protein, 217 aa, synthetic construct
  source 1..217: note = Amino acid sequence of anti-hTSLP QB11548 LC of humanized clone 135F1 after yeast display
DIQMTQSPSSLSASVGDRVTITCQSSQNVYDKDALAWYQHKPGKRPKLLIYEASRLASGVPSRFSGSGSGTHFTLTISSLQPEDAATYYCAGTRIDNIYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 339 bp, synthetic construct
  source 1..339: mol_type = other DNA; note = Nucleotide sequence encoding anti-hTSLP QB11764 VH of the final clone
gaggtgcaattggtggagtctggtgggggacttgtgcaacccggcggatcactgcggcttagctgcgcagcatcaggatttgattttagtagctattggatcagttgggttaggcaggcaccagggaaacgccccgagtggatagcaagtattgacatcgatattgatattgccgattatgcttcctgggccaaaggccgatttacaataagtcgcgataactcaaaaaacaccctttatcttcaaatgaattccctccgagcagaagataccgctgtttactactgtgtccgaaatctgggattgtggggcccaggtacccttgttacagtttcttcc

SEQ ID NO: 50 - protein, 113 aa, synthetic construct
  source 1..113: note = Amino acid sequence of anti-hTSLP QB11764 VH of the final clone
EVQLVESGGGLVQPGGSLRLSCAASGFDFSSYWISWVRQAPGKRPEWIASIDIDIDIADYASWAKGRFTISRDNSKNTLYLQMNSLRAEDTAVYYCVRNLGLWGPGTLVTVSS

SEQ ID NO: 51 - DNA, 1332 bp, synthetic construct
  source 1..1332: mol_type = other DNA; note = Nucleotide sequence encoding anti-hTSLP QB11764 IgG1-D265A of the final clone for MabPair production
gaggtgcaattggtggagtctggtgggggacttgtgcaacccggcggatcactgcggcttagctgcgcagcatcaggatttgattttagtagctattggatcagttgggttaggcaggcaccagggaaacgccccgagtggatagcaagtattgacatcgatattgatattgccgattatgcttcctgggccaaaggccgatttacaataagtcgcgataactcaaaaaacaccctttatcttcaaatgaattccctccgagcagaagataccgctgtttactactgtgtccgaaatctgggattgtggggcccaggtacccttgttacagtttcttccgcctcaactaagggaccaagcgtcttcccccttgctccctcaagcaagtccacaagcggggggaccgctgctttgggctgcctcgttgatgactactttcccgaacctgtcaccgtgtcatggaattccggagccttgacttctggggtacatacctgccctgctgtattgcagagttctggactgtattctctgagtagtgtagtgactgttccatctagctccctgggtactcagacctacatttgtaatgtcaatcacaagcctagtaacaccaaagtggataagaaagtggagcctaagtctggtgataagacacacacatgccctccctgtccagcaccagagctccttgggggaccttccgtctttcttttccctcccaaacccaaggatacacttatgattagccggacaccagaagttacttgcgtcgtcgttgcagtgagccatgaggacccagaagttaagttcaattggtacgtggatggcgtcgaggtacataatgccaagaccaagccacgtgaggagcagtacaacagtacatatagggtcgtgtccgtacttacagtgctccaccaagattggctgaatggtaaggaatataagtgtaaggttagtaataaagcactgcccgcccctatcgaaaagaccatatctaaagccaaaggccagccccgtgaaccccaggtatatacacttccaccatcccgtgaggaaatgactaaaaatcaggtatctcttacctgcctcgtaaaaggtttctacccatccgatatagcagtagagtgggaaagcaatggccaacccgagaacaattacaaaaccaccccccctgtgctgcgtagcgatggttctttttttctttactccgaacttacagtggataagtcccgttggcagcaaggaaacgtattctcttgttctgtaatgcatgaagcacttcataatcattatactcaaaagtccctgtctctctcccccggcaagtga

SEQ ID NO: 52 - protein, 443 aa, synthetic construct
  source 1..443: note = Amino acid sequence of anti-hTSLP QB11764 IgG1-D265A of the final clone for MabPair production
EVQLVESGGGLVQPGGSLRLSCAASGFDFSSYWISWVRQAPGKRPEWIASIDIDIDIADYASWAKGRFTISRDNSKNTLYLQMNSLRAEDTAVYYCVRNLGLWGPGTLVTVSSASTKGPSVFPLAPSSKSTSGGTAALGCLVDDYFPEPVTVSWNSGALTSGVHTCPAVLQSSGLYSLSSVVTVPSSSLGTQTYICNVNHKPSNTKVDKKVEPKSGDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSELTVDKSRWQQGNVFSCSVMHEALHNHYTQKSLSLSPGK

SEQ ID NO: 53 - DNA, 330 bp, synthetic construct
  source 1..330: mol_type = other DNA; note = Nucleotide sequence encoding anti-hTSLP QB11764 VL of the final clone
gacatacagatgacccagtcccccagctctttgtcagcatccgtaggagatcgggtcaccatcacctgtcaatcatctcaaaacgtttatgataaagacgcattggcctggtaccagcataagccaggtaaaaggccaaaattgctcatctacgaagctagtcggctggcatcaggggtgccatctaggttttccggtagcggaagtggtacacattttacacttaccatttcctctcttcaaccagaagatgccgccacctattattgtgccggtacaagaatcgacaatatatacactttcggagatgggacaaaggtagaaataaag

SEQ ID NO: 54 - protein, 110 aa, synthetic construct
  source 1..110: note = Amino acid sequence of anti-hTSLP QB11764 VL of the final clone
DIQMTQSPSSLSASVGDRVTITCQSSQNVYDKDALAWYQHKPGKRPKLLIYEASRLASGVPSRFSGSGSGTHFTLTISSLQPEDAATYYCAGTRIDNIYTFGDGTKVEIK

SEQ ID NO: 55 - DNA, 654 bp, synthetic construct
  source 1..654: mol_type = other DNA; note = Nucleotide sequence encoding anti-hTSLP QB11764 LC of the final clone for MabPair production
gacatacagatgacccagtcccccagctctttgtcagcatccgtaggagatcgggtcaccatcacctgtcaatcatctcaaaacgtttatgataaagacgcattggcctggtaccagcataagccaggtaaaaggccaaaattgctcatctacgaagctagtcggctggcatcaggggtgccatctaggttttccggtagcggaagtggtacacattttacacttaccatttcctctcttcaaccagaagatgccgccacctattattgtgccggtacaagaatcgacaatatatacactttcggagatgggacaaaggtagaaataaagcgtacggtggccgctcctagtgtattcatctttcccccttctgatgaacaattgaagtctgggactgcaaaggttgtatgtctccttaataacttctacccacgagaagccaaagtacagtggaaggtggataatgccctccaaagcggaaacagtcaggaatgcgtaactgaacaagacagcaaggactcaacctactctctttcctccacactcacactgtcaaaggcagattacgagaagcacaaagtctacgcttgcgaggtcactcatcagggcctcagctcacccgtcactaagtcttttaacagaggcgaatcttga

SEQ ID NO: 56 - protein, 217 aa, synthetic construct
  source 1..217: note = Amino acid sequence of anti-hTSLP QB11764 LC of the final clone for MabPair production
DIQMTQSPSSLSASVGDRVTITCQSSQNVYDKDALAWYQHKPGKRPKLLIYEASRLASGVPSRFSGSGSGTHFTLTISSLQPEDAATYYCAGTRIDNIYTFGDGTKVEIKRTVAAPSVFIFPPSDEQLKSGTAKVVCLLNNFYPREAKVQWKVDNALQSGNSQECVTEQDSKDSTYSLSSTLTLSKADYEKHKVYACEVTHQGLSSPVTKSFNRGES

SEQ ID NO: 57 - protein, 120 aa, synthetic construct
  source 1..120: note = Amino acid sequence of anti-hTSLP QB10987 VH of chimeric antibody clone 23B12
EEKLQQSGDDLVRPGAAVKMSCKASGYIFTDYAMHWVKQRPGQGLEWIGTFIPLLDTSDYNQNFKGRATLTADKSSNTAYMELSRLTSEDSAVYYCARMGVTHSYVMDAWGQGASVTVSS

SEQ ID NO: 58 - protein, 446 aa, synthetic construct
  source 1..446: note = Amino acid sequence of anti-hTSLP QB10987 HC of chimeric antibody clone 23B12
EEKLQQSGDDLVRPGAAVKMSCKASGYIFTDYAMHWVKQRPGQGLEWIGTFIPLLDTSDYNQNFKGRATLTADKSSNTAYMELSRLTSEDSAVYYCARMGVTHSYVMDAWGQGASVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLAAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 107 aa, synthetic construct
  source 1..107: note = Amino acid sequence of anti-hTSLP QB10987 VL of chimeric antibody clone 23B12
DIVLTQSPATLSVTPGESVSLSCRASQPISISVHWFQQKSNESPRLLIKFASQSISGIPSRFSGSGSGTDFTLNINRVESEDFSVYYCQQTFSLPYTFGTGTKLELK

SEQ ID NO: 60 - protein, 214 aa, synthetic construct
  source 1..214: note = Amino acid sequence of anti-hTSLP QB10987 LC of chimeric antibody clone 23B12
DIVLTQSPATLSVTPGESVSLSCRASQPISISVHWFQQKSNESPRLLIKFASQSISGIPSRFSGSGSGTDFTLNINRVESEDFSVYYCQQTFSLPYTFGTGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 120 aa, synthetic construct
  source 1..120: note = Amino acid sequence of anti-hTSLP QB10990 VH of humanized antibody clone hz-3C
QVQLVQSGDEVKKPGSSVKVSCKASGYIFTDYAMHWVRQRPGQGLEWMGTFIPLLDTSDYNQNFKGRATITADKSTSTAYMELSSLRSEDTAVYYCARMGVTHSYVMDAWGQGTSVTVSS

SEQ ID NO: 62 - protein, 446 aa, synthetic construct
  source 1..446: note = Amino acid sequence of anti-hTSLP QB10990 HC of humanized antibody clone hz-3C
QVQLVQSGDEVKKPGSSVKVSCKASGYIFTDYAMHWVRQRPGQGLEWMGTFIPLLDTSDYNQNFKGRATITADKSTSTAYMELSSLRSEDTAVYYCARMGVTHSYVMDAWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLAAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 63 - protein, 107 aa, synthetic construct
  source 1..107: note = Amino acid sequence of anti-hTSLP QB10990 VL of humanized antibody clone hz-3C, identical to QB11060 VL
EIVLTQSPGTLSLSPGERATLSCRASQPISISVHWYQQKPGEAPRLLIYFASQSISGIPDRFSGSGSGTDFTLTISRLESEDFAVYYCQQTFSLPYTFGTGTKVEIK

SEQ ID NO: 64 - protein, 214 aa, synthetic construct
  source 1..214: note = Amino acid sequence of anti-hTSLP QB10990 LC of humanized antibody clone hz-3C, identical to QB11060 LC
EIVLTQSPGTLSLSPGERATLSCRASQPISISVHWYQQKPGEAPRLLIYFASQSISGIPDRFSGSGSGTDFTLTISRLESEDFAVYYCQQTFSLPYTFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 120 aa, synthetic construct
  source 1..120: note = Amino acid sequence of anti-hTSLP QB11060 VH of humanized antibody clone hz-3C, with D9P+S108L substitutions, identical to QB11237 VH
QVQLVQSGPEVKKPGSSVKVSCKASGYIFTDYAMHWVRQRPGQGLEWMGTFIPLLDTSDYNQNFKGRATITADKSTSTAYMELSSLRSEDTAVYYCARMGVTHSYVMDAWGQGTLVTVSS

SEQ ID NO: 66 - protein, 446 aa, synthetic construct
  source 1..446: note = Amino acid sequence of anti-hTSLP QB11060 HC of humanized antibody clone hz-3C, with D9P+S108L substitutions, identical to QB11237 HC
QVQLVQSGPEVKKPGSSVKVSCKASGYIFTDYAMHWVRQRPGQGLEWMGTFIPLLDTSDYNQNFKGRATITADKSTSTAYMELSSLRSEDTAVYYCARMGVTHSYVMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLAAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 107 aa, synthetic construct
  source 1..107: note = Amino acid sequence of anti-hTSLP QB11237 VL of humanized antibody clone hz-3C after yeast display
EIVLTQSPGTLSLSPGERATLSCRASQHISRSVHWYQQKPGEAPRLLIYFASQSISGIPDRFSGSGSGTDFTLTISRLESEDFAVYYCQQSYSFPYTFGTGTKVEIK

SEQ ID NO: 68 - protein, 214 aa, synthetic construct
  source 1..214: note = Amino acid sequence of anti-hTSLP QB11237 LC of humanized antibody clone hz-3C after yeast display
EIVLTQSPGTLSLSPGERATLSCRASQHISRSVHWYQQKPGEAPRLLIYFASQSISGIPDRFSGSGSGTDFTLTISRLESEDFAVYYCQQSYSFPYTFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - DNA, 360 bp, synthetic construct
  source 1..360: mol_type = other DNA; note = Nucleotide sequence encoding anti-hTSLP QB11718 VH of the final clone
caggtccaacttgttcagtcaggcccagaagtcaaaaaacctggtagttccgtcaaggtatcctgcaaggcaagtggatacatcttcacagactatgctatgcactgggtgcgtcagagaccaggtcaggacctcgaatggatgggcactttcattccccttctggatacctcagactacaatcagaatttcaagggaagagcaaccattactgctgataagtcaacaagtactgcttatatggaactgagctccttgcgcagcgaagacacagccgtttattattgcgcccggatgggtgttacacatagttacgtgatggacgcatggggacagggaacactcgtaacagtgtcttca

SEQ ID NO: 70 - protein, 120 aa, synthetic construct
  source 1..120: note = Amino acid sequence of anti-hTSLP QB11718 VH of the final clone
QVQLVQSGPEVKKPGSSVKVSCKASGYIFTDYAMHWVRQRPGQDLEWMGTFIPLLDTSDYNQNFKGRATITADKSTSTAYMELSSLRSEDTAVYYCARMGVTHSYVMDAWGQGTLVTVSS

SEQ ID NO: 71 - DNA, 1353 bp, synthetic construct
  source 1..1353: mol_type = other DNA; note = Nucleotide sequence encoding anti-hTSLP QB11718 IgG1-D265A HC of the final clone for MabPair production
caggtccaacttgttcagtcaggcccagaagtcaaaaaacctggtagttccgtcaaggtatcctgcaaggcaagtggatacatcttcacagactatgctatgcactgggtgcgtcagagaccaggtcaggacctcgaatggatgggcactttcattccccttctggatacctcagactacaatcagaatttcaagggaagagcaaccattactgctgataagtcaacaagtactgcttatatggaactgagctccttgcgcagcgaagacacagccgtttattattgcgcccggatgggtgttacacatagttacgtgatggacgcatggggacagggaacactcgtaacagtgtcttcagctagcactaaaggccctagtgtgttcccactcgctccaagctccaagtcaacctccggcggcaccgctgccctgggttgtctcgtcgatgattattttcccgaaccagtgactgtgagctggaatagcggagctctcacaagtggcgtgcacacttgtcctgccgtattgcaatcctccggcctgtattcactcagttctgtcgttaccgtcccctcatcatccctcgggacacaaacatatatatgcaatgtcaatcacaagcctagtaacacaaaagtagataaaaaagtcgagccaaagtccggcgataaaacccatacttgcccaccctgtccagctcccgagcttctggggggacccagtgtatttctctttccaccaaagcctaaagacacactgatgattagcagaacccccgaagttacctgtgtcgtggtagcagtttcccatgaagatcctgaagtcaagttcaattggtacgtagatggggtggaggtccataacgcaaaaacaaaaccccgcgaagagcagtataactctacttaccgggttgtgtccgtgctcaccgtgctgcatcaagattggctgaatggaaaggaatacaaatgtaaggtcagcaataaggcccttcccgcacctattgaaaaaacaatctctaaagctaagggccaaccacgagagcctcaagtttataccctccctccatctagagaagagatgactaaaaaccaggtgtccctcacttgcctcgttaagggcttttacccaagtgacattgctgtggagtgggaatcaaatggacaacctgaaaacaattacaaaactacacccccagtcttgaggagcgacggctcattttttttgtactctgagcttaccgttgacaaaagccgctggcagcaaggaaacgttttttcatgcagtgtaatgcatgaggcacttcacaatcattacacacagaagtcattgagcctcagccctgggaagtga

SEQ ID NO: 72 - protein, 450 aa, synthetic construct
  source 1..450: note = Amino acid sequence of anti-hTSLP QB11718 IgG1-D265A HC of the final clone for MabPair production
QVQLVQSGPEVKKPGSSVKVSCKASGYIFTDYAMHWVRQRPGQDLEWMGTFIPLLDTSDYNQNFKGRATITADKSTSTAYMELSSLRSEDTAVYYCARMGVTHSYVMDAWGQGTLVTVSSASTKGPSVFPLAPSSKSTSGGTAALGCLVDDYFPEPVTVSWNSGALTSGVHTCPAVLQSSGLYSLSSVVTVPSSSLGTQTYICNVNHKPSNTKVDKKVEPKSGDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSELTVDKSRWQQGNVFSCSVMHEALHNHYTQKSLSLSPGK

SEQ ID NO: 73 - DNA, 321 bp, synthetic construct
  source 1..321: mol_type = other DNA; note = Nucleotide sequence encoding anti-hTSLP QB11718 VL of the final clone
gaaatagtcctcacacagtccccaggtaccctctcactgtcacccggcgaaagggccactctgtcctgtcgtgcaagccagcacatcagtcgctctgtccattggtatcaacagaagccaggtgaggccccacgtcttctgatttacttcgctagtcaatctatctccggcattcctgaccgatttagcggcagcggaagcggaaccgacttcactctcacaatatcacggcttgaatcagaggatttcgccgtctattactgccagcagagttacagcttcccctatactttcgggcgcggaactaaggtagaaattaag

SEQ ID NO: 74 - protein, 107 aa, synthetic construct
  source 1..107: note = Amino acid sequence of anti-hTSLP QB11718 VL of the final clone
EIVLTQSPGTLSLSPGERATLSCRASQHISRSVHWYQQKPGEAPRLLIYFASQSISGIPDRFSGSGSGTDFTLTISRLESEDFAVYYCQQSYSFPYTFGRGTKVEIK

SEQ ID NO: 75 - DNA, 645 bp, synthetic construct
  source 1..645: mol_type = other DNA; note = Nucleotide sequence encoding anti-hTSLP QB11718 LC of the final clone for MabPair production
gaaatagtcctcacacagtccccaggtaccctctcactgtcacccggcgaaagggccactctgtcctgtcgtgcaagccagcacatcagtcgctctgtccattggtatcaacagaagccaggtgaggccccacgtcttctgatttacttcgctagtcaatctatctccggcattcctgaccgatttagcggcagcggaagcggaaccgacttcactctcacaatatcacggcttgaatcagaggatttcgccgtctattactgccagcagagttacagcttcccctatactttcgggcgcggaactaaggtagaaattaagcgtacggtcgccgcccccagcgtattcatatttcctcctagcgatgagcagcttaaatcaggaacagcaaaggttgtctgtttgttgaacaatttctatcctcgggaagctaaggtccaatggaaagtggacaacgccttgcagagcggtaattcacaagaatgcgtgacagagcaagatagcaaagactcaacctactcactgagctctactctgacactttctaaagctgattatgagaaacacaaggtatatgcatgtgaagtgactcaccaggggctcagctcacctgtgactaaatcctttaacaggggcgaatcctga

SEQ ID NO: 76 - protein, 214 aa, synthetic construct
  source 1..214: note = Amino acid sequence of anti-hTSLP QB11718 LC of the final clone for MabPair production
EIVLTQSPGTLSLSPGERATLSCRASQHISRSVHWYQQKPGEAPRLLIYFASQSISGIPDRFSGSGSGTDFTLTISRLESEDFAVYYCQQSYSFPYTFGRGTKVEIKRTVAAPSVFIFPPSDEQLKSGTAKVVCLLNNFYPREAKVQWKVDNALQSGNSQECVTEQDSKDSTYSLSSTLTLSKADYEKHKVYACEVTHQGLSSPVTKSFNRGES

SEQ ID NO: 77 - protein, 16 aa, synthetic construct
  source 1..16: note = Amino acid sequence of CDR1 region of VL of anti-hIL-33 antibody QB11421 and QB11465
KSSESLLHSNANTYLY

SEQ ID NO: 78 - protein, 7 aa, synthetic construct
  source 1..7: note = Amino acid sequence of CDR2 region of VL of anti-hIL-33 antibody QB11421 and QB11465
RGSNLAS

SEQ ID NO: 79 - protein, 9 aa, synthetic construct
  source 1..9: note = Amino acid sequence of CDR3 region of VL of anti-hIL-33 antibody QB11421 and QB11465
MQHLRNPYT

SEQ ID NO: 80 - protein, 5 aa, synthetic construct
  source 1..5: note = Amino acid sequence of CDR1 region of VH of anti-hIL-33 antibody QB11421 and QB11465
SSAMS

SEQ ID NO: 81 - protein, 17 aa, synthetic construct
  source 1..17: note = Amino acid sequence of CDR2 region of VH of anti-hIL-33 antibody QB11421 and QB11465
TISSGGSNTYYPDSVKG

SEQ ID NO: 82 - protein, 12 aa, synthetic construct
  source 1..12: note = Amino acid sequence of CDR3 region of VH of anti-hIL-33 antibody QB11421 and QB11465
AYYGRRYDAMDY

SEQ ID NO: 83 - protein, 13 aa, synthetic construct
  source 1..13: note = Amino acid sequence of CDR1 region of VL of anti-hTSLP antibody QB11341, QB11548, QB11764
QSSQNVYDKDALA

SEQ ID NO: 84 - protein, 7 aa, synthetic construct
  source 1..7: note = Amino acid sequence of CDR2 region of VL of anti-hTSLP antibody QB11341
EASKLAS

SEQ ID NO: 85 - protein, 10 aa, synthetic construct
  source 1..10: note = Amino acid sequence of CDR3 region of VL of anti-hTSLP antibody QB11341
AGTFIDNIYT

SEQ ID NO: 86 - protein, 5 aa, synthetic construct
  source 1..5: note = Amino acid sequence of CDR1 region of VH of anti-hTSLP antibody QB11341, QB11548, QB11764
SYWIS

SEQ ID NO: 87 - protein, 17 aa, synthetic construct
  source 1..17: note = Amino acid sequence of CDR2 region of VH of anti-hTSLP antibody QB11341, QB11548, QB11764
SIDIDIDIADYASWAKG

SEQ ID NO: 88 - protein, 4 aa, synthetic construct
  source 1..4: note = Amino acid sequence of CDR3 region of VH of anti-hTSLP antibody QB11341, QB11548, QB11764
NLGL

SEQ ID NO: 89 - protein, 7 aa, synthetic construct
  source 1..7: note = Amino acid sequence of CDR2 region of VL of anti-hTSLP antibody QB11548, QB11764
EASRLAS

SEQ ID NO: 90 - protein, 10 aa, synthetic construct
  source 1..10: note = Amino acid sequence of CDR3 region of VL of anti-hTSLP antibody QB11548, QB11764
AGTRIDNIYT

SEQ ID NO: 91 - protein, 11 aa, synthetic construct
  source 1..11: note = Amino acid sequence of CDR1 region of VL of anti-hTSLP antibody QB11237, QB11718
RASQHISRSVH

SEQ ID NO: 92 - protein, 7 aa, synthetic construct
  source 1..7: note = Amino acid sequence of CDR2 region of VL of anti-hTSLP antibody QB11237, QB11718
FASQSIS

SEQ ID NO: 93 - protein, 9 aa, synthetic construct
  source 1..9: note = Amino acid sequence of CDR3 region of VL of anti-hTSLP antibody QB11237, QB11718
QQSYSFPYT

SEQ ID NO: 94 - protein, 5 aa, synthetic construct
  source 1..5: note = Amino acid sequence of CDR1 region of VH of anti-hTSLP antibody QB11237, QB11718
DYAMH

SEQ ID NO: 95 - protein, 17 aa, synthetic construct
  source 1..17: note = Amino acid sequence of CDR2 region of VH of anti-hTSLP antibody QB11237, QB11718
TFIPLLDTSDYNQNFKG

SEQ ID NO: 96 - protein, 11 aa, synthetic construct
  source 1..11: note = Amino acid sequence of CDR3 region of VH of anti-hTSLP antibody QB11237, QB11718
MGVTHSYVMDA

SEQ ID NO: 97 - protein, 232 aa, synthetic construct
  source 1..232: note = Amino acid sequence of human IgG1 Fc region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 228 aa, synthetic construct
  source 1..228: note = Amino acid sequence of human IgG2 Fc region
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 99 - protein, 279 aa, synthetic construct
  source 1..279: note = Amino acid sequence of human IgG3 Fc region
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 100 - protein, 229 aa, synthetic construct
  source 1..229: note = Amino acid sequence of human IgG4 Fc region
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 5 aa, synthetic construct
  source 1..5: note = Residues
KRRKR

SEQ ID NO: 102 - protein, 11 aa, synthetic construct
  source 1..11: note = Hinge region
CPPCPAPELLG

SEQ ID NO: 103 - protein, 9 aa, synthetic construct
  source 1..9: note = Hinge region
GPSVFLFPP

SEQ ID NO: 104 - protein, 20 aa, synthetic construct
  source 1..20: note = Hinge region
CPPCPAPELLGGPSVFLFPP

SEQ ID NO: 105 - protein, 11 aa, synthetic construct
  source 1..11: note = Hinge region
CPPCPAPEFLG

SEQ ID NO: 106 - protein, 20 aa, synthetic construct
  source 1..20: note = Hinge region
CPPCPAPEFLGGPSVFLFPP

SEQ ID NO: 107 - protein, 107 aa, synthetic construct
  source 1..107: note = Consensus sequence of kappa CLs
XXXXXPXXXXXPPXXXXXXXXXXSXVCXXXXXXPXXXXVXWXXXXXXXXXXXQXSXTXXXXXXXXXSXSSTLTLXXXXXXXXXXXXCXXXHXXXXXXXXXXXXXXXC

SEQ ID NO: 108 - protein, 107 aa, synthetic construct
  source 1..107: note = Consensus sequence of lambda CLs
  VARIANT 67: note = alanine or methionine
XXXXXPXXXXXPPXXXXXXXXXXAXVCXXXXXXPXXXXVXWXXXXXXXXXXXEXTXPXXXXXXXXXXXSSYLSLXXXXXXXXXXXXCXXXHXXXXXXXXXXXXXXXC